(19) United States
(12) Patent Application Publication
(10) Pub. No.: US 2003/0203380 A1
(43) Pub. Date: Oct. 30, 2003

(54) Gene linked to osteoarthritis

(75) Inventor: Stefan E. Stefansson, Reykjavik (IS)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: deCODE genetics ehf., Reykjavik (IS)

(21) Appl. No.: 10/351,951

(22) Filed: Jan. 24, 2003

Related U.S. Application Data
(60) Provisional application No. 60/431,538, filed on Dec. 5, 2002.
(60) Provisional application No. 60/453,705, filed on Jan. 25, 2002.

(51) Int. Cl. C12Q 1/68; C07H 21/04; C12N 9/64; C12P 21/02; C12N 5/06

(52) U.S. Cl. 435/6; 435/69.1; 435/320.1; 435/325; 435/226; 536/23.2

(57) ABSTRACT

A role of the human MATN3 gene in osteoarthritis is disclosed. Methods for diagnosis, prediction of clinical course and treatment for osteoarthritis using polymorphisms in the MATN3 gene are also disclosed.

### RELATED APPLICATIONS

This application claims the benefit of U.S. application Ser. No. 60/431,538 filed on Dec. 5, 2002 and also claims the benefit of U.S. application Ser. No. 10/057,312 filed on Jan. 25, 2002 (converted to Provisional Application No. 60/______). The entire teachings of the above applications are incorporated herein by reference.

### BACKGROUND OF THE INVENTION

Osteoarthritis (OA) is a slowly progressive, irreversible, often monoarticular disease characterized by pain and loss of function (Mankin and Brandt, Pathogenesis of Osteoarthritis in “Textbook of Rheumatology”, Kelly, et al., (eds.) 3rd edition, W. B. Saunders Co., Philadelphia, pp.14699-111471 (1989)) and Dean, Arth. Rheum. 20 (Suppl. 2):2 (1991)). The underlying cause of the pain and debilitation is the cartilage degradation that occurs as a result of the disease. A typical end-stage clinical picture includes complete erosion of the weight-bearing articular cartilage, requiring total joint replacement. There is no therapeutic approach available that will slow the clinical progression of osteoarthritis, although steroids and non-steroidal anti-inflammatory drugs are used to ameliorate the pain and inflammation associated with the disease.

### SUMMARY OF THE INVENTION

The present invention relates to isolated nucleic acid molecules associated by linkage studies to osteoarthritis (referred to herein as “a variant MATN3 nucleic acid”) and encoding protein associated with osteoarthritis. In one embodiment, the isolated nucleic acid molecule comprises a nucleotide sequence selected from the group consisting of SEQ ID NO: 1 and comprising at least one polymorphism selected from those set forth in Table 3 or FIGS. 5A-5C and the complements thereof. In a preferred embodiment, the polymorphism is present at nucleotide 47928 of SEQ ID NO: 1 (T or C allele).

The invention further relates to a nucleic acid molecule which hybridizes under high stringency conditions to a nucleotide sequence selected from the group consisting of SEQ ID NO: 1 and comprising at least one polymorphism selected from those set forth in Table 3 or FIGS. 5A-5C and the complements thereof.

The invention further provides a method for assaying the presence of a nucleic acid molecule comprising all or a portion of the gene in a sample, comprising contacting said sample with a second nucleic acid molecule comprising a nucleotide sequence selected from the group consisting of SEQ ID NO: 1 and comprising at least one polymorphism selected from those set forth in Table 3 or FIGS. 5A-5C and the complements thereof under conditions appropriate for selective hybridization.

The invention also relates to a vector comprising an isolated nucleic acid molecule selected from the group consisting of SEQ ID NO: 1 and comprising at least one polymorphism selected from those set forth in Table 3 or FIGS. 5A-5C, operatively linked to a regulatory sequence, as well as to a recombinant host cell comprising the vector. The invention also provides a method for preparing a polypeptide encoded by an isolated nucleic acid molecule of the invention selected from the group consisting of SEQ ID NO: 1 and comprising at least one polymorphism selected from those set forth in Table 3 or FIGS. 5A-5C, comprising culturing a recombinant host cell of the invention under conditions suitable for expression of said nucleic acid molecule.

The invention further provides an isolated polypeptide encoded by isolated nucleic acid molecules of the invention. In a particular embodiment, the polypeptide comprises the amino acid sequence of SEQ ID NO: 2, 3, 4, 5, 6, 7, 8 or 9. The invention also relates to an isolated polypeptide comprising an amino acid sequence which is greater than about 90 or 95 percent identical to the amino acid sequence of SEQ ID NO: 2, 3, 4, 5, 6, 7, 8 or 9.

The invention also relates to an antibody, or an antigen-binding fragment thereof, which selectively binds to a polypeptide of the invention, as well as to a method for assaying the presence of a polypeptide encoded by an isolated nucleic acid molecule of the invention in a sample, comprising contacting said sample with an antibody which specifically binds to the encoded polypeptide.

The invention further relates to methods of diagnosing a predisposition to osteoarthritis. The methods of diagnosing a predisposition to osteoarthritis in an individual include detecting the presence of an alteration in the MATN3 gene, as well as detecting alterations in expression of a MATN3 polypeptide. The alterations in expression can be quantitative, qualitative, or both quantitative and qualitative.

The invention additionally relates to an assay for identifying agents which alter (e.g., enhance or inhibit) the activity or expression of a MATN3 polypeptide. For example, a cell, cellular fraction, or solution containing MATN3 polypeptide or an active fragment or derivative thereof, can be contacted with an agent to be tested, and the level of MATN3 polypeptide expression or activity can be assessed. Agents that enhance or inhibit MATN3 polypeptide expression or activity are also included in the current invention, as are methods of altering (enhancing or inhibiting) MATN3 polypeptide expression or activity by contacting a cell containing MATN3 and/or polypeptide, or by contacting the MATN3 polypeptide, with an agent that enhances or inhibits expression or activity of MATN3 or polypeptide.

Additionally, the invention pertains to pharmaceutical compositions comprising the nucleic acids of the invention, the polypeptides of the invention, and/or the agents that alter activity of a MATN3 polypeptide. The invention further pertains to methods of treating osteoarthritis, by administering MATN3 therapeutic agents, such as nucleic acids of the invention, polypeptides of the invention, the agents that alter activity of a MATN3 polypeptide, or compositions comprising the nucleic acids, polypeptides, and/or the agents that alter activity of a MATN3 polypeptide.

The invention further provides a method of diagnosing susceptibility to osteoarthritis in an individual comprising screening for an at-risk haplotype in the MATN3 gene that is more frequently present in an individual susceptible to osteoarthritis, compared to the frequency of its presence in a healthy individual, wherein the presence of the at-risk haplotype is indicative of a susceptibility to osteoarthritis.

The invention also relates to a method of diagnosing susceptibility to osteoarthritis in an individual, comprising screening for an at-risk haplotype in the matrilin-3 gene that is more frequently present in an individual susceptible to osteoarthritis (affected), compared to the frequency of its presence in a healthy individual (control), wherein the presence of the at-risk haplotype is indicative of a susceptibility to osteoarthritis. In one embodiment, the at-risk haplotype is characterized by the presence of at least one polymorphism at nucleic acid positions 58162, 57927, 56822, 47929, 45434, 45317, 45178 and 45010, relative to SEQ ID NO: 1.

The invention further relates to a kit for diagnosing susceptibility to osteoarthritis in an individual comprising: primers for nucleic acid amplification of a region of the matrilin-3 gene comprising an at-risk haplotype, wherein the primers comprise a segment of nucleic acids of length suitable for nucleic acid amplification, selected from the group consisting of a polymorphism at nucleic acid position 58162, 57927, 56822, 47929, 45434, 45317, 45178 and 45010, relative to SEQ ID NO: 1 and combinations thereof.

### BRIEF DESCRIPTION OF THE DRAWINGS

The foregoing and other objects, features and advantages of the invention will be apparent from the following more particular description of preferred embodiments of the invention, as illustrated in the accompanying drawings.

FIGS. 1.1 to 1.35 show the nucleic acid sequence containing and surrounding the gene for human MATN3 (SEQ ID NO: 1). Coding sequences of the exons are underlined.

FIGS. 2A to 2C show the exon/intron boundaries of the gene for human MATN3 (SEQ ID NOs: 2-9 (amino acid) and SEQ ID NOs: 13-20 (nucleotide)). Characterized polymorphisms are labeled above the amino acids. The coding region is shown within the brackets. Known polymorphisms are indicated by asterisks.

FIG. 3 shows an alignment of amino acid residues for all 4 EGF domains from MATN3 from human (HuEGF1 to 4) (SEQ ID NOs: 10, 11, 12 and 43), mouse (MouEGF1 to 4) (SEQ ID NOs: 44, 45, 46 and 47) and chicken (ChEGF1 to 4) (SEQ ID NOs: 48, 49, 50 and 51). Residues conserved in all EGF domains are indicated by an asterisk. The predicted missense mutation at amino acid position 303 in MATN3 from human protein sequence changes the threonine-residue (boldface) to methionine. GenBank Accession Numbers for matrilin-3 protein sequences used in alignments herein are AJ224741 (human), Y10521 (mouse) and AJ000055 (chicken).

FIG. 4 is a pedigree showing patients with the Thr/Met mutation at position 47928. The left side of the symbol filled indicates thumb OA. The right side of the symbol filled indicates finger OA.

FIGS. 5A to 5C show inserts/deletions occurring at specific positions of the matrilin-3 gene, as indicated, which were found by sequencing genomic DNA.

FIGS. 6A to 6B shows the nucleotide sequences of primers used for PCR amplification of DNA sequences of the MATN3 gene.

### DETAILED DESCRIPTION OF THE INVENTION

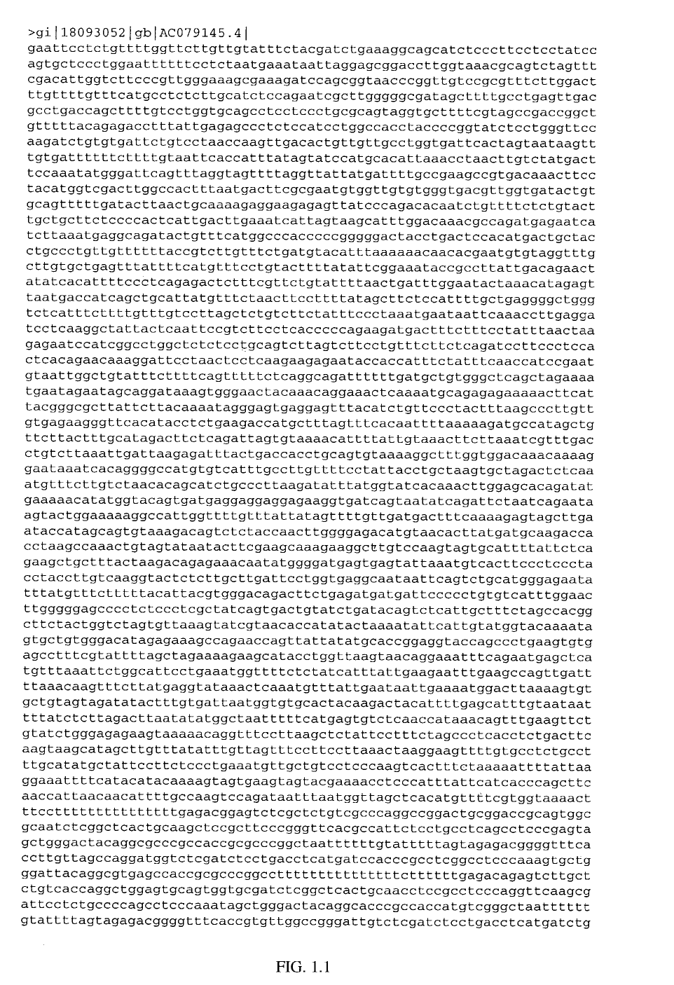

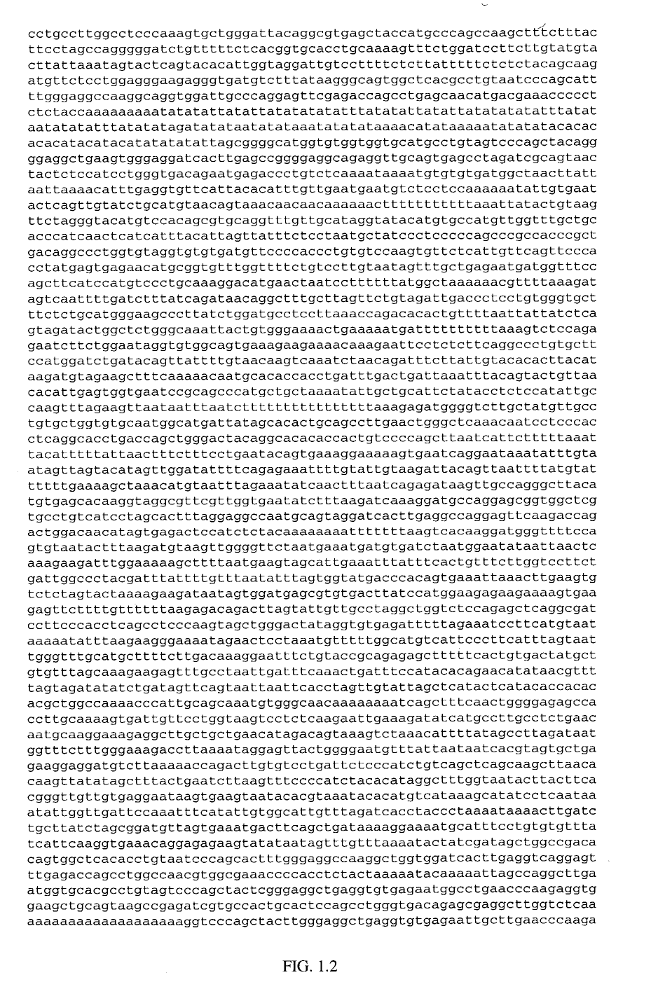

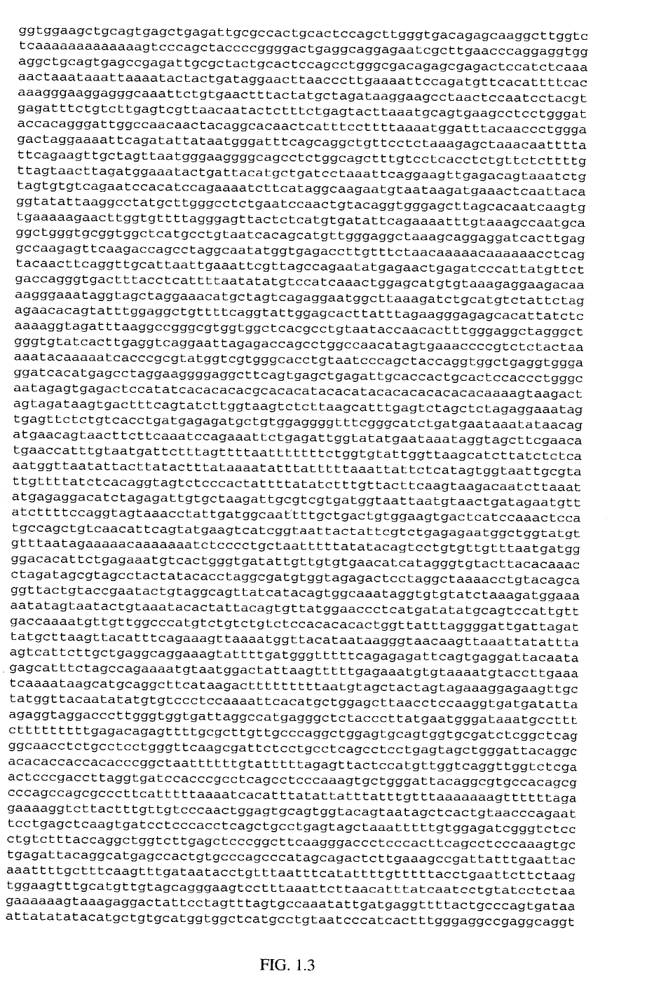

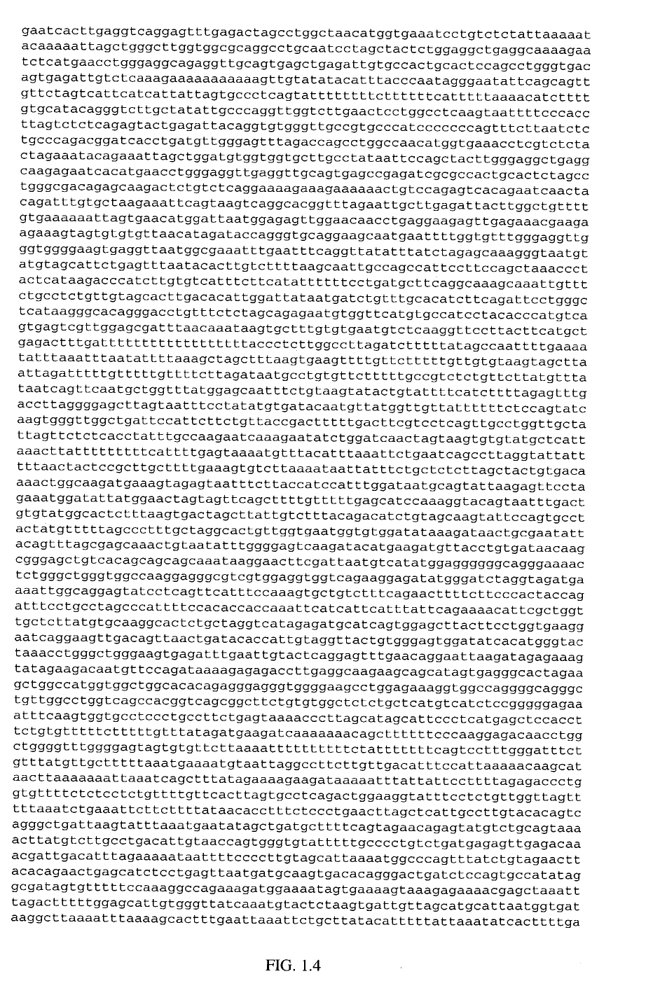

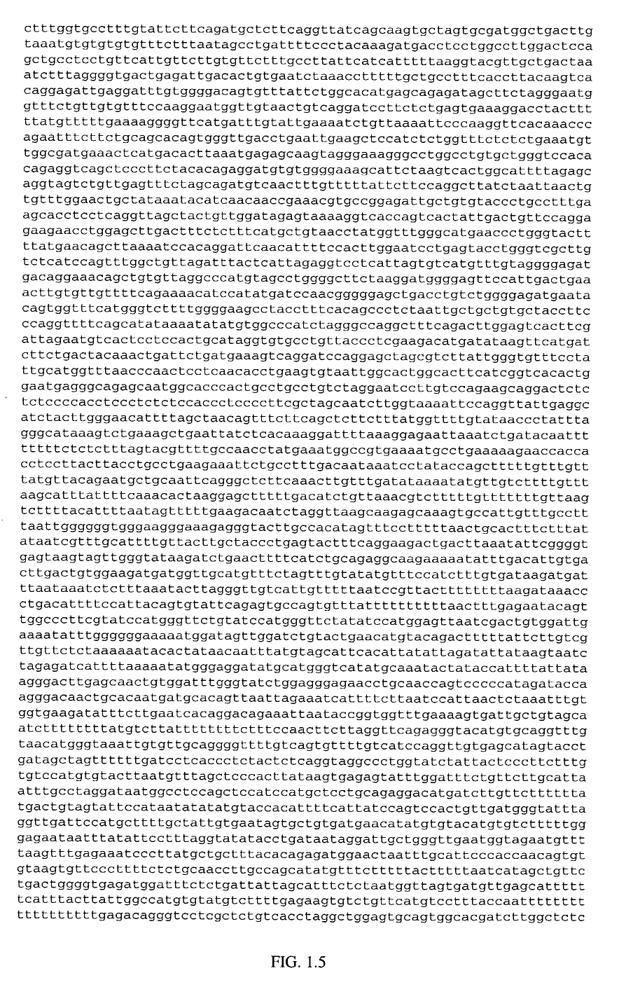

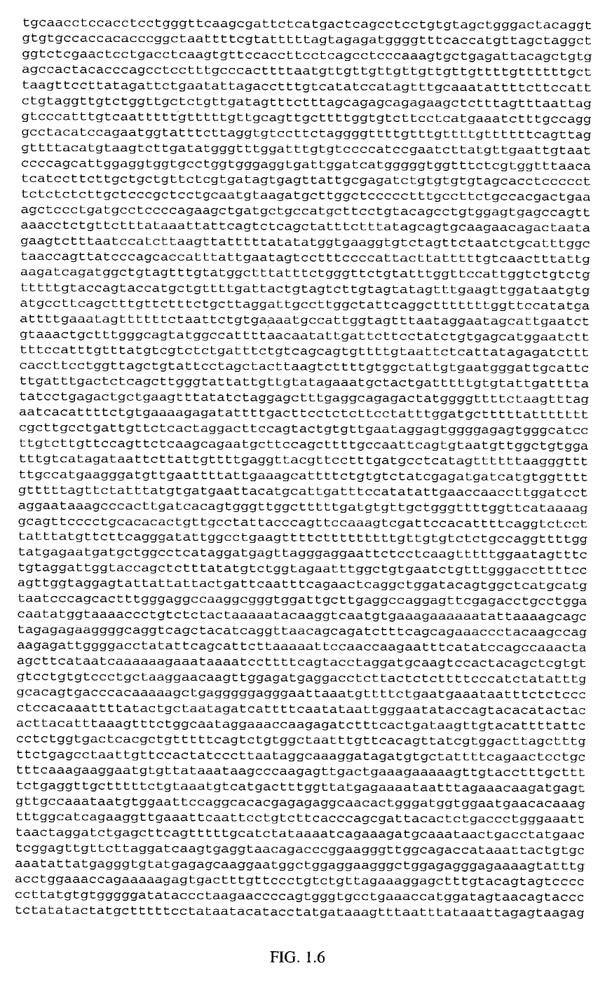

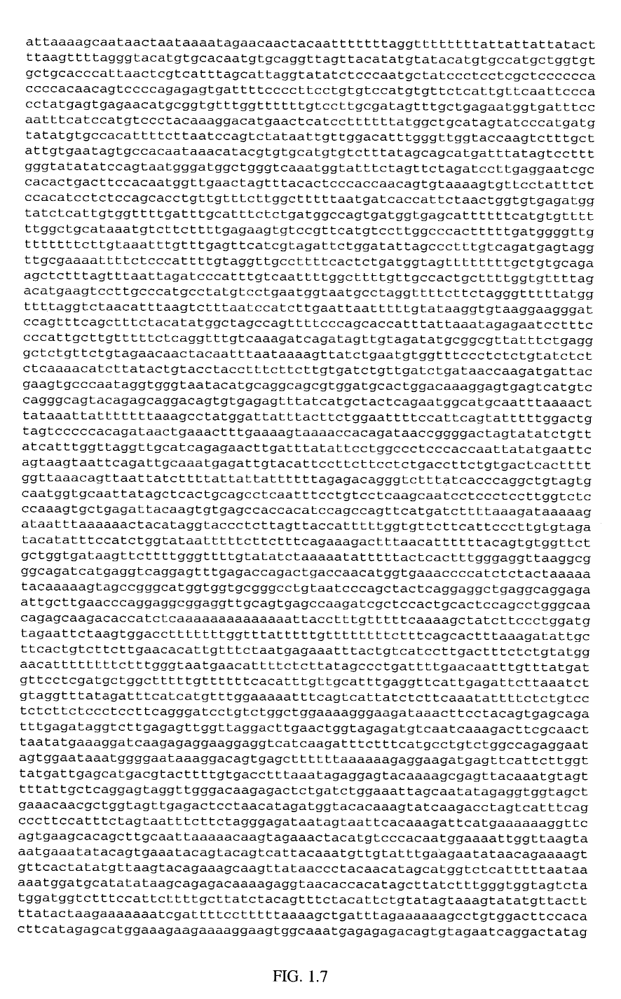

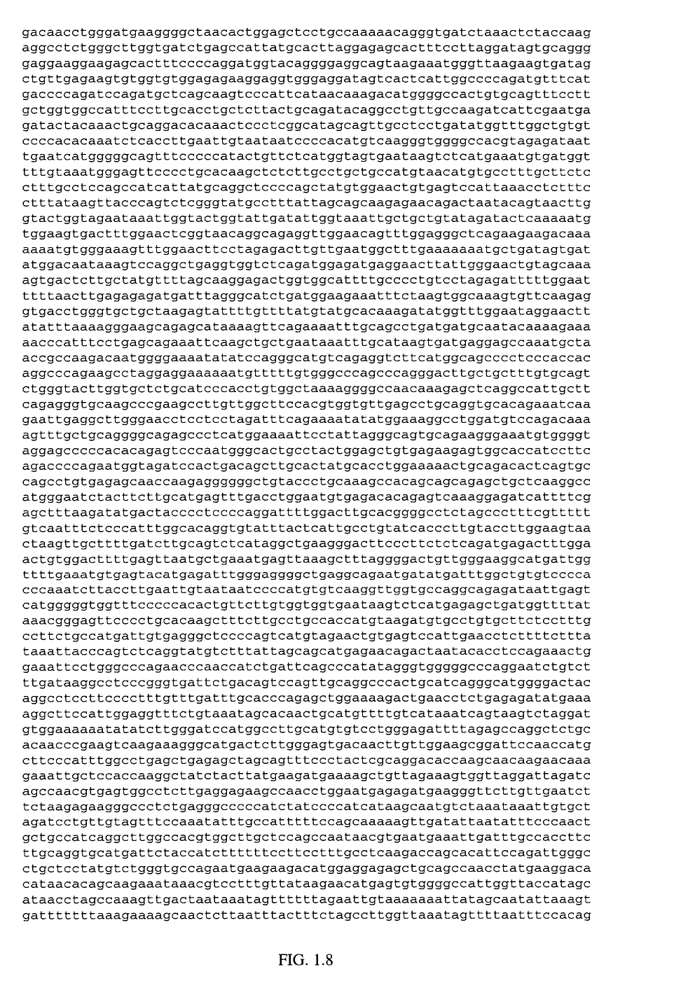

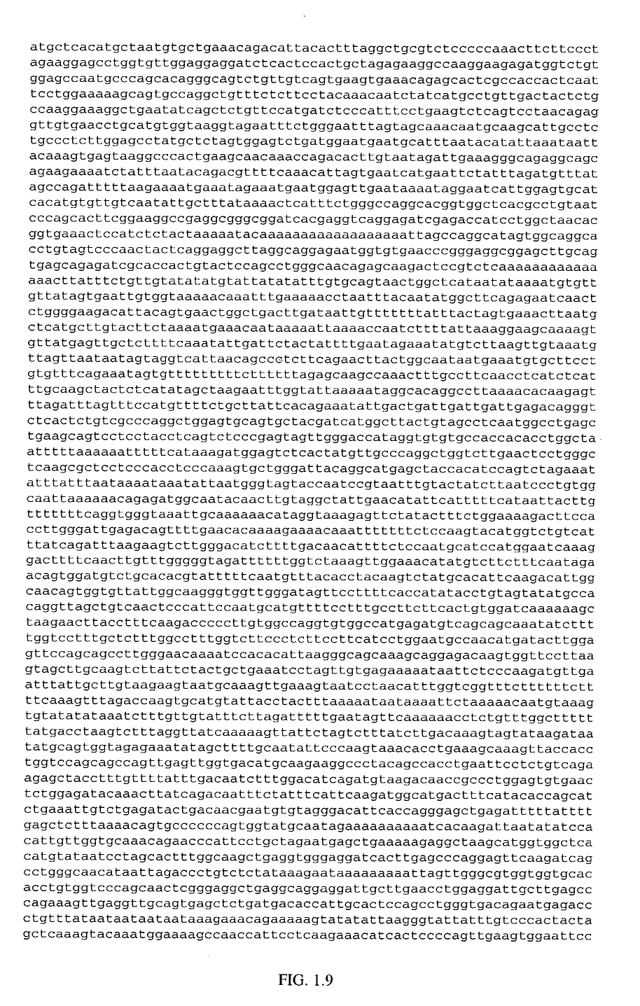

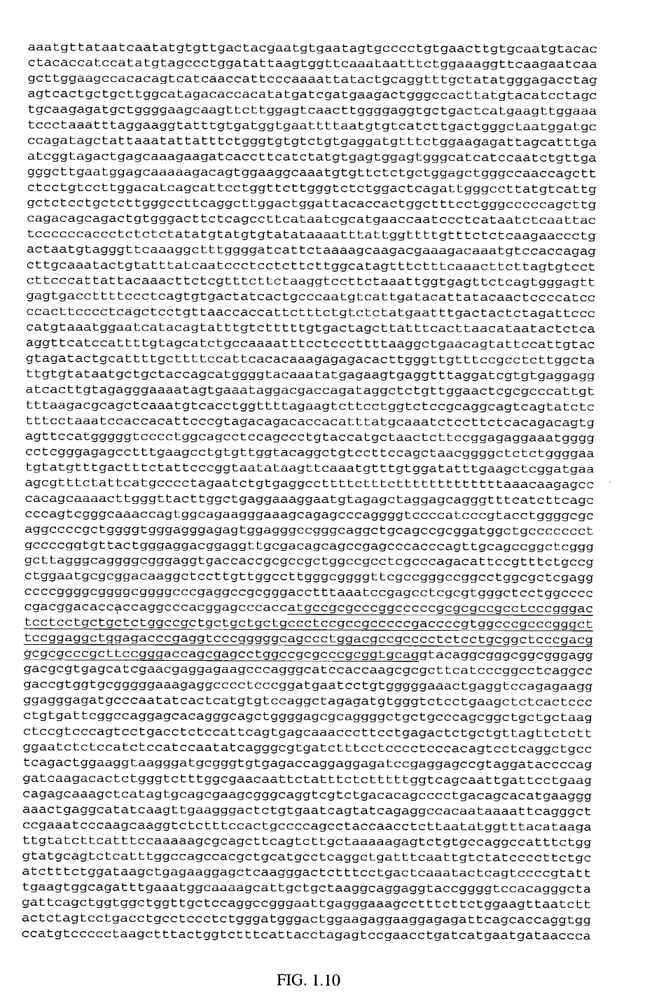

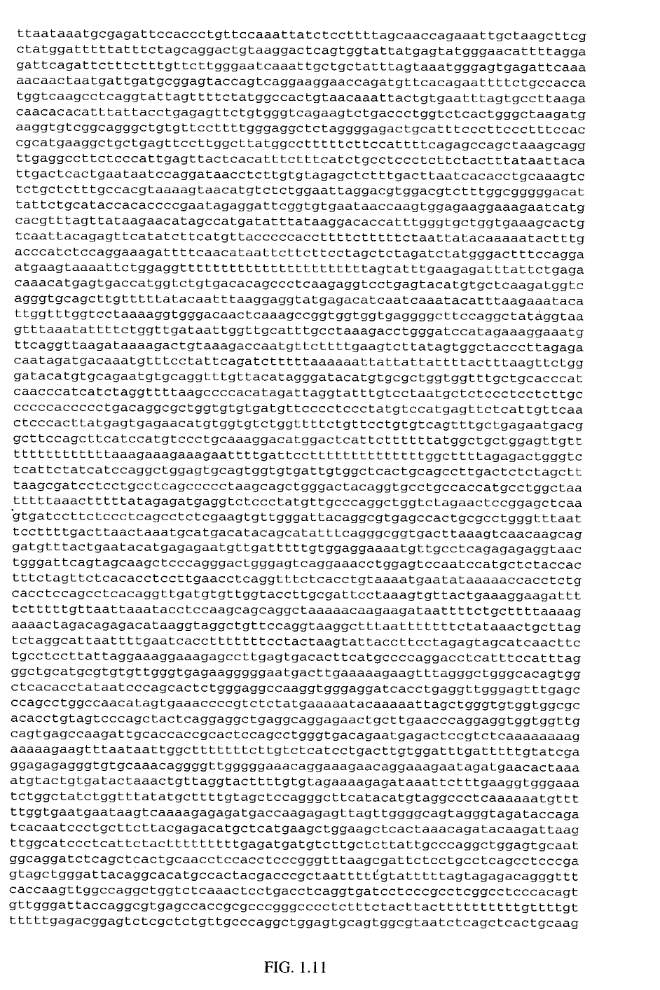

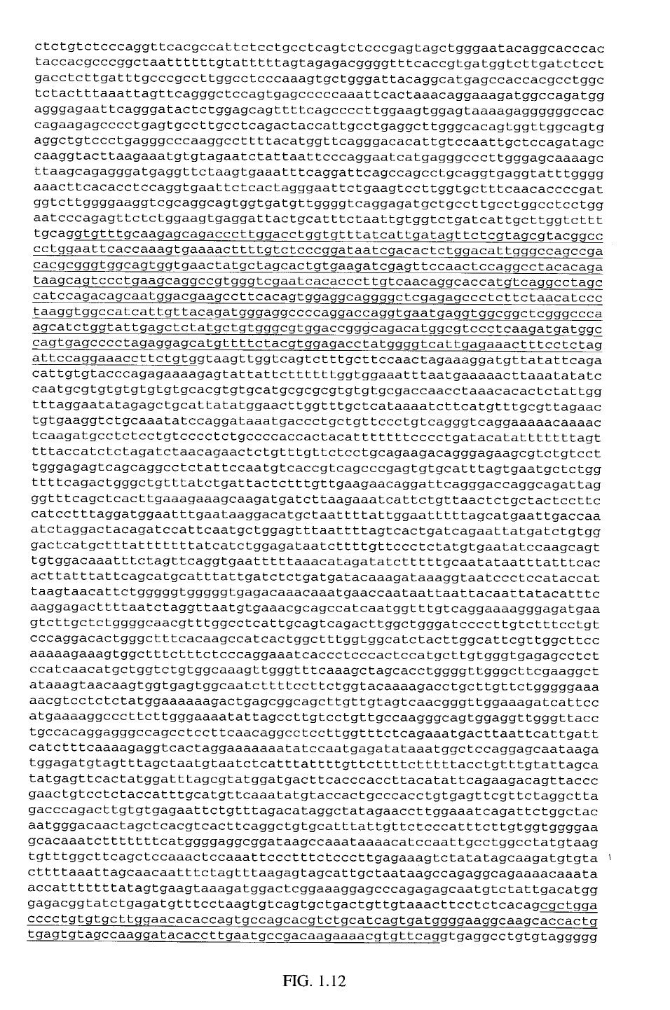

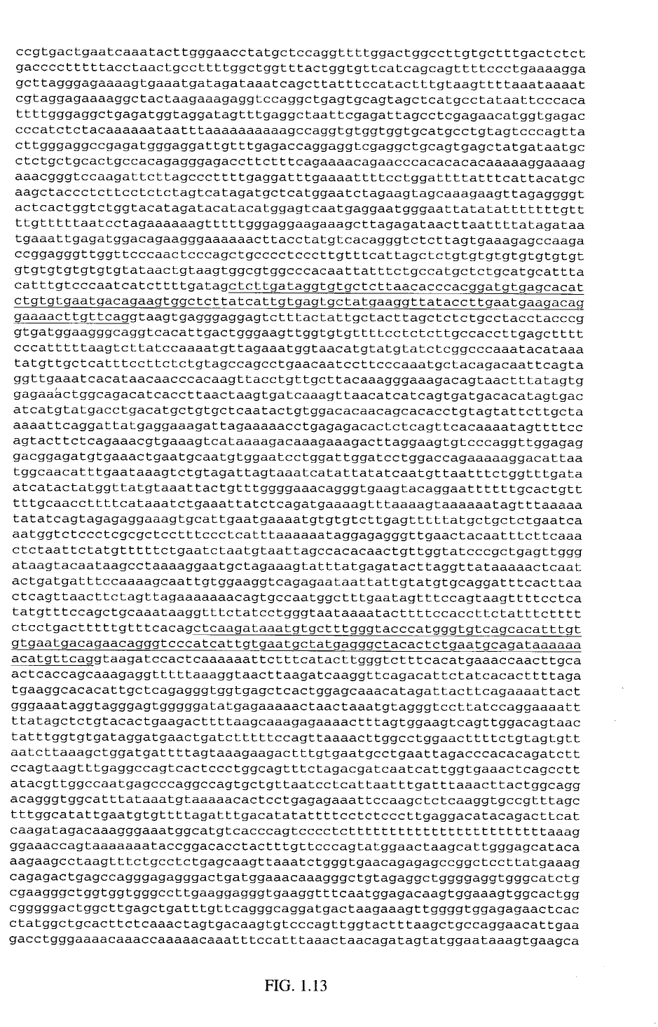

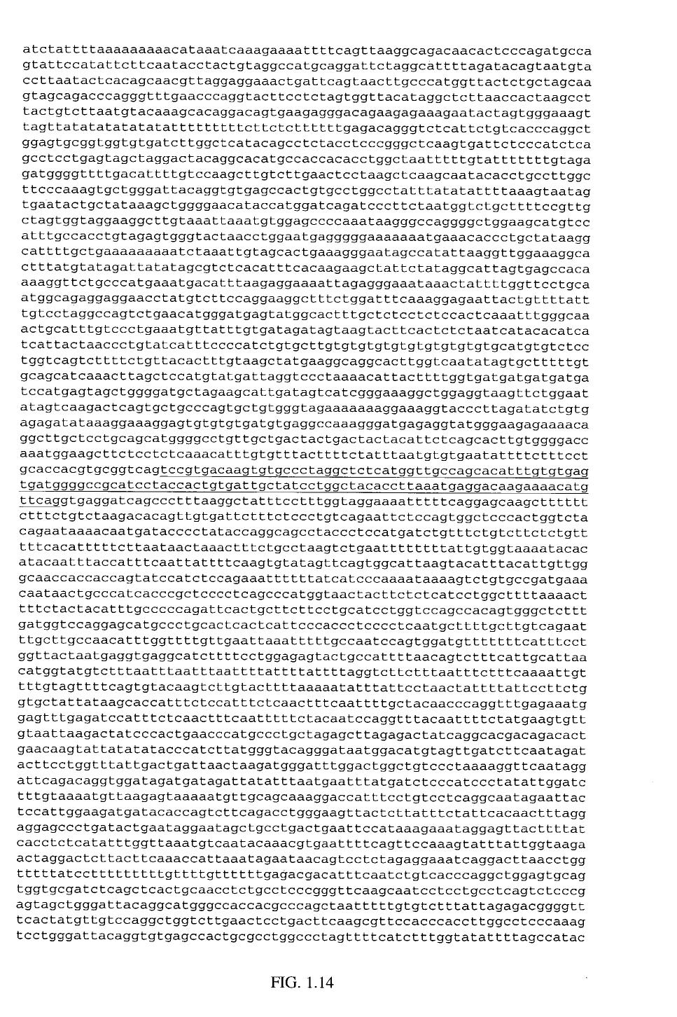

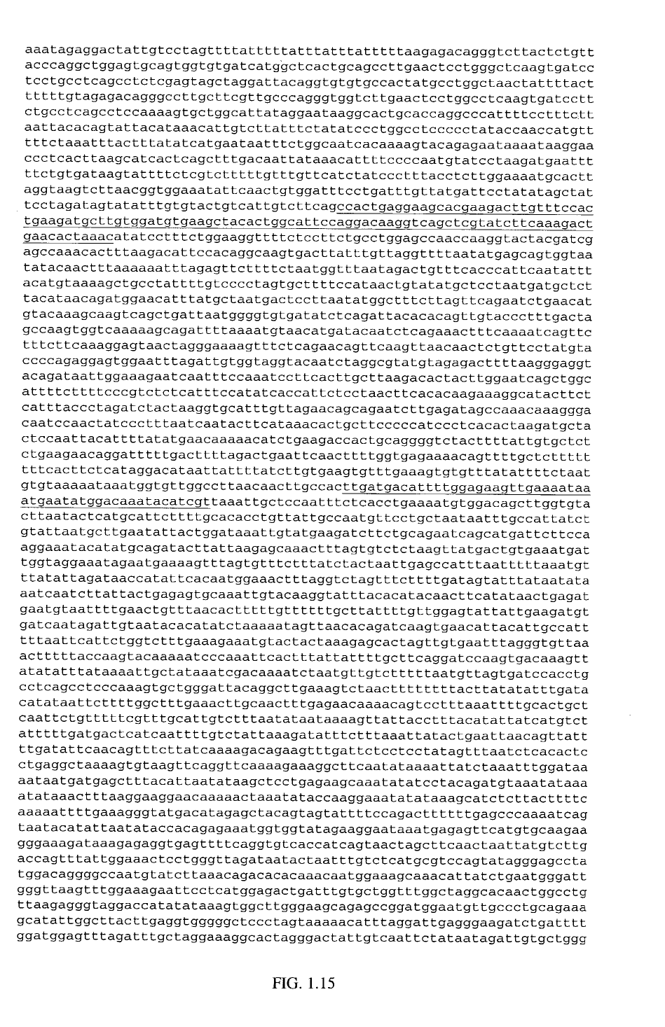

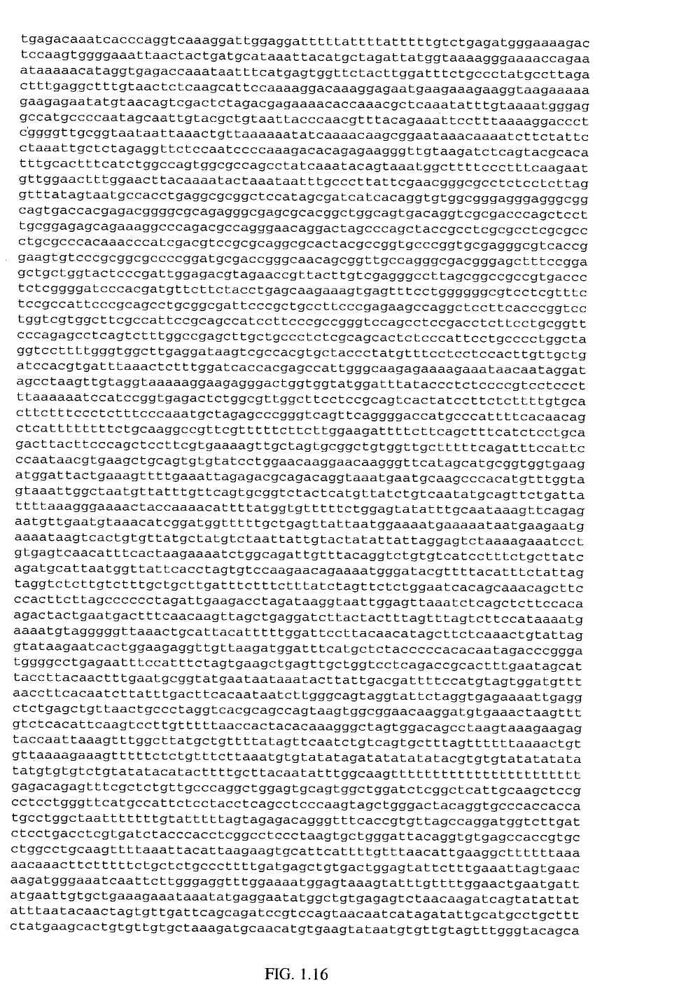

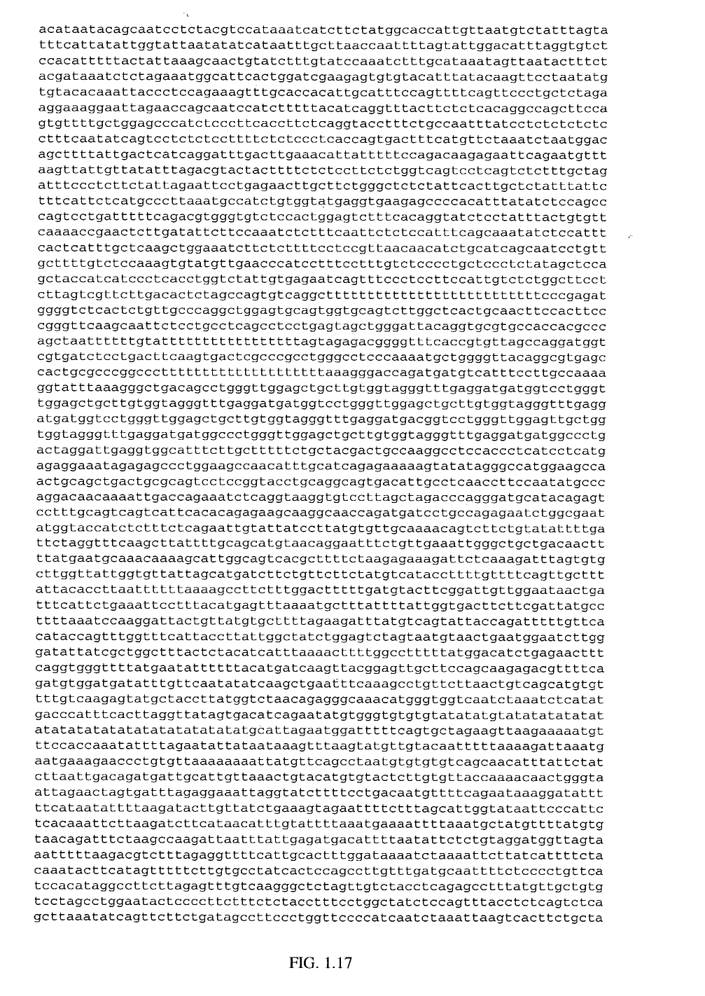

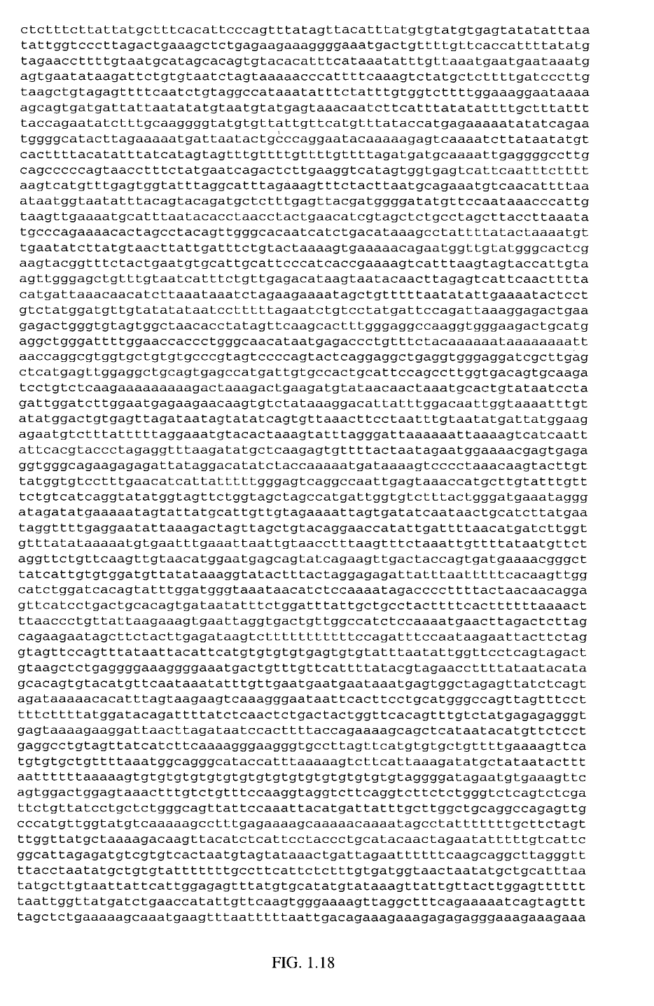

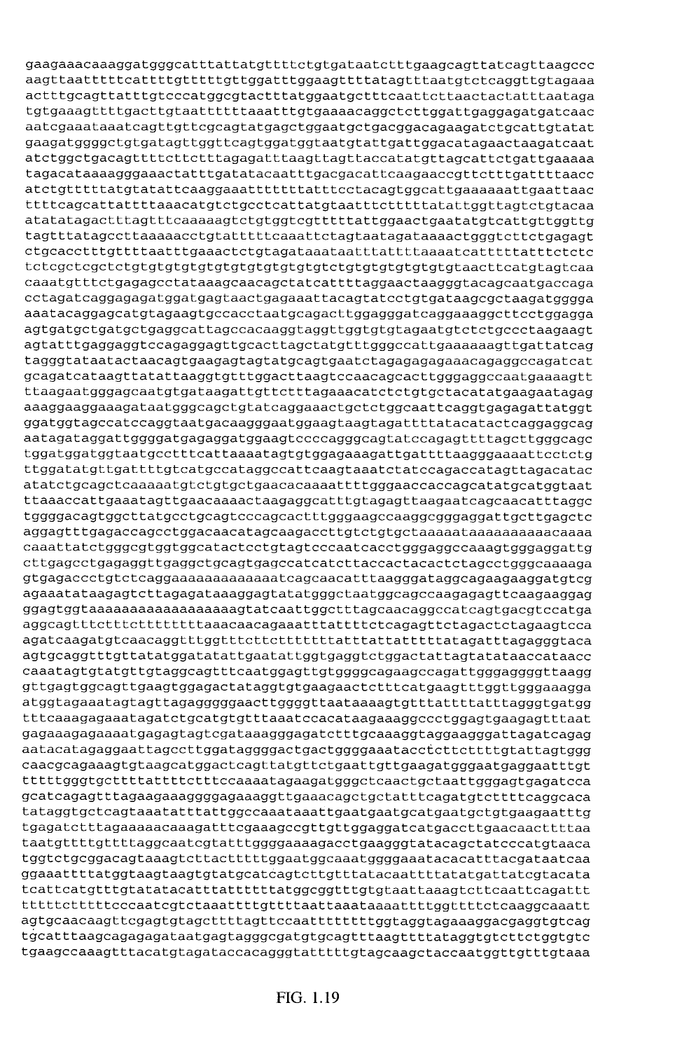

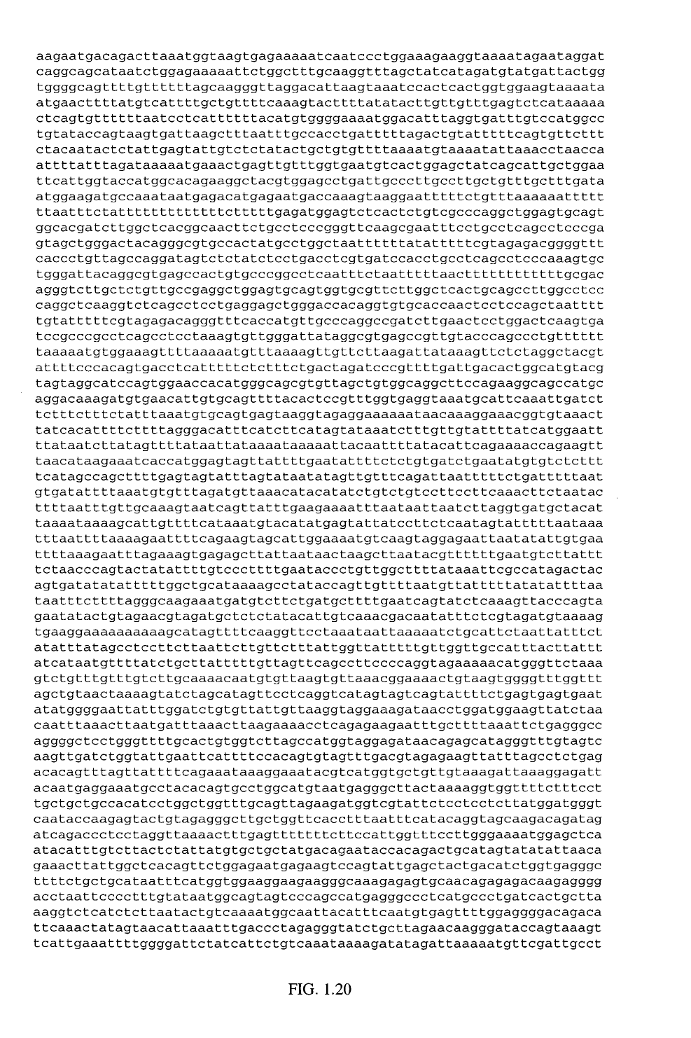

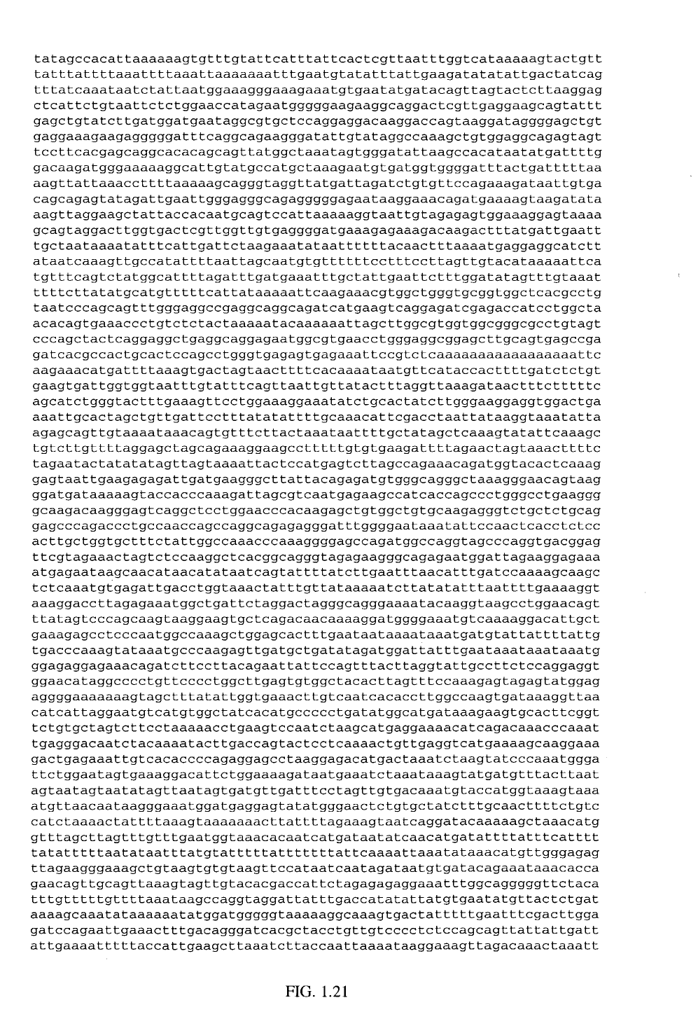

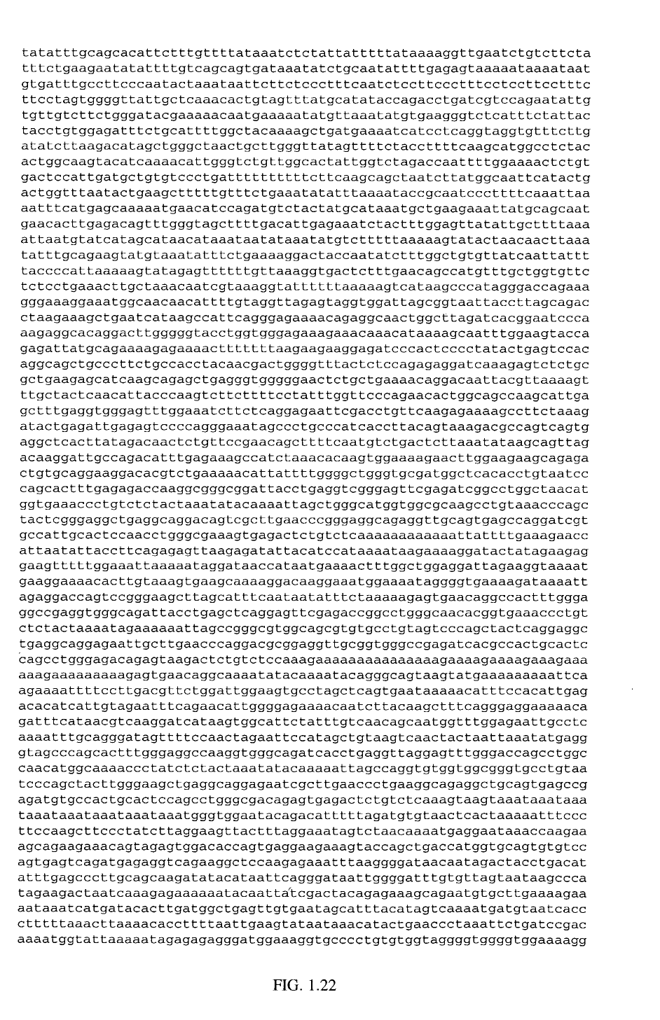

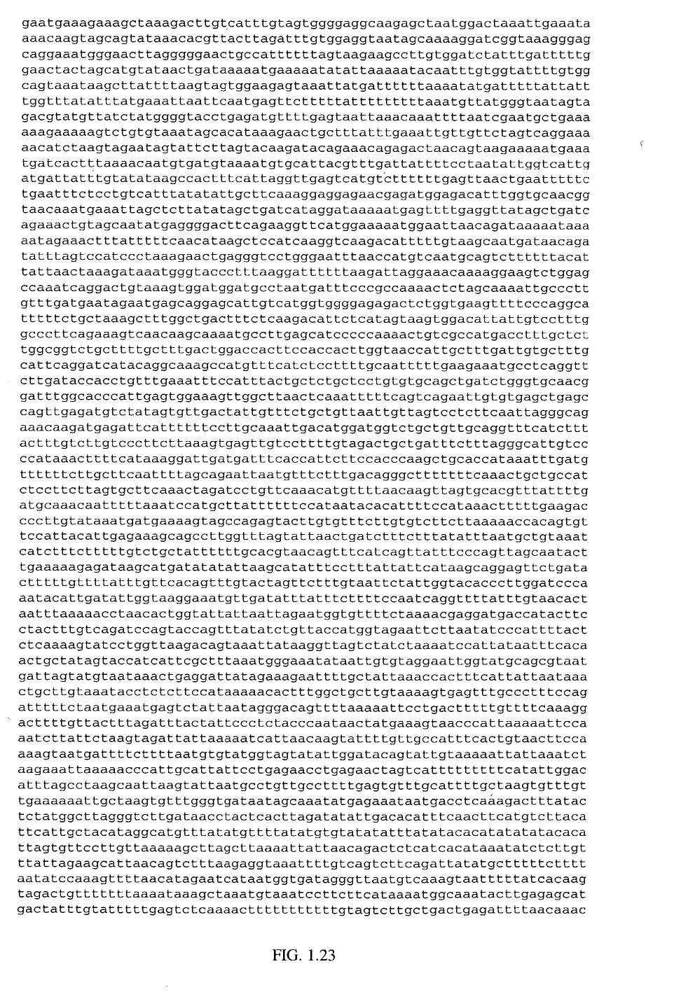

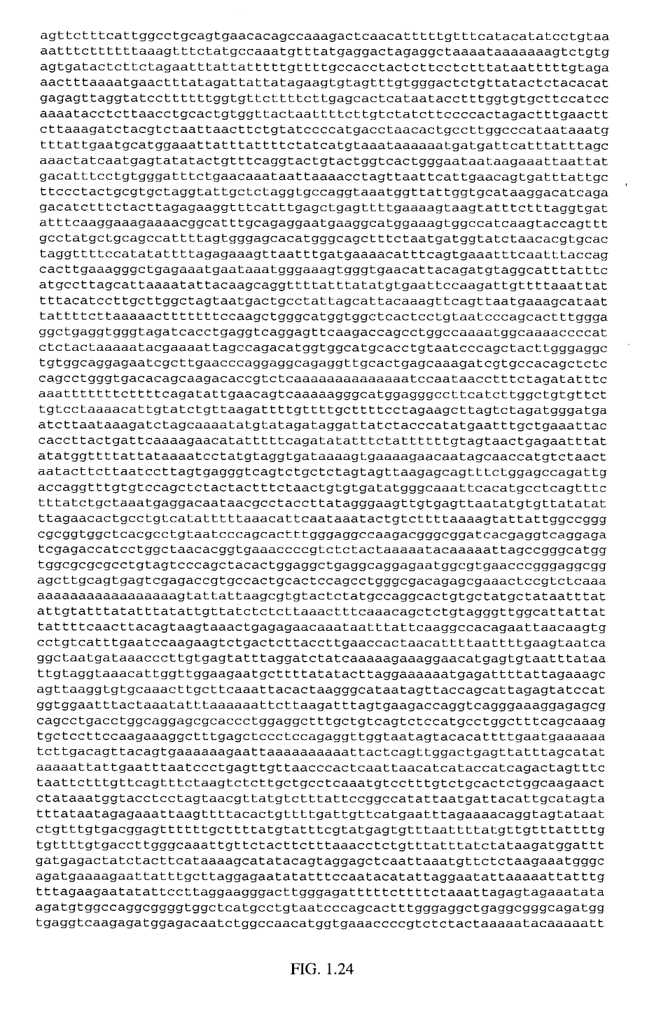

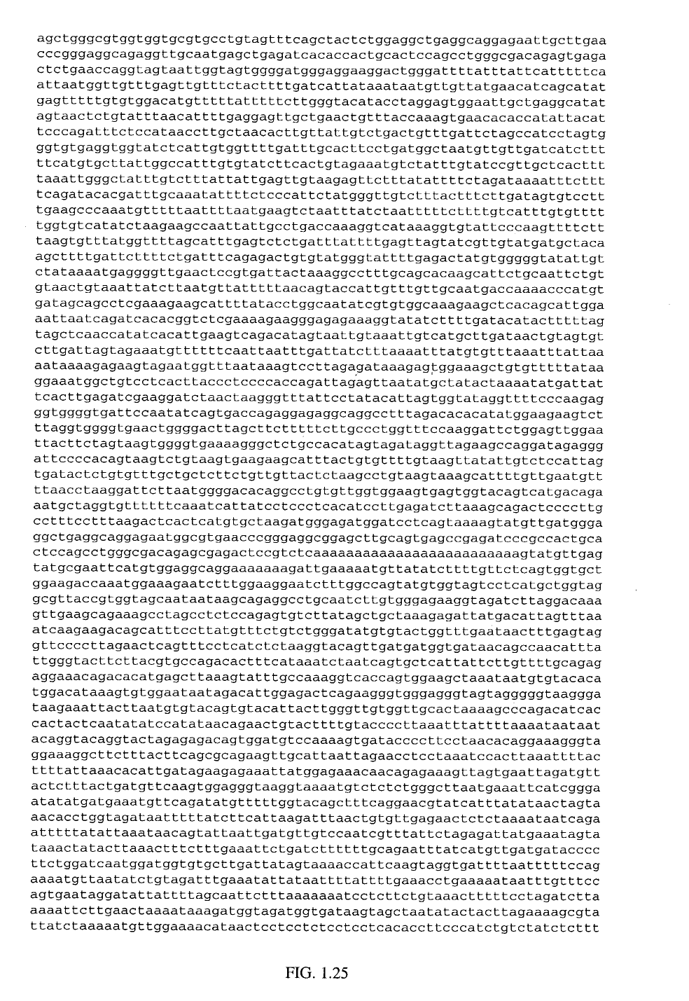

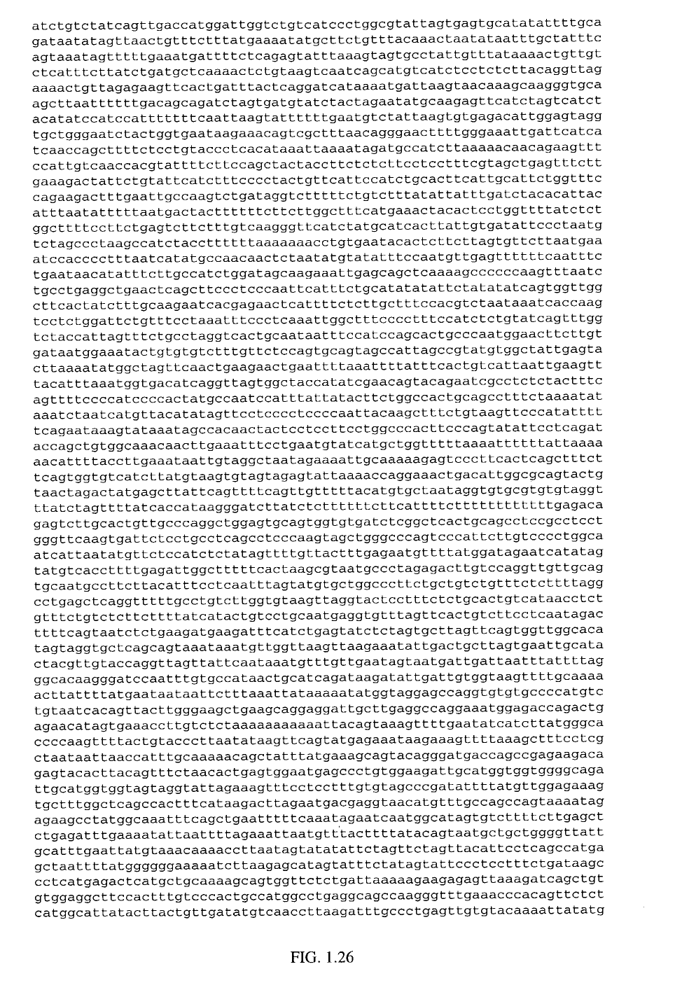

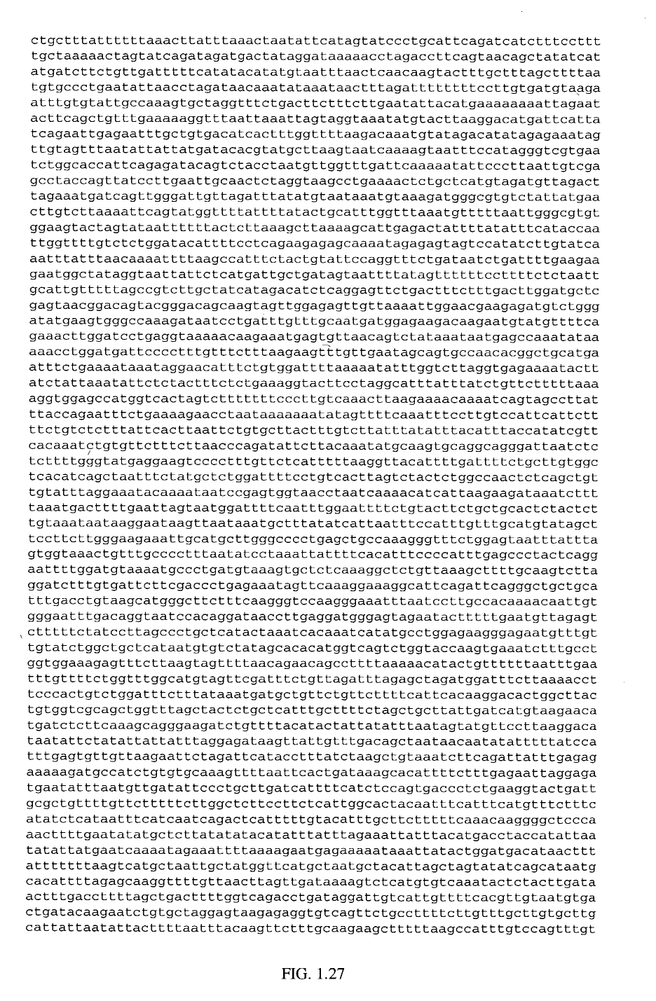

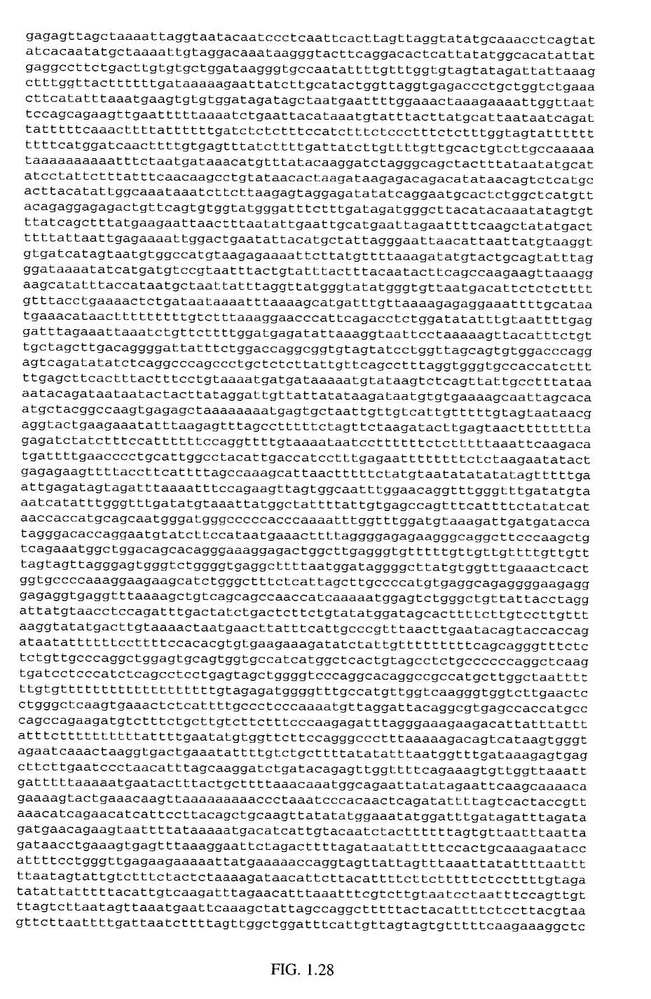

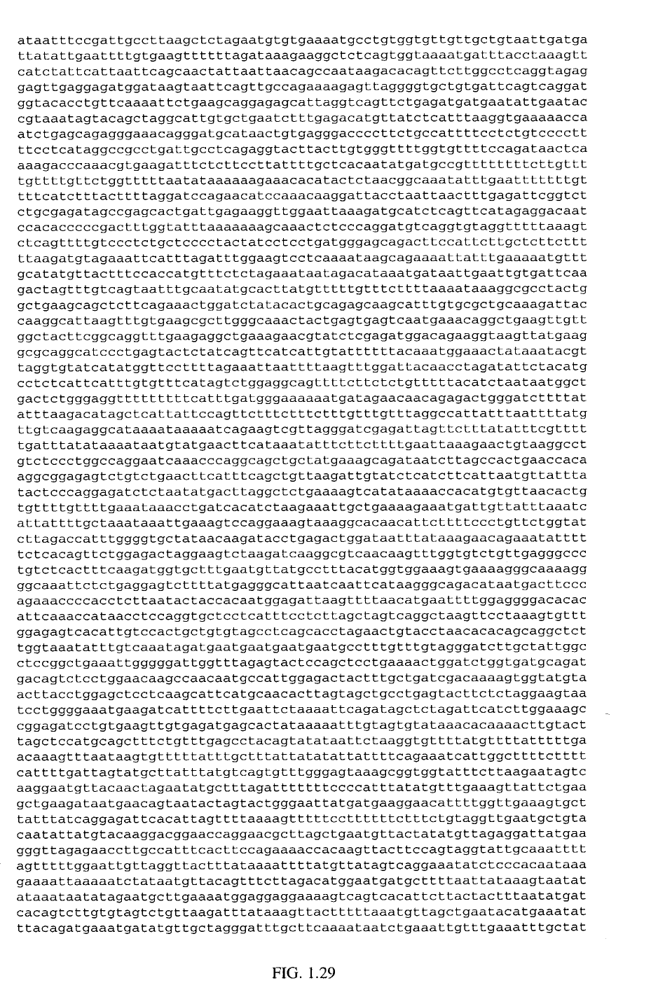

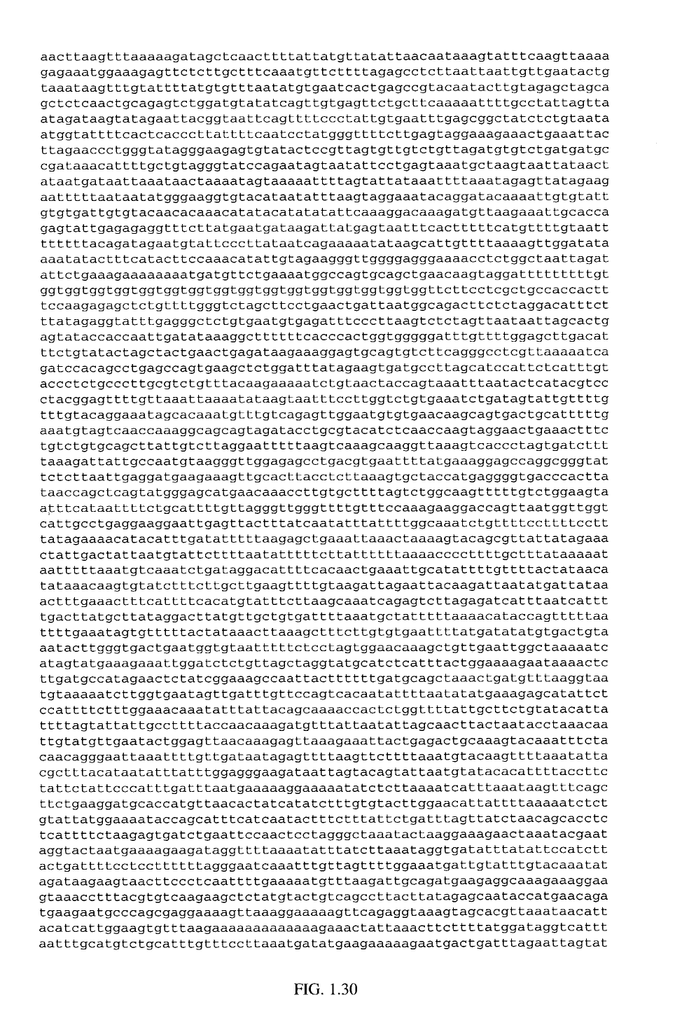

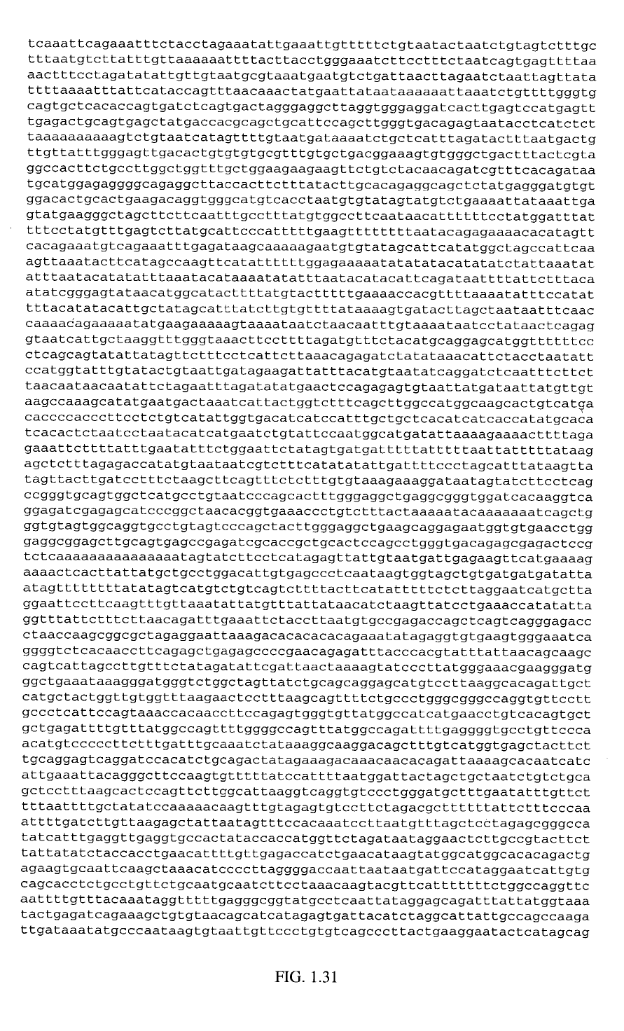

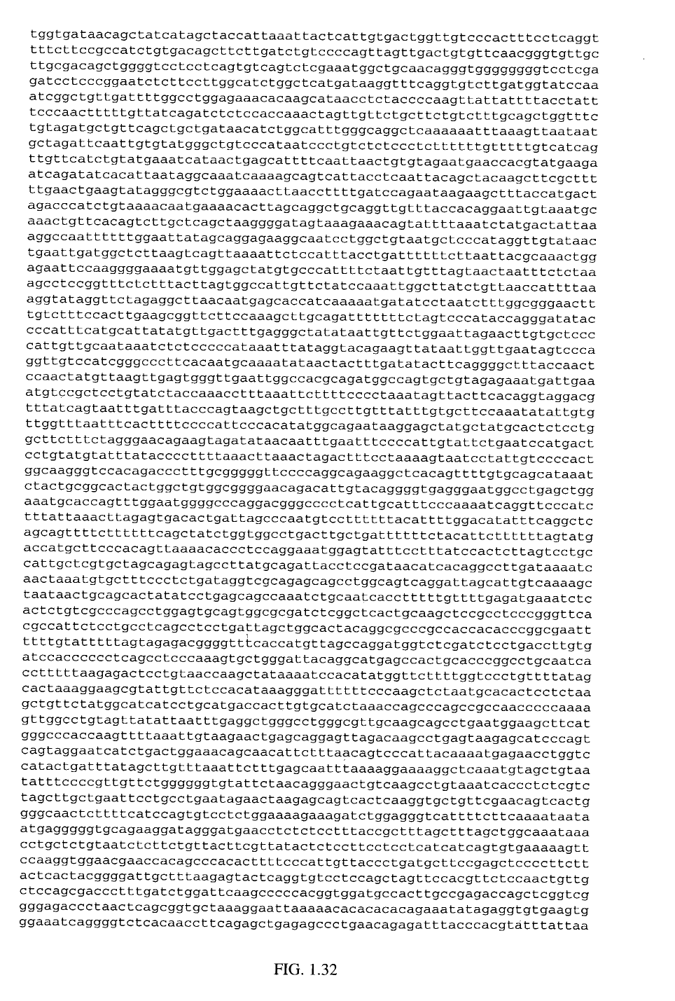

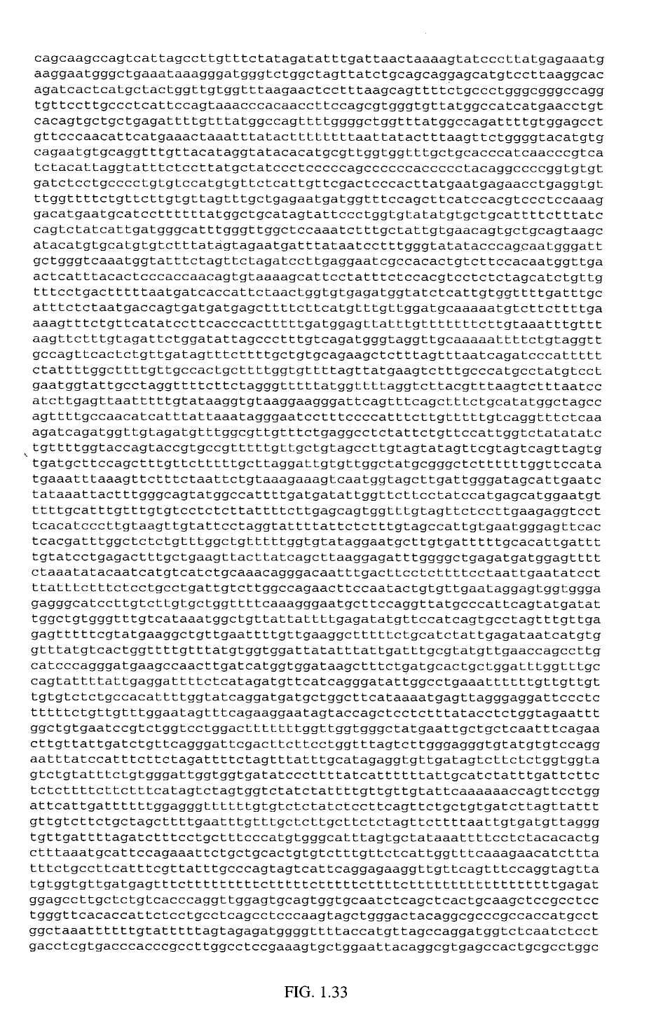

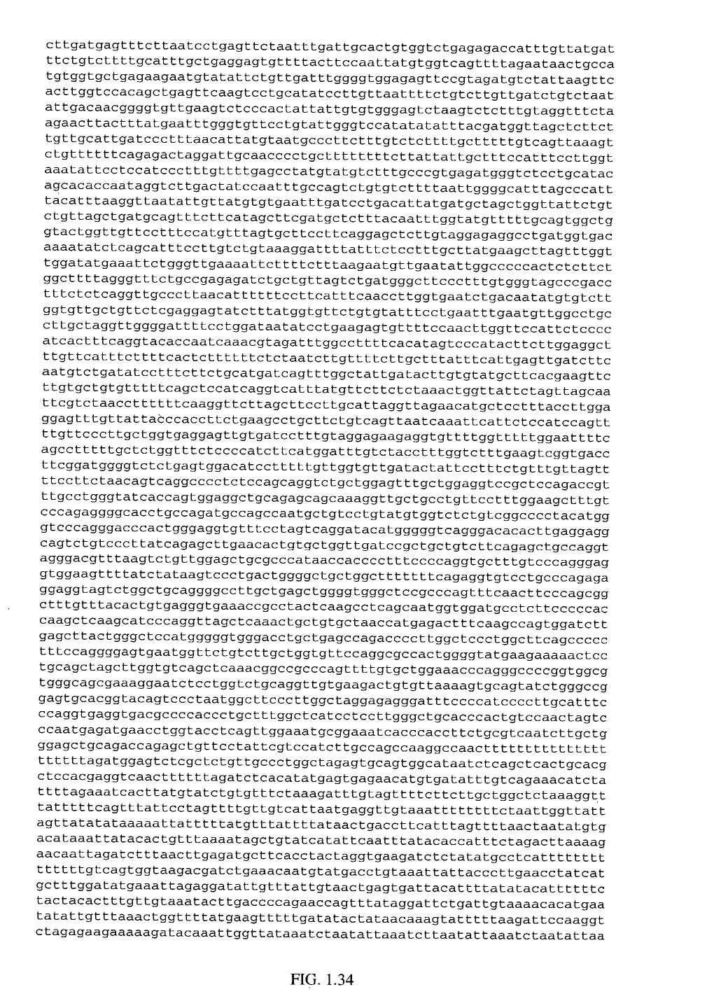

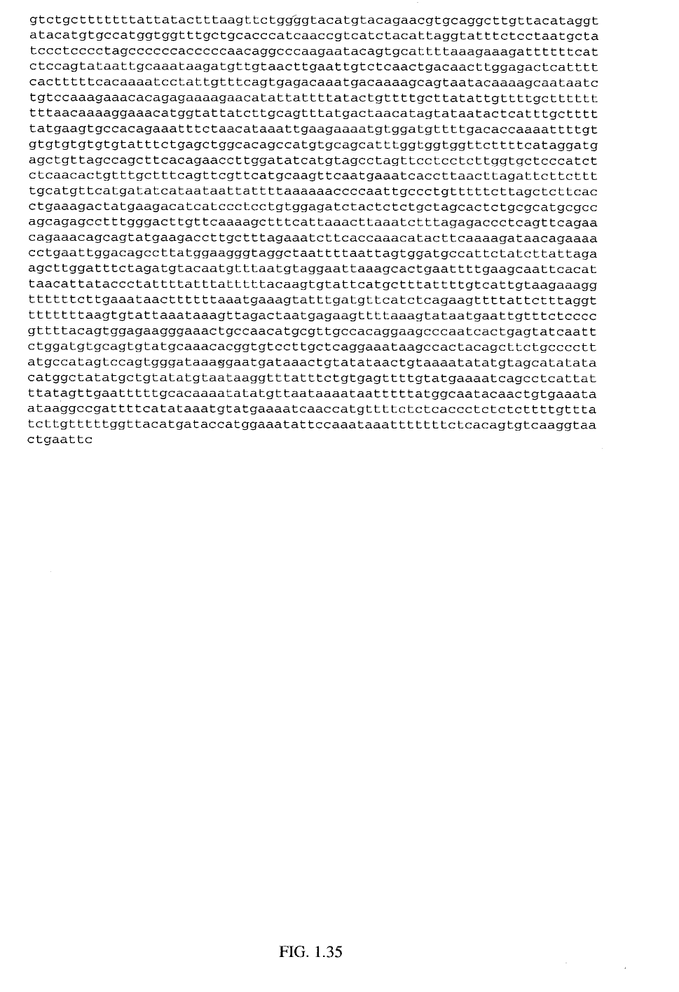

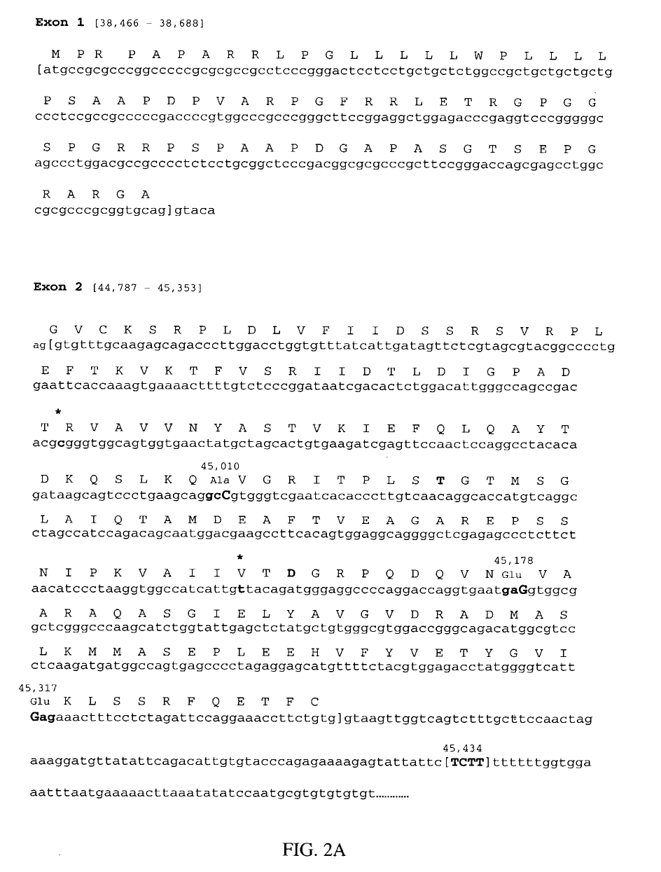

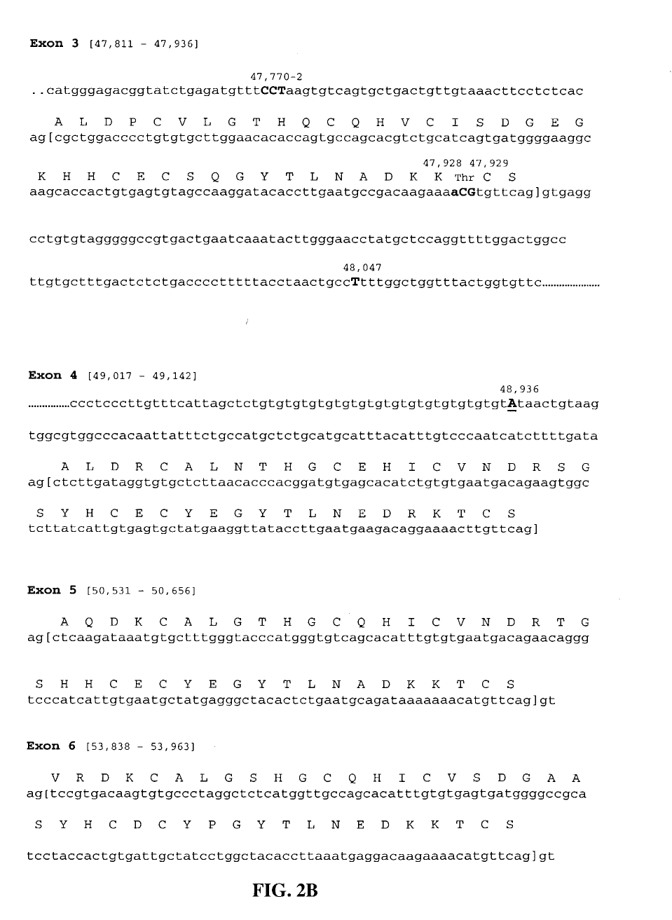

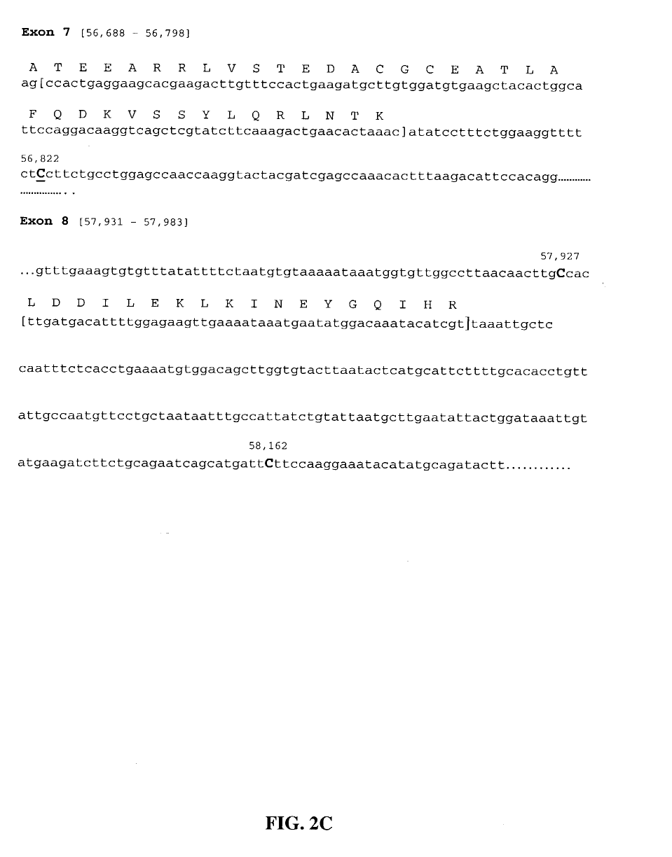

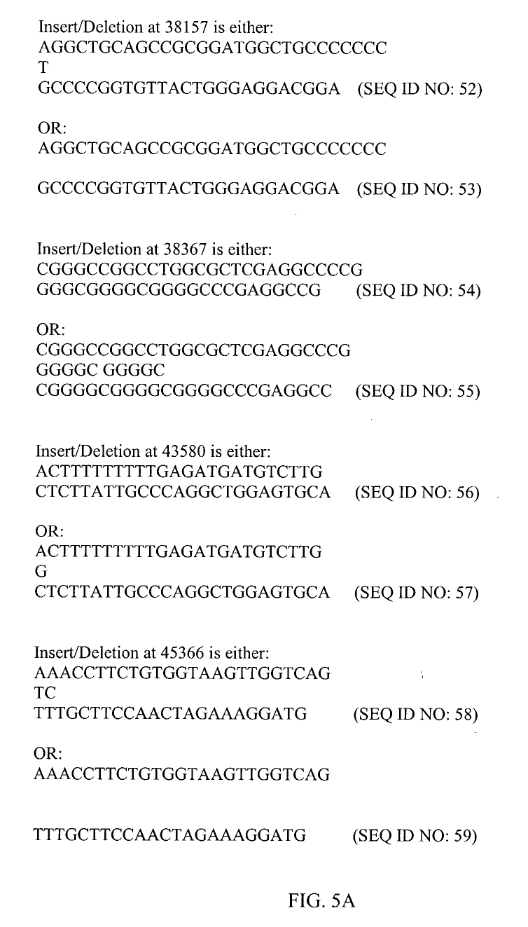

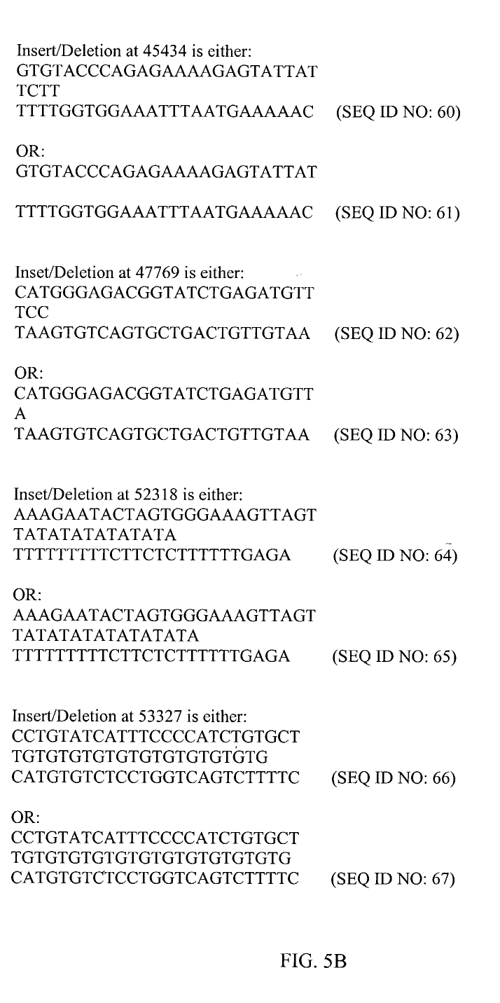

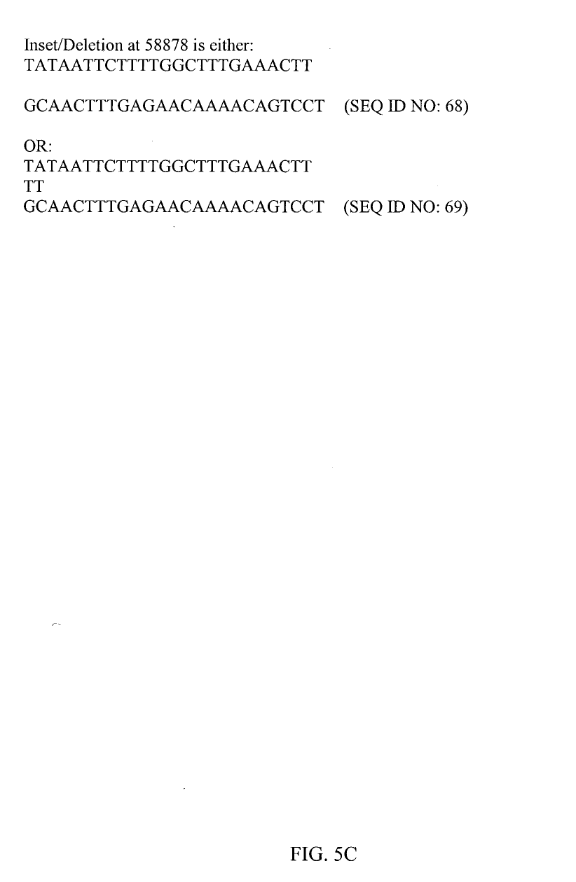

As described herein, Applicant has completed a genome wide scan on patients with two main subsets of osteoarthritis of the hand; finger and thumb. The MATN3 gene on chromosome 2 has been identified through linkage studies to be associated with osteoarthritis. Until now there have been no known linkage studies of osteoarthritis in humans showing significant connection to this region of the chromosome. Based on the linkage studies conducted, Applicant has discovered a direct relationship between the MATN3 gene and osteoarthritis. Although the MATN3 gene from normal individuals is known, there have been no studies directly investigating MATN3 and osteoarthritis. Moreover, there have been no variant forms reported that have been associated with osteoarthritis. The full sequence of the MATN3 gene (normal gene) is shown in FIGS. 1.1 to 1.35 and SEQ ID NO: 1. It should be understood that the nucleic acids and their gene products embraced by the invention include the nucleotide sequence set forth in FIGS. 1.1 to 1.35 and further comprise at least one polymorphism as shown in Table 3 or FIGS. 5A-5C, and may optionally comprise at least one polymorphism shown in FIGS. 2A to 2C.

Nucleic Acids of the Invention

MATN3 Nucleic Acids, Portions and Variants

Accordingly, the invention pertains to an isolated nucleic acid molecule comprising a variant form of the human MATN3 gene. The term, “variant MATN3”, as used herein, refers to an isolated nucleic acid molecule on chromosome 2 having at least one altered nucleotide that is associated with a susceptibility to osteoarthritis, and also to a portion or fragment of the isolated nucleic acid molecule (e.g., cDNA or the gene) that encodes MATN3 polypeptide (e.g., the polypeptide having SEQ ID NO: 2, 3, 4, 5, 6, 7, 8 or 9, as shown in FIGS. 2A to 2C and optionally comprising at least one polymorphism (e.g., a SNP, an insertion, or a deletion of one or more nucleotides) as set forth in Table 3 or FIGS. 5A-5C, or another splicing variant of a MATN3 polypeptide). In one embodiment, the isolated nucleic acid molecule comprises the sequence of SEQ ID NO: 1 or the complement of SEQ ID NO: 1, except that one or more nucleotide polymorphisms as shown in Table 3 or FIGS. 5A-5C are also present. In a particularly preferred embodiment, the isolated nucleic acid molecules comprises at least one of exons 1-8 (SEQ ID NOs: 13-20) as shown in FIGS. 2A to 2C.

The isolated nucleic acid molecules of the present invention can be RNA, for example, mRNA, or DNA, such as cDNA and genomic DNA. DNA molecules can be double-stranded or single-stranded; single stranded RNA or DNA can be either the coding, or sense, strand or the non-coding, or antisense, strand. The nucleic acid molecule can include all or a portion of the coding sequence of the gene and can further comprise additional non-coding sequences such as introns and non-coding 3′ and 5′ sequences (including regulatory sequences, for example). Additionally, the nucleic acid molecule can be fused to a marker sequence, for example, a sequence that encodes a polypeptide to assist in isolation or purification of the polypeptide. Such sequences include, but are not limited to, those which encode a glutathione-S-transferase (GST) fusion protein and those which encode a hemagglutinin A (HA) polypeptide marker from influenza.

An “isolated” nucleic acid molecule, as used herein, is one that is separated from nucleic acids which normally flank the gene or nucleotide sequence (as in genomic sequences) and/or has been completely or partially purified from other transcribed sequences (e.g., as in an RNA library). For example, an isolated nucleic acid of the invention may be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material may be purified to essential homogeneity, for example, as determined by PAGE or column chromatography such as HPLC. Preferably, an isolated nucleic acid molecule comprises at least about 50, 80, 90 or 95% (on a molar basis) of all macromolecular species present. With regard to genomic DNA, the term “isolated” also can refer to nucleic acid molecules which are separated from the chromosome with which the genomic DNA is naturally associated. For example, the isolated nucleic acid molecule can contain less than about 5 kb, 4 kb, 3 kb, 2 kb, 1 kb, 0.5 kb or 0.1 kb of nucleotides which flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence which is synthesized chemically or by recombinant means. Therefore, recombinant DNA contained in a vector are included in the definition of “isolated” as used herein. Also, isolated nucleotide sequences include recombinant DNA molecules in heterologous organisms, as well as partially or substantially purified DNA molecules in solution. In vivo and in vitro RNA transcripts of the DNA molecules of the present invention are also encompassed by “isolated” nucleotide sequences. Such isolated nucleotide sequences are useful in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis.

The present invention also pertains to variant nucleic acid molecules which are not necessarily found in nature but which encode a MATN3 polypeptide (e.g., a polypeptide having the amino acid sequence of SEQ ID NO: 2, 3, 4, 5, 6, 7, 8, or 9), or another splicing variant of MATN3 polypeptide or polymorphic variant thereof. Thus, for example, DNA molecules which comprise a sequence that is different from the naturally-occurring nucleotide sequence but which, due to the degeneracy of the genetic code, encode a MATN3 polypeptide of the present invention are also the subject of this invention. The invention also encompasses nucleotide sequences encoding portions (fragments), or encoding variant polypeptides such as analogues or derivatives of the MATN3 polypeptide. Such variants can be naturally-occurring, such as in the case of allelic variation or single nucleotide polymorphisms, or non-naturally-occurring, such as those induced by various mutagens and mutagenic processes. Intended variations include, but are not limited to, addition, deletion and substitution of one or more nucleotides which can result in conservative or non-conservative amino acid changes, including additions and deletions. Preferably the nucleotide (and/or resultant amino acid) changes are silent or conserved; that is, they do not alter the characteristics or activity of the MATN3 polypeptide. In one preferred embodiment, the nucleotide sequences are fragments that comprise one or more polymorphic microsatellite markers. In another preferred embodiment, the nucleotide sequences are fragments that comprise one or more single nucleotide polymorphisms in the MATN3 gene.

Other alterations of the nucleic acid molecules of the invention can include, for example, labeling, methylation, intemucleotide modifications such as uncharged linkages (e.g., methyl phosphonates, phosphotriesters, phosphoamidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Also included are synthetic molecules that mimic nucleic acid molecules in the ability to bind to a designated sequences via hydrogen bonding and other chemical interactions. Such molecules include, for example, those in which peptide linkages substitute for phosphate linkages in the backbone of the molecule.

The invention also pertains to nucleic acid molecules which hybridize under high stringency hybridization conditions, such as for selective hybridization, to a nucleotide sequence described herein (e.g., nucleic acid molecules which specifically hybridize to a nucleotide sequence encoding polypeptides described herein, and, optionally, have an activity of the polypeptide). In one embodiment, the invention includes variants described herein which hybridize under high stringency hybridization conditions (e.g., for selective hybridization) to a nucleotide sequence comprising a nucleotide sequence selected from SEQ ID NO: 1 and comprising at least one polymorphism as shown in Table 3 or FIGS. 5A-5C or the complements thereof. In another embodiment, the invention includes variants described herein which hybridize under high stringency hybridization conditions (e.g., for selective hybridization) to a nucleotide sequence encoding an amino acid sequence selected from SEQ ID NO: 2, 3, 4, 5, 6, 7, 8 or 9 or polymorphic variant thereof. In a preferred embodiment, the variant which hybridizes under high stringency hybridizations has an activity of MATN3. Such nucleic acid molecules can be detected and/or isolated by specific hybridization (e.g., under high stringency conditions). “Specific hybridization,” as used herein, refers to the ability of a first nucleic acid to hybridize to a second nucleic acid in a manner such that the first nucleic acid does not hybridize to any nucleic acid other than to the second nucleic acid (e.g., when the first nucleic acid has a higher similarity to the second nucleic acid than to any other nucleic acid in a sample wherein the hybridization is to be performed). “Stringency conditions” for hybridization is a term of art which refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity which is less than perfect (e.g., 70%, 75%, 85%, 90%, 95%). For example, certain high stringency conditions can be used which distinguish perfectly complementary nucleic acids from those of less complementarity. “High stringency conditions”, “moderate stringency conditions” and “low stringency conditions” for nucleic acid hybridizations are explained on pages 2.10.1-2.10.16 and pages 6.3.1-6.3.6 in Current Protocols in Molecular Biology (Ausubel, F. M. et al., “Current Protocols in Molecular Biology”, John Wiley & Sons, (1998)), the entire teachings of which are incorporated by reference herein). The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, equivalent conditions can be determined by varying one or more of these parameters while maintaining a similar degree of identity or similarity between the two nucleic acid molecules. Typically, conditions are used such that sequences at least about 60%, at least about 70%, at least about 80%, at least about 90% or at least about 95% or more identical to each other remain hybridized to one another. By varying hybridization conditions from a level of stringency at which no hybridization occurs to a level at which hybridization is first observed, conditions which will allow a given sequence to hybridize (e.g., selectively) with the most similar sequences in the sample can be determined.

Exemplary conditions are described in Krause, M. H. and S. A. Aaronson (1991) Methods in Enzymology, 200:546-556. Also, in, Ausubel, et al., “Current Protocols in Molecular Biology”, John Wiley & Sons, (1998), which describes the determination of washing conditions for moderate or low stringency conditions. Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum extent of mismatching among the sequences that hybridize. Generally, doubling the concentration of SSC results in an increase in Tₘ of ˜17° C. Using these guidelines, the washing temperature can be determined empirically for high, moderate or low stringency, depending on the level of mismatch sought.

For example, a low stringency wash can comprise washing in a solution containing 0.2×SSC/0.1% SDS for 10 min at room temperature; a moderate stringency wash can comprise washing in a prewarmed solution (42° C.) solution containing 0.2×SSC/0.1% SDS for 15 min at 42° C.; and a high stringency wash can comprise washing in prewarmed (68° C.) solution containing 0.1×SSC/0.1%SDS for 15 min at 68° C. Furthermore, washes can be performed repeatedly or sequentially to obtain a desired result as known in the art. Equivalent conditions can be determined by varying one or more of the parameters given as an example, as known in the art, while maintaining a similar degree of identity or similarity between the target nucleic acid molecule and the primer or probe used.

The percent homology or identity of two nucleotide or amino acid sequences can be determined by aligning the sequences for optimal comparison purposes (e.g., gaps can be introduced in the sequence of a first sequence for optimal alignment). The nucleotides or amino acids at corresponding positions are then compared, and the percent identity between the two sequences is a function of the number of identical positions shared by the sequences (i.e., % identity=# of identical positions/total # of positions×100). When a position in one sequence is occupied by the same nucleotide or amino acid residue as the corresponding position in the other sequence, then the molecules are homologous at that position. As used herein, nucleic acid or amino acid “homology” is equivalent to nucleic acid or amino acid “identity”. In certain embodiments, the length of a sequence aligned for comparison purposes is at least 30%, for example, at least 40%, in certain embodiments at least 60%, and in other embodiments at least 70%, 80%, 90% or 95% of the length of the reference sequence. The actual comparison of the two sequences can be accomplished by well-known methods, for example, using a mathematical algorithm. A preferred, non-limiting example of such a mathematical algorithm is described in Karlin et al. (1993) Proc. Natl. Acad. Sci. USA 90:5873-5877. Such an algorithm is incorporated into the NBLAST and XBLAST programs (version 2.0) as described in Altschul et al. (1997) Nucleic Acids Res. 25:389-3402. When utilizing BLAST and Gapped BLAST programs, the default parameters of the respective programs (e.g., NBLAST) can be used. In one embodiment, parameters for sequence comparison can be set at score=100, wordlength=12, or can be varied (e.g., W=5 or W=20).

Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (1994) Comput. Appl. Biosci., 10:3-5; and FASTA described in Pearson and Lipman (1988) Proc. Natl. Acad. Sci. USA 85:2444-2448.

In another embodiment, the percent identity between two amino acid sequences can be accomplished using the GAP program in the GCG software package (available from Accelrys, San Diego, Calif.) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package, using a gap weight of 50 and a length weight of 3.

The present invention also provides isolated nucleic acid molecules that contain a fragment or portion that hybridizes under highly stringent conditions to a nucleotide sequence comprising a nucleotide sequence selected from SEQ ID NO: 1 and comprising at least one polymorphism as shown in Table 3 or FIGS. 5A-5C and the complements thereof, and also provides isolated nucleic acid molecules that contain a fragment or portion that hybridizes under highly stringent conditions to a nucleotide sequence encoding an amino acid sequence selected from SEQ ID NO: 2, 3, 4, 5, 6, 7, 8 or 9, or a polymorphic variant thereof. The nucleic acid fragments of the invention are at least about 15, preferably at least about 18, 20, 23 or 25 nucleotides, and can be 30, 40, 50, 100, 200 or more nucleotides in length. Longer fragments, for example, 30 or more nucleotides in length, which encode antigenic polypeptides described herein are particularly useful, such as for the generation of antibodies as described below.

Probes and Primers

In a related aspect, the nucleic acid fragments of the invention are used as probes or primers in assays such as those described herein. “Probes” or “primers” are oligonucleotides that hybridize in a base-specific manner to a complementary strand of nucleic acid molecules. Such probes and primers include polypeptide nucleic acids, as described in Nielsen et al. (1991) Science, 254:1497-1500.

A probe or primer comprises a region of nucleotide sequence that hybridizes to at least about 15, for example about 20-25, and in certain embodiments about 40, 50 or 75, consecutive nucleotides of a nucleic acid molecule comprising a contiguous nucleotide sequence selected from the group consisting of SEQ ID NOs: 1 and 13-20 or a polymorphic variant thereof. In other embodiments, a probe or primer comprises 100 or fewer nucleotides, in certain embodiments from 6 to 50 nucleotides, for example from 12 to 30 nucleotides. In other embodiments, the probe or primer is at least 70% identical to the contiguous nucleotide sequence or to the complement of the contiguous nucleotide sequence, for example at least 80% identical, in certain embodiments at least 90% identical, and in other embodiments at least 95% identical, or even capable of selectively hybridizing to the contiguous nucleotide sequence or to the complement of the contiguous nucleotide sequence. Often, the probe or primer further comprises a label, e.g., radioisotope, fluorescent compound, enzyme, or enzyme co-factor.

The nucleic acid molecules of the invention such as those described above can be identified and isolated using standard molecular biology techniques and the sequence information provided herein. For example, nucleic acid molecules can be amplified and isolated by the polymerase chain reaction using synthetic oligonucleotide primers designed based on one or more of the sequences provided in SEQ ID NO: 1 and comprising at least one polymorphism shown in Table 3 or FIGS. 5A-5C, and/or the complements thereof, or designed based on nucleotides based on sequences encoding one or more of the amino acid sequences provided herein. See generally PCR Technology: Principles and Applications for DNA Amplification (ed. H. A. Erlich, Freeman Press, NY, N.Y., 1992); PCR Protocols: A Guide to Methods and Applications (Eds. Innis, et al., Academic Press, San Diego, Calif., 1990); Mattila et al. (1991) Nucleic Acids Res., 19:4967; Eckert et al. (1991) PCR Methods and Applications, 1:17; PCR (eds. McPherson et al., IRL Press, Oxford); and U.S. Pat. No. 4,683,202. The nucleic acid molecules can be amplified using cDNA, mRNA or genomic DNA as a template, cloned into an appropriate vector and characterized by DNA sequence analysis.

Other suitable amplification methods include the ligase chain reaction (LCR) (see Wu and Wallace (1989) Genomics 4:560, Landegren et al. (1988) Science 241:1077 , transcription amplification (Kwoh et al. (1989) Proc. Natl. Acad. Sci. USA 86:1173), self-sustained sequence replication (Guatelli et al. (1990) Proc. Nat. Acad. Sci. USA 87:1874) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The amplified DNA can be labeled, for example, radiolabeled, and used as a probe for screening a cDNA library derived from human cells, mRNA in zap express, ZIPLOX or other suitable vector. Corresponding clones can be isolated, DNA can obtained following in vivo excision, and the cloned insert can be sequenced in either or both orientations by art recognized methods to identify the correct reading frame encoding a polypeptide of the appropriate molecular weight. For example, the direct analysis of the nucleotide sequence of nucleic acid molecules of the present invention can be accomplished using well-known methods that are commercially available. See, for example, Sambrook et al., Molecular Cloning, A Laboratory Manual (2nd Ed., CSHP, New York 1989); Zyskind et al., Recombinant DNA Laboratory Manual, (Acad. Press, 1988). Using these or similar methods, the polypeptide and the DNA encoding the polypeptide can be isolated, sequenced and further characterized.

Antisense nucleic acid molecules of the invention can be designed using the nucleotide sequences of SEQ ID NOs: 13-20 and/or the complement of SEQ ID NOs: 13-20, and/or a portion of SEQ ID NOs: 13-20 or the complement of a portion of SEQ ID NOs: 13-20 or encoding a portion of SEQ ID NO: 1 wherein the portion of SEQ ID NO: 1 comprises at least one polymorphism as shown in Table 3 or FIGS. 5A-5C and constructed using chemical synthesis and enzymatic ligation reactions using procedures known in the art. For example, an antisense nucleic acid molecule (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Alternatively, the antisense nucleic acid molecule can be produced biologically using an expression vector into which a nucleic acid molecule has been subcloned in an antisense orientation (i.e., RNA transcribed from the inserted nucleic acid molecule will be of an antisense orientation to a target nucleic acid of interest).

In general, the isolated nucleic acid sequences of the invention can be used as molecular weight markers on Southern gels, and as chromosome markers which are labeled to map related gene positions. The nucleic acid sequences can also be used to compare with endogenous DNA sequences in patients to identify genetic disorders (e.g., a predisposition for or susceptibility to osteoarthritis), and as probes, such as to hybridize and discover related DNA sequences or to subtract out known sequences from a sample. The nucleic acid sequences can be further used to derive primers for genetic fingerprinting, to raise anti-polypeptide antibodies using DNA immunization techniques, and as an antigen to raise anti-DNA antibodies or elicit immune responses. Portions or fragments of the nucleotide sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Additionally, the nucleotide sequences of the invention can be used to identify and express recombinant polypeptides for analysis, characterization or therapeutic use, or as markers for tissues in which the corresponding polypeptide is expressed, either constitutively, during tissue differentiation, or in diseased states. The nucleic acid sequences can additionally be used as reagents in the screening and/or diagnostic assays described herein, and can also be included as components of kits (e.g., reagent kits) for use in the screening and/or diagnostic assays described herein.

Vectors

Another aspect of the invention pertains to nucleic acid constructs comprising a nucleic acid molecule of SEQ ID NO: 1 and comprising at least one polymorphism shown in Table 3 or FIGS. 5A-5C and the complements thereof (or a portion thereof). Yet another aspect of the invention pertains to nucleic acid constructs containing a nucleic acid molecule encoding the amino acid sequence of SEQ ID NO: 2-9 or polymorphic variants thereof. The constructs comprise a vector (e.g., an expression vector) into which a sequence of the invention has been inserted in a sense or antisense orientation. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids. However, the invention is intended to include such other forms of expression vectors, such as viral vectors (e.g., replication defective retroviruses, adenoviruses and adeno-associated viruses) that serve equivalent functions.

Preferred recombinant expression vectors of the invention comprise a nucleic acid molecule of the invention in a form suitable for expression of the nucleic acid molecule in a host cell. This means that the recombinant expression vectors include one or more regulatory sequences, selected on the basis of the host cells to be used for expression, which is operably linked to the nucleic acid sequence to be expressed. Within a recombinant expression vector, “operably or operatively linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed and the level of expression of polypeptide desired. The expression vectors of the invention can be introduced into host cells to thereby produce polypeptides, including fusion polypeptides, encoded by nucleic acid molecules as described herein.

The recombinant expression vectors of the invention can be designed for expression of a polypeptide of the invention in prokaryotic or eukaryotic cells, e.g., bacterial cells such as E. coli, insect cells (using baculovirus expression vectors), yeast cells or mammalian cells. Suitable host cells are discussed further in Goeddel (Gene Expression Technology: Methods in Enzymology 185, Academic Press, San Diego, Calif. (1990)). Alternatively, the recombinant expression vector can be transcribed and translated in vitro, for example, using T7 promoter regulatory sequences and T7 polymerase.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a nucleic acid molecule of the invention can be expressed in bacterial cells (e.g., E. coli), insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing a foreign nucleic acid molecule (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning, A Laboratory Manual (2nd Ed., CSHP, New York 1989)), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those that confer resistance to drugs, such as G418, hygromycin and methotrexate. Nucleic acid molecules encoding a selectable marker can be introduced into a host cell on the same vector as the nucleic acid molecule of the invention or can be introduced on a separate vector. Cells stably transfected with the introduced nucleic acid molecule can be identified by drug selection (e.g., cells that have incorporated the selectable marker gene will survive, while the other cells die).

A host cell of the invention, such as a prokaryotic or eukaryotic host cell in culture, can be used to produce (i.e., express) a polypeptide of the invention. Accordingly, the invention further provides methods for producing a polypeptide using the host cells of the invention. In one embodiment, the method comprises culturing the host cell of the invention (into which a recombinant expression vector encoding a polypeptide of the invention has been introduced) in a suitable medium such that the polypeptide is produced. In another embodiment, the method further comprises isolating the polypeptide from the medium or the host cell.

The host cells of the invention can also be used to produce nonhuman transgenic animals. For example, in one embodiment, a host cell of the invention is a fertilized oocyte or an embryonic stem cell into which a nucleic acid molecule of the invention has been introduced (e.g., an exogenous MATN3 gene, or an exogenous nucleic acid encoding MATN3 polypeptide). Such host cells can then be used to create non-human transgenic animals in which exogenous nucleotide sequences have been introduced into the genome or homologous recombinant animals in which endogenous nucleotide sequences have been altered. Such animals are useful for studying the function and/or activity of the nucleotide sequence and polypeptide encoded by the sequence and for identifying and/or evaluating modulators of their activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens and amphibians. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

Methods for generating transgenic animals via embryo manipulation and microinjection, particularly animals such as mice, have become conventional in the art and are described, for example, in U.S. Pat. Nos. 4,736,866, 4,870,009 and 4,873,191 and in Hogan, Manipulating the Mouse Embryo (Cold Spring Harbor Laboratory Press, Cold Spring Harbor, N.Y., 1986). Methods for constructing homologous recombination vectors and homologous recombinant animals are described further in Bradley (1991) Current Opinion in Bio/Technology 2:823-829 and in PCT Publication Nos. WO 90/11354, WO 91/01140, WO 92/0968 and WO 93/04169. Clones of the non-human transgenic animals described herein can also be produced according to the methods described in Wilmut et al. (1997) Nature 385:810-813 and PCT Publication Nos. WO 97/07668 and WO 97/07669.

Polypeptides of the Invention

The present invention also pertains to isolated polypeptides encoded by MATN3 (“MATN3 polypeptides”) and fragments and variants thereof, as well as polypeptides encoded by nucleotide sequences described herein (e.g., other splicing variants). The term “polypeptide” refers to a polymer of amino acids, and not to a specific length; thus, peptides, oligopeptides and proteins are included within the definition of a polypeptide. As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell (e.g., in a “fusion protein”) and still be “isolated” or “purified.”

The polypeptides of the invention can be purified to homogeneity. It is understood, however, that preparations in which the polypeptide is not purified to homogeneity are useful. The critical feature is that the preparation allows for the desired function of the polypeptide, even in the presence of considerable amounts of other components. Thus, the invention encompasses various degrees of purity. In one embodiment, the language “substantially free of cellular material” includes preparations of the polypeptide having less than about 30% (by dry weight) other proteins (i.e., contaminating protein), less than about 20% other proteins, less than about 10% other proteins, or less than about 5% other proteins.

When a polypeptide is recombinantly produced, it can also be substantially free of culture medium, i.e., culture medium represents less than about 20%, less than about 10%, or less than about 5% of the volume of the polypeptide preparation. The language “substantially free of chemical precursors or other chemicals” includes preparations of the polypeptide in which it is separated from chemical precursors or other chemicals that are involved in its synthesis. In one embodiment, the language “substantially free of chemical precursors or other chemicals” includes preparations of the polypeptide having less than about 30% (by dry weight) chemical precursors or other chemicals, less than about 20% chemical precursors or other chemicals, less than about 10% chemical precursors or other chemicals, or less than about 5% chemical precursors or other chemicals.

In one embodiment, a polypeptide of the invention comprises an amino acid sequence encoded by a nucleic acid molecule comprising a nucleotide sequence selected from the group consisting of SEQ ID NO: 1 comprising at least one polymorphism shown in Table 3 or FIGS. 5A-5C and complements and portions thereof, e.g., SEQ ID NOs: 13-20, or a portion or polymorphic variant thereof. However, the polypeptides of the invention also encompass fragment and sequence variants. Variants include a substantially homologous polypeptide encoded by the same genetic locus in an organism, i.e., an allelic variant, as well as other splicing variants. Variants also encompass polypeptides derived from other genetic loci in an organism, but having substantial homology to a polypeptide encoded by a nucleic acid molecule comprising a nucleotide sequence selected from the group consisting of SEQ ID NO: 1 comprising at least one polymorphism shown in Table 3 or FIGS. 5A-5C and complements and portions thereof, or having substantial homology to a polypeptide encoded by a nucleic acid molecule comprising a nucleotide sequence selected from the group consisting of nucleotide sequences encoding SEQ ID NO: 1, or polymorphic variants thereof. Variants also include polypeptides substantially homologous or identical to these polypeptides but derived from another organism, i.e., an ortholog. Variants also include polypeptides that are substantially homologous or identical to these polypeptides that are produced by chemical synthesis. Variants also include polypeptides that are substantially homologous or identical to these polypeptides that are produced by recombinant methods.

As used herein, two polypeptides (or a region of the polypeptides) are substantially homologous or identical when the amino acid sequences are at least about 45-55%, in certain embodiments at least about 70-75%, and in other embodiments at least about 80-85%, and in other embodiments greater than about 90% or more homologous or identical (e.g., 95%). A substantially homologous amino acid sequence, according to the present invention, will be encoded by a nucleic acid molecule of the invention, or portion thereof, under stringent conditions as more particularly described above, or will be encoded by a nucleic acid molecule hybridizing to a nucleic acid sequence of the invention, portion thereof or polymorphic variant thereof, under stringent conditions as more particularly described thereof.

The invention also encompasses polypeptides having a lower degree of identity but having sufficient similarity so as to perform one or more of the same functions performed by a polypeptide encoded by a nucleic acid molecule of the invention. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe and Tyr. Guidance concerning which amino acid changes are likely to be phenotypically silent are found in Bowie et al. (1990) Science, 247:1306-1310.

A variant polypeptide can differ in amino acid sequence by one or more substitutions, deletions, insertions, inversions, fusions, and truncations or a combination of any of these. Further, variant polypeptides can be fully functional or can lack function in one or more activities. Fully functional variants typically contain only conservative variation or variation in non-critical residues or in non-critical regions. Functional variants can also contain substitution of similar amino acids that result in no change or an insignificant change in function. Alternatively, such substitutions may positively or negatively affect function to some degree. Non-functional variants typically contain one or more non-conservative amino acid substitutions, deletions, insertions, inversions, or truncation or a substitution, insertion, inversion, or deletion in a critical residue or critical region.

Amino acids that are essential for function can be identified by methods known in the art, such as site-directed mutagenesis or alanine-scanning mutagenesis (Cunningham et al. (1989) Science, 244:1081-1085). The latter procedure introduces single alanine mutations at every residue in the molecule. The resulting mutant molecules are then tested for biological activity in vitro, or in vitro proliferative activity. Sites that are critical for polypeptide activity can also be determined by structural analysis such as crystallization, nuclear magnetic resonance or photoaffinity labeling (Smith et al. (1992) J. Mol. Biol., 224:899-904; de Vos et al. (1992) Science, 255:306-312).

The invention also includes polypeptide fragments of the polypeptides of the invention. Fragments can be derived from a polypeptide encoded by a nucleic acid described herein. However, the invention also encompasses fragments of the variants of the polypeptides described herein. As used herein, a fragment comprises at least 6 contiguous amino acids. Useful fragments include those that retain one or more of the biological activities of the polypeptide as well as fragments that can be used as an immunogen to generate polypeptide-specific antibodies.

Biologically active fragments (peptides which are, for example, 6, 9, 12, 15, 16, 20, 30, 35, 36, 37, 38, 39, 40, 50, 100 or more amino acids in length) can comprise a domain, segment, or motif that has been identified by analysis of the polypeptide sequence using well-known methods, e.g., signal peptides, extracellular domains, one or more transmembrane segments or loops, ligand binding regions, zinc finger domains, DNA binding domains, acylation sites, glycosylation sites, or phosphorylation sites.

Fragments can be discrete (not fused to other amino acids or polypeptides) or can be within a larger polypeptide. Further, several fragments can be comprised within a single larger polypeptide. In one embodiment a fragment designed for expression in a host can have heterologous pre- and pro-polypeptide regions fused to the amino terminus of the polypeptide fragment and an additional region fused to the carboxyl terminus of the fragment.

The invention thus provides chimeric or fusion polypeptides. These comprise a polypeptide of the invention operatively linked to a heterologous protein or polypeptide having an amino acid sequence not substantially homologous to the polypeptide. “Operatively linked” indicates that the polypeptide and the heterologous protein are fused in-frame. The heterologous protein can be fused to the N-terminus or C-terminus of the polypeptide. In one embodiment the fusion polypeptide does not affect function of the polypeptide per se. For example, the fusion polypeptide can be a GST-fusion polypeptide in which the polypeptide sequences are fused to the C-terminus of the GST sequences. Other types of fusion polypeptides include, but are not limited to, enzymatic fusion polypeptides, for example, β-galactosidase fusions, yeast two-hybrid GAL fusions, poly-His fusions and Ig fusions. Such fusion polypeptides, particularly poly-His fusions, can facilitate the purification of recombinant polypeptide. In certain host cells (e.g., mammalian host cells), expression and/or secretion of a polypeptide can be increased by using a heterologous signal sequence. Therefore, in another embodiment, the fusion polypeptide contains a heterologous signal sequence at its N-terminus.

EP-A-O 464 533 discloses fusion proteins comprising various portions of immunoglobulin constant regions. The Fc is useful in therapy and diagnosis and thus results, for example, in improved pharmacokinetic properties (EP-A 0232 262). In drug discovery, for example, human proteins have been fused with Fc portions for the purpose of high-throughput screening assays to identify antagonists. Bennett et al. (1995) Journal of Molecular Recognition 8:52-58 and Johanson et al. (1995) The Journal of Biological Chemistry, 270(16):9459-9471. Thus, this invention also encompasses soluble fusion polypeptides containing a polypeptide of the invention and various portions of the constant regions of heavy or light chains of immunoglobulins of various subclass (IgG, IgM, IgA, IgE).

A chimeric or fusion polypeptide can be produced by standard recombinant DNA techniques. For example, DNA fragments coding for the different polypeptide sequences are ligated together in-frame in accordance with conventional techniques. In another embodiment, the fusion gene can be synthesized by conventional techniques including automated DNA synthesizers. Alternatively, PCR amplification of nucleic acid fragments can be carried out using anchor primers which give rise to complementary overhangs between two consecutive nucleic acid fragments which can subsequently be annealed and re-amplified to generate a chimeric nucleic acid sequence (see Ausubel et al., Current Protocols in Molecular Biology, 1992). Moreover, many expression vectors are commercially available that already encode a fusion moiety (e.g., a GST protein). A nucleic acid molecule encoding a polypeptide of the invention can be cloned into such an expression vector such that the fusion moiety is linked in-frame to the polypeptide.

The isolated polypeptide can be purified from cells that naturally express it, purified from cells that have been altered to express it (recombinant), or synthesized using known protein synthesis methods. In one embodiment, the polypeptide is produced by recombinant DNA techniques. For example, a nucleic acid molecule encoding the polypeptide is cloned into an expression vector, the expression vector introduced into a host cell and the polypeptide expressed in the host cell. The polypeptide can then be isolated from the cells by an appropriate purification scheme using standard protein purification techniques.

In general, polypeptides of the present invention can be used as a molecular weight marker on SDS-PAGE gels or on molecular sieve gel filtration columns using art-recognized methods. The polypeptides of the present invention can be used to raise antibodies or to elicit an immune response. The polypeptides can also be used as a reagent, e.g., a labeled reagent, in assays to quantitatively determine levels of the polypeptide or a molecule to which it binds (e.g., a receptor or a ligand) in biological fluids. The polypeptides can also be used as markers for cells or tissues in which the corresponding polypeptide is preferentially expressed, either constitutively, during tissue differentiation, or in a diseased state. The polypeptides can be used to isolate a corresponding binding agent, e.g., receptor or ligand, such as, for example, in an interaction trap assay, and to screen for peptide or small molecule antagonists or agonists of the binding interaction.

Antibodies of the Invention

Polyclonal and/or monoclonal antibodies that specifically bind one form of the gene product but not to the other form of the gene product are also provided. Antibodies are also provided that bind a portion of either the variant or the reference gene product that contains the polymorphic site or sites. The invention provides antibodies to the polypeptides and polypeptide fragments of the invention, e.g., having an amino acid sequence encoded by SEQ ID NO: 2, 3, 4, 5, 6, 7, 8 or 9, or a portion thereof, or having an amino acid sequence encoded by a nucleic acid molecule comprising all or a portion of SEQ ID NO: 1 and comprising at least one polymorphism shown in Table 3 or FIGS. 5A-5C. The term “antibody” as used herein refers to immunoglobulin molecules and immunologically active portions of immunoglobulin molecules, i.e., molecules that contain an antigen binding site that specifically binds an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al., Monoclonal Antibodies and Cancer Therapy, Alan R. Liss, Inc., pp. 77-96 (1985)) or trioma techniques. The technology for producing hybridomas is well known (see generally Current Protocols in Immunology, Coligan et al. (eds.) John Wiley & Sons, Inc., New York, N.Y. (1994)). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, Coligan et al. (eds.) John Wiley & Sons, Inc., New York, N.Y. (1994); Galfre et al. (1977) Nature 266:55052; R. H. Kenneth, in Monoclonal Antibodies: A New Dimension In Biological Analyses, Plenum Publishing Corp., New York, N.Y. (1980); and Lerner (1981) Yale J. Biol. Med. 54:387-402). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., (1991) Bio/Technology, 9:1370-1372; Hay et al., (1992) Hum. Antibod. Hybridomas, 3:81-85; Huse et al.,(1989) Science, 246:1275-1281; and Griffiths et al., (1993) EMBO J, 12:725-734.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In addition, human antibodies are within the scope of this invention. Such human antibodies can be produced, isolated and purified by techniques known to one skilled in the art and using standard methodologies.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin and aequorin, and examples of suitable radioactive material include ¹²⁵I, ¹³¹I, ³⁵S or ³H.

Diagnostic and Screening Assays of the Invention

The present invention also pertains to a method of diagnosing or aiding in the diagnosis of osteoarthritis associated with the presence of a variant form of the MATN3 gene or gene product in an individual. Diagnostic assays can be designed for assessing MATN3 gene expression, or for assessing activity of MATN3 polypeptides of the invention. In one embodiment, the assays are used in the context of a biological sample (e.g., blood, serum, cells, tissue, synovial fluid) to thereby determine whether an individual is afflicted with osteoarthritis, or is at risk for (has a predisposition for or a susceptibility to) developing osteoarthritis. The invention also provides for prognostic (or predictive) assays for determining whether an individual is susceptible to developing osteoarthritis. For example, alterations in nucleic acids can be assayed in a biological sample. Such assays can be used for prognostic or predictive purpose to thereby prophylactically treat an individual prior to the onset of symptoms associated with osteoarthritis. Another aspect of the invention pertains to assays for monitoring the influence of agents (e.g., drugs, compounds or other agents) on the expression or activity of polypeptides of the invention, as well as to assays for identifying agents which bind to MATN3 polypeptides. These and other assays and agents are described in further detail in the following sections.

Diagnostic Assays

The nucleic acids, probes, primers, polypeptides and antibodies described herein can be used in methods of diagnosis of a susceptibility to osteoarthritis as well as kits comprising same.

In one embodiment of the invention, diagnosis of a susceptibility to osteoarthritis is made by detecting a polymorphism in MATN3 as described herein. The polymorphism can be a change in MATN3, such as the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of the gene; duplication of all or a part of the gene; transposition of all or a part of the gene; or rearrangement of all or a part of the gene. More than one such change may be present in a single gene. Such sequence changes cause a difference in the polypeptide encoded by a MATN3 nucleic acid. For example, if the alteration is a frame shift mutation, the frame shift can result in a change in the encoded amino acids, and/or can result in the generation of a premature stop codon, causing generation of a truncated polypeptide. Alternatively, a polymorphism associated with a susceptibility to osteoarthritis can be a synonymous alteration in one or more nucleotides (i.e., an alteration that does not result in a change in the polypeptide encoded by a MATN3 nucleic acid). Such a polymorphism may alter splicing sites, affect the stability or transport of mRNA, or otherwise affect the transcription or translation of the gene. A MATN3 nucleic acid that has any of the alterations described above is referred to herein as an “altered nucleic acid.”

In a first method of diagnosing a susceptibility to osteoarthritis, hybridization methods, such as Southern analysis, Northern analysis, or in situ hybridizations, can be used (see Current Protocols in Molecular Biology, Ausubel, F. et al., eds., John Wiley & Sons, including all supplements through 1999). For example, a biological sample from a test subject (a “test sample”) of genomic DNA, RNA, or cDNA, is obtained from an individual suspected of having, being susceptible to or predisposed for, or carrying a defect for, osteoarthritis (the “test individual”). The individual can be an adult, child, or fetus. The test sample can be from any source which contains genomic DNA, such as a blood sample, sample of synovial fluid, sample of amniotic fluid, sample of cerebrospinal fluid, or tissue sample from skin, muscle, buccal or conjunctival mucosa, placenta, gastrointestinal tract or other organs. A test sample of DNA from fetal cells or tissue can be obtained by appropriate methods, such as by amniocentesis or chorionic villus sampling. The DNA, RNA, or cDNA sample is then examined to determine whether a polymorphism in MATN3 is present, and/or to determine which splicing variant(s) encoded by MATN3 is present. The presence of the polymorphism or splicing variant(s) can be indicated by hybridization of the gene in the genomic DNA, RNA, or cDNA to a nucleic acid probe. A “nucleic acid probe”, as used herein, can be a DNA probe or an RNA probe; the nucleic acid probe can contain at least one polymorphism in MATN3 or contains a nucleic acid encoding a particular splicing variant of MATN3. The probe can be any of the nucleic acid molecules described above (e.g., the gene or nucleic acid, a fragment, a vector comprising the gene, a probe or primer, etc.).

To diagnose a susceptibility to osteoarthritis, a hybridization sample is formed by contacting the test sample containing MATN3, with at least one nucleic acid probe. A preferred probe for detecting mRNA or genomic DNA is a labeled nucleic acid probe capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate mRNA or genomic DNA. For example, the nucleic acid probe can be all or a portion of SEQ ID NO: 1 and comprising at least one polymorphism shown in Table 3 or FIGS. 5A-5C, or the complements thereof, or a portion thereof; or can be a nucleic acid encoding a portion thereof. Other suitable probes for use in the diagnostic assays of the invention are described above (see e.g., probes and primers discussed under the heading, “Nucleic Acids of the Invention”).

The hybridization sample is maintained under conditions which are sufficient to allow specific hybridization of the nucleic acid probe to MATN3. “Specific hybridization”, as used herein, indicates exact hybridization (e.g., with no mismatches). Specific hybridization can be performed under high stringency conditions or moderate stringency conditions, for example, as described above. In a particularly preferred embodiment, the hybridization conditions for specific hybridization are high stringency.

Specific hybridization, if present, is then detected using standard methods. If specific hybridization occurs between the nucleic acid probe and MATN3 in the test sample, then MATN3 has the polymorphism, or is the splicing variant, that is present in the nucleic acid probe. More than one nucleic acid probe can also be used concurrently in this method. Specific hybridization of any one of the nucleic acid probes is indicative of a polymorphism in MATN3, or of the presence of a particular splicing variant encoding MATN3 and is therefore diagnostic for a susceptibility to osteoarthritis.

In Northern analysis (see Ausubel, F. et al., eds., “Current Protocols in Molecular Biology”, John Wiley & Sons, (1998)) the hybridization methods described above are used to identify the presence of a polymorphism or a particular splicing variant, associated with a susceptibility to osteoarthritis. For Northern analysis, a test sample of RNA is obtained from the individual by appropriate means. Specific hybridization of a nucleic acid probe, as described above, to RNA from the individual is indicative of a polymorphism in MATN3, or of the presence of a particular splicing variant encoded by MATN3, and is therefore diagnostic for a susceptibility to osteoarthritis.

For representative examples of use of nucleic acid probes, see, for example, U.S. Pat. Nos. 5,288,611 and 4,851,330.

Alternatively, a peptide nucleic acid (PNA) probe can be used instead of a nucleic acid probe in the hybridization methods described above. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P. E. et al. (1994) Bioconjugate Chemistry 5, American Chemical Society, p. 1). The PNA probe can be designed to specifically hybridize to a gene having a polymorphism associated with a susceptibility to osteoarthritis. Hybridization of the PNA probe to MATN3 is diagnostic for a susceptibility to osteoarthritis.

In another method of the invention, mutation analysis by restriction digestion can be used to detect an altered nucleic acid or gene, or genes containing a polymorphism(s), if the alteration or polymorphism in the gene results in the creation or elimination of a restriction site. A test sample containing genomic DNA is obtained from the individual. Polymerase chain reaction (PCR) can be used to amplify MATN3 (and, if necessary, the flanking sequences) in the test sample of genomic DNA from the test individual. RFLP analysis is conducted as described (see “Current Protocols in Molecular Biology”, John Wiley & Sons, (1998)). The digestion pattern of the relevant DNA fragment indicates the presence or absence of the mutation or polymorphism in MATN3, and therefore indicates the presence or absence of this susceptibility to osteoarthritis.

Sequence analysis can also be used to detect specific polymorphisms in MATN3. A test sample of DNA or RNA is obtained from the test individual. PCR or other appropriate methods can be used to amplify the gene, and/or its flanking sequences, if desired. The sequence of MATN3, or a fragment of the gene, or cDNA, or fragment of the cDNA, or mRNA, or fragment of the mRNA, is determined, using standard methods. The sequence of the gene, gene fragment, cDNA, cDNA fragment, mRNA, or mRNA fragment is compared with the known nucleic acid sequence of the gene, cDNA or mRNA, as appropriate. The presence of a polymorphism in MATN3 indicates that the individual has a susceptibility to osteoarthritis.

Allele-specific oligonucleotides can also be used to detect the presence of a polymorphism in MATN3, through the use of dot-blot hybridization of amplified oligonucleotides with allele-specific oligonucleotide (ASO) probes (see, for example, Saiki et al. (1986) Nature (London) 324:163-166). An “allele-specific oligonucleotide” (also referred to herein as an “allele-specific oligonucleotide probe”) is an oligonucleotide of approximately 10-50 base pairs, preferably approximately 15-30 base pairs, that specifically hybridizes to MATN3, and that contains a polymorphism associated with a susceptibility to osteoarthritis. An allele-specific oligonucleotide probe that is specific for particular polymorphisms in MATN3 can be prepared, using standard methods (see Ausubel, F. et al., eds., “Current Protocols in Molecular Biology”, John Wiley & Sons, (1998)). To identify polymorphisms in the gene that are associated with a susceptibility to osteoarthritis, a test sample of DNA is obtained from the individual. PCR can be used to amplify all or a fragment of MATN3, and its flanking sequences. The DNA containing the amplified MATN3 (or fragment of the gene) is dot-blotted, using standard methods (see Ausubel, F. et al., eds., “Current Protocols in Molecular Biology”, John Wiley & Sons, (1998)), and the blot is contacted with the oligonucleotide probe. The presence of specific hybridization of the probe to the amplified MATN3 is then detected. Specific hybridization of an allele-specific oligonucleotide probe to DNA from the individual is indicative of a polymorphism in MATN3, and is therefore indicative of a susceptibility to osteoarthritis.

The invention further provides allele-specific oligonucleotides that hybridize to the reference or variant allele of a gene or nucleic acid comprising a single nucleotide polymorphism or to the complement thereof. These oligonucleotides can be probes or primers.

An allele-specific primer hybridizes to a site on target DNA overlapping a polymorphism and only primes amplification of an allelic form to which the primer exhibits perfect complementarity. See Gibbs (1989) Nucleic Acids Res. 17:2427-2448. This primer is used in conjunction with a second primer, which hybridizes at a distal site. Amplification proceeds from the two primers, resulting in a detectable product, which indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

In another embodiment, arrays of oligonucleotide probes that are complementary to target nucleic acid sequence segments from an individual, can be used to identify polymorphisms in MATN3. For example, in one embodiment, an oligonucleotide array can be used. Oligonucleotide arrays typically comprise a plurality of different oligonucleotide probes that are coupled to a surface of a substrate in different known locations. These oligonucleotide arrays, also described as “Genechips.TM.,” have been generally described in the art, for example, U.S. Pat. No. 5,143,854 and PCT patent publication Nos. WO 90/15070 and 92/10092. These arrays can generally be produced using mechanical synthesis methods or light directed synthesis methods which incorporate a combination of photolithographic methods and solid phase oligonucleotide synthesis methods. See Fodor et al. (1991) Science 251:767-777, Pirrung et al., U.S. Pat. No. 5,143,854 (see also PCT Application No. WO 90/15070) and Fodor et al., PCT Publication No. WO 92/10092 and U.S. Pat. No. 5,424,186, the entire teachings of each of which are incorporated by reference herein. Techniques for the synthesis of these arrays using mechanical synthesis methods are described in, e.g., U.S. Pat. No. 5,384,261, the entire teachings of which are incorporated by reference herein. In another example, linear arrays can be utilized.

Once an oligonucleotide array is prepared, a nucleic acid of interest is hybridized with the array and scanned for polymorphisms. Hybridization and scanning are generally carried out by methods described herein and also in, e.g., Published PCT Application Nos. WO 92/10092 and WO 95/11995 and U.S. Pat. No. 5,424,186, the entire teachings of which are incorporated by reference herein. In brief, a target nucleic acid sequence which includes one or more previously identified polymorphic markers is amplified by well known amplification techniques, e.g., PCR. Typically, this involves the use of primer sequences that are complementary to the two strands of the target sequence both upstream and downstream from the polymorphism. Asymmetric PCR techniques may also be used. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Although primarily described in terms of a single detection block, e.g., for detection of a single polymorphism, arrays can include multiple detection blocks, and thus be capable of analyzing multiple, specific polymorphisms. In alternate arrangements, it will generally be understood that detection blocks may be grouped within a single array or in multiple, separate arrays so that varying, optimal conditions may be used during the hybridization of the target to the array. For example, it may often be desirable to provide for the detection of those polymorphisms that fall within G-C rich stretches of a genomic sequence, separately from those falling in A-T rich segments. This allows for the separate optimization of hybridization conditions for each situation.

Additional description of use of oligonucleotide arrays for detection of polymorphisms can be found, for example, in U.S. Pat. Nos. 5,858,659 and 5,837,832, the entire teachings of which are incorporated by reference herein.

Other methods of nucleic acid analysis can be used to detect polymorphisms in MATN3 or splicing variants encoding by MATN3. Representative methods include direct manual sequencing (Church and Gilbert (1988) Proc. Natl. Acad. Sci. USA 81:1991-1995; Sanger et al. (1977) Proc. Natl. Acad. Sci. 74:5463-5467; Beavis et al. U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield et al. (1989) Proc. Natl. Acad. Sci. USA 86:232-236), mobility shift analysis (Orita et al. (1989) Proc. Natl. Acad. Sci. USA 86:2766-2770), restriction enzyme analysis (Flavell et al. (1978) Cell 15:25; Geever et al. (1981) Proc. Natl. Acad. Sci. USA 78:5081); heteroduplex analysis; chemical mismatch cleavage (CMC) (Cotton et al. (1985) Proc. Natl. Acad. Sci. USA 85:4397-4401); RNase protection assays (Myers et al. (1985) Science 230:1242); use of polypeptides which recognize nucleotide mismatches, such as E. coli mutS protein; and allele-specific PCR.

In one embodiment of the invention, diagnosis of a disease or condition associated with a MATN3 nucleic acid (e.g., osteoarthritis) or a susceptibility to a disease or condition associated with a MATN3 nucleic acid (e.g., osteoarthritis) can also be made by expression analysis by quantitative PCR (kinetic thermal cycling). This technique, utilizing TaqMan®, can be used to allow the identification of polymorphisms and whether a patient is homozygous or heterozygous. The technique can assess the presence of an alteration in the expression or composition of the polypeptide encoded by a MATN3 nucleic acid or splicing variants encoded by a MATN3 nucleic acid. Further, the expression of the variants can be quantified as physically or functionally different.

In another embodiment of the invention, diagnosis of a susceptibility to osteoarthritis can also be made by examining expression and/or composition of a MATN3 polypeptide, by a variety of methods, including enzyme linked immunosorbent assays (ELISAs), Western blots, immunoprecipitations and immunofluorescence. A test sample from an individual is assessed for the presence of an alteration in the expression and/or an alteration in composition of the polypeptide encoded by MATN3, or for the presence of a particular variant encoded by MATN3. An alteration in expression of a polypeptide encoded by MATN3 can be, for example, an alteration in the quantitative polypeptide expression (i.e., the amount of polypeptide produced); an alteration in the composition of a polypeptide encoded by MATN3 is an alteration in the qualitative polypeptide expression (e.g., expression of a mutant MATN3 polypeptide or of a different splicing variant). In a preferred embodiment, diagnosis of a susceptibility to osteoarthritis is made by detecting a particular splicing variant encoded by MATN3, or a particular pattern of splicing variants.

Both such alterations (quantitative and qualitative) can also be present. An “alteration” in the polypeptide expression or composition, as used herein, refers to an alteration in expression or composition in a test sample, as compared with the expression or composition of polypeptide by MATN3 in a control sample. A control sample is a sample that corresponds to the test sample (e.g., is from the same type of cells), and is from an individual who is not affected by osteoarthritis. An alteration in the expression or composition of the polypeptide in the test sample, as compared with the control sample, is indicative of a susceptibility to osteoarthritis. Similarly, the presence of one or more different splicing variants in the test sample, or the presence of significantly different amounts of different splicing variants in the test sample, as compared with the control sample, is indicative of a susceptibility to osteoarthritis. Various means of examining expression or composition of the polypeptide encoded by MATN3 can be used, including spectroscopy, colorimetry, electrophoresis, isoelectric focusing, and immunoassays (e.g., David et al., U.S. Pat. No. 4,376,110) such as immunoblotting (see also Current Protocols in Molecular Biology, particularly chapter 10). For example, in one embodiment, an antibody capable of binding to the polypeptide (e.g., as described above), preferably an antibody with a detectable label, can be used. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Western blotting analysis, using an antibody as described above that specifically binds to a polypeptide encoded by a mutant MATN3, or an antibody that specifically binds to a polypeptide encoded by a non-mutant gene, or an antibody that specifically binds to a particular splicing variant encoded by MATN3, can be used to identify the presence in a test sample of a particular splicing variant or of a polypeptide encoded by a polymorphic or mutant MATN3, or the absence in a test sample of a particular splicing variant or of a polypeptide encoded by a non-polymorphic or non-mutant gene. The presence of a polypeptide encoded by a polymorphic or mutant gene, or the absence of a polypeptide encoded by a non-polymorphic or non-mutant gene, is diagnostic for a susceptibility to osteoarthritis, as is the presence (or absence) of particular splicing variants encoded by the MATN3 nucleic acid.

In one embodiment of this method, the level or amount of polypeptide encoded by MATN3 in a test sample is compared with the level or amount of the polypeptide encoded by MATN3 in a control sample. A level or amount of the polypeptide in the test sample that is higher or lower than the level or amount of the polypeptide in the control sample, such that the difference is statistically significant, is indicative of an alteration in the expression of the polypeptide encoded by MATN3, and is diagnostic for a susceptibility to osteoarthritis. Alternatively, the composition of the polypeptide encoded by MATN3 in a test sample is compared with the composition of the polypeptide encoded by MATN3 in a control sample (e.g., the presence of different splicing variants). A difference in the composition of the polypeptide in the test sample, as compared with the composition of the polypeptide in the control sample, is diagnostic for a susceptibility to osteoarthritis. In another embodiment, both the level or amount and the composition of the polypeptide can be assessed in the test sample and in the control sample. A difference in the amount or level of the polypeptide in the test sample, compared to the control sample; a difference in composition in the test sample, compared to the control sample; or both a difference in the amount or level, and a difference in the composition, is indicative of a susceptibility to osteoarthritis.

The invention also pertains to methods of diagnosing a susceptibility to osteoarthritis in an individual, comprising screening for an at-risk haplotype in the MATN3 gene that is more frequently present in an individual susceptible to osteoarthritis (affected), compared to the frequency of its presence in a healthy individual (control), wherein the presence of the haplotype is indicative of susceptibility to osteoarthritis. Standard techniques for genotyping for the presence of SNPs and/or microsatellite markers that are associated with osteoarthritis can be used, such as fluorescent based techniques (Chen et al. (1999) Genome Res. 9:492), PCR, LCR, Nested PCR, kinetic thermal cycling to determine whether the patient is heterozygous or homozygous for a particular genotype and other techniques for nucleic acid amplification. In a preferred embodiment, the method comprises assessing in an individual the presence or frequency of SNPs and/or microsatellites in the MATN3 gene that are associated with osteoarthritis, wherein an excess or higher frequency of the SNPs and/or microsatellites compared to a healthy control individual is indicative that the individual is susceptible to osteoarthritis.

See Table 3, FIGS. 5A-5C, Table 6 and Table 7 for SNPs and markers that comprise haplotypes that can be used as screening tools. SNPs and markers from these lists represent at-risk haplotypes and can be used to design diagnostic tests for determining a susceptibility to osteoarthritis.

In one embodiment, the at-risk haplotype is characterized by the presence of the polymorphism(s) represented by one or a combination of single nucleotide polymorphisms at nucleic acid positions 58162, 57927, 56822, 47929, 45434, 45317, 45178 and 45010, relative to SEQ ID NO: 1. In another embodiment, a diagnostic method for susceptibility to osteoarthritis can comprise determining the presence of at-risk haplotype represented by one or a combination of single nucleotide polymorphisms and microsatellie markers at nucleic acid positions 58162, 57927, 56822, 47929, 45434, 45317, 45178 and 45010, relative to SEQ ID NO: 1.

Kits (e.g., reagent kits) useful in the methods of diagnosis comprise components useful in any of the methods described herein, including, for example, hybridization probes or primers as described herein (e.g., labeled probes or primers), reagents for detection of labeled molecules, restriction enzymes (e.g., for RFLP analysis), allele-specific oligonucleotides, antibodies which bind to altered or to non-altered (native) MATN3 polypeptide, means for amplification of nucleic acids comprising MATN3, or means for analyzing the nucleic acid sequence of MATN3 or for analyzing the amino acid sequence of a MATN3 polypeptide, etc. In one embodiment, a kit for diagnosing susceptibility to osteoarthritis can comprise primers for nucleic acid amplification of a region in the MATN3 gene comprising an at-risk haplotype that is more frequently present in an individual susceptible to osteoarthritis. The primers can be designed using portions of the nucleic acids flanking SNPs that are indicative of osteoarthritis. In a particularly preferred embodiment, the primers are designed to amplify regions of the MATN3 gene associated with an at-risk haplotype for osteoarthritis, shown in Tables 6 and 7. In another embodiment of the invention, a kit for diagnosing susceptibility to osteoarthritis can further comprise probes designed to hybridize to regions of the MATN3 gene associated with an at-risk haplotype for osteoarthritis, shown in, for example, Table 6 and Table 7. The at risk haplotype can be characterized, for example, by the presence of at least one single nucleotide polymorphism at nucleic acid positions 58162, 57927, 56822, 47929, 45434, 45317, 45178 and 45010, relative to SEQ ID NO: 1.

In another embodiment of the invention, a method for the diagnosis and identification of susceptibility to osteoarthritis in an individual is provided by identifying an at-risk haplotype in MATN3. In one embodiment, the at-risk haplotype is a haplotype for which the presence of the haplotype increases the risk of osteoarthritis significantly. Although it is to be understood that identifying whether a risk is significant may depend on a variety of factors, including the specific disease, the haplotype, and often, environmental factors, the significance may be measured by an odds ratio or a percentage. In one embodiment, a significant risk is measured as an odds ratio of at least about 1.1, including but not limited to: 1.2, 1.3, 1.4, 1.5, 1.6, 1.7, 1.8 and 1.9. In a further embodiment, an odds ratio of at least 1.2 is significant. In a further embodiment, an odds ratio of at least about 1.5 is significant. In another embodiment, an odds ratio of at least about 1.7 is significant. In a further embodiment, a significant increase in risk is at least about 20%, including but not limited to: 25%, 30%, 35%, 40%, 45%, 50%, 55%, 60%, 65%, 70%, 75%, 80%, 85%, 90%, 95% and 98%.

Screening Assays and Agents Identified Thereby

The invention provides methods (also referred to herein as “screening assays”) for identifying the presence of a nucleotide that hybridizes to a nucleic acid of the invention, as well as for identifying the presence of a polypeptide encoded by a nucleic acid of the invention. In one embodiment, the presence (or absence) of a nucleic acid molecule of interest (e.g., a nucleic acid that has significant homology with a nucleic acid of the invention) in a sample can be assessed by contacting the sample with a nucleic acid comprising a nucleic acid of the invention (e.g., a nucleic acid having the sequence of SEQ ID NO: 1 and comprising at least one polymorphism shown in Table 3 or FIGS. 5A-5C, or the complements thereof, or a nucleic acid encoding an amino acid having the sequence of SEQ ID NO: 2, 3, 4, 5, 6, 7, 8 or 9, or a fragment or variant of such nucleic acids), under stringent conditions as described above, and then assessing the sample for the presence (or absence) of hybridization. In a preferred embodiment, high stringency conditions are conditions appropriate for selective hybridization. In another embodiment, a sample containing the nucleic acid molecule of interest is contacted with a nucleic acid containing a contiguous nucleotide sequence (e.g., a primer or a probe as described above) that is at least partially complementary to a part of the nucleic acid molecule of interest (e.g., a MATN3 nucleic acid), and the contacted sample is assessed for the presence or absence of hybridization. In a preferred embodiment, the nucleic acid containing a contiguous nucleotide sequence is completely complementary to a part of the nucleic acid molecule of interest.

In any of these embodiments, all or a portion of the nucleic acid of interest can be subjected to amplification prior to performing the hybridization.

In another embodiment, the presence (or absence) of a polypeptide of interest, such as a polypeptide of the invention or a fragment or variant thereof, in a sample can be assessed by contacting the sample with an antibody that specifically binds to the polypeptide of interest (e.g., an antibody such as those described above), and then assessing the sample for the presence (or absence) of binding of the antibody to the polypeptide of interest.

In another embodiment, the invention provides methods for identifying agents (e.g., fusion proteins, polypeptides, peptidomimetics, prodrugs, receptors, binding agents, antibodies, small molecules or other drugs, or ribozymes which alter (e.g., increase or decrease) the activity of the polypeptides described herein, or which otherwise interact with the polypeptides herein. For example, such agents can be agents which bind to polypeptides described herein (e.g., MATN3 binding agents); which have a stimulatory or inhibitory effect on, for example, activity of polypeptides of the invention; or which change (e.g., enhance or inhibit) the ability of the polypeptides of the invention to interact with MATN3 binding agents (e.g., receptors or other binding agents); or which alter posttranslational processing of the MATN3 polypeptide (e.g., agents that alter proteolytic processing to direct the polypeptide from where it is normally synthesized to another location in the cell, such as the cell surface; agents that alter proteolytic processing such that more polypeptide is released from the cell, etc.).

In one embodiment, the invention provides assays for screening candidate or test agents that bind to or modulate the activity of polypeptides described herein (or biologically active portion(s) thereof), as well as agents identifiable by the assays. Test agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to polypeptide libraries, while the other four approaches are applicable to polypeptide, non-peptide oligomer or small molecule libraries of compounds (Lam, K. S. (1997) Anticancer Drug Des. 12:145).

In one embodiment, to identify agents which alter the activity of a MATN3 polypeptide, a cell, cell lysate, or solution containing or expressing a MATN3 polypeptide (e.g., SEQ ID NO: 2, 3, 4, 5, 6, 7, 8 or 9, or another splicing variant encoded by MATN3), or a fragment or derivative thereof (as described above), can be contacted with an agent to be tested; alternatively, the polypeptide can be contacted directly with the agent to be tested. The level (amount) of MATN3 activity is assessed (e.g., the level (amount) of MATN3 activity is measured, either directly or indirectly), and is compared with the level of activity in a control (i.e., the level of activity of the MATN3 polypeptide or active fragment or derivative thereof in the absence of the agent to be tested). If the level of the activity in the presence of the agent differs, by an amount that is statistically significant, from the level of the activity in the absence of the agent, then the agent is an agent that alters the activity of MATN3 polypeptide. An increase in the level of MATN3 activity relative to a control, indicates that the agent is an agent that enhances (is an agonist of) MATN3 activity. Similarly, a decrease in the level of MATN3 activity relative to a control, indicates that the agent is an agent that inhibits (is an antagonist of) MATN3 activity. In another embodiment, the level of activity of a MATN3 polypeptide or derivative or fragment thereof in the presence of the agent to be tested, is compared with a control level that has previously been established. A level of the activity in the presence of the agent that differs from the control level by an amount that is statistically significant indicates that the agent alters MATN3 activity.

The present invention also relates to an assay for identifying agents which alter the expression of the MATN3 nucleic acid (e.g., antisense nucleic acids, fusion proteins, polypeptides, peptidomimetics, prodrugs, receptors, binding agents, antibodies, small molecules or other drugs, or ribozymes) which alter (e.g., increase or decrease) expression (e.g., transcription or translation) of the gene or which otherwise interact with the nucleic acids described herein, as well as agents identifiable by the assays. For example, a solution containing a nucleic acid encoding MATN3 polypeptide (e.g., MATN3 nucleic acid) can be contacted with an agent to be tested. The solution can comprise, for example, cells containing the nucleic acid or cell lysate containing the nucleic acid; alternatively, the solution can be another solution which comprises elements necessary for transcription/translation of the nucleic acid. Cells not suspended in solution can also be employed, if desired. The level and/or pattern of MATN3 expression (e.g., the level and/or pattern of mRNA or of protein expressed, such as the level and/or pattern of different splicing variants) is assessed, and is compared with the level and/or pattern of expression in a control (i.e., the level and/or pattern of the MATN3 expression in the absence of the agent to be tested). If the level and/or pattern in the presence of the agent differs, by an amount or in a manner that is statistically significant, from the level and/or pattern in the absence of the agent, then the agent is an agent that alters the expression of MATN3. Enhancement of MATN3 expression indicates that the agent is an agonist of MATN3 activity. Similarly, inhibition of MATN3 expression indicates that the agent is an antagonist of MATN3 activity. In another embodiment, the level and/or pattern of MATN3 polypeptide(s) (e.g., different splicing variants) in the presence of the agent to be tested, is compared with a control level and/or pattern that has previously been established. A level and/or pattern in the presence of the agent that differs from the control level and/or pattern by an amount or in a manner that is statistically significant indicates that the agent alters MATN3 expression.

In another embodiment of the invention, agents which alter the expression of the MATN3 nucleic acid or which otherwise interact with the nucleic acids described herein, can be identified using a cell, cell lysate, or solution containing a nucleic acid encoding the promoter region of the MATN3 gene operably linked to a reporter gene. After contact with an agent to be tested, the level of expression of the reporter gene (e.g., the level of mRNA or of protein expressed) is assessed, and is compared with the level of expression in a control (i.e., the level of the expression of the reporter gene in the absence of the agent to be tested). If the level in the presence of the agent differs, by an amount or in a manner that is statistically significant, from the level in the absence of the agent, then the agent is an agent that alters the expression of MATN3, as indicated by its ability to alter expression of a gene that is operably linked to the MATN3 gene promoter. Enhancement of the expression of the reporter indicates that the agent is an agonist of MATN3 activity. Similarly, inhibition of the expression of the reporter indicates that the agent is an antagonist of MATN3 activity. In another embodiment, the level of expression of the reporter in the presence of the agent to be tested, is compared with a control level that has previously been established. A level in the presence of the agent that differs from the control level by an amount or in a manner that is statistically significant indicates that the agent alters MATN3 expression.

Agents which alter the amounts of different splicing variants encoded by MATN3 (e.g., an agent which enhances activity of a first splicing variant, and which inhibits activity of a second splicing variant), as well as agents which are agonists of activity of a first splicing variant and antagonists of activity of a second splicing variant, can easily be identified using these methods described above.

In other embodiments of the invention, assays can be used to assess the impact of a test agent on the activity of a polypeptide in relation to a MATN3 binding agent. For example, a cell that expresses a compound that interacts with MATN3 (herein referred to as a “MATN3 binding agent”, which can be a polypeptide or other molecule that interacts with MATN3, such as a receptor) is contacted with MATN3 in the presence of a test agent, and the ability of the test agent to alter the interaction between MATN3 and the MATN3 binding agent is determined. Alternatively, a cell lysate or a solution containing the MATN3 binding agent, can be used. An agent which binds to MATN3 or the MATN3 binding agent can alter the interaction by interfering with, or enhancing the ability of MATN3 to bind to, associate with, or otherwise interact with the MATN3 binding agent. Determining the ability of the test agent to bind to MATN3 or a MATN3 binding agent can be accomplished, for example, by coupling the test agent with a radioisotope or enzymatic label such that binding of the test agent to the polypeptide can be determined by detecting the labeled with ¹²⁵I, ³⁵S, ¹⁴C or ³H, either directly or indirectly, and the radioisotope detected by direct counting of radioemmission or by scintillation counting. Alternatively, test agents can be enzymatically labeled with, for example, horseradish peroxidase, alkaline phosphatase, or luciferase, and the enzymatic label detected by determination of conversion of an appropriate substrate to product. It is also within the scope of this invention to determine the ability of a test agent to interact with the polypeptide without the labeling of any of the interactants. For example, a microphysiometer can be used to detect the interaction of a test agent with MATN3 or a MATN3 binding agent without the labeling of either the test agent, MATN3, or the MATN3 binding agent. McConnell, H. M. et al. (1992) Science 257:1906-1912. As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between ligand and polypeptide. See the Exemplification Section for a discussion of known MATN3 binding partners. Thus, these receptors can be used to screen for compounds that are MATN3 receptor agonists for use in treating osteoarthritis or MATN3 receptor antagonists for studying osteoarthritis. The linkage data provided herein, for the first time, provides such correction to osteoarthritis. Drugs could be designed to regulate MATN3 receptor activation which in turn can be used to regulate signaling pathways and transcription events of genes downstream, such as Cbfa1.

In another embodiment of the invention, assays can be used to identify polypeptides that interact with one or more MATN3 polypeptides, as described herein. For example, a yeast two-hybrid system such as that described by Fields and Song (Fields and Song (1989) Nature 340:245-246) can be used to identify polypeptides that interact with one or more MATN3 polypeptides. In such a yeast two-hybrid system, vectors are constructed based on the flexibility of a transcription factor which has two functional domains (a DNA binding domain and a transcription activation domain). If the two domains are separated but fused to two different proteins that interact with one another, transcriptional activation can be achieved, and transcription of specific markers (e.g., nutritional markers such as His and Ade, or color markers such as lacZ) can be used to identify the presence of interaction and transcriptional activation. For example, in the methods of the invention, a first vector is used which includes a nucleic acid encoding a DNA binding domain and also a MATN3 polypeptide, splicing variant, or fragment or derivative thereof, and a second vector is used which includes a nucleic acid encoding a transcription activation domain and also a nucleic acid encoding a polypeptide which potentially may interact with the MATN3 polypeptide, splicing variant, or fragment or derivative thereof (e.g., a MATN3 polypeptide binding agent or receptor). Incubation of yeast containing the first vector and the second vector under appropriate conditions (e.g., mating conditions such as used in the Matchmaker™ system from Clontech) allows identification of colonies which express the markers of interest. These colonies can be examined to identify the polypeptide(s) which interact with the MATN3 polypeptide or fragment or derivative thereof. Such polypeptides may be useful as agents which alter the activity of expression of a MATN3 polypeptide, as described above.

In more than one embodiment of the above assay methods of the present invention, it may be desirable to immobilize either MATN3, the MATN3 binding agent, or other components of the assay on a solid support, in order to facilitate separation of complexed from uncomplexed forms of one or both of the polypeptides, as well as to accommodate automation of the assay. Binding of a test agent to the polypeptide, or interaction of the polypeptide with a binding agent in the presence and absence of a test agent, can be accomplished in any vessel suitable for containing the reactants. Examples of such vessels include microtitre plates, test tubes, and micro-centrifuge tubes. In one embodiment, a fusion protein (e.g., a glutathione-S-transferase fusion protein) can be provided which adds a domain that allows MATN3 or a MATN3 binding agent to be bound to a matrix or other solid support.

In another embodiment, modulators of expression of nucleic acid molecules of the invention are identified in a method wherein a cell, cell lysate, or solution containing a nucleic acid encoding MATN3 is contacted with a test agent and the expression of appropriate mRNA or polypeptide (e.g., splicing variant(s)) in the cell, cell lysate, or solution, is determined. The level of expression of appropriate mRNA or polypeptide(s) in the presence of the test agent is compared to the level of expression of mRNA or polypeptide(s) in the absence of the test agent. The test agent can then be identified as a modulator of expression based on this comparison. For example, when expression of mRNA or polypeptide is greater (statistically significantly greater) in the presence of the test agent than in its absence, the test agent is identified as a stimulator or enhancer of the mRNA or polypeptide expression. Alternatively, when expression of the mRNA or polypeptide is less (statistically significantly less) in the presence of the test agent than in its absence, the test agent is identified as an inhibitor of the mRNA or polypeptide expression. The level of mRNA or polypeptide expression in the cells can be determined by methods described herein for detecting mRNA or polypeptide.

This invention further pertains to novel agents identified by the above-described screening assays. Accordingly, it is within the scope of this invention to further use an agent identified as described herein in an appropriate animal model. For example, an agent identified as described herein (e.g., a test agent that is a modulating agent, an antisense nucleic acid molecule, a specific antibody, or a polypeptide-binding agent) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein. In addition, an agent identified as described herein can be used to alter activity of a polypeptide encoded by MATN3, or to alter expression of MATN3, by contacting the polypeptide or the gene (or contacting a cell comprising the polypeptide or the gene) with the agent identified as described herein.

Pharmaceutical Compositions

The present invention also pertains to pharmaceutical compositions comprising nucleic acids described herein, particularly nucleotides encoding the polypeptides described herein; comprising the normal (not associated with osteoarthritis) MATN 3 gene product, polypeptides described herein (e.g., one or more of SEQ ID NO: 2, 3, 4, 5, 6, 7, 8 or 9); and/or comprising other splicing variants encoded by MATN3; and/or an agent that alters (e.g., enhances or inhibits) MATN3 gene expression or MATN3 polypeptide activity as described herein. For instance, a polypeptide, protein (e.g., a MATN3 receptor), an agent that alters MATN3 gene expression, or a MATN3 binding agent or binding partner, fragment, fusion protein or prodrug thereof, or a nucleotide or nucleic acid construct (vector) comprising a nucleotide of the present invention, or an agent that alters MATN3 polypeptide activity, can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyro-lidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devices (“gene guns”) and slow release polymeric devices. The pharmaceutical compositions of this invention can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, non-sprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms of osteoarthritis, and should be decided according to the judgment of a practitioner and each patient's circumstances. Effective doses may be extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the agents can be separated, mixed together in any combination, present in a single vial or tablet. Agents assembled in a blister pack or other dispensing means is preferred. For the purpose of this invention, unit dosage is intended to mean a dosage that is dependent on the individual pharmacodynamics of each agent and administered in FDA approved dosages in standard time courses.

Methods of Therapy

The present invention also pertains to methods of treatment (prophylactic and/or therapeutic) for osteoarthritis using a MATN3 therapeutic agent. A “MATN3 therapeutic agent” is an agent that alters (e.g., enhances or inhibits) MATN3 polypeptide activity and/or MATN3 nucleic acid expression, as described herein (e.g., a MATN3 agonist or antagonist). MATN3 therapeutic agents can alter MATN3 polypeptide activity or gene expression by a variety of means, such as, for example, by providing additional MATN3 polypeptide or by upregulating the transcription or translation of the MATN3 nucleic acid; by altering posttranslational processing of the MATN3 polypeptide; by altering transcription of MATN3 splicing variants; or by interfering with MATN3 polypeptide activity (e.g., by binding to a MATN3 polypeptide), or by downregulating the transcription or translation of the MATN3 nucleic acid. Representative MATN3 therapeutic agents include the following: nucleic acids or fragments or derivatives thereof described herein, particularly nucleotides encoding the polypeptides described herein and vectors comprising such nucleic acids (e.g., a gene, cDNA, and/or mRNA, such as a nucleic acid encoding a MATN3 polypeptide or active fragment or derivative thereof, or an oligonucleotide; for example, non-altered MATN3); polypeptides described herein (e.g., non-altered MATN3); other polypeptides (e.g., MATN3 receptors); MATN3 binding agents; peptidomimetics; fusion proteins or prodrugs thereof, antibodies (e.g., an antibody to an altered MATN3 polypeptide, or an antibody to a non-altered MATN3 polypeptide, or an antibody to a particular splicing variant encoded by MATN3, as described above); ribozymes; other small molecules; and other agents that alter (e.g., enhance or inhibit) MATN3 gene expression or polypeptide activity, or that regulate transcription of MATN3 splicing variants (e.g., agents that affect which splicing variants are expressed, or that affect the amount of each splicing variant that is expressed.

More than one MATN3 therapeutic agent can be used concurrently, if desired.

The MATN3 therapeutic agent that is a nucleic acid is used in the treatment of osteoarthritis. The term, “treatment” as used herein, refers not only to ameliorating symptoms associated with the disease, but also preventing or delaying the onset of the disease, and also lessening the severity or frequency of symptoms of the disease. The therapy is designed to alter (e.g., inhibit or enhance), replace or supplement activity of a MATN3 polypeptide in an individual. For example, a MATN3 therapeutic agent can be administered in order to upregulate or increase the expression or availability of the MATN3 gene or of specific splicing variants of MATN3, or, conversely, to downregulate or decrease the expression or availability of the MATN3 gene or specific splicing variants of MATN3. Upregulation or increasing expression or availability of a native MATN3 gene or of a particular splicing variant could interfere with or compensate for the expression or activity of a defective gene or another splicing variant; downregulation or decreasing expression or availability of a native MATN3 gene or of a particular splicing variant could minimize the expression or activity of a defective gene or the particular splicing variant and thereby minimize the impact of the defective gene or the particular splicing variant.

The MATN3 therapeutic agent(s) are administered in a therapeutically effective amount (i.e., an amount that is sufficient to treat the disease, such as by ameliorating symptoms associated with the disease, preventing or delaying the onset of the disease, and/or also lessening the severity or frequency of symptoms of the disease). The amount which will be therapeutically effective in the treatment of a particular individual's disorder or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

In one embodiment, a nucleic acid of the invention (e.g., a nucleic acid encoding a MATN3 polypeptide, such as SEQ ID NO: 1 or another nucleic acid that encodes a MATN3 polypeptide, derivative or fragment thereof) can be used, either alone or in a pharmaceutical composition as described above. For example, MATN3 or a cDNA encoding the MATN3 polypeptide, either by itself or included within a vector, can be introduced into cells (either in vitro or in vivo) such that the cells produce native MATN3 polypeptide. If necessary, cells that have been transformed with the gene or cDNA or a vector comprising the gene or CDNA can be introduced (or re-introduced) into an individual affected with the disease. Thus, cells which, in nature, lack native MATN3 expression and activity, or have mutant MATN3 expression and activity, or have expression of a disease-associated MATN3 splicing variant, can be engineered to express MATN3 polypeptide or an active fragment of the MATN3 polypeptide (or a different variant of MATN3 polypeptide). In a preferred embodiment, nucleic acid encoding the MATN3 polypeptide, or an active fragment or derivative thereof, can be introduced into an expression vector, such as a viral vector, and the vector can be introduced into appropriate cells in an animal. Other gene transfer systems, including viral and nonviral transfer systems, can be used. Alternatively, nonviral gene transfer methods, such as calcium phosphate coprecipitation, mechanical techniques (e.g., microinjection); membrane fusion-mediated transfer via liposomes; or direct DNA uptake, can also be used.

Alternatively, in another embodiment of the invention, a nucleic acid of the invention; a nucleic acid complementary to a nucleic acid of the invention; or a portion of such a nucleic acid (e.g., an oligonucleotide as described below), can be used in “antisense” therapy, in which a nucleic acid (e.g., an oligonucleotide) which specifically hybridizes to the mRNA and/or genomic DNA of MATN3 is administered or generated in situ. The antisense nucleic acid that specifically hybridizes to the mRNA and/or DNA inhibits expression of the MATN3 polypeptide, e.g., by inhibiting translation and/or transcription. Binding of the antisense nucleic acid can be by conventional base pair complementarity, or, for example, in the case of binding to DNA duplexes, through specific interaction in the major groove of the double helix.

An antisense construct of the present invention can be delivered, for example, as an expression plasmid as described above. When the plasmid is transcribed in the cell, it produces RNA which is complementary to a portion of the mRNA and/or DNA which encodes MATN3 polypeptide. Alternatively, the antisense construct can be an oligonucleotide probe which is generated ex vivo and introduced into cells; it then inhibits expression by hybridizing with the mRNA and/or genomic DNA of MATN3. In one embodiment, the oligonucleotide probes are modified oligonucleotides which are resistant to endogenous nucleases, e.g., exonucleases and/or endonucleases, thereby rendering them stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy are also described, for example, by Van der Krol et al. ((1988) Biotechniques 6:958-976); and Stein et al. ((1988) Cancer Res 48:2659-2668). With respect to antisense DNA, oligodeoxyribonucleotides derived from the translation initiation site, e.g., between the −10 and +10 regions of MATN3 sequence, are preferred.

To perform antisense therapy, oligonucleotides (mRNA, cDNA or DNA) are designed that are complementary to mRNA encoding MATN3. The antisense oligonucleotides bind to MATN3 mRNA transcripts and prevent translation. Absolute complementarity, although preferred, is not required. A sequence “complementary” to a portion of an RNA, as referred to herein, indicates that a sequence has sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid, as described in detail above. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures.

The oligonucleotides used in antisense therapy can be DNA, RNA, or chimeric mixtures or derivatives or modified versions thereof, single-stranded or double-stranded. The oligonucleotides can be modified at the base moiety, sugar moiety, or phosphate backbone, for example, to improve stability of the molecule, hybridization, etc. The oligonucleotides can include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad Sci. USA 84:648-652; PCT International Publication No. WO 88/09810) or the blood-brain barrier (see, e.g., PCT International Publication No. WO 89/10134), or hybridization-triggered cleavage agents (see, e.g., Krol et al. (1988) BioTechniques 6:958-976) or intercalating agents. (See, e.g., Zon (1988), Pharm. Res. 5:539-549). To this end, the oligonucleotide may be conjugated to another molecule (e.g., a peptide, hybridization triggered cross-linking agent, transport agent, hybridization-triggered cleavage agent).

The antisense molecules are delivered to cells which express MATN3 in vivo. A number of methods can be used for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically. Alternatively, in a preferred embodiment, a recombinant DNA construct is utilized in which the antisense oligonucleotide is placed under the control of a strong promoter (e.g., pol III or pol II). The use of such a construct to transfect target cells in the patient results in the transcription of sufficient amounts of single stranded RNAs that will form complementary base pairs with the endogenous MATN3 transcripts and thereby prevent translation of the MATN3 mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art and described above. For example, a plasmid, cosmid, YAC or viral vector can be used to prepare the recombinant DNA construct which can be introduced directly into the tissue site. Alternatively, viral vectors can be used which selectively infect the desired tissue, in which case administration may be accomplished by another route (e.g., systemically).

Endogenous MATN3 expression can also be reduced by inactivating or “knocking out” MATN3 or its promoter using targeted homologous recombination (e.g., see Smithies et al. (1985) Nature 317:230-234; Thomas and Capecchi (1987) Cell 51:503-512; Thompson et al. (1989) Cell 5:313-321). For example, a mutant, non-functional MATN3 (or a completely unrelated DNA sequence) flanked by DNA homologous to the endogenous MATN3 (either the coding regions or regulatory regions of MATN3) can be used, with or without a selectable marker and/or a negative selectable marker, to transfect cells that express MATN3 in vivo. Insertion of the DNA construct, via targeted homologous recombination, results in inactivation of MATN3. The recombinant DNA constructs can be directly administered or targeted to the required site in vivo using appropriate vectors, as described above. Alternatively, expression of non-mutant MATN3 can be increased using a similar method: targeted homologous recombination can be used to insert a DNA construct comprising a non-mutant, functional MATN3 (e.g., a gene having SEQ ID NO: 1 which may optionally comprise at least one polymorphism shown in Table 3 or FIGS. 5A-5C), or a portion thereof, in place of a mutant MATN3 in the cell, as described above. In another embodiment, targeted homologous recombination can be used to insert a DNA construct comprising a nucleic acid that encodes a MATN3 polypeptide variant that differs from that present in the cell.

Alternatively, endogenous MATN3 expression can be reduced by targeting deoxyribonucleotide sequences complementary to the regulatory region of MATN3 (i.e., the MATN3 promoter and/or enhancers) to form triple helical structures that prevent transcription of MATN3 in target cells in the body. (See generally, Helene (1991) Anticancer Drug Des. 6(6):569-84; Helene et al. (1992) Ann. N.Y. Acad. Sci. 660:27-36; and Maher, L. J. (1992) Bioassays 14(12):807-15). Likewise, the antisense constructs described herein, by antagonizing the normal biological activity of one of the MATN3 proteins, can be used in the manipulation of tissue, e.g., tissue differentiation, both in vivo and for ex vivo tissue cultures. Furthermore, the anti-sense techniques (e.g., microinjection of antisense molecules, or transfection with plasmids whose transcripts are anti-sense with regard to a MATN3 mRNA or gene sequence) can be used to investigate role of MATN3 in developmental events, as well as the normal cellular function of MATN3 in adult tissue. Such techniques can be utilized in cell culture, but can also be used in the creation of transgenic animals.

In yet another embodiment of the invention, other MATN3 therapeutic agents as described herein can also be used in the treatment or prevention of osteoarthritis. The therapeutic agents can be delivered in a composition, as described above, or by themselves. They can be administered systemically, or can be targeted to a particular tissue. The therapeutic agents can be produced by a variety of means, including chemical synthesis; recombinant production; in vivo production (e.g., a transgenic animal, such as U.S. Pat. No. 4,873,316 to Meade et al.), for example, and can be isolated using standard means such as those described herein.

A combination of any of the above methods of treatment (e.g., administration of non-mutant MATN3 polypeptide in conjunction with antisense therapy targeting mutant MATN3 mRNA; administration of a first splicing variant encoded by MATN3 in conjunction with antisense therapy targeting a second splicing encoded by MATN3), can also be used.

The invention will be further described by the following non-limiting examples. The teachings of all publications cited herein are incorporated herein by reference in their entirety.

### Exemplification

Identification of the MATN3 Gene with Linkage to Osteoarthritis

Genome-Wide Scan and Linkage Analysis

Selection of Patients

A genome-wide linkage scan was performed for 329 families containing 1143 individuals with primary hand OA, along with 939 genotyped relatives. A list of patients with OA of the hand was obtained based on patients' records at hospitals and health care centers in Iceland. The encrypted patient list was cross-referenced with the comprehensive Icelandic genealogy database (Gulcher and Stefansson (1998) Clin. Chem. Lab. Med. 36:523-527; and Gulcher et al. (2000) Eur. J. Hum. Genet. 8:739-742) and pedigrees with two or more affected relatives, related within a distance of five meioses or less were identified. Patients within these families, and up to three first-degree relatives, were recruited and examined by a rheumatologist or an orthopedic surgeon. Additionally, a group of patients and their relatives from another on-going study of hip OA and knee OA also had their hands examined for this study. Individuals were classified as having primary hand OA if they met either or both of the following two criteria: 1) OA with a minimum of two nodes at the distal interphalangeal (DIP) joints on each hand (DIP phenotype); or 2) OA of the thumb with squaring or dislocation of a first carpometacarpal (CMC1) joint (CMC1 phenotype). Only individuals with idiopathic OA were included in the patient cohorts.

Microsatellite Markers and Maps

The framework genomewide scan used a 1000 microsatellite marker set that contained markers from the ABI Linkage Marker (version 2) screening set and the ABI Linkage Marker (version 2) intercalating set, in combination with 500 custom-made markers. All markers were extensively tested for robustness, ease of scoring, and efficiency in multiplex PCR. Marker positions were obtained from the genetic map described by Kong and Cox ((1997) American Journal of Human Genetics 61(5):1179-1188). In the framework set, the average spacing between markers is approximately 4 cM. PCR amplifications were set up, run and pooled on Gilson Cyberlab robots. The reaction volume was 5 μl, and for each PCR reaction 20 ng of genomic DNA was amplified in the presence of 2 pmol of each primer, 0.25 U AmpliTaq Gold, 0.2 mM dNTPs and 2.5 mM MgCl₂ (buffer was supplied by manufacturer, Applera). Cycling conditions were: 95° C. for 10 minutes, followed by 37 cycles of 94° C. for 15 seconds, annealing for 30 seconds at 55° C. and 1 minute extension at 72° C. The PCR products were supplemented with the internal size standard and the pools were separated and detected on 3700 Sequencers using Genescan v3.0 peak calling software (Applera). Alleles were automatically called using DAC, an allele-calling program developed at deCODE genetics (Fjalldal et al. (2001) Proc. Int. Joint. Conf. Neural Networks A1-A6), and the program DecodeGT was used to fractionate called genotypes according to quality and edit when necessary (Palsson et al. (1999) Genome Res. 9:1002-1012).

Statistical Methods for Linkage Analysis

Multipoint, affected-only allele-sharing methods were used to assess the evidence for linkage. All results, including LOD and NPL scores, were obtained using the program ALLEGRO (Gudbjartsson et al. (2000), Nature Genetics 25(1):12-13). The Sₚₐᵢᵣₛ scoring function (Kruglyak et al. (1996) American Journal of Human Genetics. 58(6):1347-1363; and Whittemore and Halpern (1994) Biometrics 50:118-127) and the exponential allele-sharing model (Kong and Cox (1997) American Journal of Human Genetics 61(5):1179-1188) were used to generate the relevant one degree of freedom statistics. When combining the family scores to obtain an overall score, instead of weighting the families equally, the default of GENEHUNTER (Kong and Cox (1997) American Journal of Human Genetics 61(5):1179-1188), or weighting the affected pairs equally, a weighting scheme that is half way between the two in the log scale was used; the family weights are the geometric means of the weights of the two schemes. While not identical, this weighting scheme tends to give similar results to that proposed by Weeks and Lange ((1988) Am. J. Hum. Genet. 42:315-326) as an extension of a weighting scheme of Hodge ((1984) Genet. Epidemiol. 1:109-122) designed for sibships. The P value was computed two different ways and the less significant one is reported. The first P value was computed based on large sample theory; Zₗᵣ=✓(2 logₑ (10) LOD) is distributed approximately as a standard normal random variable under the null hypothesis of no linkage (Kong and Cox (1997) American Journal of Human Genetics 61(5): 1179-1188). Furthermore, because of the concern with small sample behavior, a second P value was computed by comparing the observed LOD score to its complete data sampling distribution under the null hypothesis (Gudbjartsson et al. (2000) Nature Genetics 25(1):12-13). When a data set consists of more than a handful of families, which is the case herein, these two P values tend to be very similar. To ensure that the results were a true reflection of the information contained in the material, for a linkage result to be considered significant, not only was it required that the P value be smaller than 2×10⁻⁵ (Lander and Kruglyak (1995) Nature Genetics 11(3):241-247), but also that the information content in the region was at least 85%. For the families in this study, an information content of 85% corresponded to a marker density of approximately one marker every centimorgan. The information measure used has been defined previously (Nicolae (1999) Allele sharing models in gene mapping: a likelihood approach. PhD thesis. University of Chicago, Chicago) and implemented in ALLEGRO. This measure is closely related to a classical measure of information (Dempster et al. (1977) J. R. Stat. Soc. B. 39:1-38), having the property that it is between zero, if the marker genotypes are completely uninformative, and one, if the genotypes determine the exact amount of allele sharing by descent among the affected relatives.

Results of Linkage Analysis

An LOD score of 1.48 was observed on chromosome 2. In order to study the effects of the sub-phenotypes on the linkage results, three additional genome-wide scans were performed, in which individuals were considered affected if they had the DIP phenotype (DIP cohort), the CMC1 phenotype (CMC1 cohort), or both the DIP and the CMC1 phenotypes (DIP/CMC1 cohort). The DIP cohort scan included 944 affecteds in 274 families; the CMC1 cohort scan included 558 affecteds in 204 families; the DIP/CMC1 cohort scan included 382 affecteds in 142 families. Table 1 indicates the location and size of peaks on chromosome 2 with an LOD score above 1 for the primary hand OA cohort, the DIP cohort, and the CMC1 cohort. No LOD scores above 1 were observed for the DIP/CMC1 cohort on chromosome 2.

TABLE 1

| Cohort | LOD Score | Marker | Location (cM) |
|---|---|---|---|
| Primary | 1.48 | D2S146 | 51.5 |
| Hand OA | | | |
| Primary | 1.14 | D2S2277 | 160.4 |
| Hand OA | | | |
| DIP | 1.13 | D2S2324 | 160.8 |
| CMC1 | 2.23 | D2S2168 | 48.0 |

Only one location on chromosome 2, achieved an LOD score of two or greater in at least one of the four scans (at D2S2168 for the CMC1 cohort). Markers in this region were added to increase the information content on allele-sharing among affected relatives.

Table 2 summarizes the finemapping linkage results for the chromosome 2 locus, indicating the peak markers, along with their genetic locations. Increased evidence for linkage on chromosome 2 for all cohorts was seen, with an LOD score of 4.97 between D2S175 and D2S2201 for the CMC1 cohort (p-value of 8.5×10⁻⁷). This LOD score remained significant even after correction for the four genome-wide scans. For the CMC1 cohort, the size of the region on chromosome 2 that has an LOD score within one of the peak LOD score is a little over 5 cM from D2S175 (41.9 cM) to D2S1324 (47.1 cM).

TABLE 2

| Cohort | LOD Score | Peak Location | Peak Marker(s) |
|---|---|---|---|
| Primary | 2.42 | 44.0 | D2S175, |
| Hand OA | | | D2S2201 |
| DIP | 2.44 | 44.0 | D2S175, |
| | | | D2S2201 |
| CMC1 | 4.97 | 44.0 | D2S175, |
| | | | D2S2201 |
| DIP/CMC1 | 4.44 | 44.0 | D2S175, |
| | | | D2S2201 |

Screening for Mutations in the MATN3 Gene

Based on these results, and primarily on the result in the CMC1 cohort, association analysis at the chromosome 2 locus is ongoing. Six publicly characterized genes were found to be within a 4 Mb region centered on the chromosome 2 peak. One of the genes, MATN3, is located within 100 Kb of the LOD score peak, and a recent publication implicated mutations in this gene to a class of dysplasias of large joints with associated early-onset OA (Chapman et al. (2001) Nature Genetics 28(4):393-396).

To identify polymorphisms within the MATN3 gene, primers (described in detail below) were designed for PCR amplification of all known exons and the promoter sequence of the MATN3 gene, as well as most of the intronic sequence. DNA from 76 patients belonging to families scoring positive in a non-parametric-linkage analysis for the markers under the peak of the LOD score and 18 controls were initially sequenced for polymorphism within the gene. Both the forward and reverse strands were sequenced on ABI prism 3700 DNA analyzer.

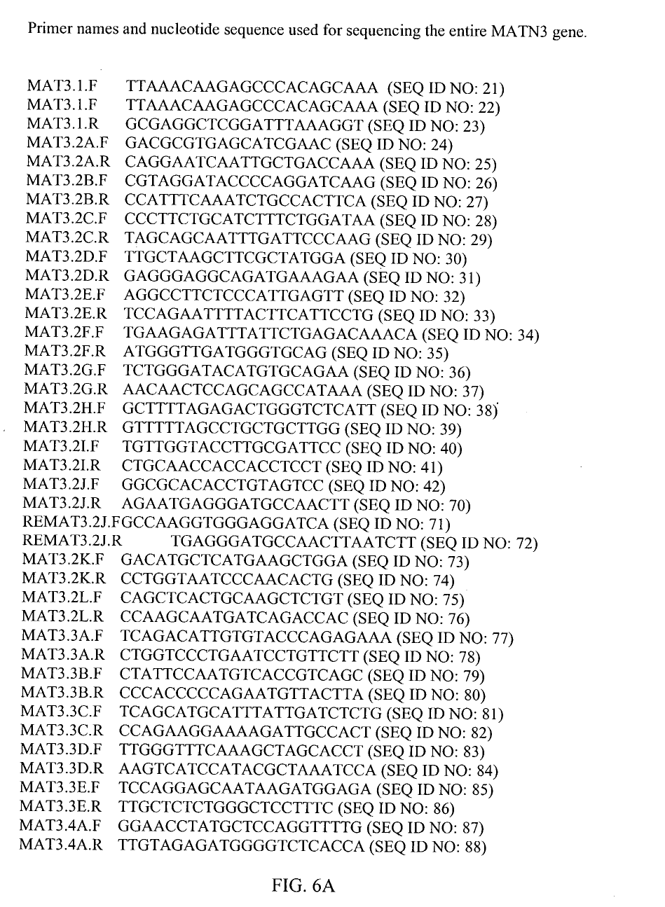

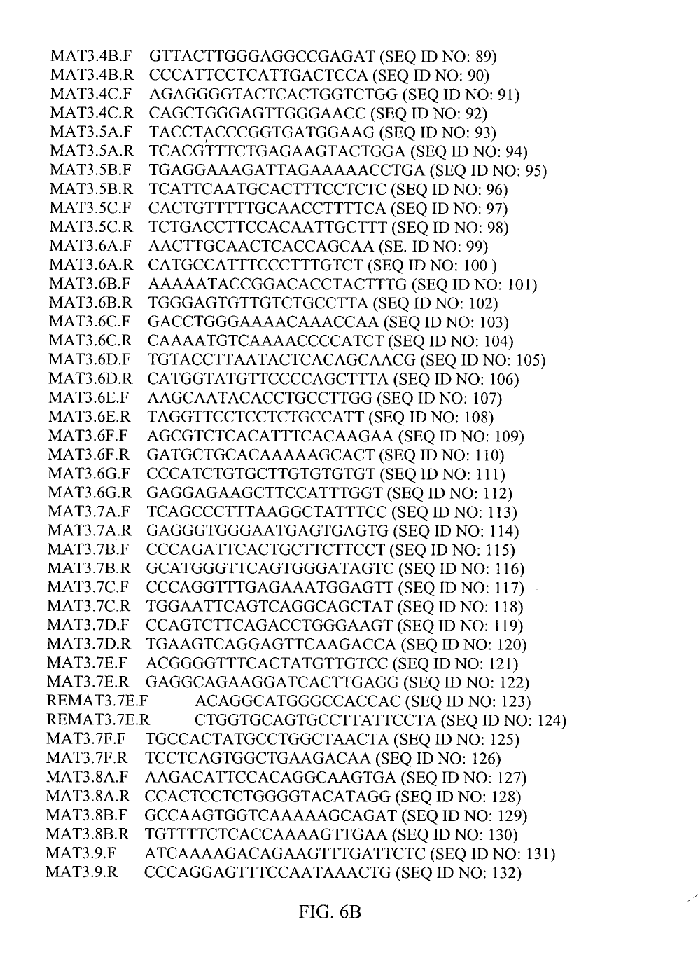

FIGS. 6A to 6B list all primers used for PCR amplification of DNA sequences of the MATN3 gene. Subsequent sequencing of both forward and reverse strands revealed the nucleotide variations listed in Table 3.

Results of Mutational and Association Analyses

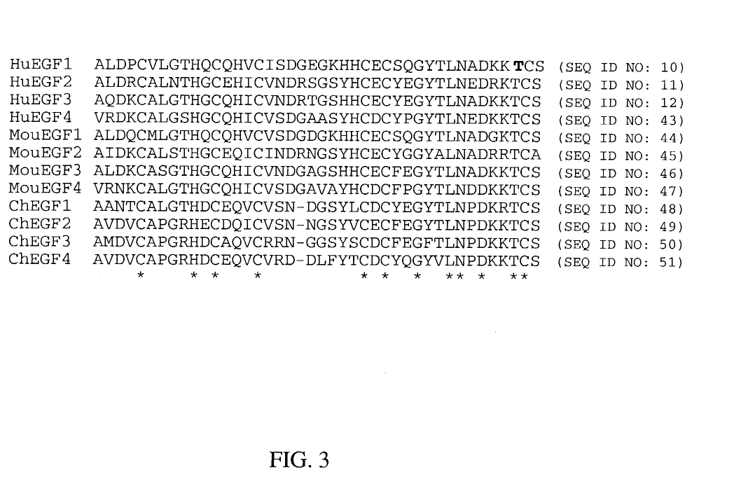

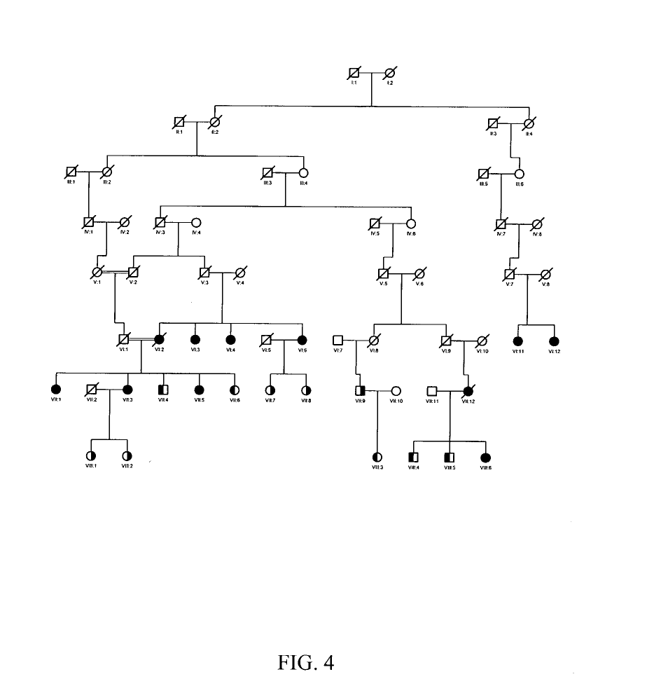

In the initial mutational analysis carried out on the exons of MATN3 in samples from 76 patients and 18 controls, a novel coding SNP, which showed excess in patients was identified. This mutation involves a nucleotide change from cytidine to thymidine in the third exon of the gene, predicting a substitution of a threonine by a methionine residue at amino acid position 303 in the first epidermal growth factor-like (EGF) domain of the MATN3 protein. The gene for MATN3 contains 4 repeats with homology to the EGF domains (Wagener et al. (2000) Mammalian Genome 11(2):85-90). Alignnent of partial EGF amino acid sequences (FIG. 3) shows that the threonine resudue is conserved within all of the EGF domains in this position, in human (HuEGF1-4), mouse (MouEGF1-4) and chicken (ChEGF1-4). The mutated threonine is shown in bold, and asterisks denote conserved amino acids.

The excess in patients, with a relative risk of around two, persisted after genotyping this SNP for 190 more patients and 190 more controls, but the frequency of the mutation was very small, only a little over 1% in patients.

In order to more fully investigate the contribution of this mutation to hand OA risk in Iceland, the entire patient set was typed using a fluorescent polarization method (Chen et al. (1999) Genome Res. 9:492). A total of 2162 patients and 873 controls were typed for this coding SNP. Among the patients, 1312 of them had the CMC1 phenotype. The results of the mutation screening for the MATN3 gene are shown in Table 3.

TABLE 3

| Location | Base no. | Polymorphism | Amino acid change |
|---|---|---|---|
| 5′prime | 37916 | c/t | |
| 5′prime | 38157 | 0/t | |
| 5′prime | 38211 | c/t | |
| 5′prime | 38270 | c/t | |
| 5′prime | 38367 | 0/ggggcggggc | |
| 5′prime | 38374 | g/a | |
| 5′prime | 38390 | a/c | |
| Exon 1 | 38496 | c/t | Pro/Ser |
| Exon 1 | 38527 | g/t | Leu/Arg |
| Exon 1 | 38652 | g/t | Ser/Ala |
| Intron 1 | 39565 | a/c | |
| Intron 1 | 41240 | a/g | |
| Intron 1 | 41763 | c/t | |
| Intron 1 | 42446 | c/t | |
| Intron 1 | 42795 | a/g | |
| Intron 1 | 43239 | c/t | |
| Intron 1 | 43580 | 0/g | |
| Exon 2 | 44927 | g/a | Val/Met |
| Exon 2 | 45010 | c/t | |
| Exon 2 | 45171 | c/t | Val/Ala |
| Exon 2 | 45178 | g/a | |
| Exon 2 | 45246 | c/t | Ala/Val |
| Exon 2 | 45317 | a/g | Glu/Lys |
| Intron 2 | 45366 | 0/tc | |
| Intron 2 | 45434 | 0/tctt | |
| Intron 2 | 45506 | a/g | |
| Intron 2 | 45507 | c/t | |
| Intron 2 | 45584 | c/t | |
| Intron 2 | 45588 | a/g | |
| Intron 2 | 45661 | g/t | |
| Intron 2 | 45808 | a/g | |
| Intron 2 | 45852 | a/g | |
| Intron 2 | 46055 | a/t | |
| Intron 2 | 46104 | c/t | |
| lntron 2 | 46168 | g/t | |
| Intron 2 | 46404 | a/g | |
| Intron 2 | 46804 | a/g | |
| Intron 2 | 46960 | a/g | |
| Intron 2 | 47482 | c/t | |
| Intron 2 | 47712 | c/t | |
| Intron 2 | 47753 | c/t | |
| Intron 2 | 47769 | a/tcc | |
| Exon 3 | 47812 | a/g | |
| Exon 3 | 47852 | a/g | Val/Ile |
| Exon 3 | 47864 | c/g | Asp/His |
| Exon 3 | 47928 | c/t | Thr/Met |
| Exon 3 | 47929 | a/g | |
| Intron 3 | 47950 | a/g | |
| Intron 3 | 48047 | c/t | |
| Intron 3 | 48064 | c/g | |
| Intron 3 | 48120 | a/g | |
| Intron 3 | 48936 | a/g | |
| Exon 4 | 49045 | c/t | |
| Intron 4 | 49400 | c/t | |
| Exon 5 | 50584 | g/t | Asp/Tyr |
| Intron 5 | 51769 | c/t | |
| Intron 5 | 52318 | 0/at | |
| Intron 5 | 52356 | c/t | |
| Intron 5 | 52757 | a/g | |
| Intron 5 | 52792 | c/t | |
| Intron 5 | 53007 | a/t | |
| Intron 5 | 53327 | 0/gt | |
| Intron 5 | 53482 | a/c | |
| Intron 5 | 53828 | a/g | |
| Exon 6 | 53862 | c/t | Ser/Phe |
| Exon 6 | 53900 | a/g | Ala/Thr |
| Intron 6 | 54077 | c/t | |
| Intron 6 | 54747 | a/t | |
| Intron 6 | 54752 | a/t | |
| Intron 6 | 55775 | g/t | |
| Intron 6 | 56260 | g/t | |
| Intron 7 | 56822 | c/g | |
| Intron 7 | 56911 | c/t | |
| Intron 7 | 57032 | c/t | |
| Intron 7 | 57927 | c/t | |
| Exon 8 | 57981 | c/t | Arg/Cys |
| 3′ | 58045 | c/t | |
| 3′ | 58162 | a/t | |
| 3′ | 58407 | a/t | |
| 3′ | 58521 | g/t | |
| 3′ | 58721 | c/t | |
| 3′ | 58818 | a/g | |
| 3′ | 58878 | 0/tt | |
| 3′ | 58883 | g/t | |
| 3′ | 58892 | c/t | |
| 3′ | 59224 | a/g | |
| 3′ | 59426 | a/g | |
| 3′ | 60875 | a/g | |
| 3′ | 60877 | a/c | |
| 3′ | 60965 | a/g | |
| 3′ | 61007 | c/t | |
| 3′ | 61213 | c/t | |
| 3′ | 61256 | a/g | |
| 3′ | 61261 | c/t | |

Nine of the 873 controls were heterozygous for the mutation, none were homozygous, whereas 43 of the 2162 patients were heterozygous, and 2 were homozygous. The relative risk for OA in individuals with the thymidine residue at position 47928 was also determined, as shown in Table 4.

TABLE 4
Association Analysis of Mutation at Nucleic Acid 47928

| | | | | | Cntrl. |
|---|---|---|---|---|---|
| Cohort | RRisk | # Affected | Aff. Freq. | # Controls | Freq. |
| Primary Hand OA | 2.12 | 2162 | 0.0109 | 873 | 0.0052 |
| Knee OA (DIP) | 2.06 | 1801 | 0.0106 | 873 | 0.0052 |
| Thumb OA | 2.61 | 1312 | 0.0133 | 873 | 0.0052 |
| (CMC1) | | | | | |
| Both Finger & | 2.67 | 951 | 0.0137 | 873 | 0.0052 |
| Thumb | | | | | |
| (DIP/CMC1) | | | | | |

The mutation was present in 2.1 % of patients with hand OA in the Icelandic population. The estimated relative risk (RRisk) of primary hand OA for carriers of a single copy of the mutation compared to the non-carrier under the multiplicative model is 2.12. Both of the homozygous carriers and 31 of the 43 patients heterozygous for the mutation had the CMC1 phenotype. This led to an estimated relative risk for the CMC1 phenotype of 2.61, which is slightly higher than that for primary hand OA. The highest relative risk was observed for patients with OA in both finger and thumbs (2.67). The Affected Frequency and the Control Frequency in Table 4 were also determined as described by Chen et al. ((1999) Genome Res. 9:492)).

In addition, people with the methionine mutation at position 47928 have the phenotypes as shown in Table 5.

TABLE 5

| Phenotype | # of People |
|---|---|
| Finger OA | 30 |
| Knee OA | 36 |
| Thumb OA | 33 |
| Hip OA | 34 |
| Back OA | 17 |

Though this mutation alone could not account for the significant linkage result for the CMC1 cohort, it was observed that 30 of the 45 patient carriers of this mutation were in the linkage families, including both homozygous carriers. A linkage analysis for the CMC1 cohort less the mutation carriers was performed, in order to assess the effect of these carriers on the locus. The chromosome 2 peak LOD score dropped to 3.80, which demonstrates that although these carriers have a significant impact on the linkage, there are likely, as yet undetected, associations of hand OA to either MATN3 or other genes in the region.

Identification of a Haplotype for Increased Risk of Osteoarthritis

By sequencing the gene and surrounding sequences, several novel nucleotide variants were identified (see Table 3). Using these polymorphic nucleotides and reconstructing the haplotypes, an independent haplotype from the mutation, which carries an increased risk in patients, was detected (Table 6).

TABLE 6

| Polymorphism | | | | | | | | | | | | | |
|---|---|---|---|---|---|---|---|---|---|---|---|---|---|
| 58162 | 57927 | 56822 | 47929 | 45434 | 45317 | 45178 | 45010 | p-val | aff.frq | N_aff | ctrl.frq | N_ctrl | rel risk |
| T | T | | | | G | | | 0.00193 | 0.3155 | 718 | 0.2523 | 392 | 1.36576 |
| | T | | | | G | A | | 0.00217 | 0.3152 | 721 | 0.2529 | 392 | 1.35996 |
| | T | | | | G | | T | 0.00234 | 0.3134 | 723 | 0.2517 | 392 | 1.3565 |
| | | G | A | TCTT | | | | 0.00277 | 0.2782 | 724 | 0.2195 | 389 | 1.37035 |
| | | | A | | | A | T | 0.00326 | 0.2771 | 723 | 0.2194 | 389 | 1.36342 |

In addition to the mutation at position 47928, a significant attributed risk haplotype across the gene for MATN3 was found. Polymorphism refers to the position of the SNP (Table 3), used to detect the attributed risk haplotype, aff.frq and ctrl.frq are haplotype frequencies in affected and controls, respectively. N_aff and N_ctr are the numbers of affected and controls, respectively. The rel_risk and p-val are relative risk and P-value for the haplotype, respectively. This haplotype, which does not carry the thr/met mutation, is in 28-32% haplotypes from patients, but only 22-25% haplotypes from controls.

Additional SNPs in the MATN3 Gene and Surrounding Sequences

Insert and deletions are further described in FIGS. 5A-5C. These additional polymorphisms around and in the MATN3 gene are likely to be associated to the disease, either alone or as a part of a haplotype.

Matrilin-3

Matrilin-3 is a candidate for an osteoarthritis gene. It is a non-collagenous extracellular matrix protein that is one of a class of 4 related proteins termed matrilins 1 through 4. All 4 matrilins are expressed in the developing skeletal system but matrilin-3 exhibits the expression pattern most restricted to developing cartilage, especially the epiphyseal cartilage. The matrilins are made up of von Willebrand factor (VWF) A domains, EGF-like repeats, and a C-terminal alpha helical coiled-coil domain. Matrilin-3 has a single N-terminal VWF A domain followed by 4 EGF repeats and the coiled coil domains while the other matrilins each have two VFW A domains separated by 1 to 10 EGF repeats and then the C terminal coiled coil domain. The coiled-coil domains mediate covalent multimer formation among the matrilins through their heptad repeats and two cysteines. The matrilins form homomultimers and heteromultimers in almost every combination with each other in proportion to the concentration of each subunit. Matrilin-3 forms heteromultimers only with matrilin-1 and these are heterotetramers with two subunits of each. The VWF A domain is a collagen binding domain in other proteins and matrilin-1 has been shown to bind to Type II collagen fibrils in cartilage in a periodic pattern.

Matrilin-1 also interacts with aggrecan and may also bind to integrin α1β1 (Makihira et al. (1999) J. Biol. Chem. 274:11417-11423). Therefore, matrilin-1 may represent the link between the collagen fibril network and the proteoglycan network as well as a connection to the chondrocytes. Indeed, mice without matrilin-1 develop normally but show ultrastructural changes in fibril networks in cartilage (Huang et al. (1999) Dev. Dyn. 216:434-441). Matrilin-1 is an excellent candidate gene for OA given its interaction with the collagen and proteoglycan networks. Several groups have analyzed association of microsatellite polymorphism in the 3′ untranslated region of the matrilin-1 gene and hip osteoarthritis. A Dutch cohort stratified on males found a significant association of allelic polymorphism to hip OA. In contrast, British and Argentinean cohort studies failed to replicate these results in their population (Strusberg et al. (2002) Clin. Exp. Rheumatol. 20:543-545; Loughlin et al. (2000) Arthritis Rheum. 43:1423-1425; and Meulenbelt et al. (1997) Arthritis Rheum. 40:1760-1765).

Matrilin-3 forms heterotetramers with matrilin-1 with higher affinity than either of the respective homomultimers, therefore it is reasonable to believe that it may serve a modulating role in the cross-linking function of matrilin-1. While matrilin-1 and matrilin-3 expression patterns overlap in cartilage, matrilin-3 expression is higher than matrilin-1 in the proliferation zone where there are mainly non-collagenous filaments while matrilin-1 has the higher expression in the mature zone where the collagen-proteoglycan network is more extensive. If matrilin-1 facilitates collagen fibril formation and binding to proteoglycan, then it is reasonable to believe that matrilin-3 may play an inhibitory role in normal development and maintenance of cartilage and bone. If there is too much matrilin-3 because of increased synthesis or decreased breakdown, then these matrilin-1 roles may be impaired.

While this invention has been particularly shown and described with reference to preferred embodiments thereof, it will be understood by those skilled in the art that various changes in form and details may be made therein without departing from the spirit and scope of the invention as defined by the appended claims.

What is claimed is:

1. An isolated nucleic acid molecule comprising a matrilin-3 gene, or a fragment or variant thereof, the nucleotide sequence of SEQ ID NO: 1 and comprising at least one polymorphism as shown in Table 3 or FIGS. 5A-5C.

2. A nucleic acid encoding a polypeptide having an amino acid sequence selected from the group consisting of SEQ ID NOs: 2, 3, 4, 5, 6, 7, 8 and 9.

3. A method for assaying the presence of a first nucleic acid molecule in a sample, comprising contacting said sample with a second nucleic acid molecule comprising a nucleotide sequence selected from the group consisting of SEQ ID NO: 1 comprising at least one polymorphism as shown in Table 3 or FIGS. 5A-5C and complements thereof, under high stringency conditions.

4. A vector comprising an isolated nucleic acid molecule selected from the group consisting of: SEQ ID NO: 1 and comprising at least one polymorphism shown in Table 3 or FIGS. 5A-5C, and complements thereof, operatively linked to a regulatory sequence.

5. A recombinant host cell comprising the vector of claim 4.

6. A method for producing a polypeptide encoded by an isolated nucleic acid molecule, comprising culturing the recombinant host cell of claim 5 under conditions suitable for expression of said nucleic acid molecule.

7. An isolated polypeptide encoded by a matrilin-3 gene, wherein the matrilin-3 gene has the sequence of SEQ ID NO: 1 comprising at least one polymorphism as shown in Table 3 or FIGS. 5A-5C, or complements thereof.

8. The isolated polypeptide of claim 7, wherein the polypeptide has an amino acid sequence selected from the group consisting of SEQ ID NOs: 2-9.

9. A fusion protein comprising an isolated polypeptide of claim 7.

10. An antibody, or an antigen-binding fragment thereof, which selectively binds to a polypeptide of claim 7.

11. An antibody, or an antigen-binding fragment thereof, which selectively binds to an amino acid sequence selected from the group consisting of SEQ ID NOs: 2-9, or to a fragment or variant of said amino acid sequence.

12. A method for assaying the presence of a polypeptide encoded by an isolated nucleic acid molecule according to claim 1 in a sample, comprising contacting said sample with an antibody which specifically binds to the encoded polypeptide.

13. A method of diagnosing a susceptibility to osteoarthritis in an individual, comprising detecting a polymorphism in a matrilin-3 gene, wherein the presence of the polymorphism in the gene is indicative of a susceptibility to osteoarthritis.

14. A method of diagnosing a susceptibility to osteoarthritis, comprising detecting an alteration in the expression or composition of a polypeptide encoded by a matrilin-3 gene in a test sample, in comparison with the expression or composition of a polypeptide encoded by matrilin-3 gene in a control sample, wherein the presence of an alteration in expression or composition of the polypeptide in the test sample is indicative of a susceptibility to osteoarthritis.

15. The method of claim 14, wherein the alteration in the expression or composition of a polypeptide encoded by matrilin-3 gene comprises expression of a splicing variant polypeptide in a test sample that differs from a splicing variant polypeptide expressed in a control sample.

16. A method of identifying an agent which alters activity of a polypeptide of claim 7, comprising:
a) contacting the polypeptide or a derivative or fragment thereof, with an agent to be tested;
b) assessing the level of activity of the polypeptide or derivative or fragment thereof; and
c) comparing the level of activity with a level of activity of the polypeptide or active derivative or fragment thereof in the absence of the agent,
wherein if the level of activity of the polypeptide or derivative or fragment thereof in the presence of the agent differs, by an amount that is statistically significant, from the level in the absence of the agent, then the agent is an agent that alters activity of the polypeptide.

17. A method of identifying an agent which alters interaction of the polypeptide of claim 7 with a matrilin-3 gene binding agent, comprising:
a) contacting the polypeptide or a derivative or fragment thereof, the binding agent and with an agent to be tested;
b) assessing the interaction of the polypeptide or derivative or fragment thereof with the binding agent; and
c) comparing the level of interaction with a level of interaction of the polypeptide or derivative or fragment thereof with the binding agent in the absence of the agent,
wherein if the level of interaction of the polypeptide or derivative or fragment thereof in the presence of the agent differs, by an amount that is statistically significant, from the level of interaction in the absence of the agent, then the agent is an agent that alters interaction of the polypeptide with the binding agent.

18. An agent which alters interaction of a matrilin-3 gene polypeptide with a matrilin-3 gene binding agent, identifiable according to the method of claim 17.

19. A method of identifying an agent which alters expression of a matrilin-3 gene, comprising the steps of:
a) contacting a solution containing a nucleic acid of claim 1 or a derivative or fragment thereof with an agent to be tested;
b) assessing the level of expression of the nucleic acid, derivative or fragment; and
c) comparing the level of expression with a level of expression of the nucleic acid, derivative or fragment in the absence of the agent,
wherein if the level of expression of the nucleotide, derivative or fragment in the presence of the agent differs, by an amount that is statistically significant, from the expression in the absence of the agent, then the agent is an agent that alters expression of a matrilin-3 gene.

20. A method of identifying an agent which alters expression of a matrilin-3 gene, comprising the steps of:
a) contacting a solution containing a nucleic acid comprising the promoter region of matrilin-3 gene operably linked to a reporter gene, with an agent to be tested;
b) assessing the level of expression of the reporter gene; and
c) comparing the level of expression with a level of expression of the reporter gene in the absence of the agent,
wherein if the level of expression of the reporter gene in the presence of the agent differs, by an amount that is statistically significant, from the level of expression in the absence of the agent, then the agent is an agent that alters expression of a matrilin-3 gene.

21. A method of identifying an agent which alters expression of a matrilin-3 gene, comprising the steps of:
a) contacting a solution containing a nucleic acid of claim 1 or a derivative or fragment thereof with an agent to be tested;
b) assessing expression of the nucleic acid, derivative or fragment; and
c) comparing expression with expression of the nucleic acid, derivative or fragment in the absence of the agent,
wherein if expression of the nucleotide, derivative or fragment in the presence of the agent differs, by an amount that is statistically significant, from the expression in the absence of the agent, then the agent is an agent that alters expression of a matrilin-3 gene.

22. The method of claim 21, wherein the expression of the nucleotide, derivative or fragment in the presence of the agent comprises expression of one or more splicing variant(s) that differ in kind or in quantity from the expression of one or more splicing variant(s) the absence of the agent.

23. A method of altering expression of a matrilin-3 gene, comprising contacting a cell containing said matrilin-3 gene with an agent identifiable by the method of claim 21.

24. A method of identifying a polypeptide which interacts with a matrilin-3 gene polypeptide, comprising employing a yeast two-hybrid system using a first vector which comprises a nucleic acid encoding a DNA binding domain and a matrilin-3 gene polypeptide, splicing variant, fragment or derivative thereof, and a second vector which comprises a nucleic acid encoding a transcription activation domain and a nucleic acid encoding a test polypeptide, wherein if transcriptional activation occurs in the yeast two-hybrid system, the test polypeptide is a polypeptide which interacts with a matrilin-3 polypeptide.

25. A matrilin-3 gene therapeutic agent selected from the group consisting of: a matrilin-3 gene or fragment or derivative thereof; a polypeptide encoded by matrilin-3 gene; a matrilin-3 gene receptor; a matrilin-3 gene binding agent; a peptidomimetic; a fusion protein; a prodrug; an antibody; an agent that alters matrilin-3 gene expression; an agent that alters activity of a polypeptide encoded by matrilin-3 gene; an agent that alters posttranscriptional processing of a polypeptide encoded by matrilin-3 gene; an agent that alters interaction of a matrilin-3 gene with a matrilin-3 gene binding agent; an agent that alters transcription of splicing variants encoded by matrilin-3 gene; and a ribozyme.

26. A pharmaceutical composition comprising a matrilin-3 gene therapeutic agent of claim 25.

27. The pharmaceutical composition of claim 26, wherein the matrilin-3 gene therapeutic agent is an isolated nucleic acid molecule comprising a matrilin-3 gene or fragment or derivative thereof.

28. The pharmaceutical composition of claim 26, wherein the matrilin-3 gene therapeutic agent is a polypeptide encoded by the matrilin-3 gene.

29. A method of treating osteoarthritis in an individual, comprising administering a matrilin-3 gene therapeutic agent to the individual, in a therapeutically effective amount.

30. The method of claim 29, wherein the matrilin-3 gene therapeutic agent is a matrilin-3 gene agonist.

31. The method of claim 30, wherein the matrilin-3 gene therapeutic agent is a matrilin-3 gene antagonist.

32. A transgenic animal comprising a nucleic acid selected from the group consisting of: an exogenous matrilin-3 gene and a nucleic acid encoding a matrilin-3 gene polypeptide.

33. A method for assaying a sample for the presence of a matrilin-3 gene nucleic acid, comprising:
a) contacting said sample with a nucleic acid comprising a contiguous nucleotide sequence, which is a at least partially complementary to a part of the sequence of said matrilin-3 gene nucleic acid under conditions suitable for hybridization, and
b) assessing whether hybridization has occurred between a matrilin-3 gene nucleic acid and said nucleic acid comprising a contiguous nucleotide sequence which is at least partially complementary to a part of the sequence of said matrilin-3 gene nucleic acid.

34. The method of claim 33, wherein said nucleic acid comprising a contiguous nucleotide sequence is completely complementary to a part of the sequence of said matrilin-3 gene nucleic acid.

35. The method of claim 33, comprising amplification of at least part of said matrilin-3 gene nucleic acid.

36. The method of claim 33, wherein said contiguous nucleotide sequence is 100 or fewer nucleotides in length and is either: a) at least 80% identical to a contiguous sequence of nucleotides in SEQ ID NO: 1 comprising at least one polymorphism as shown in Table 3 or FIGS. 5A-5C; b) at least 80% identical to the complement of a contiguous sequence of nucleotides in SEQ ID NO: 1 comprising at least one polymorphism as shown in Table 3 or FIGS. 5A-5C; or c) capable of selectively hybridizing to said matrilin-3 gene nucleic acid.

37. A reagent for assaying a sample for the presence of a matrilin-3 gene nucleic acid, said reagent comprising a nucleic acid comprising a contiguous nucleotide sequence which is at least partially complementary to a part of the nucleotide sequence of said matrilin-3 gene nucleic acid.

38. The reagent of claim 37, wherein the nucleic acid comprises a contiguous nucleotide sequence which is completely complementary to a part of the nucleotide sequence of said matrilin-3 gene nucleic acid.

39. A reagent kit for assaying a sample for the presence of a matrilin-3 gene nucleic acid comprising in separate containers:
a) one or more labeled nucleic acids comprising a contiguous nucleotide sequence which is at least partially complementary to a part of the nucleotide sequence of said matrilin-3 gene nucleic acid; and
b) reagents for detection of said label.

40. The reagent kit of claim 39, wherein the labeled nucleic acid comprises a contiguous nucleotide sequences which is completely complementary to a part of the nucleotide sequence of said matrilin-3 gene nucleic acid.

41. A reagent kit for assaying a sample for the presence of a matrilin-3 gene nucleic acid comprising one or more nucleic acids comprising a contiguous nucleotide sequence which is at least partially complementary to a part of the nucleotide sequence of said matrilin-3 gene nucleic acid, and which is capable of acting as a primer for said matrilin-3 gene nucleic acid when maintained under conditions for primer extension.

42. A method of diagnosing susceptibility to osteoarthritis in an individual, comprising screening for an at-risk haplotype in the matrilin-3 gene that is more frequently present in an individual susceptible to osteoarthritis compared to a healthy individual, wherein the at-risk haplotype increases risk of osteoarthritis significantly.

43. The method of claim 42, wherein the significant increase is at least about 20%.

44. The method of claim 42, wherein the significant increase is identified as an odds ratio of at least about 1.2.

45. A method of diagnosing susceptibility to osteoarthritis in an individual, comprising screening for an at-risk haplotype in the matrilin-3 gene that is more frequently present in an individual susceptible to osteoarthritis (affected), compared to the frequency of its presence in a healthy individual (control), wherein the presence of the at-risk haplotype is indicative of a susceptibility to osteoarthritis.

46. The method of claim 45, wherein the at-risk haplotype is characterized by the presence of at least one polymorphism at nucleic acid positions 58162, 57927, 56822, 47929, 45434, 45317, 45178 and 45010, relative to SEQ ID NO: 1.

47. The method of claim 45, wherein screening for the presence of an at-risk haplotype in the matrilin-3 gene comprises enzymatic amplification of nucleic acid from said individual.

48. The method of claim 45, wherein the nucleic acid is DNA.

49. The method of claim 48, wherein the DNA is mammalian.

50. The method of claim 49, wherein the DNA is human.

51. The method of claim 45, wherein screening for the presence of an at-risk haplotype in the matrilin-3 gene comprises:
(a) obtaining material containing nucleic acid from the individual;
(b) amplifying said nucleic acid; and
(c) determining the presence or absence of an at-risk haplotype in said amplified nucleic acid.

52. The method of claim 51, wherein determining the presence of an at-risk haplotype is performed by electrophoretic analysis.

53. The method of claim 51, wherein determining the presence of an at-risk haplotype is performed by restriction length polymorphism analysis.

54. The method of claim 51, wherein determining the presence of an at-risk haplotype is performed by sequence analysis.

55. The method of claim 51, wherein determining the presence of an at-risk haplotype is performed by hybridization analysis.

56. A kit for diagnosing susceptibility to osteoarthritis in an individual comprising:
primers for nucleic acid amplification of a region of the matrilin-3 gene comprising an at-risk haplotype, wherein the primers comprise a segment of nucleic acids of length suitable for nucleic acid amplification, selected from the group consisting of: a polymorphism at nucleic acid position 58162, 57927, 56822, 47929, 45434, 45317, 45178 and 45010, relative to SEQ ID NO: 1 and combinations thereof.


SEQUENCE LISTING (132 sequences)

SEQ ID NO: 1 - DNA, 137870 bp, Homo sapiens
gaattcctctgttttggttcttgttgtatttctacgatctgaaaggcagcatctcccttcctcctatccagtgctccctggaattttttcctctaatgaaataattaggagcggaccttggtaaacgcagtctagtttcgacattggtcttcccgttgggaaagcgaaagatccagcggtaacccggttgtccgcgtttcttggactttgttttgtttcatgcctctcttgcatctccagaatcgcttgggggcgatagcttttgcctgagttgacgcctgaccagcttttgtcctggtgcagcctcctccctgcgcagtaggtgcttttcgtagccgaccggctgtttttacagagacctttattgagagccctctccatcctggccacctaccccggtatctcctgggttccaagatctgtgtgattctgtcctaaccaagttgacactgttgttgcctggtgattcactagtaataagtttgtgattttttcttttgtaattcaccatttatagtatccatgcacattaaacctaacttgtctatgacttccaaatatgggattcagtttaggtagttttaggttattatgattttgccgaagccgtgacaaacttcctacatggtcgacttggccactttaatgacttcgcgaatgtggttgtgtgggtgacgttggtgatactgtgcagtttttgatacttaactgcaaaagaggaagagagttatcccagacacaatctgttttctctgtacttgctgcttctccccactcattgacttgaaatcattagtaagcatttggacaaacgccagatgagaatcatcttaaatgaggcagatactgtttcatggcccacccccgggggactacctgactccacatgactgctacctgccctgttgttttttaccgtcttgtttctgatgtacatttaaaaaacaacacgaatgtgtaggtttgcttgtgctgagtttattttcatgtttcctgtacttttatattcggaaataccgccttattgacagaactatatcacattttccctcagagactctttcgttctgtattttaactgatttggaatactaaacatagagttaatgaccatcagctgcattatgtttctaacttccttttatagcttctccattttgctgaggggctgggtctcatttcttttgtttgtccttagctctgtcttctatttccctaaatgaataattcaaaccttgaggatcctcaaggctattactcaattccgtcttcctcacccccagaagatgactttctttcctatttaactaagagaatccatcggcctggctctctcctgcagtcttagtcttcctgtttcttctcagatccttccctccactcacagaacaaaggattcctaactcctcaagaagagaataccaccatttctatttcaaccatccgaatgtaattggctgtatttcttttcagtttttctcaggcagattttttgatgctgtgggctcagctagaaaatgaatagaatagcaggataaagtgggaactacaaacaggaaactcaaaatgcagagagaaaaacttcattacgggcgcttattcttacaaaatagggagtgaggagtttacatctgttccctactttaagcccttgttgtgagaagggttcacatacctctgaagaccatgctttagtttcacaattttaaaaagatgccatagctgttcttactttgcatagacttctcagattagtgtaaaacattttattgtaaacttcttaaatcgtttgacctgtcttaaattgattaagagatttactgaccacctgcagtgtaaaaggctttggtggacaaacaaaaggaataaatcacaggggccatgtgtcatttgccttgttttcctattacctgctaagtgctagactctcaaatgtttcttgtctaacacagcatctgcccttaagatatttatggtatcacaaacttggagcacagatatgaaaaacatatggtacagtgatgaggaggaggagaaggtgatcagtaatatcagattctaatcagaataagtactggaaaaaggccattggttttgtttattatagttttgttgatgactttcaaaagagtagcttgaataccatagcagtgtaaagacagtctctaccaacttggggagacatgtaacacttatgatgcaagaccacctaagccaaactgtagtataatacttcgaagcaaagaaggcttgtccaagtagtgcattttattctcagaagctgctttactaagacagagaaacaatatggggatgagtgagtattaaatgtcacttccctccctacctaccttgtcaaggtactctcttgcttgattcctggtgaggcaataattcagtctgcatgggagaatatttatgtttctttttacattacgtgggacagacttctgagatgatgattccccctgtgtcatttggaacttgggggagcccctctccctcgctatcagtgactgtatctgatacagtctcattgctttctagccacggcttctactggtctagtgttaaagtatcgtaacaccatatactaaaatattcattgtatggtacaaaatagtgctgtgggacatagagaaagccagaaccagttattatatgcaccggaggtaccagccctgaagtgtgagcctttcgtattttagctagaaaagaagcatacctggttaagtaacaggaaatttcagaatgagctcatgtttaaattctggcattcctgaaatggttttctctatcatttattgaagaatttgaagccagttgattttaaacaagtttcttatgaggtataaactcaaatgtttattgaataattgaaaatggacttaaaagtgtgctgtagtagatatactttgtgattaatggtgtgcactacaagactacattttgagcatttgtaataattttatctcttagacttaatatatggctaatttttcatgagtgtctcaaccataaacagtttgaagttctgtatctgggagagaagtaaaaacaggtttccttaagctctattcctttctagccctcacctctgacttcaagtaagcatagcttgtttatatttgttagtttccttccttaaactaaggaagttttgtgcctctgcctttgcatatgctattccttctccctgaaatgttgctgtcctcccaagtcactttctaaaaattttattaaggaaattttcatacatacaaaagtagtgaagtagtacgaaaacctcccatttattcatcacccagcttcaaccattaacaacattttgccaagtccagataatttaatggttagctcacatgttttcgtggtaaaactttcctttttttttttttttgagacggagtctcgctctgtcgcccaggccggactgcggaccgcagtggcgcaatctcggctcactgcaagctccgcttcccgggttcacgccattctcctgcctcagcctcccgagtagctgggactacaggcgcccgccaccgcgcccggctaattttttgtatttttagtagagacggggtttcaccttgttagccaggatggtctcgatctcctgacctcatgatccacccgcctcggcctcccaaagtgctgggattacaggcgtgagccaccgcgcccggcctttttttttttttttcttttttgagacagagtcttgctctgtcaccaggctggagtgcagtggtgcgatctcggctcactgcaacctccgcctcccaggttcaagcgattcctctgccccagcctcccaaatagctgggactacaggcacccgccaccatgtcgggctaattttttgtattttagtagagacggggtttcaccgtgttggccgggattgtctcgatctcctgacctcatgatctgcctgccttggcctcccaaagtgctgggattacaggcgtgagctaccatgcccagccaagctttctttacttcctagccagggggatctgtttttctcacggtgcacctgcaaaagtttctggatccttcttgtatgtacttattaaatagtactcagtacacattggtaggattgtccttttctcttatttttctctctacagcaagatgttctcctggagggaagagggtgatgtctttataagggcagtggctcacgcctgtaatcccagcattttgggaggccaaggcaggtggattgcccaggagttcgagaccagcctgagcaacatgacgaaaccccctctctaccaaaaaaaaatatatattatattatatatatatttatatattatattatatatatatttatataatatatatttatatatagatatataatatataaatatatataaaacatataaaaatatatatacacacacacatacatacatatatatattagcggggcatggtgtggtggtgcatgcctgtagtcccagctacaggggaggctgaagtgggaggatcacttgagccggggaggcagaggttgcagtgagcctagatcgcagtaactactctccatcctgggtgacagaatgagaccctgtctcaaaataaaatgtgtgtgatggctaacttattaattaaaacatttgaggtgttcattacacatttgttgaatgaatgtctcctccaaaaaatattgtgaatactcagttgtatctgcatgtaacagtaaacaacaacaaaaaactttttttttttaaattatactgtaagttctagggtacatgtccacagcgtgcaggtttgttgcataggtatacatgtgccatgttggtttgctgcacccatcaactcatcatttacattagttatttctcctaatgctatccctcccccagcccgccacccgctgacaggccctggtgtaggtgtgtgatgttccccaccctgtgtccaagtgttctcattgttcagttcccacctatgagtgagaacatgcggtgtttggttttctgtccttgtaatagtttgctgagaatgatggtttccagcttcatccatgtccctgcaaaggacatgaactaatccttttttatggctaaaaaacgttttaaagatagtcaattttgatctttatcagataacaggctttgcttagttctgtagattgaccctcctgtgggtgctttctctgcatgggaagcccttatctggatgcctccttaaaccagacacactgttttaattattatctcagtagatactggctctgggcaaattactgtgggaaaactgaaaaatgattttttttttaaagtctccagagaatcttctggaataggtgtggcagtgaaagaagaaaacaaagaattcctctcttcaggccctgtgcttccatggatctgatacagttattttgtaacaagtcaaatctaacagatttcttattgtacacacttacataagatgtagaagctttcaaaaacaatgcacaccacctgatttgactgattaaatttacagtactgttaacacattgagtggtgaatccgcagcccatgctgctaaaatattgctgcattctatacctctccatattgccaagtttagaagttaataatttaatcttttttttttttttttaaagagatggggtcttgctatgttgcctgtgctggtgtgcaatggcatgattatagcacactgcagccttgaactgggctcaaacaatcctcccacctcaggcacctgaccagctgggactacaggcacacaccactgtccccagcttaatcattctttttaaattacatttttattaactttctttcctgaatacagtgaaaggaaaaagtgaatcaggaataaatatttgtaatagttagtacatagttggatattttcagagaaattttgtattgtaagattacagttaattttatgtattttttgaaaagctaaacatgtaatttagaaatatcaactttaatcagagataagttgccagggcttacatgtgagcacaaggtaggcgttcgttggtgaatatctttaagatcaaaggatgccaggagcggtggctcgtgcctgtcatcctagcactttaggaggccaatgcagtaggatcacttgaggccaggagttcaagaccagactggacaacatagtgagactccatctctacaaaaaaaatttttttaagtcacaaggatgggttttccagtgtaatactttaagatgtaagttggggttctaatgaaatgatgtgatctaatggaatataattaactcaaagaagatttggaaaaagcttttaatgaagtagcattgaaatttatttcactgtttcttggtccttctgattggccctacgatttattttgtttaatatttagtggtatgacccacagtgaaattaaacttgaagtgtctctagtactaaaagaagataatagtggatgagcgtgtgacttatccatggaagagaagaaaagtgaagagttcttttgttttttaagagacagacttagtattgttgcctaggctggtctccagagctcaggcgatccttcccacctcagcctcccaagtagctgggactataggtgtgagatttttagaaatccttcatgtaataaaaatatttaagaagggaaaatagaactcctaaatgtttttggcatgtcattcccttcatttagtaattgggtttgcatgcttttcttgacaaaggaatttctgtaccgcagagagctttttcactgtgactatgctgtgtttagcaaagaagagtttgcctaattgatttcaaactgatttccatacacagaacatataacgttttagtagatatatctgatagttcagtaattaattcacctagttgtattagctcatactcatacaccacacacgctggccaaaacccattgcagcaaatgtgggcaacaaaaaaaatcagctttcaactggggagagccaccttgcaaaagtgattgttcctggtaagtcctctcaagaattgaaagatatcatgccttgcctctgaacaatgcaaggaaagaggcttgctgctgaacatagacagtaaagtctaaacattttatagccttagataatggtttctttgggaaagaccttaaaataggagttactggggaatgtttattaataatcacgtagtgctgagaaggaggatgtcttaaaaaccagacttgtgtcctgattctcccatctgtcagctcagcaagcttaacacaagttatatagctttactgaatcttaagtttccccatctacacataggctttggtaatacttacttcacgggttgttgtgaggaataagtgaagtaatacacgtaaatacacatgtcataaagcatatcctcaataaatattggttgattccaaatttcatattgtggcattgtttagatcacctaccctaaaataaaacttgatctgcttatctagcggatgttagtgaaatgacttcagctgataaaaggaaaatgcatttcctgtgtgtttatcattcaaggtgaaacaggagagaagtatataatagtttgtttaaaatactatcgatagctggccgacacagtggctcacacctgtaatcccagcactttgggaggccaaggctggtggatcacttgaggtcaggagtttgagaccagcctggccaacgtggcgaaaccccacctctactaaaaatacaaaaattagccaggcttgaatggtgcacgcctgtagtcccagctactcgggaggctgaggtgtgagaatggcctgaacccaagaggtggaagctgcagtaagccgagatcgtgccactgcactccagcctgggtgacagagcgaggcttggtctcaaaaaaaaaaaaaaaaaaaaaggtcccagctacttgggaggctgaggtgtgagaattgcttgaacccaagaggtggaagctgcagtgagctgagattgcgccactgcactccagcttgggtgacagagcaaggcttggtctcaaaaaaaaaaaaagtcccagctaccccggggactgaggcaggagaatcgcttgaacccaggaggtggaggctgcagtgagccgagattgcgctactgcactccagcctgggcgacagagcgagactccatctcaaaaactaaataaattaaaatactactgataggaacttaacccttgaaaattccagatgttcacattttcacaaagggaaggagggcaaattctgtgaactttactatgctagataaggaagcctaactccaatcctacgtgagatttctgtcttgagtcgttaacaatactctttctgagtacttaaatgcagtgaagcctcctgggataccacagggattggccaacaactacaggcacaactcatttccttttaaaatggatttacaaccctgggagactaggaaaattcagatattataatgggatttcagcaggctgttcctctaaagagctaaacaattttattcagaagttgctagttaatgggaaggggcagcctctggcagctttgtcctcacctctgttctcttttgttagtaacttagatggaaatactgattacatgctgatcctaaattcaggaagttgagacagtaaatctgtagtgtgtcagaatccacatccagaaaatcttcataggcaagaatgtaataagatgaaactcaattacaggtatattaaggcctatgcttgggcctctgaatccaactgtacaggtgggagcttagcacaatcaagtgtgaaaaagaacttggtgttttagggagttactctcatgtgatattcagaaaatttgtaaagccaatgcaggctgggtgcggtggctcatgcctgtaatcacagcatgttgggaggctaaagcaggaggatcacttgaggccaagagttcaagaccagcctaggcaatatggtgagaccttgtttctaacaaaaacaaaaaacctcagtacaacttcaggttgcattaattgaaattcgttagccagaatatgagaactgagatcccattatgttctgaccagggtgactttacctcattttaatatatgtccatcaaactggagcatgtgtaaagaggaagacaaaagggaaataggtagctaggaaacatgctagtcagaggaatggcttaaagatctgcatgtctattctagagaacacagtatttggaggctgttttcaggtattggagcacttatttagaagggagagcacattatctcaaaaggtagatttaaggccgggcgtggtggctcacgcctgtaataccaacactttgggaggctagggctgggtgtatcacttgaggtcaggaattagagaccagcctggccaacatagtgaaaccccgtctctactaaaaatacaaaaatcacccgcgtatggtcgtgggcacctgtaatcccagctaccaggtggctgaggtgggaggatcacatgagcctaggaaggggaggcttcagtgagctgagattgcaccactgcactccaccctgggcaatagagtgagactccatatcacacacacgcacacatacacatacacacacacacacaaaagtaagactagtagataagtgactttcagtatcttggtaagtctcttaagcatttgagtctagctctagaggaaatagtgagttctctgtcacctgatgagagatgctgtggaggggtttcgggcatctgatgaataaatataacagatgaacagtaacttcttcaaatccagaaattctgagattggtatatgaataaataggtagcttcgaacatgaaccatttgtaatgattctttagttttaatttttttctggtgtattggttaagcatcttatctctcaaatggttaatattacttatactttataaaatatttatttttaaattattctcatagtggtaattgcgtattgttttatctcacaggtagtctcccactattttatatctttgttacttcaagtaagacaatcttaaatatgagaggacatctagagattgtgctaagattgcgtcgtgatggtaattaatgtaactgatagaatgttatcttttccaggtagtaaacctattgatggcaattttgctgactgtggaagtgactcatccaaactccatgccagctgtcaacattcagtatgaagtcatcggtaattactattcgtctgagagaatggctggtatgtgtttaatagaaaaacaaaaaaatctcccctgctaatttttatatacagtcctgtgttgtttaatgatggggacacattctgagaaatgtcactgggtgatattgttgtgtgaacatcatagggtgtacttacacaaacctagatagcgtagcctactatacacctaggcgatgtggtagagactcctaggctaaaacctgtacagcaggttactgtaccgaatactgtaggcagttatcatacagtggcaaataggtgtgtatctaaagatggaaaaatatagtaatactgtaaatacactattacagtgttatggaaccctcatgatatatgcagtccattgttgaccaaaatgttgttggcccatgtctgtctgtctccacacacactggttatttaggggattgattagattatgcttaagttacatttcagaaagttaaaatggttacataataagggtaacaagttaaattatatttaagtcattcttgctgaggcaggaaagtattttgatgggtttttcagagagattcagtgaggattacaatagagcatttctagccagaaaatgtaatggactattaagtttttgagaaatgtgtaaaatgtaccttgaaatcaaaataagcatgcaggcttcataagactttttttttaatgtagctactagtagaaaggagaagttgctatggttacaatatatgtgtccctccaaaattcacatgctggagcttaacctccaaggtgatgatattaagaggtaggacccttgggtggtgattaggccatgagggctctacccttatgaatgggataaatgcctttctttttttttgagacagagttttgcgcttgttgcccaggctggagtgcagtggtgcgatctcggctcagggcaacctctgcctcctgggttcaagcgattctcctgcctcagcctcctgagtagctgggattacaggcacacaccaccacacccggctaattttttgtatttttagagttactccatgttggtcaggttggtctcgaactcccgaccttaggtgatccacccgcctcagcctcccaaagtgctgggattacaggcgtgccacagcgcccagccagcgcccttcatttttaaaatcacatttatattatttatttgtttaaaaaaagttttttagagaaaaggtcttactttgttgtcccaactggagtgcagtggtacagtaatagctcactgtaacccagaattcctgagctcaagtgatcctcccacctcagctgcctgagtagctaaatttttgtggagatcgggtctccctgtctttaccaggctggtcttgagctcccggcttcaagggaccctcccacttcagcctcccaaagtgctgagattacaggcatgagccactgtgcccagcccatagcagactcttgaaagccgattatttgaattacaaattttgctttcaagtttgataatacctgtttaatttcatattttgtttttacctgaattcttctaagtggaagtttgcatgttgtagcagggaagtcctttaaattcttaacatttatcaatcctgtatcctctaagaaaaaagtaaagaggactattcctagtttagtgccaaatattgatgaggttttactgcccagtgataaattatatatacatgctgtgcatggtggctcatgcctgtaatcccatcactttgggaggccgaggcaggtgaatcacttgaggtcaggagtttgagactagcctggctaacatggtgaaatcctgtctctattaaaaatacaaaaattagctgggcttggtggcgcaggcctgcaatcctagctactctggaggctgaggcaaaagaatctcatgaacctgggaggcagaggttgcagtgagctgagattgtgccactgcactccagcctgggtgacagtgagattgtctcaaagaaaaaaaaaaagttgtatatacatttacccaatagggaatattcagcagttgttctagtcattcatcattattagtgccctcagtattttttttcttttttcatttttaaaacatcttttgtgcatacagggtcttgctatattgcccaggttggtcttgaactcctggcctcaagtaattttcccaccttagtctctcagagtactgagattacaggtgtgggttgccgtgcccatcccccccagtttcttaatctctgcccagacggatcacctgatgttgggagtttagaccagcctggccaacatggtgaaacctcgtctctactagaaatacagaaattagctggatgtggtggtgcttgcctataattccagctacttgggaggctgaggcaagagaatcacatgaacctgggaggttgaggttgcagtgagccgagatcgcgccactgcactctagcctgggcgacagagcaagactctgtctcaggaaaagaaagaaaaaactgtccagagtcacagaatcaactacagatttgtgctaagaaattcagtaagtcaggcacggtttagaattgcttgagattacttggctgttttgtgaaaaaattagtgaacatggattaatggagagttggaacaacctgaggaagagttgagaaacgaagaagaaagtagtgtgtgttaacatagataccagggtgcaggaagcaatgaattttggtgtttgggaggttgggtggggaagtgaggttaatggcgaaatttgaatttcaggttatatttatctagagcaaagggtaatgtatgtagcattctgagtttaatacacttgtcttttaagcaattgccagccattccttccagctaaaccctactcataagacccatcttgtgtcatttcttcatattttttcctgatgcttcaggcaaagcaaattgtttctgcctctgttgtagcacttgacacattggattataatgatctgtttgcacatcttcagattcctgggctcataagggcacagggacctgtttctctagcagagaatgtggttcatgtgccatcctacacccatgtcagtgagtcgttggagcgatttaacaaataagtgctttgtgtgaatgtctcaaggttccttacttcatgctgagactttgattttttttttttttttttaccctcttggccttagatctttttatagccaattttgaaaatatttaaatttaatattttaaagctagctttaagtgaagttttgttctttttgttgtgtaagtagcttaattagatttttgtttttgttttcttagataatgcctgtgttctttttgccgtctctgttcttatgtttataatcagttcaatgctggtttatggagcaatttctgtaagtatactgtattttcatcttttagagtttgaccttaggggagcttagtaatttcctatatgtgatacaatgttatggttgttattttttctccagtatcaagtgggttggctgattccattcttctgttaccgactttttgacttcgtcctcagttgcctggttgctattagttctctcacctatttgccaagaatcaaagaatatctggatcaactagtaagtgtgtatgctcattaaacttatttttttttcattttgagtaaaatgtttacatttaaattctgaatcagccttaggtattatttttaactactccgcttgcttttgaaagtgtcttaaaataattatttctgctctcttagctactgtgacaaaactggcaagatgaaagtagagtaatttcttaccatccatttggataatgcagtattaagagttcctagaaatggatattatggaactagtagttcagcttttgtttttgagcatccaaaggtacagtaatttgactgtgtatggcactctttaagtgactagcttattgtctttacagacatctgtagcaagtattccagtgcctactatgtttttagccctttgctaggcactgttggtgaatggtgtggatataaagataactgcgaatattacagtttagcgagcaaactgtaatatttggggagtcaagatacatgaagatgttacctgtgataacaagcgggagctgtcacagcagcagcaaataaggaacttcgattaatgtcatatggaggggggcagggaaaactctgggctgggtggccaaggagggcgtcgtggaggtggtcagaaggagatatgggatctaggtagatgaaaattggcaggagtatcctcagttcatttccaaagtgctgtctttcagaacttttcttcccactaccagatttcctgcctagcccattttccacaccaccaaattcatcattcatttattcagaaaacattcgctggttgctcttatgtgcaaggcactctgctaggtcatagagatgcatcagtggagcttacttcctggtgaaggaatcaggaagttgacagttaactgatacaccattgtaggttactgtgggagtggatatcacatgggtactaaacctgggctgggaagtgagatttgaattgtactcaggagtttgaacaggaattaagatagagaaagtatagaagacaatgttccagataaaagagagaccttgaggcaagaagcagcatagtgagggcactagaagctggccatggtggctggcacacagagggagggtggggaagcctggagaaaggtggccaggggcagggctgttggcctggtcagccacggtcagcggcttctgtgtggctctctgctcatgtcatctccgggggagaaatttcaagtggtgcctccctgccttctgagtaaaacccttagcatagcattccctcatgagctccaccttctgtgtttttctttttgtttatagatgaagatcaaaaaaacagcttttttcccaaggagacaacctggctggggtttggggagtagtgtgttcttaaaattttttttttctatttttttcagtcctttgggatttctgtttatgttgctttttaaatgaaaatgtaattaggccttcttgttgacatttccattaaaaacaagcataacttaaaaaaattaaatcagctttatagaaaagaagataaaaatttattattccttttagagaccctggtgttttctctcctctgttttgttcacttagtgcctcagactggaaggtatttcctctgttggttagtttttaaatctgaaattcttcttttataacacctttctccctgaacttagctcattgccttgtacacagtcagggctgattaagtatttaaatgaatatagctgatgcttttcagtagaacagagtatgtctgcagtaaaacttatgtcttgcctgacattgtaaccagtgggtgtatttttgcccctgtctgatgagagttgagacaaacgattgacatttagaaaaataattttccccttgtagcattaaaatggcccagtttatctgtagaacttacacagaactgagcatctcctgagttaatgatgcaagtgacacagggactgatctccagtgccatataggcgatagtgtttttccaaaggccagaaagatggaaaatagtgaaaagtaaagagaaaacgagctaaatttagactttttggagcattgtgggttatcaaatgtactctaagtgattgttagcatgcattaatggtgataaggcttaaaatttaaaagcactttgaattaaattctgcttatacatttttattaaatatcacttttgactttggtgcctttgtattcttcagatgctcttcaggttatcagcaagtgctagtgcgatggctgacttgtaaatgtgtgtgtgtttctttaatagcctgattttccctacaaagatgacctcctggccttggactccagctgcctcctgttcattgttcttgtgttctttgccttattcatcatttttaaggtacgttgctgactaaatctttaggggtgactgagattgacactgtgaatctaaacctttttgctgcctttcaccttacaagtcacaggagattgaggatttgtggggacagtgtttattctggcacatgagcagagatagcttctagggaatggtttctgttgtgtttccaaggaatggttgtaactgtcaggatccttctctgagtgaaaggacctactttttatgtttttgaaaaggggttcatgatttgtattgaaaatctgttaaaattcccaaggttcacaaacccagaatttcttctgcagcacagtgggttgacctgaattgaagctccatctctggtttctctctgaaatgttggcgatgaaactcatgacacttaaatgagagcaagtagggaaagggcctggcctgtgctgggtccacacagaggtcagctcccttctacacagaggatgtgtggggaaagcattctaagtcactggcattttagagcaggtagtctgttgagtttctagcagatgtcaactttgtttttattcttccaggcttatctaattaactgtgtttggaactgctataaatacatcaacaaccgaaacgtgccggagattgctgtgtaccctgcctttgaagcacctcctcaggttagctactgttggatagagtaaaaggtcaccagtcactattgactgttccaggagaagaacctggagcttgactttctctttcatgctgtaacctatggtttgggcatgaaccctgggtacttttatgaacagcttaaaatccacaggattcaacattttccacttggaatcctgagtacctgggtcgcttgtctcatccagtttggctgttagatttactcattagaggtcctcattagtgtcatgtttgtaggggagatgacaggaaacagctgtgttaggcccatgtagcctggggcttctaaggatggggagttccattgactgaaacttgtgttgttttcagaaaacatccatatgatccaacgggggagctgacctgtctggggagatgaatacagtggtttcatgggtcttttggggaagcctacctttcacagccctctaattgctgctgtgctaccttcccaggttttcagcatataaaatatatgtggcccatctagggccaggctttcagacttggagtcacttcgattagaatgtcactcctccactgcataggtgtgcctgttaccctcgaagacatgatataagttcatgatcttctgactacaaactgattctgatgaaagtcaggatccaggagctagcgtcttattgggtgtttcctattgcatggtttaacccaactcctcaacacctgaagtgtaattggcactggcacttcatcggtcacactggaatgagggcagagcaatggcacccactgcctgcctgtctaggaatccttgtccagaagcaggactctctctccccacctccctctctccaccctccccttcgctagcaatcttggtaaaattccaggttattgaggcatctacttgggaacattttagctaacagtttcttcagctcttctttatggttttgtataaccctatttagggcataaagtctgaaagctgaattatctcacaaaggattttaaaggagaattaaatctgatacaattttttttctctctttagtacgttttgccaacctatgaaatggccgtgaaaatgcctgaaaaagaaccaccacctccttacttacctgcctgaagaaattctgcctttgacaataaatcctataccagctttttgtttgtttatgttacagaatgctgcaattcagggctcttcaaacttgtttgatataaaatatgttgtcttttgtttaagcatttattttcaaacactaaggagctttttgacatctgttaaacgtctttttgtttttttgttaagtcttttacattttaatagtttttgaagacaatctaggttaagcaagagcaaagtgccattgtttgcctttaattggggggtgggaagggaaagagggtacttgccacatagtttcctttttaactgcactttctttatataatcgtttgcattttgttacttgctaccctgagtactttcaggaagactgacttaaatattcggggtgagtaagtagttgggtataagatctgaacttttcatctgcagaggcaagaaaaatatttgacattgtgacttgactgtggaagatgatggttgcatgtttctagtttgtatatgtttccatctttgtgataagatgatttaataaatctctttaaatacttagggttgtcattgtttttaatccgttacttttttttaagataaaccctgacattttccattacagtgtattcagagtgccagtgtttattttttttttaactttgagaatacagttggcccttcgtatccatgggttctgtatccatgggttctatatccatggagttaatcgactgtggattgaaaatatttggggggaaaaatggatagttggatctgtactgaacatgtacagactttttattcttgtcgttgttctctaaaaaatacactataacaatttatgtagcattcacattatattagatattataagtaatctagagatcattttaaaaatatgggaggatatgcatgggtcatatgcaaatactataccattttattataagggacttgagcaactgtggatttgggtatctggagggagaacctgcaaccagtcccccatagataccaagggacaactgcacaatgatgcacagttaattagaaatcattttcttaatccattaactctaaatttgtggtgaagatatttcttgaatcacaggacagaaattaataccggtggtttgaaaagtgattgctgtagcaatcttttttttatgtcttattttttttctttccaacttcttaggttcagagggtacatgtgcaggtttgtaacatgggtaaattgtgttgcaggggttttgtcagtgttttgtcatccaggttgtgagcatagtacctgatagctagttttttgatcctcaccctctactctcaggtaggccctggtatctattactcccttctttgtgtccatgtgtacttaatgtttagctcccacttataagtgagagtatttggatttctgttcttgcattaatttgcctaggataatggcctccagctccatccatgctcctgcagaggacatgatcttgttcttttttatgactgtagtattccataatatatatgtaccacattttcattatccagtccactgttgatgggtatttaggttgattccatgcttttgctattgtgaatagtgctgtgatgaacatatgtgtacatgtgtctttttgggagaataatttatattcctttaggtatatacctgataataggattgctgggttgaatggtagaatgttttaagtttgagaaatcccttatgctgctttacacagagatggaactaatttgcattcccaccaacagtgtgtaagtgttcccttttctctgcaaccttgccagcatatgtttctttttactttttaatcatagctgttctgactggggtgagatggatttctctgattattagcatttctctaatggttagtgatgttgagcattttttcatttacttattggccatgtgtatgtcttttgagaagtgtctgttcatgtcctttaccaattttttttttttttttttgagacagggtcctcgctctgtcacctaggctggagtgcagtggcacgatcttggctctctgcaacctccacctcctgggttcaagcgattctcatgactcagcctcctgtgtagctgggactacaggtgtgtgccaccacacccggctaattttcgtatttttagtagagatggggtttcaccatgttagctaggctggtctcgaactcctgacctcaagtgttccaccttcctcagcctcccaaagtgctgagattacagctgtgagccactacacccagcctcctttgcccacttttaatgttgttgttgttgttgttgttttgttttttgcttaagttccttatagattctgaatattagacctttgtcatatccatagtttgcaaatattttcttccattctgtaggttgtctggttgctctgttgatagtttctttagcagagcagagaagctctttagtttaattaggtcccatttgtcaatttttgtttttgttgcagttgcttttggtgtcttcctcatgaaatctttgccagggcctacatccagaatggtatttcttaggtgtccttctaggggttttgtttgttttgttttttcagttaggttttacatgtaagtcttgatatgggtttggatttgtgtccccatccgaatcttatgttgaattgtaatccccagcattggaggtggtgcctggtgggaggtgattggatcatgggggtggtttctcgtggtttaacatcatccttcttgctgctgttctcgtgatagtgagttattgcgagatctgtgtgtgtagcacctcccccttctctctcttgctcccgctcctgcaatgtaagatgcttggctccccctttgccttctgccacgactgaaagctccctgatgcctccccagaagctgatgctgccatgcttcctgtacagcctgtggagtgagccagttaaacctctgttctttataaattattcagtctcagctatttctttatagcagtgcaagaacagactaatagaagtctttaatccatcttaagttatttttatatatggtgaaggtgtctagttctaatctgcatttggctaaccagttatcccagcaccatttattgaatagtcctttccccattacttatttttgtcaactttattgaagatcagatggctgtagtttgtatggctttatttctgggttctgtatttggttccattggtctgtctgtttttgtaccagtaccatgctgttttgattactgtagtcttgtagtatagtttgaagttggataatgtgatgccttcagctttgttctttctgcttaggattgccttggctattcaggctttttttggttccatatgaattttgaaatagttttttctaattctgtgaaaatgccattggtagtttaataggaatagcattgaatctgtaaactgctttgggcagtatggccattttaacaatattgattcttcctatctgtgagcatggaatctttttccatttgtttatgtcgtctctgatttctgtcagcagtgttttgtaattctcattatagagatctttcaccttcctggttagctgtattcctagctacttaagtcttttgtggctattgtgaatgggattgcattcttgatttgactctcagcttgggtattattgttgtatagaaatgctactgatttttgtgtattgattttatatcctgagactgctgaagtttatatctaggagctttgaggcagagactatggggttttctaagtttagaatcacattttctgtgaaaagagatattttgacttcctctcttcctatttggatgctttttatttttttcgcttgcctgattgttctcactaggacttccagtactgtgttgaataggagtggggagagtgggcatccttgtcttgttccagttctcaagcagaatgcttccagcttttgccaattcagtgtaatgttggctgtggatttgtcatagataattcttattgttttgaggttacgttcctttgatgcctcatagttttttaagggtttttgccatgaagggatgttgaattttattgaaagcattttctgtgtctatcgagatgatcatgtggttttgtttttagttctatttatgtgatgaattacatgcattgatttccatatattgaaccaaccttggatcctaggaataaagcccacttgatcacagtgggttggctttttgatgtgttgctgggttttggttcataaaaggcagttcccctgcacacactgttgcctattacccagttccaaagtcgattccacattttcaggtctccttatttatgttcttcagggatattggcctgaagttttctttttttttgttgtgtctctgccaggttttggtatgagaatgatgctggcctcataggatgagttagggaggaattctcctcaagtttttggaatagtttctgtaggattggtaccagctctttatatgtctggtagaatttggctgtgaatctgtttgggaccttttccagttggtaggagtattattattactgattcaatttcagaactcaggctggatacagtggctcatgcatgtaatcccagcactttgggaggccaaggcgggtggattgcttgaggccaggagttcgagacctgcctggacaatatggtaaaaccctgtctctactaaaaatacaaggtcaatgtgaaagaaaaaatattaaaagcagctagagagaaggggcaggtcagctacatcaggttaacagcagatctttcagcagaaaccctacaagccagaagagattggggacctatattcagcattcttaaaaattccaaccaagaatttcatatccagccaaactaagcttcataatcaaaaaagaaataaaatccttttcagtacctaggatgcaagtccactacagctcgtgtgtcctgtgtccctgctaaggaacaagttggagatgaggacctcttactctcttttcccatctatatttggcacagtgacccacaaaaagctgagggggagggaattaaatgttttctgaatgaaataatttctctcccctccacaaattttatactgctaatagatcattttcaatataattgggaatataccagtacacatactacacttacatttaaagtttctggcaataggaaaccaagagatctttcactgataagttgtacattttattccctctggtgactcacgctgtttttcagtctgtggctaatttgttcacagttatcgtggacttagctttgttctgagcctaattgttccactatcccttaataggcaaaggatagatgtgctattttcagaactcctgctttcaaagaaggaatgtgttataaataagcccaagagttgactgaaagaaaaagttgtacctttgcttttctgaggttgctttttctgtaaatgtcatgactttggttatgagaaaataatttagaaacaagatgagtgttgccaaataatgtggaattccaggcacacgagagaggcaacactgggatggtggaatgaacacaaagtttggcatcagaaggttgaaattcaattcctgtcttcacccagcgattacactctgaccctgggaaatttaactaggatctgagcttcagtttttgcatctataaaatcagaaagatgcaaataactgacctatgaactcggagttgttcttaggatcaagtgaggtaacagacccggaagggttggcagaccataaattactgtgcaaatattatgagggtgtatgagagcaaggaatggctggaggaagggctggagagggagaaaagtatttgacctggaaaccagaaaaagagtgactttgttccctgtctgttagaaaggagctttgtacagtagtccccccttatgtgtgggggatataccctaagaaccccagtgggtgcctgaaaccatggatagtaacagtaccctctatatactatgctttttcctataatacatacctatgataaagtttaatttataaattagagtaagagattaaaagcaataactaataaaatagaacaactacaatttttttaggttttttttattattattatactttaagttttagggtacatgtgcacaatgtgcaggttagttacatatgtatacatgtgccatgctggtgtgctgcacccattaactcgtcatttagcattaggtatatctcccaatgctatccctcctcgctccccccaccccacaacagtccccagagagtgattttccccttcctgtgtccatgtgttctcattgttcaattcccacctatgagtgagaacatgcggtgtttggttttttgtccttgcgatagtttgctgagaatggtgatttccaatttcatccatgtccctacaaaggacatgaactcatccttttttatggctgcatagtatcccatgatgtatatgtgccacattttcttaatccagtctataattgttggacatttgggttggtaccaagtctttgctattgtgaatagtgccacaataaacatacgtgtgcatgtgtctttatagcagcatgatttatagtcctttgggtatatatccagtaatgggatggctgggtcaaatggtatttctagttctagatccttgaggaatcgccacactgacttccacaatggttgaactagtttacactcccaccaacagtgtaaaagtgttcctatttctccacatcctctccagcacctgttgtttcttggctttttaatgatcaccattctaactggtgtgagatggtatctcattgtggttttgatttgcatttctctgatggccagtgatggtgagcattttttcatgtgttttttggctgcataaatgtcttcttttgagaagtgtccgttcatgtccttggcccactttttgatggggttgtttttttcttgtaaatttgtttgagttcatcgtagattctggatattagccctttgtcagatgagtaggttgcgaaaattttctcccattttgtaggttgccttttcactctgatggtagttttttttgctgtgcagaagctctttagtttaattagatcccatttgtcaattttggcttttgttgccactgcttttggtgttttagacatgaagtccttgcccatgcctatgtcctgaatggtaatgcctaggttttcttctagggtttttatggttttaggtctaacatttaagtctttaatccatcttgaattaatttttgtataaggtgtaaggaagggatccagtttcagctttctacatatggctagccagttttcccagcaccatttattaaatagagaatcctttccccattgcttgtttttctcaggtttgtcaaagatcagatagttgtagatatgcggcgttatttctgagggctctgttctgtagaacaactacaatttaataaaagttatctgaatgtggtttccctctctgtatctctctcaaaacatcttatactgtacctacctttcttcttgtgatctgttgatctgataaccaagatgattacgaagtgcccaataggtgggtaatacatgcaggcagcgtggatgcactggacaaaggagtgagtcatgtccagggcagtacagagcaggacagtgtgagagtttatcatgctactcagaatggcatgcaatttaaaacttataaattatttttttaaagcctatggattatttacttctggaattttccattcagtatttttggactgtagtcccccacagataactgaaactttgaaaagtaaaaccacagataaccggggactagtatatctgttatcatttggttaggttgcatcagagaacttgatttatattcctggccctcccaccaattatatgaattcagtaagtaattcagattgcaaatgagattgtacattccttcttcctctgaccttctgtgactcacttttggttaaacagttaattatcttttattattattttttagagacagggtctttatcacccaggctgtagtgcaatggtgcaattatagctcactgcagcctcaatttcctgtcctcaagcaatcctccctccttggtctcccaaagtgctgagattacaagtgtgagccaccacatccagccagttcatgatcttttaaagataaaaagataatttaaaaaactacataggtaccctcttagttaccatttttggtgttcttcattcccttgtgtagatacatatttccatctggtataatttttcttctttcagaaagactttaacattttttacagtgtggttctgctggtgataagttcttttgggttttgtatatctaaaaatatttttactcactttgggaggttaaggcgggcagatcatgaggtcaggagtttgagaccagactgaccaacatggtgaaaccccatctctactaaaaatacaaaaagtagccgggcatggtggtgcgggcctgtaatcccagctactcaggaggctgaggcaggagaattgcttgaacccaggaggcggaggttgcagtgagccaagatcgctccactgcactccagcctgggcaacagagcaagacaccatctcaaaaaaaaaaaaaattacctttgtttttcaaaagctatcttccctggatgtagaattctaagtggacctttttttggtttatttttgttttttttctttcagcactttaaagatattgcttcactgtcttcttgaacacattgtttctaatgagaaatttactgtcatccttgactttctctgtatggaacattttttttctttgggtaatgaacattttctcttatagccctgattttgaacaatttgtttatgatgttcctcgatgctggctttttgttttttcacatttgttgcatttgaggttcattgagattcttaaatctgtaggtttatagatttcatcatgtttggaaaaatttcagtcattatctcttcaaatattttctctgtcctctcttctccctccttcagggatcctgtctggctggaaaagggaagataaacttcctacagtgagcagatttgagataggtcttgagagttggttaggacttgaactggtagagatgtcaatcaaagacttcgcaacttaatatgaaaggatcaagagaggaaggaggtcatcaagatttctttcatgcctgtctggccagaggaatagtggaataaatggggaataaaggacagtgagcttttttaaaaaagaggaagatgagttcattcttggttatgattgagcatgacgtacttttgtgacctttaaatagaggagtacaaaagcgagttacaaatgtagttttattgctcaggagtaggttgggacaagagactctgatctggaaattagcaatatagaggtggtagctgaaacaacgctggtagttgagactcctaacatagatggtacacaaagtatcaagacctagtcatttcagcccttccatttctagtaatttcttctagggagataatagtaattcacaaagattcatgaaaaaaggttcagtgaagcacagcttgcaattaaaaacaagtagaaactacatgtcccacaatggaaaattggttaagtaaatgaaatatacagtgaaatacagtacagtcattacaaatgttgtatttgaagaatataacagaaaagtgttcactatatgttaagtacagaaagcaagttataaccctacaacatagcatggtctcatttttaataaaaatggatgcatatataagcagagacaaaagaggtaacaccacatagcttatctttgggtggtagtctatggatggtctttccattcttttgcttatctacagtttctacattctgtatagtaaagtatatgttacttttatactaagaaaaaaatcgattttcctttttaaaagctgatttagaaaaaagcctgtggacttccacacttcatagagcatggaaagaagaaaaggaagtggcaaatgagagagacagtgtagaatcaggactataggacaacctgggatgaaggggctaacactggagctcctgccaaaaacagggtgatctaaactctaccaagaggcctctgggcttggtgatctgagccattatgcacttaggagagcactttccttaggatagtgcaggggaggaaggaagagcactttccccaggatggtacaggggaggcagtaagaaatgggttaagaagtgatagctgttgagaagtgtggtgtggagagaaggaggtgggaggatagtcactcattggccccagatgtttcatgaccccagatccagatgctcagcaagtcccattcataacaaagacatggggccactgtgcagtttccttgctggtggccatttccttgcacctgctcttactgcagatacaggcctgttgccaagatcattcgaatgagatactacaaactgcaggacacaaactccctcggcatagcagttgcctcctgatatggtttggctgtgtccccacacaaatctcaccttgaattgtaataatccccacatgtcaagggtggggccacgtagagataattgaatcatgggggcagtttcccccatactgttctcatggtagtgaataagtctcatgaaatgtgatggttttgtaaatgggagttcccctgcacaagctctcttgcctgctgccatgtaacatgtgcctttgcttctcctttgcctccagccatcattatgcaggctccccagctatgtggaactgtgagtccattaaacctctttcctttataagttacccagtctcgggtatgcctttattagcagcaagagaacagactaatacagtaacttggtactggtagaataaattggtactggtattgatattggtaaattgctgctgtatagatactcaaaaatgtggaagtgactttggaactcggtaacaggcagaggttggaacagtttggagggctcagaagaagacaaaaaaatgtgggaaagtttggaacttcctagagacttgttgaatggctttgaaaaaaatgctgatagtgatatggacaataaagtccaggctgaggtggtctcagatggagatgaggaacttattgggaactgtagcaaaagtgactcttgctatgttttagcaaggagactggtggcattttgcccctgtcctagagatttttggaatttttaacttgagagagatgatttagggcatctgatggaagaaatttctaagtggcaaagtgttcaagaggtgacctgggtgctgctaagagtattttgttttatgtatgcacaaagatatggtttggaataggaacttatatttaaaagggaagcagagcataaaagttcagaaaatttgcagcctgatgatgcaatacaaaagaaaaacccatttcctgagcagaaattcaagctgctgaataaatttgcataagtgatgaggagccaaatgctaaccgccaagacaatggggaaaatatatccagggcatgtcagaggtcttcatggcagcccctcccaccacaggcccagaagcctaggaggaaaaaatgtttttgtgggcccagcccagggacttgctgctttgtgcagtctgggtacttggtgctctgcatcccacctgtggctaaaaggggccaacaaagagctcaggccattgcttcagagggtgcaagcccgaagccttgttggcttccacgtggtgttgagcctgcaggtgcacagaaatcaagaattgaggcttgggaacctcctcctagatttcagaaaatatatggaaaggcctggatgtccagacaaaagtttgctgcaggggcagagccctcatggaaaattcctattagggcagtgcagaagggaaatgtggggtaggagcccccacacagagtcccaatgggcactgcctactggagctgtgagaagagtggcaccatccttcagaccccagaatggtagatccactgacagcttgcactatgcacctggaaaaactgcagacactcagtgccagcctgtgagagcaaccaagaggggggctgtaccctgcaaagccacagcagcagagctgctcaaggccatgggaatctacttcttgcatgagtttgacctggaatgtgagacacagagtcaaaggagatcattttcgagctttaagatatgactacccctccccaggattttggacttgcacggggcctctagccctttcgtttttgtcaatttctcccatttggcacaggtgtatttactcattgcctgtatcacccttgtaccttggaagtaactaagttgcttttgatcttgcagtctcataggctgaagggacttcccttctctcagatgagactttggaactgtggacttttgagttaatgctgaaatgagttaaagctttaggggactgttgggaaggcatgattggttttgaaatgtgagtacatgagatttgggaggggctgaggcagaatgatatgatttggctgtgtccccacccaaatcttaccttgaattgtaataatccccatgtgtcaaggttggtgccaggcagagataattgagtcatgggggtggtttcccccacactgttcttgtggtggtgaataagtctcatgagagctgatggttttataaacgggagttcccctgcacaagctttcttgcctgccaccatgtaagatgtgcctgtgcttctcctttgccttctgccatgattgtgagggctccccagtcatgtagaactgtgagtccattgaacctcttttctttataaattacccagtctcaggtatgtctttattagcagcatgagaacagactaatacacctccagaaactggaaattcctgggcccagaacccaaccatctgattcagcccatatagggtgggggcccaggaatctgtctttgataaggcctcccgggtgattctgacagtccagttgcaggcccactgcatcagggcatggggactacaggcctccttcccctttgtttgatttgcacccagagctggaaaagactgaacctctgagagatatgaaaaggcttccattggaggtttctgtaaatagcacaactgcatgttttgtcataaatcagtaagtctaggatgtggaaaaaatatatcttgggatccatggccttgcatgtgtcctgggagattttagagccaggctctgcacaacccgaagtcaagaaagggcatgactcttgggagtgacaacttgttggaagcggattccaaccatgcttcccatttggcctgagctgagagctagcagtttccctactcgcaggacaccaagcaacaagaacaaagaaattgctccaccaaggctatctacttatgaagatgaaaagctgttagaaagtggttaggattagatcagccaacgtgagtggcctcttgaggagaagccaacctggaatgagagatgaagggttcttgttgaatcttctaagagaagggccctctgagggcccccatctatccccatcataagcaatgtctaaataaattgtgctagatcctgttgtagtttccaaatatttgccatttttccagcaaaaagttgatattaatatttcccaactgctgccatcaggcttggccacgtggcttgctccagccaataacgtgaatgaaattgatttgccaccttcttgcaggtgcatgattctaccatcttttttccttcctttgcctcaagaccagcacattccagattgggcctgctcctatgtctgggtgccagaatgaagaagacatggaggagagctgcagccaacctatgaaggacacataacacagcaagaaataaacgtcctttgttataagaacatgagtgtggggccattggttaccatagcataacctagccaaagttgactaataaatagttttttagaattgtaaaaaaattatagcaatattaaagtgatttttttaaagaaaagcaactcttaatttactttctagccttggttaaatagttttaatttccacagatgctcacatgctaatgtgctgaaacagacattacactttaggctgcgtctcccccaaacttcttccctagaaggagcctggtgttggaggaggatctcactccactgctagagaaggccaaggaagagatggtctgtggagccaatgcccagcacagggcagtctgttgtcagtgaagtgaaacagagcactcgccaccactcaattcctggaaaaagcagtgccaggctgtttctcttcctacaaacaatctatcatgcctgttgactactctgccaaggaaaggctgaatatcagctctgttccatgatctcccatttcctgaagtctcagtcctaacagaggttgtgaacctgcatgtggtaaggtagaatttctgggaatttagtagcaaacaatgcaagcattgcctctgccctcttggagcctatgctctagtggagtctgatggaatgaatgcatttaatacatattaaataattacaaagtgagtaaggcccactgaagcaacaaaccagacacttgtaatagattgaaagggcagaggcagcagaagaaaatctatttaatacagacgttttcaaacattagtgaatcatgaattctatttagatgtttatagccagatttttaagaaaatgaaatagaaatgaatggagttgaataaaataggaatcattggagtgcatcacatgtgttgtcaatattgctttataaaactcatttctgggccaggcacggtggctcacgcctgtaatcccagcacttcggaaggccgaggcgggcggatcacgaggtcaggagatcgagaccatcctggctaacacggtgaaactccatctctactaaaaatacaaaaaaaaaaaaaaaaaattagccaggcatagtggcaggcacctgtagtcccaactactcaggaggcttaggcaggagaatggtgtgaacccgggaggcggagcttgcagtgagcagagatcgcaccactgtactccagcctgggcaacagagcaagactccgtctcaaaaaaaaaaaaaaacttatttctgttgtatatatgtattatatatttgtgcagtaactggctcataatataaaatgtgttgttatagtgaattgtggtaaaaacaaatttgaaaaacctaatttacaatatggcttcagagaatcaactctggggaagacattacagtgaactggctgacttgataattgtttttttatttactagtgaaacttaatgctcatgcttgtacttctaaaatgaaacaataaaaattaaaaccaatcttttattaaaggaagcaaaagtgttatgagttgctcttttcaaatattgattctactattttgaatagaaatatgtcttaagttgtaaatgttagttaataatagtaggtcattaacagccctcttcagaacttactggcaataatgaaatgtgcttcctgtgtttcagaaatagtgtttttttttcttttttagagcaagccaaactttgccttcaacctcatctcatttgcaagctactctcatatagctaagaatttggtattaaaaataggcacaggccttaaaacacaagagtttagatttagtttccatgttttctgcttattcacagaaatattgactgattgattgattgagacagggtctcactctgtcgcccaggctggagtgcagtgctacgatcatggcttactgtagcctcaatggcctgagctgaagcagtcctcctacctcagtctcccgagtagttgggaccataggtgtgtgccaccacacctggctaatttttaaaaaatttttcataaagatggagtctcactatgttgcccaggctggtcttgaactcctgggctcaagcgctcctcccacctcccaaagtgctgggattacaggcatgagctaccacatccagtctagaaatatttatttaataaaataaatattaatgggtagtaccaatccgtaatttgtactatcttaatccctgtggcaattaaaaaacagagatggcaatacaacttgtaggctattgaacatattcatttttcataattacttgtttttttcaggtgggtaaattgcaaaaaacataggtaaagagttctatactttctggaaaagacttccaccttgggattgagacagttttgaacacaaaagaaaacaaatttttttctccaagtacatggtctgtcatttatcagatttaagaagtcttgggacatcttttgacaacattttctccaatgcatccatggaatcaaaggacttttcaacttgtttgggggtagattttttggtctaaagttggaaacatatgtcttctttcaatagaacagtggatgtctgcacacgtatttttcaatgtttacacctacaagtctatgcacattcaagacattggcaacagtggtgttattggcaagggtggttgggatagttccttttcaccatatacctgtagtatatgccacaggttagctgtcaactcccattccaatgcatgttttcctttgccttcttcactgtggatcaaaaaagctaagaacttacctttcaagacccccttgtggccaggtgtggccatgagatgtcagcagcaaatatcttttggtcctttgctctttggcctttggtcttccctcttccttcatcctggaatgccaacatgatacttggagttccagcagccttgggaacaaaatccacacattaagggcagcaaagcaggagacaagtggttccttaagtagcttgcaagtcttattctactgctgaaatcctagttgtgagaaaaataattctcccaagatgttgaatttattgcttgtaagaagtaatgcaaagttgaaagtaatcctaacatttggtcggtttcttttttcttttcaaagtttagaccaagtgcatgtattacctactttaaaaataataaaattctaaaaacaatgtaaagtgtatatataaatctttgttgtatttcttagatttttgaatagttcaaaaaacctctgtttggcttttttatgacctaagtctttaggttatcaaaaagttattctagtctttatcttgacaaagtagtataagataatatgcagtggtagagaaatatagcttttgcaatattcccaagtaaacacctgaaagcaaagttaccacctggtccagcagccagttgagttggtgacatgcaagaaggccctacagccacctgaattcctctgtcagaagagctacctttgttttatttgacaatctttggacatcagatgtaagacaaccgccctggagtgtgaactctggagatacaaacttatcagacaatttctatttcattcaagatggcatgactttcatacaccagcatctgaaattgtctgagatactgacaacgaatgtgtagggacattcaccagggagctgagatttttattttgagctctttaaaacagtgccccccagtggtatgcaatagaaaaaaaaaatcacaagattaatatatccacattgttggtgcaaacagaacccattcctgctagaatgagctgaaaaagaggctaagcatggtggctcacatgtataatcctagcactttggcaagctgaggtgggaggatcacttgagcccaggagttcaagatcagcctgggcaacataattagaccctgtctctataaagaataaaaaaaaattagttgggcgtggtggtgcacacctgtggtcccagcaactcgggaggctgaggcaggaggattgcttgaacctggaggattgcttgagcccagaaagttgaggttgcagtgagctctgatgacaccattgcactccagcctgggtgacagaatgagaccctgtttataataataataataaagaaacagaaaaagtatatattaagggtattatttgtcccactactagctcaaagtacaaatggaaaagccaaccattcctcaagaaacatcactccccagttgaagtggaattccaaatgttataatcaatatgtgttgactacgaatgtgaatagtgcccctgtgaacttgtgcaatgtacacctacaccatccatatgtagccctggatattaagtggttcaaataatttctggaaaggttcaagaatcaagcttggaagccacacagtcatcaaccattcccaaaattatactgcaggtttgctatatgggagacctagagtcactgctgcttggcatagacaccacatatgatcgatgaagactgggccacttatgtacatcctagctgcaagagatgctggggaagcaagttcttggagtcaacttggggaggtgctgactcatgaagttggaaatccctaaatttaggaaggtatttgtgatggtgaattttaatgtgtcatcttgactgggctaatggatgcccagatagctattaaatattatttctgggtgtgtctgtgaggatgtttctggaagagattagcatttgaatcggtagactgagcaaagaagatcaccttcatctatgtgagtggagtgggcatcatccaatctgttgagggcttgaatggagcaaaaagacagtggaaggcaaatgtgttctctgctggagctgggccaaccagcttctcctgtccttggacatcagcattcctggttcttgggtctctggactcagattgggccttatgtcattggctctcctgctcttgggccttcaggcttggactggattacaccactggctttcctgggcccccagcttgcagacagcagactgtgggacttctcagccttcataatcgcatgaaccaatccctcataatctcaattactccccccaccctctctctatatgtatgtgtatataaaatttattggttttgtttctctcaagaaccctgactaatgtagggttcaaaggctttggggatcattctaaaagcaagacgaaagacaaatgtccaccagagcttgcaaatactgtatttatcaatccctcctcttcttggcatagtttctttcaaacttcttagtgtcctcttcccattattacaaacttctcgtttcttctaaggtccttctaaattggtgagttctcagtgggagttgagtgaccttttccctcagtgtgactatcactgcccaatgtcattgatacattatacaactccccatccccacttcccctcagctcctgttaaccaccattctttctgtctctatgaatttgactactctagattccccatgtaaatggaatcatacagtatttgtctttttgtgactagcttatttcacttaacataatactctcaaggttcatccattttgtagcatctgccaaaatttcctcccttttaaggctgaacagtattccattgtacgtagatactgcattttgcttttccattcacacaaagagagacacttgggttgtttccgcctcttggctattgtgtataatgctgctaccagcatggggtacaaatatgagaagtgaggtttaggatcgtgtgaggaggatcacttgtagagggaaaatagtgaaataggacgaccagataggctctgttggaactcgcgcccattgttttaagacgcagctcaaatgtcacctggttttagaagtcttcctggtctccgcaggcagtcagtatctctttcctaaatccaccacattcccgtagacagacaccacatttatgcaaatctccttctcacagacagtgagttccatgggggtcccctggcagcctccagccctgtaccatgctaactcttccggagaggaaatggggcctcgggagagcctttgaagcctgtgttggtacaggctgtccttccagctaacggggctctctggggaatgtatgtttgactttctattcccggtaatataagttcaaatgtttgtggatatttgaagctcggatgaaagcgtttctattcatgcccctagaatctgtgaggccttttctttctttttttttttttaaacaagagcccacagcaaaacttgggttacttggctgaggaaaggaatgtagagctaggagcagggtttcatcttcagccccagtcgggcaaaccagtggcagaagggaaagcagagcccaggggtccccatcccgtacctggggcgcaggccccgctggggtgggagggagagtggagggccgggcaggctgcagccgcggatggctgccccccctgccccggtgttactgggaggacggaggttgcgacagcagccgagcccacccagttgcagccggctcggggcttagggcaggggcgggaggtgaccaccgcgccgctggccgcctcgcccagacattccgtttctgccgctggaatgcgcggacaaggctccttgttggccttgggcggggttcgccgggccggcctggcgctcgaggccccggggcggggcggggcccgaggccgcgggacctttaaatccgagcctcgcgtgggctcctggcccccgacggacaccaccaggcccacggagcccaccatgccgcgcccggcccccgcgcgccgcctcccgggactcctcctgctgctctggccgctgctgctgctgccctccgccgcccccgaccccgtggcccgcccgggcttccggaggctggagacccgaggtcccgggggcagccctggacgccgcccctctcctgcggctcccgacggcgcgcccgcttccgggaccagcgagcctggccgcgcccgcggtgcaggtacaggcgggcggcgggagggacgcgtgagcatcgaacgaggagaagcccagggcatccaccaagcgcgcttcatcccggcctcaggccgaccgtggtgcgggggaaagaggcccctcccggatgaatcctgtgggggaaactgaggtccagagaaggggagggagatgcccaatatcactcatgtgtccaggctagagatgtgggtctcctgaagctctcactcccctgtgattcggccaggagcacagggcagctggggagcgcaggggctgctgcccagcggctgctgctaagctccgtcccagtcctgacctctccattcagtgagcaaacccttcctgagactctgctgttagttctcttggaatctctccatctccatccaatatcagggcgtgatctttcctcccctcccacagtcctcaggctgcctcagactggaaggtaagggatgcgggtgtgagaccaggaggagatccgaggagccgtaggataccccaggatcaagacactctgggtctttggcgaacaattctatttctctttttggtcagcaattgattcctgaagcagagcaaagctcatagtgcagcgaagcgggcaggtcgtctgacacagcccctgacagcacatgaagggaaactgaggcatatcaagttgaagggactctgtgaatcagtatcagaggccacaataaaattcagggctccgaaatcccaagcaaggtctctttccactgccccagcctaccaacctcttaatatggtttacataagattgtatcttcatttccaaaaagcgcagcttcagtcttgctaaaaagagtctgtgccaggccatttctgggtatgcagtctcatttggccagccacgctgcatgcctcaggctgatttcaattgtctatccccttctgcatctttctggataagctgagaaggagctcaagggactctttcctgactcaaatactcagtccccgtatttgaagtggcagatttgaaatggcaaaagcattgctgctaaggcaggaggtaccggggtccacagggctagattcagctggtggctggttgctccaggccgggaattgagggaaagcctttcttctggaagttaatcttactctagtcctgacctgcctccctctgggatgggactggaagaggaaggagagattcagcaccaggtggccatgtccccctaagctttactggtctttcattacctagagtccgaacctgatcatgaatgataacccattaataaatgcgagattccaccctgttccaaattatctccttttagcaaccagaaattgctaagcttcgctatggatttttatttctagcaggactgtaaggactcagtggtattatgagtatgggaacattttaggagattcagattctttctttgttcttgggaatcaaattgctgctatttagtaaatgggagtgagattcaaaaacaactaatgattgatgcggagtaccagtcaggaaggaaccagatgttcacagaattttctgccaccatggtcaagcctcaggtattagttttctatggccactgtaacaaattactgtgaatttagtgccttaagacaacacacatttattacctgagagttctgtgggtcagaagtctgaccctggtctcactgggctaagatgaaggtgtcggcagggctgtgttccttttgggaggctctaggggagactgcatttcccttccctttccaccgcatgaaggctgctgagttccttggcttatggcctttttcttccattttcagagccagctaaagcaggttgaggccttctcccattgagttactcacatttctttcatctgcctccctcttctactttataattacattgactcactgaataatccaggataacctcttgtgtagagctctttgacttaatcacacctgcaaagtctctgctctttgccacgtaaaagtaacatgtctctggaattaggacgtggacgtctttggcgggggacattattctgcataccacaccccgaatagaggattcggtgtgaataaccaagtggagaaggaaagaatcatgcacgtttagttataagaacatagccatgatatttataaggacaccatttgggtgctggtgaaagcactgtcaattacagagttcatatcttcatgttacccccaccttttctttttctaattatacaaaaatactttgacccatctccaggaaagattttcaacataattcttcttcctagctctagatctatgggactttccaggaatgaagtaaaattctggaggtttttttttttttttttttttttagtatttgaagagatttattctgagacaaacatgagtgaccatggtctgtgacacagccctcaagaggtcctgagtacatgtgctcaagatggtcagggtgcagcttgtttttatacaatttaaggaggtatgagacatcaatcaaatacatttaagaaatacattggtttggtcctaaaaggtgggacaactcaaagccggtggtggtgaggggcttccaggctataggtaagtttaaatattttctggttgataattggttgcatttgcctaaagacctgggatccatagaaaggaaatgttcaggttaagataaaagactgtaaagaccaatgttcttttgaagtcttatagtggctacccttagagacaatagatgacaaatgtttcctattcagatctttttaaaaaattattattattttactttaagttctgggatacatgtgcagaatgtgcaggtttgttacatagggatacatgtgcgctggtggtttgctgcacccatcaacccatcatctaggttttaagccccacatagattaggtatttgtcctaatgctctccctcctcttgccccccaccccctgacaggcgctggtgtgtgatgttcccctccctatgtccatgagttctcattgttcaactcccacttatgagtgagaacatgtggtgtctggttttctgttcctgtgtcagtttgctgagaatgacggcttccagcttcatccatgtccctgcaaaggacatggactcattcttttttatggctgctggagttgttttttttttttttaaagaaagaaagaattttgattccttttttttttttttggcttttagagactgggtctcattctatcatccaggctggagtgcagtggtgtgattgtggctcactgcagccttgactctctagctttaagcgatcctcctgcctcagccccctaagcagctgggactacaggtgcctgccaccatgcctggctaatttttaaactttttatagagatgaggtctccctatgttgcccaggctggtctagaactccggagctcaagtgatccttctccctcagcctctcgaagtgttgggattacaggcgtgagccactgcgcctgggtttaattccttttgacttaactaaatgcatgacatacagcatatttcagggcggtgacttaaagtcaacaagcaggatgtttactgaatacatgagagaatgttgatttttgtggaggaaaatgttgcctcagagagaggtaactgggattcagtagcaagctcccagggactgggagtcaggaaacctggagtccaatccatgctctaccactttctagttctcacacctccttgaacctcaggtttctcacctgtaaaatgaatataaaaaccacctctgcacctccagcctcacaggttgatgtgttggtaccttgcgattcctaaagtgttactgaaaggaagattttctttttgttaattaaatacctccaagcagcaggctaaaaacaagaagataattttctgcttttaaaagaaaactagacagagacataaggtaggctgttccaggtaaggctttaatttttttctataaactgcttagtctaggcattaattttgaatcacctttttttcctactaagtattaccttcctagagtagcatcaacttctgcctccttattaggaaaggaaagagccttgagtgacacttcatgccccaggacctcatttccatttagggctgcatgcgtgtgttgggtgagaagggggaatgacttgaaaaagaagtttagggctgggcacagtggctcacacctataatcccagcactctgggaggccaaggtgggaggatcacctgaggttgggagtttgagcccagcctggccaacatagtgaaaccccgtctctatgaaaaatacaaaaattagctgggtgtggtggcgcacacctgtagtcccagctactcaggaggctgaggcaggagaactgcttgaacccaggaggtggtggttgcagtgagccaagattgcaccaccgcactccagcctgggtgacagaatgagactccgtctcaaaaaaaagaaaaagaagtttaataattggctttttttcttgtctcatcctgacttgtggatttgatttttgtatcgaggagagagggtgtgcaaacaggggttgggggaaacaggaaagaacaggaaagaatagatgaacactaaaatgtactgtgatactaaactgttaggtacttttgtgtagaaaagagataaattctttgaaggtgggaaatctggctatctggtttatatgcttttgtagctccagggcttcatacatgtaggccctcaaaaaatgtttttggtgaatgaataagtcaaaagagagatgaccaagagagttagttggggcagtagggtagataccagatcacaatccctgcttcttacgagacatgctcatgaagctggaagctcactaaacagatacaagattaagttggcatccctcattctactttttttttgagatgatgtcttgctcttattgcccaggctggagtgcaatggcaggatctcagctcactgcaacctccacctcccgggtttaagcgattctcctgcctcagcctcccgagtagctgggattacaggcacatgccactacgacccgctaatttttgtatttttagtagagacagggtttcaccaagttggccaggctggtctcaaactcctgacctcaggtgatcctcccgcctcggcctcccacagtgttgggattaccaggcgtgagccaccgcgcccgggcccctctttctacttacttttttttttgttttgttttttgagacggagtctcgctctgttgcccaggctggagtgcagtggcgtaatctcagctcactgcaagctctgtctcccaggttcacgccattctcctgcctcagtctcccgagtagctgggaatacaggcacccactaccacgcccggctaattttttgtatttttagtagagacggggtttcaccgtgatggtcttgatctcctgacctcttgatttgcccgccttggcctcccaaagtgctgggattacaggcatgagccaccacgcctggctctactttaaattagttcagggctccagtgagcccccaaattcactaaacaggaaagatggccagatggagggagaattcagggatactctggagcagttttcagccccttggaagtggagtaaaagaggggggccaccagaagagcccctgagtgccttgcctcagactaccattgcctgaggcttgggcacagtggttggcagtgaggctgtccctgagggcccaaggccttttacatggttcagggacacattgtccaattgctccagatagccaaggtacttaagaaatgtgtagaatctattaattcccaggaatcatgagggcccttgggagcaaaagcttaagcagagggatgaggttctaagtgaaatttcaggattcagccagcctgcaggtgaggtatttggggaaacttcacacctccaggtgaattctcactagggaattctgaagtccttggtgctttcaacaccccgatggtcttggggaaggtcgcaggcagtggtgatgttggggtcaggagatgctgccttgcctggcctcctggaatcccagagttctctggaagtgaggattactgcatttctaattgtggtctgatcattgcttggtcttttgcaggtgtttgcaagagcagacccttggacctggtgtttatcattgatagttctcgtagcgtacggcccctggaattcaccaaagtgaaaacttttgtctcccggataatcgacactctggacattgggccagccgacacgcgggtggcagtggtgaactatgctagcactgtgaagatcgagttccaactccaggcctacacagataagcagtccctgaagcaggccgtgggtcgaatcacacccttgtcaacaggcaccatgtcaggcctagccatccagacagcaatggacgaagccttcacagtggaggcaggggctcgagagccctcttctaacatccctaaggtggccatcattgttacagatgggaggccccaggaccaggtgaatgaggtggcggctcgggcccaagcatctggtattgagctctatgctgtgggcgtggaccgggcagacatggcgtccctcaagatgatggccagtgagcccctagaggagcatgttttctacgtggagacctatggggtcattgagaaactttcctctagattccaggaaaccttctgtggtaagttggtcagtctttgcttccaactagaaaggatgttatattcagacattgtgtacccagagaaaagagtattattcttttttggtggaaatttaatgaaaaacttaaatatatccaatgcgtgtgtgtgtgtgcacgtgtgcatgcgcgcgtgtgtgcgaccaacctaaacacactctattggtttaggaatatagagctgcattatatggaacttggtttgctcataaaatcttcatgtttgcgttagaactgtgaaggtctgcaaatatccaggataaatgaccctgctgttccctgtcagggtcaggaaaaacaaaactcaagatgcctctcctgtcccctctgccccaccactacatttttttcccctgatacatatttttttagttttaccatctctagatctaacagaactctgtttgttctcctgcagaagacagggagaagcgtctgtccttgggagagtcagcaggcctctattccaatgtcaccgtcagcccgagtgtgcatttagtgaatgctctggttttcagactgggctgtttatctgattactctttgttgaagaacaggattcagggaccaggcagattagggtttcagctcacttgaaagaaagcaagatgatcttaagaaatcattctgttaactctgctactccttccatcctttaggatggaatttgaataaggacatgctaattttattggaatttttagcatgaattgaccaaatctaggactacagatccattcaatgctggagtttaattttagtcactgatcagaattatgatctgtgggactcatgctttatttttttatcatctggagataatcttttgttccctctatgtgaatatccaagcagttgtggacaaatttctagttcaggtgaatttttaaacatagatatctttttgcaatataatttatttcacacttatttattcagcatgcatttattgatctctgatgatacaaagataaaggtaatccctccataccattaagtaacattctgggggtgggggtgagacaaacaaatgaaccaataattaattacaattatacatttcaaggagacttttaatctaggttaatgtgaaacgcagccatcaatggtttgtcaggaaaagggagatgaagtcttgctctggggcaacgtttggcctcattgcagtcagacttggctgggatccccttgtctttcctgtcccaggacactgggctttcacaagccatcactggctttggtggcatctacttggcattcgttggcttccaaaaagaaagtggctttctttctcccaggaaatcaccctcccactccatgcttgtgggtgagagcctctccatcaacatgctggtctgtggcaaagttgggtttcaaagctagcacctggggttgggcttcgaaggctataaagtaacaagtggtgagtggcaatcttttccttctggtacaaaagacctgcttgttctgggggaaaaacgtcctctctatggaaaaaagactgagcggcagcttgttgtagtcaacgggttggaaagatcattccatgaaaaggcccttcttgggaaaatattagccttgtcctgttgccaagggcagtggaggttgggttacctgccacaggagggccagcctccttcaacaggcctccttggtttctcagaaatgacttaattcattgattcatctttcaaaagaggtcactaggaaaaaaatatccaatgagatataaatggctccaggagcaataagatggagatgtagtttagctaatgtaatctcatttattttgttcttttctttttacctgtttgtattagcatatgagttcactatggatttagcgtatggatgacttcacccaccttacatattcagaagacagttacccgaactgtcctctaccatttgcatgttcaaatatgtaccactgcccacctgtgagttcgttctaggcttagacccagacttgtgtgagaattctgtttagacataggctatagaaccttggaaatcagattctggctacaatgggacaactagctcacgtcacttcaggctgtgcatttattgttctcccatttcttgtggtggggaagcacaaatctttttttcatggggaggcggataagccaaataaaacatccaattgcctggcctatgtaagtgtttggcttcagctccaaactccaaattccctttctcccttgagaaagtctatatagcaagatgtgtacttttaaattagcaacaatttctagtttaagagtagcattgctaataagccagaggcagaaaacaaataaccatttttttatagtgaagtaaagatggactcggaaaggagcccagagagcaatgtctattgacatgggagacggtatctgagatgtttcctaagtgtcagtgctgactgttgtaaacttcctctcacagcgctggacccctgtgtgcttggaacacaccagtgccagcacgtctgcatcagtgatggggaaggcaagcaccactgtgagtgtagccaaggatacaccttgaatgccgacaagaaaacgtgttcaggtgaggcctgtgtagggggccgtgactgaatcaaatacttgggaacctatgctccaggttttggactggccttgtgctttgactctctgacccctttttacctaactgccttttggctggtttactggtgttcatcagcagttttccctgaaaaggagcttagggagaaaagtgaaatgatagataaatcagcttatttccatactttgtaagttttaaataaaatcgtaggagaaaaggctactaagaaagaggtccaggctgagtgcagtagctcatgcctataattcccacattttgggaggctgagatggtaggatagtttgaggctaattcgagattagcctcgagaacatggtgagaccccatctctacaaaaaataatttaaaaaaaaaagccaggtgtggtggtgcatgcctgtagtcccagttacttgggaggccgagatgggaggattgtttgagaccaggaggtcgaggctgcagtgagctatgataatgcctctgctgcactgccacagagggagaccttctttcagaaaacagaacccacacacacaaaaaggaaaagaaacgggtccaagattcttagcccttttgaggatttgaaaattttcctggattttatttcattacatgcaagctaccctcttcctctctagtcatagatgctcatggaatctagaagtagcaaagaagttagaggggtactcactggtctggtacatagatacatacatggagtcaatgaggaatgggaattatatatttttttgttttgtttttaatcctagaaaaaagtttttgggaggaagaaagcttagagataacttaattttatagataatgaaattgagatggacagaagggaaaaaacttacctatgtcacagggtctcttagtgaaagagccaagaccggagggttggttcccaactcccagctgcccctcccttgtttcattagctctgtgtgtgtgtgtgtgtgtgtgtgtgtgtgtataactgtaagtggcgtggcccacaattatttctgccatgctctgcatgcatttacatttgtcccaatcatcttttgatagctcttgataggtgtgctcttaacacccacggatgtgagcacatctgtgtgaatgacagaagtggctcttatcattgtgagtgctatgaaggttataccttgaatgaagacaggaaaacttgttcaggtaagtgagggaggagtctttactattgctacttagctctctgcctacctacccggtgatggaagggcaggtcacattgactgggaagttggtgtgttttcctctcttgccaccttgagcttttcccatttttaagtcttatccaaaatgttagaaatggtaacatgtatgtatctcggcccaaatacataaatatgttgctcatttccttctctgtagccagcctgaacaatccttcccaaatgctacagacaattcagtaggttgaaatcacataacaacccacaagttacctgttgcttacaaagggaaagacagtaactttatagtggagaaactggcagacatcaccttaactaagtgatcaaagttaacatcatcagtgatgacacatagtgacatcatgtatgacctgacatgctgtgctcaatactgtggacacaacagcacacctgtagtattcttgctaaaaattcaggattatgaggaaagattagaaaaacctgagagacactctcagttcacaaaatagttttccagtacttctcagaaacgtgaaagtcataaaagacaaagaaagacttaggaagtgtcccaggttggagaggacggagatgtgaaactgaatgcaatgtggaatcctggattggatcctggaccagaaaaaggacattaatggcaacatttgaataaagtctgtagattagtaaatcatattatatcaatgttaatttctggtttgataatcatactatggttatgtaaattactgtttggggaaacagggtgaagtacaggaattttttgcactgtttttgcaaccttttcataaatctgaaattatctcagatgaaaagtttaaaagtaaaaaatagtttaaaaatatatcagtagagaggaaagtgcattgaatgaaaatgtgtgtcttgagtttttatgctgctctgaatcaaatggtctccctcgcgctcctttccctcatttaaaaaataggagagggttgaactacaatttcttcaaactctaattctatgtttttctgaatctaatgtaattagccacacaactgttggtatcccgctgagttgggataagtacaataagcctaaaaggaatgctagaaagtatttatgagatacttaggttataaaaactcaatactgatgatttccaaaagcaattgtggaaggtcagagaataattattgtatgtgcaggatttcacttaactcagttaacttctagttagaaaaaaacagtgccaatggctttgaatagtttccagtaagttttcctcatatgtttccagctgcaaataaggtttctatcctgggtaataaaatacttttccaccttctatttcttttctcctgactttttgtttcacagctcaagataaatgtgctttgggtacccatgggtgtcagcacatttgtgtgaatgacagaacagggtcccatcattgtgaatgctatgagggctacactctgaatgcagataaaaaaacatgttcaggtaagatccactcaaaaaattctttcatacttgggtctttcacatgaaaccaacttgcaactcaccagcaaagaggtttttaaaggtaacttaagatcaaggttcagacattctatcacacttttagatgaaggcacacattgctcagagggtggtgagctcactggagcaaacatagattacttcagaaaattactgggaaataggtagggagtgggggatatgagaaaaactaactaaatgtagggtccttatccaggaaaattttatagctctgtacactgaagacttttaagcaaagagaaaactttagtggaagtcagttggacagtaactatttggtgtgataggatgaactgatctttttccagttaaaacttggcctggaacttttctgtagtgttaatcttaaagctggatgattttagtaaagaagactttgtgaatgcctgaattagacccacacagatcttccagtaagtttgaggccagtcactccctggcagtttctagacgatcaatcattggtgaaactcagccttatacgttggccaatgagcccaggccagtgctgttaatcctcattaatttgatttaaacttactggcaggacagggtggcatttataaatgtaaaaacactcctgagagaaattccaagctctcaaggtgccgtttagctttggcatattgaatgtgttttagatttgacatatattttcctctcccttgaggacatacagacttcatcaagatagacaaagggaaatggcatgtcacccagtcccctcttttttttttttttttttttttttaaagggaaaccagtaaaaaaataccggacacctactttgttcccagtatggaactaagcattgggagcatacaaagaagcctaagtttctgcctctgagcaagttaaatctgggtgaacagagagccggctccttatgaaagcagagactgagccagggagagggactgatggaaacaaagggctgtagaggctggggaggtgggcatctgcgaagggctggtggtgggccttgaaggagggtgaaggtttcaatggagacaagtggaaagtggcactggcgggggactggcttgagctgatttgttcagggcaggatgactaagaaagttggggtggagagaactcacctatggctgcacttctcaaactagtgacaagtgtcccagttggtactttaagctgccaggaacattgaagacctgggaaaacaaaccaaaaacaaatttccatttaaactaacagatagtatggaataaagtgaagcaatctattttaaaaaaaaacataaatcaaagaaaattttcagttaaggcagacaacactcccagatgccagtattccatattcttcaatacctactgtaggccatgcaggattctaggcattttagatacagtaatgtaccttaatactcacagcaacgttaggaggaaactgattcagtaacttgcccatggttactctgctagcaagtagcagacccagggtttgaacccaggtacttcctctagtggttacataggctcttaaccactaagccttactgtcttaatgtacaaagcacaggacagtgaagagggacagaagagaaagaatactagtgggaaagttagttatatatatatatatttttttttcttctcttttttgagacagggtctcattctgtcacccaggctggagtgcggtggtgtgatcttggctcatacagcctctacctcccgggctcaagtgattctcccatctcagcctcctgagtagctaggactacaggcacatgccaccacacctggctaatttttgtatttttttgtagagatggggttttgacattttgtccaagcttgtcttgaactcctaagctcaagcaatacacctgccttggcttcccaaagtgctgggattacaggtgtgagccactgtgcctggcctatttatatattttaaagtaatagtgaatactgctataaagctggggaacataccatggatcagatcccttctaatggtctgcttttccgttgctagtggtaggaaggcttgtaaattaaatgtggagccccaaataagggccaggggctggaagcatgtccatttgccacctgtagagtgggtactaacctggaatgagggggaaaaaaatgaaacaccctgctataaggcattttgctgaaaaaaaaatctaaattgtagcactgaaagggaatagccatattaaggttggaaaggcactttatgtatagattatatagcgtctcacatttcacaagaagctattctataggcattagtgagccacaaaaggttctgcccatgaaatgacatttaagaggaaaattagagggaaataaactattttggttcctgcaatggcagaggaggaacctatgtcttccaggaaggctttctggatttcaaaggagaattactgttttatttgtcctaggccagtctgaacatgggatgagtatggcactttgctctcctctccactcaaatttgggcaaactgcatttgtccctgaaatgttatttgtgatagatagtaagtacttcactctctaatcatacacatcatcattactaaccctgtatcatttccccatctgtgcttgtgtgtgtgtgtgtgtgtgtgcatgtgtctcctggtcagtcttttctgttacactttgtaagctatgaaggcaggcacttggtcaatatagtgctttttgtgcagcatcaaacttagctccatgtatgattaggtccctaaaacattacttttggtgatgatgatgatgatccatgagtagctggggatgctagaagcattgatagtcatcgggaaaggctggaggtaagttctggaatatagtcaagactcagtgctgcccagtgctgtgggtagaaaaaaaggaaaggtacccttagatatctgtgagagatataaaggaaaggagtgtgtgtgatgtgaggccaaagggatgagaggtatgggaagagaaaacaggcttgctcctgcagcatggggcctgttgctgactactgactactacattctcagcacttgtggggaccaaatggaagcttctcctctcaaacatttgtgtttacttttctatttaatgtgtgaatattttctttcctgcaccacgtgcggtcagtccgtgacaagtgtgccctaggctctcatggttgccagcacatttgtgtgagtgatggggccgcatcctaccactgtgattgctatcctggctacaccttaaatgaggacaagaaaacatgttcaggtgaggatcagccctttaaggctatttcctttggtaggaaaatttttcaggagcaagcttttttctttctgtctaagacacagttgtgattctttctccctgtcagaattctccagtggctcccactggtctacagaataaaacaatgatacccctataccaggcagcctaccctccatgatctgtttctgtcttctctgtttttcacatttttcttaataactaaactttctgcctaagtctgaattttttttattgtggtaaaatacacatacaatttaccatttcaattattttcaagtgtatagttcagtggcattaagtacatttacattgttgggcaaccaccaccagtatccatctccagaaattttttatcatcccaaaataaaagtctgtgccgatgaaacaataactgcccatcacccgctcccctcagcccatggtaactacttctctcatcctggcttttaaaacttttctactacatttgcccccagattcactgcttcttcctgcatcctggtccagccacagtgggctctttgatggtccaggagcatgccctgcactcactcattcccaccctcccctcaatgcttttgcttgtcagaatttgcttgccaacatttggttttgttgaattaaatttttgccaatccagtggatgtttttttcatttcctggttactaatgaggtgaggcatcttttcctggagagtactgccattttaacagtctttcattgcattaacatggtatgtctttaatttaatttaattttattttattttaggtcttctttaatttctttcaaaattgttttgtagttttcagtgtacaagtcttgtacttttaaaaatatttattcctaactattttattccttctggtgctattataagcaccatttctccatttctcaactttcaattttgctacaacccaggtttgagaaatggagtttgagatccatttctcaactttcaatttttctacaatccaggtttacaattttctatgaagtgttgtaattaagactatcccactgaacccatgccctgctagagcttagagactatcaggcacgacagacactgaacaagtattatatatacccatcttatgggtacagggataatggacatgtagttgatcttcaatagatacttcctggtttattgactgattaactaagatgggatttggactggctgtccctaaaaggttcaataggattcagacaggtggatagatgatagattatatttaatgaatttatgatctcccatccctatattggatctttgtaaaatgttaagagtaaaaatgttgcagcaaaggaccatttcctgtcctcaggcaatagaattactccattggaagatgatacaccagtcttcagacctgggaagttactcttatttctattcacaactttaggaggagccctgatactgaataggaatagctgcctgactgaattccataaagaaataggagttacttttatcacctctcatatttggttaaatgtcaatacaaacgtgaattttcagttccaaagtatttattggtaagaactaggactcttacttcaaaccattaaatagaataacagtcctctagaggaaatcaggacttaacctggtttttatcctttttttttgttttgttttttgagacgacatttcaatctgtcacccaggctggagtgcagtggtgcgatctcagctcactgcaacctctgcctcccgggttcaagcaatcctcctgcctcagtctcccgagtagctgggattacaggcatgggccaccacgcccagctaatttttgtgtctttattagagacggggtttcactatgttgtccaggctggtcttgaactcctgacttcaagcgttccacccaccttggcctcccaaagtcctgggattacaggtgtgagccactgcgcctggccctagttttcatctttggtatattttagccatacaaatagaggactattgtcctagttttatttttatttatttatttttaagagacagggtcttactctgttacccaggctggagtgcagtggtgtgatcatggctcactgcagccttgaactcctgggctcaagtgatcctcctgcctcagcctctcgagtagctaggattacaggtgtgtgccactatgcctggctaactattttacttttttgtagagacagggccttgcttcgttgcccagggtggtcttgaactcctggcctcaagtgatccttctgcctcagcctccaaaagtgctggcattataggaataaggcactgcaccaggcccattttcctttcttaattacacagtattacataaacattgtcttatttctatatccctggcctccccctataccaaccatgtttttctaaatttactttatatcatgaataatttctggcaatcacaaaagtacagagaataaaataaggaaccctcacttaagcatcactcagctttgacaattataaacattttccccaatgtatcctaagatgaatttttctgtgataagtattttctcgtctttttgtttgttcatctatccctttacctcttggaaaatgcacttaggtaagtcttaacggtggaaatattcaactgtggatttcctgatttgttatgattcctatatagctattcctagatagtatatttgtgtactgtcattgtcttcagccactgaggaagcacgaagacttgtttccactgaagatgcttgtggatgtgaagctacactggcattccaggacaaggtcagctcgtatcttcaaagactgaacactaaacatatcctttctggaaggttttctccttctgcctggagccaaccaaggtactacgatcgagccaaacactttaagacattccacaggcaagtgacttatttgttaggttttaatatgagcagtggtaatatacaactttaaaaaatttagagttcttttctaatggtttaatagactgtttcacccattcaatatttacatgtaaaagctgcctattttgtcccctagtgcttttccataactgtatatgctcctaatgatgctcttacataacagatggaacatttatgctaatgactccttaatatggctttcttagttcagaatctgaacatgtacaaagcaagtcagctgattaatggggtgtgatatctcagattacacacagttgtaccctttgactagccaagtggtcaaaaagcagattttaaaatgtaacatgatacaatctcagaaactttcaaaatcagttctttcttcaaaggagtaactagggaaaagtttctcagaacagttcaagttaacaactctgttcctatgtaccccagaggagtggaatttagattgtggtaggtacaatctaggcgtatgtagagacttttaagggaggtacagataattggaaagaatcaatttccaaatccttcacttgcttaagacactacttggaatcagctggcattttcttttcccgtctctcatttccatatcaccattctcctaacttcacacaagaaaggcatacttctcatttaccctagatctactaaggtgcatttgttagaacagcagaatcttgagatagccaaacaaagggacaatccaactatccctttaatcaatacttcataaacactgcttcccccatccctcacactaagatgctactccaattacattttatatgaacaaaaacatctgaagaccactgcaggggtctacttttattgtgctctctgaagaacaggatttttgacttttagactgaattcaacttttggtgagaaaacagttttgctcttttttttcacttctcataggacataattattttatcttgtgaagtgtttgaaagtgtgtttatattttctaatgtgtaaaaataaatggtgttggccttaacaacttgccacttgatgacattttggagaagttgaaaataaatgaatatggacaaatacatcgttaaattgctccaatttctcacctgaaaatgtggacagcttggtgtacttaatactcatgcattcttttgcacacctgttattgccaatgttcctgctaataatttgccattatctgtattaatgcttgaatattactggataaattgtatgaagatcttctgcagaatcagcatgattcttccaaggaaatacatatgcagatacttattaagagcaaactttagtgtctctaagttatgactgtgaaatgattggtaggaaatagaatgaaaagtttagtgtttctttatctactaattgagccatttaatttttaaatgtttatattagataaccatattcacaatggaaactttaggtctagtttcttttgatagtatttataatataaatcaatcttattactgagagtgcaaattgtacaaggtatttacacatacaacttcatataactgagatgaatgtaattttgaactgtttaacactttttgttttttgcttattttgttggagtattattgaagatgtgatcaatagattgtaatacacatatctaaaaatagttaacacagatcaagtgaacattacattgccatttttaattcattctggtctttgaaagaaatgtactactaaagagcactagttgtgaatttagggtgttaaactttttaccaagtacaaaaatcccaaattcactttattattttgcttcaggatccaagtgacaaagttatatatttataaaattgctataaatcgacaaaatctaatgttgtctttttaatgttagtgatccacctgcctcagcctcccaaagtgctgggattacaggcttgaaagtctaacttttttttacttatatatttgatacatataattcttttggctttgaaacttgcaactttgagaacaaaacagtcctttaaattttgcactgctcaattctgtttttcgtttgcattgtctttaatataataaaagttattacctttacatattatcatgtctatttttgatgactcatcaattttgtctattaaagatatttctttaaattatactgaattaacagttattttgatattcaacagtttcttatcaaaagacagaagtttgattctcctcctatagtttaatctcacactcctgaggctaaaagtgtaagttcaggttcaaaagaaaggcttcaatataaaattatctaaatttggataaaataatgatgagctttacattaatataagctcctgagaagcaaatatatcctacagatgtaaatataaaatataaactttaaggaaggaacaaaaactaaatataccaaggaaatatataaagcatctcttacttttcaaaaattttgaaagggtatgacatagagctacagtagtattttccagacttttttgagcccaaaatcagtaatacatattaatataccacagagaaatggtggtatagaaggaataaatgagagttcatgtgcaagaagggaaagataaagagaggtgagttttcaggtgtcaccatcagtaactagcttcaactaattatgtcttgaccagtttattggaaactcctgggttagataatactaatttgtctcatgcgtccagtatagggagcctatggacaggggccaatgtatcttaaacagacacacaaacaatggaaagcaaacattatctgaatgggattgggttaagtttggaaagaattcctcatggagactgatttgtgctggtttggctaggcacaactggcctgttaagagggtaggaccatatataaagtggcttgggaagcagagccggatggaatgttgccctgcagaaagcatattggcttacttgaggtgggggctccctagtaaaaacatttaggattgagggaagatctgattttggatggagtttagatttgctaggaaaggcactagggactattgtcaattctataatagattgtgctgggtgagacaaatcacccaggtcaaaggattggaggatttttattttatttttgtctgagatgggaaaagactccaagtggggaaattaactactgatgcataaattacatgctagattatggtaaaagggaaaaccagaaataaaaacataggtgagaccaaataatttcatgagtggttctacttggatttctgccctatgccttagactttgaggctttgtaactctcaagcattccaaaaggacaaaggagaatgaagaaagaaggtaagaaaaagaagagaatatgtaacagtcgactctagacgagaaaacaccaaacgctcaaatatttgtaaaatgggaggccatgccccaatagcaattgtacgctgtaattacccaacgtttacagaaattcctttaaaaggaccctcggggttgcggtaataattaaactgttaaaaaatatcaaaacaagcggaataaacaaaatcttctattcctaaattgctctagaggttctccaatccccaaagacacagagaagggttgtaagatctcagtacgcacatttgcactttcatctggccagtggcgccagcctatcaaatacagtaaatggcttttccctttcaagaatgttggaactttggaacttacaaaatactaaataatttgcccttattcgaacgggcgcctctcctcttaggtttatagtaatgccacctgaggcgcggctccatagcgatcatcacaggtgtggcgggagggagggcggcagtgaccacgagacggggcgcagagggcgagcgcacggctggcagtgacaggtcgcgacccagctccttgcggagagcagaaaggcccagacgccagggaacaggactagcccagctaccgcctcgcgcctcgcgccctgcgcccacaaacccatcgacgtccgcgcaggcgcactacgccggtgcccggtgcgagggcgtcaccggaagtgtcccgcggcgccccggatgcgaccgggcaacagcggttgccagggcgacgggagctttccggagctgctggtactcccgattggagacgtagaaccgttacttgtcgagggccttagcggccgccgtgaccctctcggggatcccacgatgttcttctacctgagcaagaaagtgagtttcctggggggcgtcctcgtttctccgccattcccgcagcctgcggcgattcccgctgccttcccgagaagccaggctccttcacccggtcctggtcgtggcttcgccattccgcagccatccttcccgccgggtccagcctccgacctcttcctgcggttcccagagcctcagtctttggccgagcttgctgccctctcgcagcactctcccattcctgcccctggctaggtccttttgggtggcttgaggataagtcgccacgtgctaccctatgtttcctcctccacttgttgctgatccacgtgatttaaactctttggatcaccacgagccattgggcaagagaaaagaaataacaataggatagcctaagttgtaggtaaaaaggaagagggactggtggtatggatttataccctctccccgtcctccctttaaaaaatccatccggtgagactctggcgttggcttcctccgcagtcactatccttctcttttgtgcacttctttccctctttcccaaatgctagagcccgggtcagttcaggggaccatgcccattttcacaacagctcattttttttctgcaaggccgttcgtttttcttcttggaagattttcttcagctttcatctcctgcagacttacttcccagctccttcgtgaaaagttgctagtgcggctgtggttgctttttcagatttccattcccaataacgtgaagctgcagtgtgtatcctggaacaaggaacaagggttcatagcatgcggtggtgaagatggattactgaaagttttgaaattagagacgcagacaggtaaatgaatgcaagcccacatgtttggtagtaaattggctaatgttatttgttcagtgcggtctactcatgttatctgtcaatatgcagttctgattattttaaagggaaaactaccaaaacattttatggtgtttttctggagtatatttgcaataaagttcagagaatgttgaatgtaaacatcggatggtttttgctgagttattaatggaaaatgaaaaataatgaagaatgaaaataagtcactgtgttatgctatgtctaattattgtactatattattaggagtctaaaagaaatcctgtgagtcaacatttcactaagaaaatctggcagattgtttacaggtctgtgtcatcctttctgcttatcagatgcattaatggttattcacctagtgtccaagaacagaaaatgggatacgttttacatttctattagtaggtctcttgtctttgctgcttgatttctttctttatctagttctctggaatcacagcaaacagcttcccacttcttagcccccctagattgaagacctagataaggtaattggagttaaatctcagctcttccacaagactactgaatgactttcaacaagttagctgaggatcttactactttagtttagtcttccataaaatgaaaatgtagggggttaaactgcattacatttttggattccttacaacatagcttctcaaactgtattaggtataagaatcactggaagaggttgttaagatggatttcatgctctacccccacacaatagacccgggatggggcctgagaatttccatttctagtgaagctgagttgctggtcctcagaccgcactttgaatagcattaccttacaactttgaatgcggtatgaataataaatacttattgacgattttccatgtagtggatgtttaaccttcacaatcttatttgacttcacaataatcttgggcagtaggtattctaggtgagaaaattgaggctctgagctgttaactgccctaggtcacgcagccagtaagtggcggaacaaggatgtgaaactaagtttgtctcacattcaagtccttgtttttaaccactacacaaagggctagtggacagcctaagtaaagaagagtaccaattaaagtttggcttatgctgttttatagttcaatctgtcagtgctttagttttttaaaactgtgttaaaagaaagtttttctctgtttcttaaatgtgtatatagatatatatatacgtgtgtatatatatatatgtgtgtctgtatatacatacttttgcttacaatatttggcaagtttttttttttttttttttttttgagacagagtttcgctctgttgcccaggctggagtgcagtggctggatctcggctcattgcaagctccgcctcctgggttcatgccattctcctacctcagcctcccaagtagctgggactacaggtgcccaccaccatgcctggctaatttttttgtatttttagtagagacagggtttcaccgtgttagccaggatggtcttgatctcctgacctcgtgatctacccacctcggcctccctaagtgctgggattacaggtgtgagccaccgtgcctggcctgcaagttttaaattacattaagaagtgcattcattttgtttaacattgaaggcttttttaaaaacaaacttctttttctgctctgcccttttgatgagctgtgactggagtattctttgaaattagtgaacaagatgggaaatcaattcttgggaggtttggaaaatggagtaaagtatttgttttggaactgaatgattatgaattgtgctgaaagaaataaatatgaggaatatggctgtgagagtctaacaagatcagtatattatatttaatacaactagtgttgattcagcagatccgtccagtaacaatcatagatattgcatgcctgctttctatgaagcactgtgttgtgctaaagatgcaacatgtgaagtataatgtgttgtagtttgggtacagcaacataatacagcaatcctctacgtccataaatcatcttctatggcaccattgttaatgtctatttagtatttcattatattggtattaatatatcataatttgcttaaccaattttagtattggacatttaggtgtctccacatttttactattaaagcaactgtatctttgtatccaaatctttgcataaatagttaatactttctacgataaatctctagaaatggcattcactggatcgaagagtgtgtacatttatacaagttcctaatatgtgtacacaaattaccctccagaaagtttgcaccacattgcatttccagttttcagttccctgctctagaaggaaaggaattagaaccagcaatccatctttttacatcaggtttacttctctcacaggccagcttccagtgttttgctggagcccatctcccttcaccttctcaggtacctttctgccaatttatcctctctctctcctttcaatatcagtcctctctccttttctctccctcaccagtgactttcatgttctaaatctaatggacagcttttattgactcatcaggatttgacttgaaacattatttttccagacaagagaattcagaatgtttaagttattgttatatttagacgtactacttttctctccttctctggtcagtcctcagtctctttgctagatttccctcttctattagaattcctgagaacttgcttctgggctctctattcacttgctctatttattctttcattctcatgcccttaaatgccatctgtggtatgaggtgaagagccccacatttatatctccagcccagtcctgatttttcagacgtgggtgtctccactggagtctttcacaggtatctcctatttactgtgttcaaaaccgaactcttgatattcttccaaatctctttcaattctctccatttcagcaaatatctccatttcactcatttgctcaagctggaaatcttctcttttcctccgttaacaacatctgcatcagcaatcctgttgcttttgtctccaaagtgtatgttgaacccatcctttcctttgtctcccctgctccctctatagctccagctaccatcatccctcacctggtctattgtgagaatcagtttccctccttccattgtctctggcttcctcttagtcgttcttgacactctagccagtgtcaggcttttttttttttttttttttttttttcccgagatggggtctcactctgttgcccaggctggagtgcagtggtgcagtcttggctcactgcaacttccacttcccgggttcaagcaattctcctgcctcagcctcctgagtagctgggattacaggtgcgtgccaccacgcccagctaattttttgtatttttttttttttttttagtagagacggggtttcaccgtgttagccaggatggtcgtgatctcctgacttcaagtgactcgcccgcctgggcctcccaaaatgctggggttacaggcgtgagccactgcgcccggcccttttttttttttttttttttaaagggaccagatgatgtcatttccttgccaaaaggtatttaaagggctgacagcctgggttggagctgcttgtggtagggtttgaggatgatggtcctgggttggagctgcttgtggtagggtttgaggatgatggtcctgggttggagctgcttgtggtagggtttgaggatgatggtcctgggttggagctgcttgtggtagggtttgaggatgacggtcctgggttggagttgctggtggtagggtttgaggatgatggccctgggttggagctgcttgtggtagggtttgaggatgatggccctgactaggattgaggtggcatttcttgctttttctgctacgactgccaaggcctccaccctcatcctcatgagaggaaatagagagccctggaagccaacatttgcatcagagaaaaagtatatagggccatggaagccaactgcagctgactgcgcagtcctccggtacctgcaggcagtgacattgcctcaaccttccaatatgcccaggacaacaaaattgaccagaaatctcaggtaaggtgtccttagctagacccagggatgcatacagagtcctttgcagtcagtcattcacacagagaagcaaggcaaccagatgatcctgccagagaatctggcgaatatggtaccatctctttctcagaattgtattatccttatgtgttgcaaaacagtcttctgtatattttgattctaggtttcaagcttattttgcagcatgtaacaggaatttctgttgaaattgggctgctgacaacttttatgaatgcaaacaaaagcattggcagtcacgcttttctaagagaaagattctcaaagatttagtgtgcttggttattggtgttattagcatgatcttctgttcttctatgtcataccttttgttttcagttgctttattacaccttaattttttaaaagccttctttggactttttgatgtacttcggattgttggaataactgatttcattctgaaattcctttacatgagtttaaaatgctttattttattggtgacttcttcgattatgccttttaaatccaaggattactgttatgtgcttttagaagatttatgtcagtattaccagatttttgttcacataccagtttggtttcattaccttattggctatctggagtctagtaatgtaactgaatggaatcttgggatattatcgctggctttactctacatcatttaaaacttttggcctttttatggacatctgagaactttcaggtgggttttatgaatattttttacatgatcaagttacggagttgcttccagcaagagacgttttcagatgtggatgatatttgttcaatatatcaagctgaatttcaaagcctgttcttaactgtcagcatgtgttttgtcaagagtatgctaccttatggtctaacagagggcaaacatgggtggtcaatctaaatctcatatgacccatttcacttaggttatagtgacatcagaatatgtgggtgtgtgtatatatgtatatatatatatatatatatatatatatatatatatatgcattagaatggatttttcagtgctagaagttaagaaaaatgtttccaccaaatattttagaatattataataaagtttaagtatgttgtacaatttttaaaagattaaatgaatgaaagaaccctgtgttaaaaaaaattatgttcagcctaatgtgtgtgtcagcaacatttattctatcttaattgacagatgattgcattgttaaactgtacatgtgtactcttgtgttaccaaaacaactgggtaattagaactagtgatttagaggaaattaggtatcttttcctgacaatgttttcagaataaaggatatttttcataatattttaagatacttgttatctgaaagtagaattttctttagcattggtataattcccattctcacaaattcttaagatcttcataacatttgtattttaaatgaaaattttaaatgctatgttttatgtgtaacagatttctaagccaagattaatttattgagatgacattttaatattctctgtaggatggttagtaaatttttaagacgtctttagaggttttcattgcactttggataaaatctaaaattcttatcattttctacaaatacttcatagtttttcttgtgcctatcactccagccttgtttgatgcaattttctcccctgttcatccacataggccttcttagagtttgtcaagggctctagttgtctacctcagagcctttatgttgctgtgtcctagcctggaatactccccttctttctctacctttcctggctatctccagtttacctctcagtctcagcttaaatatcagttcttctgatagccttccctggttccccatcaatctaaattaagtcacttctgctactctttcttattatgctttcacattcccagtttatagttacatttatgtgtatgtgagtatatatttaatattggtcccttagactgaaagctctgagaagaaaggggaaatgactgttttgttcaccattttatatgtagaaccttttgtaatgcatagcacagtgtacacatttcataaatatttgttaaatgaatgaataaatgagtgaatataagattctgtgtaatctagtaaaaacccattttcaaagtctatgctcttttgatcccttgtaagctgtagagttttcaatctgtaggccataaatatttctatttgtggtcttttggaaaggaataaaaagcagtgatgattattaatatatgtaatgtatgagtaaacaatcttcatttatatattttgctttattttaccagaatatctttgcaaggggtatgtgttattgttcatgtttataccatgagaaaaatatatcagaatggggcatacttagaaaaatgattaatactgcccaggaatacaaaaagagtcaaaatcttataatatgtcacttttacatatttatcatagtagtttgttttgttttgttttagatgatgcaaaattgaggggccttgcagcccccagtaacctttctatgaatcagactcttgaaggtcatagtggtgagtcattcaatttcttttaagtcatgtttgagtggtatttaggcatttagaaagtttctacttaatgcagaaatgtcaacattttaaataatggtaatatttacagtacagatgctctttgagttacgatggggatatgttccaataaacccattgtaagttgaaaatgcatttaatacacctaacctactgaacatcgtagctctgcctagcttaccttaaatatgcccagaaaacactagcctacagttgggcacaatcatctgacataaagcctattttatactaaaatgttgaatatcttatgtaacttattgatttctgtactaaaagtgaaaaacagaatggttgtatgggcactcgaagtacggtttctactgaatgtgcattgcattcccatcaccgaaaagtcatttaagtagtaccattgtaagttgggagctgtttgtaatcatttctgttgagacataagtaatacaacttagagtcattcaacttttacatgattaaacaacatcttaaataaatctagaagaaaatagctgtttttaatatattgaaaatactcctgtctatggatgttgtatatataatcctttttagaatctgtcctatgattccagattaaaggagactgaagagactgggtgtagtggctaacacctatagttcaagcactttgggaggccaaggtgggaagactgcatgaggctgggattttggaaccaccctgggcaacataatgagaccctgtttctacaaaaaataaaaaaaattaaccaggcgtggtgctgtgtgcccgtagtccccagtactcaggaggctgaggtgggaggatcgcttgagctcatgagttggaggctgcagtgagccatgattgtgccactgcattccagccttggtgacagtgcaagatcctgtctcaagaaaaaaaaagactaaagactgaagatgtataacaactaaatgcactgtataatcctagattggatcttggaatgagaagaacaagtgtctataaaggacattatttggacaattggtaaaatttgtatatggactgtgagttagataatagtatatcagtgttaaacttcctaatttgtaatatgattatggaagagaatgtctttatttttaggaaatgtacactaaagtatttagggattaaaaaattaaaagtcatcaattattcacgtaccctagaggtttaagatatgctcaagagtgttttactaatagaatggaaaacgagtgagaggtgggcagaagagagattataggacatatctaccaaaaatgataaaagtcccctaaacaagtacttgttatggtgtcctttgaacatcattatttttgggagtcaggccaattgagtaaaccatgcttgtatttgtttctgtcatcaggtatatggtagttctggtagctagccatgattggtgtctttactgggatgaaatagggatagatatgaaaaatagtattatgcattgttgtagaaaattagtgatatcaataactgcatcttatgaataggttttgaggaatattaaagactagttagctgtacaggaaccatattgattttaacatgatcttggtgtttatataaaaatgtgaatttgaaattaattgtaacctttaagtttctaaattgttttataatgttctaggttctgttcaagttgtaacatggaatgagcagtatcagaagttgactaccagtgatgaaaacgggcttatcattgtgtggatgttatataaaggtatactttactaggagagattatttaatttttcacaagttggcatctggatcacagtatttggatgggtaaataacatctccaaaatagaccccttttactaacaacaggagttcatcctgactgcacagtgataatatttctggatttattgctgcctacttttcacttttttaaaactttaaccctgttattaagaaagtgaattaggtgactgttggccatctccaaaatgaacttagactcttagcagaagaatagcttctacttgagataagtctttttttttttccagatttccaataagaattacttctaggtagttccagtttataattacattcatgtgtgtgtgagtgtgtatttaatattggttcctcagtagactgtaagctctgaggggaaaggggaaatgactgtttgttcattttatacgtagaaccttttataatacatagcacagtgtacatgttcaataaatatttgttgaatgaatgaataaatgagtggctagagttatctcagtagataaaaacacatttagtaagaagtcaaagggaataattcacttcctgcatgggccagttagtttccttttcttttatggatacagattttatctcaactctgactactggttcacagtttgtctatgagagagggtgagtaaaagaaggattaacttagataatccacttttaccagaaaagcagctcataatacatgttctcctgaggcctgtagttatcatcttcaaaagggaagggtgccttagttcatgtgtgctgttttgaaaagttcatgtgtgctgttttaaatggcagggcataccatttaaaaagtcttcattaaagatatgctataatactttaattttttaaaaagtgtgtgtgtgtgtgtgtgtgtgtgtgtgtgtgtaggggatagaatgtgaaagttcagtggactggagtaaactttgtctgtttccaaggtaggtcttcaggtcttctctgggtctcagtctcgattctgttatcctgctctgggcagttattccaaattacatgattatttgcttggctgcaggccagagttgcccatgttggtatgtcaaaaagcctttgagaaaagcaaaaacaaaatagcctatttttttgcttctagtttggttatgctaaaagacaagttacatctcattcctaccctgcatacaactagaatatttttgtcattcggcattagagatgtcgtgtcactaatgtagtataaactgattagaattttttcaagcaggcttagggttttacctaatatgctgtgtatttttttgccttcattctctttgtgatggtaactaatatgctgcatttaatatgcttgtaattattcattggagagtttatgtgcatatgtataaagttattgttacttggagtttttttaattggttatgatctgaaccatattgttcaagtgggaaaagttaggctttcagaaaaatcagtagttttagctctgaaaaagcaaatgaagtttaatttttaattgacagaaagaaagagagagggaaagaaagaaagaagaaacaaaggatgggcatttattatgttttctgtgataatctttgaagcagttatcagttaagcccaagttaatttttcattttgtttttgttggatttggaagttttatagtttaatgtctcaggttgtagaaaactttgcagttatttgtcccatggcgtactttatggaatgctttcaattcttaactactatttaatagatgtgaaagttttgacttgtaattttttaaatttgtgaaaacaggctcttggattgaggagatgatcaacaatcgaaataaatcagttgttcgcagtatgagctggaatgctgacggacagaagatctgcattgtatatgaagatggggctgtgatagttggttcagtggatggtaatgtattgattggacatagaactaagatcaatatctggctgacagttttcttctttagagatttaagttagttaccatatgttagcattctgattgaaaaatagacataaaagggaaactatttgatatacaatttgacgacattcaagaaccgttctttgattttaaccatctgtttttatgtatattcaaggaaatttttttatttcctacagtggcattgaaaaaattgaattaacttttcagcattattttaaacatgtctgcctcattatgtaatttctttttatattggttagtctgtacaaatatatagactttagtttcaaaaagtctgtggtcgtttttattggaactgaatatgtcattgttggttgtagtttatagccttaaaaacctgtatttttcaaattctagtaatagataaaactgggtcttctgagagtctgcacctttgttttaatttgaaactctgtagataaataatttattttaaaatcatttttatttctctctctcgctcgctctgtgtgtgtgtgtgtgtgtgtgtgtctgtgtgtgtgtgtgtaacttcatgtagtcaacaaatgtttctgagagcctataaagcaacagctatcattttaggaactaagggtacagcaatgaccagacctagatcaggagagatggatgagtaactgagaaattacagtatcctgtgataagcgctaagatggggaaaatacaggagcatgtagaagtgccacctaatgcagacttggagggatcaggaaaggcttcctggaggaagtgatgctgatgctgaggcattagccacaaggtaggttggtgtgtagaatgtctctgccctaagaagtagtatttgaggaggtccagaggagttgcacttagctatgtttgggccattgaaaaaagttgattatcagtagggtataatactaacagtgaagagtagtatgcagtgaatctagagagagaaacagaggccagatcatgcagatcataagttatattaaggtgtttggacttaagtccaacagcacttgggaggccaatgaaaagttttaagaatgggagcaatgtgataagattgttctttagaaacatctctgtgctacatatgaagaatagagaaaggaaggaaagataatgggcagctgtatcaggaaactgctctggcaattcaggtgagagattatggtggatggtagccatccaggtaatgacaagggaatggaagtaagtagattttatacatactcaggaggcagaatagataggattggggatgagaggatggaagtccccagggcagtatccagagttttagcttgggcagctggatggatggtaatgcctttcattaaaatagtgtggagaaagattgattttaagggaaaattcctctgttggatatgttgattttgtcatgccataggccattcaagtaaatctatccagaccatagttagacatacatatctgcagctcaaaaatgtctgtgctgaacacaaaattttgggaaccaccagcatatgcatggtaatttaaaccattgaaatagttgaacaaaactaagaggcatttgtagagttaagaatcagcaacatttaggctggggacagtggcttatgcctgcagtcccagcactttgggaagccaaggcgggaggattgcttgagctcaggagtttgagaccagcctggacaacatagcaagaccttgtctgtgctaaaaataaaaaaaaaacaaaacaaattatctgggcgtggtggcatactcctgtagtcccaatcacctgggaggccaaagtgggaggattgcttgagcctgagaggttgaggctgcagtgagccatcatcttaccactacactctagcctgggcaaaagagtgagaccctgtctcaggaaaaaaaaaaaaatcagcaacatttaagggataggcagaagaaggatgtcgagaaatataagagtcttagagataaaggagtatatgggctaatggcagccaagagagttcaagaaggagggagtggtaaaaaaaaaaaaaaaaaagtatcaattggctttagcaacaggccatcagtgacgtccatgaaggcagtttctttcttttttttaaacaacagaaatttattttctcagagttctagactctagaagtccaagatcaagatgtcaacaggtttggtttcttctttttttatttattatttttatagatttagagggtacaagtgcaggtttgttatatggatatattgaatattggtgaggtctggactattagtatataaccataacccaaatagtgtatgttgtaggcagtttcaatggagttgtggggcagaagccagattgggaggggttaagggttgagtggcagttgaagtggagactataggtgtgaagaactctttcatgaagtttggttgggaaaggaatggtagaaatagtagttagagggggaacttggggttaataaaagtgtttattttatttagggtgatggtttcaaagagaaatagatctgcatgtgtttaaatccacataagaaaggccctggagtgaagagtttaatgagaaagagaaaatgagagtagtcgataaagggagatctttgcaaaggtaggaagggattagatcagagaatacatagaggaattagccttggataggggactgactggggaaatacctcttcttttgtattagtgggcaacgcagaaagtgtaagcatggactcagttatgttctgaattgttgaagatgggaatgaggaatttgttttttgggtgcttttattttctttccaaaatagaagatgggctcaactgctaattgggagtgagatccagcatcagagtttagaagaaaggggagaaaggttgaaacagctgctatttcagatgtcttttcaggcacatataggtgctcagtaaatatttattggccaaataaattgaatgaatgcatgaatgctgtgaagaatttgtgagatctttagaaaaacaaagatttcgaaagccgttgttggaggatcatgaccttgaacaacttttaataatgttttgttttaggcaatcgtatttggggaaaagacctgaagggtatacagctatcccatgtaacatggtctgcggacagtaaagtcttactttttggaatggcaaatggggaaatacacatttacgataatcaaggaaattttatggtaagtaagtgtatgcatcagtcttgtttatacaattttatatgattatcgtacatatcattcatgtttgtatatacatttattttttatggcggtttgtgtaattaaagtcttcaattcagattttttttctttttcccaatcgtctaaattttgttttaattaaataaaattttggttttctcaaggcaaattagtgcaacaagttcgagtgtagcttttagttccaattttttttggtaggtagaaaggacgaggtgtcagtgcatttaagcagagagataatgagtagggcgatgtgcagtttaagttttataggtgtcttctggtgtctgaagccaaagtttacatgtagataccacagggtatttttgtagcaagctaccaatggttgtttgtaaaaagaatgacagacttaaatggtaagtgagaaaaatcaatccctggaaagaaggtaaaatagaataggatcaggcagcataatctggagaaaaattctggctttgcaaggtttagctatcatagatgtatgattactggtggggcagttttgttttttagcaagggttaggacattaagtaaatccactcactggtggaagtaaaataatgaacttttatgtcattttgctgttttcaaagtacttttatatacttgttgtttgagtctcataaaaactcagtgttttttaatcctcattttttacatgtggggaaaatggacatttaggtgatttgtccatggcctgtataccagtaagtgattaagctttaatttgccacctgatttttagactgtatttttcagtgttctttctacaatactctattgagtattgtctctatactgctgtgttttaaaatgtaaaatattaaacctaaccaattttatttagataaaaatgaaactgagttgtttggtgaatgtcactggagctatcagcattgctggaattcattggtaccatggcacagaaggctacgtggagcctgattgcccttgccttgctgtttgctttgataatggaagatgccaaataatgagacatgagaatgaccaaagtaaggaatttttctgtttaaaaaatttttttaatttctatttttttttttttctttttgagatggagtctcactctgtcgcccaggctggagtgcagtggcacgatcttggctcacggcaacttctgcctcccgggttcaagcgaatttcctgcctcagcctcccgagtagctgggactacagggcgtgccactatgcctggctaattttttatatttttcgtagagacggggtttcaccctgttagccaggatagtctctatctcctgacctcgtgatccacctgcctcagcctcccaaagtgctgggattacaggcgtgagccactgtgcccggcctcaatttctaatttttaacttttttttttttgcgacagggtcttgctctgttgccgaggctggagtgcagtggtgcgttcttggctcactgcagccttggcctcccaggctcaaggtctcagcctcctgaggagctgggaccacaggtgtgcaccaactcctccagctaatttttgtatttttcgtagagacagggtttcaccatgttgcccaggccgatcttgaactcctggactcaagtgatccgcccgcctcagcctcctaaagtgttgggattataggcgtgagccgttgtacccagccctgtttttttaaaaatgtggaaagttttaaaaatgtttaaaagttgttcttaagattataaagttctctaggctacgtattttcccacagtgacctcatttttctctttctgactagatcccgttttgattgacactggcatgtacgtagtaggcatccagtggaaccacatgggcagcgtgttagctgtggcaggcttccagaaggcagccatgcaggacaaagatgtgaacattgtgcagttttacactccgtttggtgaggtaaatgcattcaaattgatcttctttctttctatttaaatgtgcagtgagtaaggtagaggaaaaaataacaaaggaaacggtgtaaacttatcacattttcttttagggacatttcatcttcatagtataaatctttgttgtattttatcatggaattttataatcttatagttttataattataaaataaaaattacaattttatacattcagaaaaccagaagtttaacataagaaatcaccatggagtagttattttgaatattttctctgtgatctgaatatgtgtctcttttcatagccagcttttgagtagtatttagtataatatagttgtttcagattaatttttctgatttttaatgtgatattttaaatgtgtttagatgttaaacatacatatctgtctgtccttccttcaaacttctaatacttttaatttgttgcaaagtaatcagttatttgaagaaaatttaataattaatcttaggtgatgctacattaaaataaaagcattgttttcataaatgtacatatgagtattatccttctcaatagtatttttaataaatttaattttaaaagaattttcagaagtagcattggaaaatgtcaagtaggagaattaatatattgtgaattttaaagaatttagaaagtgagagcttattaataactaagcttaatacgttttttgaatgtcttattttctaacccagtactatattttgtcccttttgaataccctgttggcttttataaattcgccatagactacagtgatatatatttttggctgcataaaagcctataccagttgttttaatgttatttttatatattttaataatttcttttagggcaagaaatgatgtcttctgatgcttttgaatcagtatctcaaagttacccagtagaatatactgtagaacgtagatgctctctatacattgtcaaacgacaatatttctcgtagatgtaaaagtgaaggaaaaaaaaaagcatagttttcaaggttcctaaataattaaaaatctgcattctaattatttctatatttatagcctccttcttaattcttgttctttattggttatttttgttggttgccatttacttatttatcataatgttttatctgcttatttttgttagttcagccttccccaggtagaaaaacatgggttctaaagtctgtttgtttgtcttgcaaaacaatgtgttaagtgttaaacggaaaactgtaagtggggtttggtttagctgtaactaaaagtatctagcatagttcctcaggtcatagtagtcagtattttctgagtgagtgaatatatggggaattatttggatctgtgttattgttaaggtaggaaagataacctggatggaagttatctaacaatttaaacttaatgatttaaacttaagaaaacctcagagaagaatttgcttttaaattctgagggccaggggctcctgggttttgcactgtggtcttagccatggtaggagataacagagcatagggtttgtagtcaagttgatctggtattgaattcattttccacagtgtagtttgacgtagagaagttatttagcctctgagacacagtttagttattttcagaaataaaggaaatacgtcatggtgctgttgtaaagattaaaggagattacaatgaggaaatgcctacacagtgcctggcatgtaatgagggcttactaaaaggtggttttctttccttgctgctgccacatcctggctggtttgcagttagaagatggtcgtattctcctcctcttatggatgggtcaataccaagagtactgtagagggcttgctggttcacctttaatttcatacaggtagcaagacagatagatcagaccctcctaggttaaaactttgagtttttttcttccattggtttccttgggaaaatggagctcaatacatttgtcttactctattatgtgctgctatgacagaataccacagactgcatagtatatattaacagaaacttattggctcacagttctggagaatgagaagtccagtattgagctactgacatctggtgagggcttttctgctgcataatttcatggtggaaggaagaagggcaaagagagtgcaacagagagacaagaggggacctaattcccctttgtataatggcagtagtcccagccatgagggccctcatgccctgatcactgcttaaaggtctcatctcttaatactgtcaaaatggcaattacatttcaatgtgagttttggaggggacagacattcaaactatagtaacattaaatttgaccctagagggtatctgcttagaacaagggataccagtaaagttcattgaaattttggggattctatcattctgtcaaataaaagatatagattaaaaatgttcgattgccttatagccacattaaaaaagtgtttgtattcatttattcactcgttaatttggtcataaaaagtactgtttatttattttaaattttaaattaaaaaaatttgaatgtatatttattgaagatatatattgactatcagtttatcaaataatctattaatggaaagggaaagaaatgtgaatatgatacagttagtactcttaaggagctcattctgtaattctctggaaccatagaatgggggaagaaggcaggactcgttgaggaagcagtatttgagctgtatcttgatggatgaataggcgtgctccaggaggacaaggaccagtaaggataggggagctgtgaggaaagaagagggggatttcaggcagaagggatattgtataggccaaagctgtggaggcagagtagttccttcacgagcaggcacacagcagttatggctaaatagtgggatattaagccacataatatgattttggacaagatgggaaaaaggcattgtatgccatgctaaagaatgtgatggtggggatttactgatttttaaaagttattaaaccttttaaaaagcagggtaggttatgattagatctgtgttccagaaagataattgtgacagcagagtatagattgaattgggagggcagagggggagaataaggaaacagatgaaaagtaagatataaagttaggaagctattaccacaatgcagtccattaaaaaggtaattgtagagagtggaaaggagtaaaagcagtaggacttggtgactcgttggttgtgaggggatgaaagagaaagacaagactttatgattgaatttgctaataaaatatttcattgattctaagaaatataattttttacaactttaaaatgaggaggcatcttataatcaaagttgccatattttaattagcaatgtgttttttcctttccttagttgtacataaaaattcatgtttcagtctatggcattttagatttgatgaaatttgctattgaattctttggatatagtttgtaaatttttcttatatgcatgtttttcattataaaaattcaagaaacgtggctgggtgcggtggctcacgcctgtaatcccagcagtttgggaggccgaggcaggcagatcatgaagtcaggagatcgagaccatcctggctaacacagtgaaaccctgtctctactaaaaatacaaaaaattagcttggcgtggtggcgggcgcctgtagtcccagctactcaggaggctgaggcaggagaatggcgtgaacctgggaggcggagcttgcagtgagccgagatcacgccactgcactccagcctgggtgagagtgagaaattccgtctcaaaaaaaaaaaaaaaaattcaagaaacatgattttaaagtgactagtaacttttcacaaaataatgttcataccacttttgatctctgtgaagtgattggtggtaatttgtatttcagttaattgttatactttaggttaaagataactttctttttcagcatctgggtactttgaaagttcctggaaaggaaatatctgcactatcttgggaaggaggtggactgaaaattgcactagctgttgattcctttatatattttgcaaacattcgacctaattataaggtaaatattaagagcagttgtaaaataaacagtgtttcttactaaataattttgctatagctcaaagtatattcaaagctgtcttgttttaggagctagcagaaaggaagcctttttgtgtgaagattttagaactagtaaacttttctagaatactatatatagttagtaaaattactccatgagtcttagccagaaacagatggtacactcaaaggagtaattgaagagagattgatgaagggcttattacagagatgtgggcagggctaaagggaacagtaagggatgataaaaagtaccacccaaagattagcgtcaatgagaagccatcaccagccctgggcctgaaggggcaagacaagggagtcaggctcctggaacccacaagagctgtggctgtgcaagagggtctgctctgcaggagcccagaccctgccaaccagccaggcagagagggatttggggaataaatattccaactcacctctccacttgctggtgctttctattggccaaacccaaaggggagccagatggccaggtagcccaggtgacggagttcgtagaaactagtctccaaggctcacggcagggtagagaagggcagagaatggattagaaggagaaaatgagaataagcaacataacatataatcagtattttatcttgaatttaacatttgatccaaaagcaagctctcaaatgtgagattgacctggtaaactatttgttataaaaatcttatatatttaattttgaaaaggtaaaggaccttagagaaatggctgattctaggactagggcagggaaaatacaaggtaagcctggaacagtttatagtcccagcaagtaaggaagtgctcagacaacaaaaggatggggaaatgtcaaaaggacattgctgaaagagcctcccaatggccaaagctggagcactttgaataataaaataaatgatgtattattttattgtgacccaaagtataaatgcccaagagttgatgctgatatagatggattatttgaataaataaataaatgggagaggagaaacagatcttccttacagaattattccagtttacttaggtattgccttctccaggaggtggaacataggcccctgttcccctggcttgagtgtggctacacttagtttccaaagagtagagtatggagaggggaaaaaaagtagctttatattggtgaaacttgtcaatcacaccttggccaagtgataaaggttaacatcattaggaatgtcatgtggctatcacatgccccctgatatggcatgataaagaagtgcacttcggttctgtgctagtcttcctaaaaacctgaagtccaatctaagcatgaggaaaacatcagacaaacccaaattgagggacaatctacaaaatacttgaccagtactcctcaaaactgttgaggtcatgaaaagcaaggaaagactgagaaattgtcacaccccagaggagcctaaggagacatgactaaatctaagtatcccaaatgggattctggaatagtgaaaggacattctggaaaagataatgaaatctaaataaagtatgatgtttacttaatagtaatagtaatatagttaatagtgatgttgatttcctagttgtgacaaatgtaccatggtaaagtaaaatgttaacaataagggaaatggatgaggagtatatgggaactctgtgctatctttgcaacttttctgtccatctaaaactattttaaagtaaaaaaacttattttagaaagtaatcaggatacaaaaagctaaacatggtttagcttagtttgtttgaatggtaaacacaatcatgataatatcaacatgatattttatttcatttttatatttttaatataatttatgtatttttatttttttattcaaaattaaatataaacatgttgggagagttagaagggaaagctgtaagtgtgtaagttccataatcaatagataatgtgatacagaaataaacaccagaacagttgcagttaaagtagttgtacacgaccattctagagagaggaaatttggcagggggttctacatttgtttttgttttaaataagccaggtaggattatttgaccatatattatgtgaatatgttactctgataaaagcaaatataaaaaatatggatgggggtaaaaaggcaaagtgactatttttgaatttcgacttggagatccagaattgaaactttgacagggatcacgctacctgttgtcccctctccagcagttattattgattattgaaaatttttaccattgaagcttaaatcttaccaattaaaataaggaaagttagacaaactaaatttatatttgcagcacattctttgttttataaatctctattatttttataaaaggttgaatctgtcttctatttctgaagaatatattttgtcagcagtgataaatatctgcaatattttgagagtaaaaataaaataatgtgatttgccttcccaatactaaataattcttctccctttcaatctccttccctttcctccttcctttcttcctagtggggttattgctcaaacactgtagtttatgcatataccagacctgatcgtccagaatattgtgttgtcttctgggatacgaaaaacaatgaaaaatatgttaaatatgtgaagggtctcatttctattactacctgtggagatttctgcattttggctacaaaagctgatgaaaatcatcctcaggtaggtgtttcttgatatcttaagacatagctgggctaactgcttgggttatagttttctaccttttcaagcatggcctctacactggcaagtacatcaaaacattgggtctgttggcactattggtctagaccaattttggaaaactctgtgactccattgatgctgtgtccctgattttttttttcttcaagcagctaatcttatggcaattcatactgactggtttaatactgaagctttttgtttctgaaatatatttaaaataccgcaatcccttttcaaattaaaatttcatgagcaaaaatgaacatccagatgtctactatgcataaatgctgaagaaattatgcagcaatgaacacttgagacagtttgggtagcttttgacattgagaaatctactttggagttatattgcttttaaaattaatgtatcatagcataacataaataatataaatatgtctttttaaaaagtatactaacaacttaaatatttgcagaagtatgtaaatatttctgaaaaggactaccaatatctttggctgtgttatcaattattttaccccattaaaaagtatagagttttttgttaaaggtgactctttgaacagccatgtttgctggtgttctctcctgaaacttgctaaacaatcgtaaaggtattttttaaaaagtcataagcccatagggaccagaaagggaaaggaaatggcaacaacattttgtaggttagagtaggtggattagcggtaattaccttagcagacctaagaaagctgaatcataagccattcagggagaaaacagaggcaactggcttagatcacggaatcccaaagaggcacaggacttgggggtacctggtgggagaaagaaacaaacataaaagcaatttggaagtaccagagattatgcagaaaagagaaaactttttttaagaagaaggagatcccactcccctatactgagtccacaggcagctgcccttctgccacctacaacgactggggtttactctccagagaggatcaaagagtctctgcgctgaagagcatcaagcagagctgagggtgggggaactctgctgaaaacaggacaattacgttaaaagtttgctactcaacattacccaagtcttcttttcctatttggttcccagaacactggcagccaagcattgagctttgaggtgggagtttggaaatcttctcaggagaattcgacctgttcaagagaaaagccttctaaagatactgagattgagagtccccagggaaatagccctgcccatcaccttacagtaaagacgccagtcagtgaggctcacttatagacaactctgttccgaacagcttttcaatgtctgactcttaaatataagcagttagacaaggattgccagacatttgagaaagccatctaaacacaagtggaaaagaacttggaagaagcagagactgtgcaggaaggacacgtctgaaaaacattattttggggctgggtgcgatggctcacacctgtaatcccagcactttgagagaccaaggcgggcggattacctgaggtcgggagttcgagatcggcctggctaacatggtgaaaccctgtctctactaaatatacaaaattagctgggcatggtggcgcaagcctgtaaacccagctactcgggaggctgaggcaggacagtcgcttgaacccgggaggcagaggttgcagtgagccaggatcgtgccattgcactccaacctgggcgaaagtgagactctgtctcaaaaaaaaaaaattattttgaaagaaccattaatattaccttcagagagttaagagatattacatccataaaataagaaaaggatactatagaagaggaagtttttggaaattaaaaataggataaccataatgaaaactttggctggaggattagaaggtaaaatgaaggaaaacacttgtaaagtgaagcaaaaggacaaggaaatggaaaataggggtgaaaagataaaattagaggaccagtccgggaagcttagcatttcaataatatttctaaaaagagtgaacaggccactttgggaggccgaggtgggcagattacctgagctcaggagttcgagaccggcctgggcaacacggtgaaaccctgtctctactaaaatagaaaaaattagccgggcgtggcagcgtgtgcctgtagtcccagctactcaggaggctgaggcaggagaattgcttgaacccaggacgcggaggttgcggtgggccgagatcacgccactgcactccagcctgggagacagagtaagactctgtctccaaagaaaaaaaaaaaaaaagaaaagaaaagaaagaaaaaagaaaaaaaaagagtgaacaggcaaaatatacaaaatacagggcagtaagtatgaaaaaaaaattcaagaaaattttccttgacgttctggattggaagtgcctagctcagtgaataaaaacatttccacattgagacacatcattgtagaatttcagaacattggggagaaaacaatcttacaagctttcagggaggaaaaacagatttcataacgtcaaggatcataagtggcattctatttgtcaacagcaatggtttggagaattgcctcaaaatttgcagggatagttttccaactagaattccatagctgtaagtcaactactaattaaatatgagggtagcccagcactttgggaggccaaggtgggcagatcacctgaggttaggagtttgggaccagcctggccaacatggcaaaaccctatctctactaaatatacaaaaattagccaggtgtggtggcgggtgcctgtaatcccagctacttgggaagctgaggcaggagaatcgcttgaaccctgaaggcagaggctgcagtgagccgagatgtgccactgcactccagcctgggcgacagagtgagactctgtctcaaagtaagtaaataaataaataaataaataaataaataaatgggtggaatacagacatttttagatgtgtaactcactaaaaatttcccttccaagcttccctatcttaggaagttactttaggaaatagtctaacaaaatgaggaataaaccaagaaagcagaagaaacagtagagtggacaccagtgaggaagaaagtaccagctgaccatggtgcagtgtgtccagtgagtcagatgagaggtcagaaggctccaagagaaatttaaggggataacaatagactacctgacatatttgagcccttgcagcaagatatacataattcagggataattggggatttgtgttagtaataagcccatagaagactaatcaaagagaaaaaatacaattatcgactacagagaaagcagaatgtgcttgaaaagaaaataaatcatgatacacttgatggctgagttgtgaatagcatttacatagtcaaaatgatgtaatcaccctttttaaacttaaaacaccttttaattgaagtataataaacatactgaaccctaaattctgatccgacaaaatggtattaaaaatagagagagggatggaaaggtgcccctgtgtggtaggggtggggtggaaaagggaatgaaagaaagctaaagacttgtcatttgtagtggggaggcaagagctaatggactaaattgaaataaaacaagtagcagtataaacacgttacttagatttgtggaggtaatagcaaaaggatcggtaaagggagcaggaaatgggaacttagggggaactgccattttttagtaagaagccttgtggatctatttgatttttggaactactagcatgtataactgataaaaatgaaaaatatattaaaaatacaatttgtggtattttgtggcagtaaataagcttattttaagtagtggaagagtaaattatgattttttaaaatatgatttttattatttggtttatatttatgaaattaattcaatgagttctttttatttttttttaaatgttatgggtaatagtagacgtatgttatctatggggtacctgagatgttttgagtaattaaacaaattttaatcgaatgctgaaaaaagaaaaagtctgtgtaaatagcacataaagaactgctttatttgaaattgttgttctagtcaggaaaaacatctaagtagaatagtattcttagtacaagatacagaaacagagactaacagtaagaaaaatgaaatgatcactttaaaacaatgtgatgtaaaatgtgcattacgtttgattattttcctaatattggtcattgatgattatttgtatataagccactttcattaggttgagtcatgtcttttttgagttaactgaatttttctgaatttctcctgtcatttatatattgcttcaaaggaggagaacgagatggagacatttggtgcaacggtaacaaatgaaattagctcttatatagctgatcataggataaaaatgagttttgaggttatagctgatcagaaactgtagcaatatgaggggacttcagaaggttcatggaaaaatggaattaacagataaaaataaaaatagaaactttatttttcaacataagctccatcaaggtcaagacatttttgtaagcaatgataacagatatttagtccatccctaaagaactgagggtcctgggaatttaaccatgtcaatgcagtcttttttacattattaactaaagataaatgggtaccctttaaggattttttaagattaggaaacaaaaggaagtctggagccaaatcaggactgtaaagtggatggatgcctaatgatttcccgccaaaactctagcaaaattgcccttgtttgatgaatagaatgagcaggagcattgtcatggtggggagagactctggtgaagttttcccaggcatttttctgctaaagctttggctgactttctcaagacattctcatagtaagtggacattattgtcctttggcccttcagaaagtcaacaagcaaaatgccttgagcatcccccaaaactgtcgccatgacctttgctcttggcggtctgcttttgctttgactggaccacttccaccacttggtaaccattgctttgattgtgctttgcattcaggatcatacaggcaaagccatgtttcatctccttttgcaatttttgaagaaatgcctcaggttcttgataccacctgtttgaaatttccatttactgctctgctcctgtgtgcagctgatctgggtgcaacggatttggcacccattgagtggaaagttggcttaactcaaatttttcagtcagaattgtgtgagctgagccagttgagatgtctatagtgttgactattgtttctgctgttaattgttagtcctcttcaattagggcagaaacaagatgagattcattttttccttgcaaattgacatggatggtctgctgttgcaggtttcatctttactttgtcttgtcccttcttaaagtgagttgtccttttgtagactgctgatttctttagggcattgtccccataaacttttcataaaggattgatgatttcaccattcttccacccaagctgcaccataaatttgatgttttttcttgcttcaattttagcagaattaatgtttctttgacagggctttttttcaaactgctgccatctccttcttagtgcttcaaactagatcctgttcaaacatgttttaacaagttagtgcacgtttattttgatgcaaacaatttttaaatccatgcttattttttccataatacacattttccataaactttttgaagaccccttgtataaatgatgaaaagtagccagagtacttgtgtttcttgtgtcttcttaaaaaccacagtgttccattacattgagaaagcagccttggtttagtattaactgatctttctttatatttaatgctgtaaatcatctttctttttgtctgctattttttgcacgtaacagtttcatcagttatttcccagttagcaatacttgaaaaagagataagcatgatatatattaagcatatttcctttattattcataagcaggagttctgatactttttgttttatttgttcacagtttgtactagttctttgtaattctattggtacacccttggatcccaaatacattgatattggtaaggaaatgttgatatttatttcttttccaatcaggttttatttgtaacactaatttaaaaacctaacactggtattattaattagaatggtgttttctaaaacgaggatgaccatacttcctactttgtcagatccagtaccagtttatatctgttaccatggtagaattcttaatatcccattttactctcaaaagtatcctggttaagacagtaaattataaggttagtctatctaaaatccattataatttcacaactgctatagtaccatcattcgctttaaatgggaaatataattgtgtaggaattggtatgcagcgtaatgattagtatgtaataaactgaggattatagaaagaattttgctattaaaccactttcattattaataaactgcttgtaaatacctctcttccataaaaacactttggctgcttgtaaaagtgagtttgccctttccagatttttctaatgaaatgagtctattaatagggacagttttaaaaattcctgactttttgttttcaaaggacttttgttactttagatttactattccctctacccaataactatgaaagtaacccattaaaaattccaaatcttattctaagtagattattaaaaatcattaacaagtattttgttgccatttcactgtaacttccaaaagtaatgattttcttttaatgtgtatggtagtatattggatacagtattgtaaaaattattaaatctaagaaattaaaaacccattgcattattcctgagaacctgagaactagtcatttttttttcatattggacatttagcctaagcaattaagtattaatgcctgttgccttttgagtgtttgcattttgctaagtgtttgttgaaaaaattgctaagtgtttgggtgataatagcaaatatgagaaataatgacctcaaagactttatactctatggcttagggtcttgataacctactcacttagatatattgacacatttcaacttcatgtcttacattcattgctacataggcatgtttatatgttttatatgtgtatatatttatatacacatatatatacacattagtgttccttgttaaaaagcttagcttaaaattattaacagactctcatcacataaatatctcttgtttattagaagcattaacagtctttaagaggtaaattttgtcagtcttcagattatatgctttttcttttaatatccaaagttttaacatagaatcataatggtgatagggttaatgtcaaagtaatttttatcacaagtagactgtttttttaaaataaagctaaatgtaaatccttcttcataaaatggcaaatacttgagagcatgactatttgtatttttgagtctcaaaactttttttttttgtagtcttgctgactgagattttaacaaacagttctttcattggcctgcagtgaacacagccaaagactcaacatttttgtttcatacatatcctgtaaaatttcttttttaaagtttctatgccaaatgtttatgaggactagaggctaaaataaaaaaagtctgtgagtgatactcttctagaatttattatttttgttttgccacctactcttcctctttataatttttgtagaaactttaaaatgaactttatagattattatagaagtgtagtttgtgggactctgttatactctacacatgagagttaggtatccttttttggtgttcttttcttgagcactcataatacctttggtgtgcttccatccaaaatacctcttaacctgcactgtggttactaattttcttgtctatcttccccactagactttgaacttcttaaagatctacgtctaattaacttctgtatccccatgacctaacactgccttggcccataataaatgtttattgaatgcatggaaattatttattttctatcatgtaaataaaaaatgatgattcatttatttagcaaactatcaatgagtatatactgtttcaggtactgtactggtcactgggaataataagaaattaattatgacatttcctgtgggatttctgaacaaataattaaaacctagttaattcattgaacagtgatttattgcttccctactgcgtgctaggtattgctctaggtgccaggtaaatggttattggtgcataaggacatcagagacatctttctacttagagaaggtttcatttgagctgagttttgaaaagtaagtatttctttaggtgatatttcaaggaaagaaaacggcatttgcagaggaatgaaggcatggaaagtggccatcaagtaccagtttgcctatgctgcagccattttagtgggagcacatgggcagctttctaatgatggtatctaacacgtgcactaggttttccatatattttagagaaagttaatttgatgaaaacatttcagtgaaatttcaatttaccagcacttgaaagggctgagaaatgaataaatgggaaagtgggtgaacattacagatgtaggcatttatttcatgccttagcattaaaatattacaagcaggttttatttatatgtgaattccaagattgttttaaattattttacatccttgcttggctagtaatgactgcctattagcattacaaagttcagttaatgaaagcataattattttcttaaaaactttttttccaagctgggcatggtggctcactcctgtaatcccagcactttgggaggctgaggtgggtagatcacctgaggtcaggagttcaagaccagcctggccaaaatggcaaaaccccatctctactaaaaatacgaaaattagccagacatggtggcatgcacctgtaatcccagctacttgggaggctgtggcaggagaatcgcttgaacccaggaggcagaggttgcactgagcaaagatcgtgccacagctctccagcctgggtgacacagcaagacaccgtctcaaaaaaaaaaaaaatccaataacctttctagatatttcaaatttttttcttttcagatattgaacagtcaaaaagggcatggagggccttcatcttggctgtgttcttgtcctaaaacattgtatctgttaagattttgttttgcttttcctagaagcttagtctagatgggatgaatcttaataaagatctagcaaaatatgtatagataggattatctacccatatgaatttgctgaaattaccaccttactgattcaaaagaacatatttttcagatatatttctattttttgtagtaactgagaatttatatatggttttattataaaatcctatgtaggtgataaaagtgaaaagaacaatagcaaccatgtctaactaatacttcttaatccttagtgagggtcagtctgctctagtagttaagagcagtttctggagccagattgaccaggtttgtgtccagctctactactttctaactgtgtgatatgggcaaattcacatgcctcagtttctttatctgctaaatgaggacaataacgcctaccttatagggaagttgtgagttaatatgtgttatatatttagaacactgcctgtcatatttttaaacattcaataaatactgtcttttaaaagtattattggccgggcgcggtggctcacgcctgtaatcccagcactttgggaggccaagacgggcggatcacgaggtcaggagatcgagaccatcctggctaacacggtgaaaccccgtctctactaaaaatacaaaaattagccgggcatggtggcgcgcgcctgtagtcccagctacactggaggctgaggcaggagaatggcgtgaacccgggaggcggagcttgcagtgagtcgagaccgtgccactgcactccagcctgggcgacagagcgaaactccgtctcaaaaaaaaaaaaaaaaaaaagtattattaagcgtgtactctatgccaggcactgtgctatgctataatttatattgtatttatatttatattgttatctctcttaaactttcaaacagctctgtagggttggcattattattattttcaacttacagtaagtaaactgagagaacaaataatttattcaaggccacagaattaacaagtgcctgtcatttgaatccaagaagtctgactcttaccttgaaccactaacattttaattttgaagtaatcaggctaatgataaacccttgtgagtatttaggatctatcaaaaagaaaggaacatgagtgtaatttataattgtaggtaaacattggttggaagaatgcttttatatacttaggaaaaaatgagattttattagaaagcagttaaggtgtgcaaacttgcttcaaattacactaagggcataatagttaccagcattagagtatccatggtggaatttactaaatatttaaaaaattcttaagatttagtgaagaccaggtcagggaaaggagagcgcagcctgacctggcaggagcgcaccctggaggctttgctgtcagtctccatgcctggctttcagcaaagtgctccttccaagaaaggctttgagctccctccagaggttggtaatagtacacattttgaatgaaaaaatcttgacagttacagtgaaaaaagaattaaaaaaaaaattactcagttggactgagttatttagcatataaaaattattgaatttaatccctgagttgttaacccactcaattaacatcataccatcagactagtttctaattctttgttcagtttctaagtctcttgctgcctcaaatgtcctttgtctgcactctggcaagaactctataaatggtacctcctagtaacgttatgtctttattccggccatattaatgattacattgcatagtatttataatagagaaattaagttttacactgttttgattgttcatgaatttagaaaacaggtagtataatctgtttgtgacggagttttttgcttttatgtatttcgtatgagtgtttaattttatgttgtttattttgtgttttgtgaccttgggcaaattgttctacttctttaaacctctgtttatttatctataagatggatttgatgagactatctacttcataaaagcatatacagtaggagctcaattaaatgttctctaagaaatgggcagatgaaaagaattatttgcttaggagaatatatttccaatacatattaggaatattaaaaattatttgtttagaagaatatattccttaggaagggacttgggagatttttcttttctaaattagagtagaaatataagatgtggccaggcggggtggctcatgcctgtaatcccagcactttgggaggctgaggcgggcagatggtgaggtcaagagatggagacaatctggccaacatggtgaaaccccgtctctactaaaaatacaaaaattagctgggcgtggtggtgcgtgcctgtagtttcagctactctggaggctgaggcaggagaattgcttgaacccgggaggcagaggttgcaatgagctgagatcacaccactgcactccagcctgggcgacagagtgagactctgaaccaggtagtaattggtagtggggatgggaggaaggactgggattttatttattcatttttcaattaatggttgtttgagttgtttctacttttgatcattataaataatgttgttatgaacatcagcatatgagtttttgtgtggacatgtttttatttttcttgggtacatacctaggagtggaattgctgaggcatatagtaactctgtatttaacattttgaggagttgctgaactgtttaccaaagtgaacacaccatattacattcccagatttctccataaccttgctaacacttgttattgtctgactgtttgattctagccatcctagtgggtgtgaggtggtatctcattgtggttttgatttgcacttcctgatggctaatgttgttgatcatctttttcatgtgcttattggccatttgtgtatcttcactgtagaaatgtctatttgtatccgttgctcacttttaaattgggctatttgtctttattattgagttgtaagagttctttatattttctagataaaatttcttttcagatacacgatttgcaaatattttctcccattctatgggttgtctttactttcttgatagtgtcctttgaagcccaaatgtttttaattttaatgaagtctaatttatctaatttttcttttgtcatttgtgtttttggtgtcatatctaagaagccaattattgcctgaccaaaggtcataaaggtgtattcccaagttttctttaagtgtttatggttttagcatttgagtctctgatttattttgagttagtatcgttgtatgatgctacaagcttttgattcttttctgatttcagagactgtgtatgggtattttgagactatgtgggggtatattgtctataaaatgaggggttgaactccgtgattactaaaggcctttgcagcacaagcattctgcaattctgtgtaactgtaaattatcttaatgttatttttaacagtaccattgtttgttgcaatgaccaaaacccatgtgatagcagcctcgaaagaagcattttatacctggcaatatcgtgtggcaaagaagctcacagcattggaaattaatcagatcacacggtctcgaaaagaagggagagaaaggtatatcttttgatacatactttttagtagctcaaccatatcacattgaagtcagacatagtaattgtaaattgtcatgcttgataactgtagtgtcttgattagtagaaatgttttttcaattaatttgattatctttaaaatttatgtgtttaaatttattaaaataaaagagaagtagaatggtttaataaagtccttagagataaagagtggaaagctgtgtttttataaggaaatggctgtcctcacttaccctccccaccagattagagttaatatgctatactaaaatatgattattcacttgagatcgaaggatctaactaagggtttattcctatacattagtggtataggttttcccaagagggtggggtgattccaatatcagtgaccagaggagaggcaggcctttagacacacatatggaagaagtctttaggtggggtgaactggggacttagcttctttttcttgccctggtttccaaggattctggagttggaattacttctagtaagtggggtgaaaagggctctgccacatagtagataggttagaagccaggatagagggattccccacagtaagtctgtaagtgaagaagcatttactgtgttttgtaagttatattgtctccattagtgatactctgtgtttgctgctcttctgttgttactctaagcctgtaagtaaagcattttgttgaatgttttaacctaaggattcttaatggggacacaggcctgtgttggtggaagtgagtggtacagtcatgacagaaatgctaggtgttttttcaaatcattatcctccctcacatccttgagatcttaaagcagactccccttgcctttcctttaagactcactcatgtgctaagatgggagatggatcctcagtaaaagtatgttgatgggaggctgaggcaggagaatggcgtgaacccgggaggcggagcttgcagtgagccgagatcccgccactgcactccagcctgggcgacagagcgagactccgtctcaaaaaaaaaaaaaaaaaaaaaaaaagtatgttgagtatgcgaattcatgtggaggcaggaaaaaaagattgaaaaatgttatatcttttgttctcagtggtgctggaagaccaaatggaaagaatctttggaaggaatctttggccagtatgtggtagtcctcatgctggtaggcgttaccgtggtagcaataataagcagaggcctgcaatcttgtgggagaaggtagatcttaggacaaagttgaagcagaaagcctagcctctccagagtgtcttatagctgctaaagagattatgacattagtttaaatcaagaagacagcatttccttatgtttctgtctgggatatgtgtactggtttgaataactttgagtaggttccccttagaactcagtttcctcatctctaaggtacagttgatgatggtgataacagccaacatttattgggtacttcttacgtgccagacactttcataaatctaatcagtgctcattattcttgttttgcagagaggaaacagacacatgagcttaaagtatttgccaaaggtcaccagtggaagctaaataatgtgtacacatggacataaagtgtggaataatagacattggagactcagaagggtgggagggtagtagggggtaagggataagaaattacttaatgtgtacagtgtacattacttgggttgtggttgcactaaaagcccagacatcaccactactcaatatatccatataacagaactgtacttttgtaccccttaaatttattttaaaataataatacaggtacaggtactagagagacagtggatgtccaaaagtgataccccttcctaacacaggaaagggtaggaaaggcttctttacttcagcgcagaagttgcattaattagaacctcctaaatccacttaaattttacttttattaaacacattgatagaagagaaattatggagaaacaacagagaaagttagtgaattagatgttactctttactgatgttcaagtggagggtaaggtaaaatgtctctctgggcttaatgaaattcatcgggaatatatgatgaaatgttcagatatgtttttggtacagctttcaggaacgtatcatttatataactagtaaacacctggtagataatttttatcttcattaagatttaactgtgttgagaactctctaaaataatcagaatttttatattaaataacagtattaattgatgttgtccaatcgtttattctagagattatgaaatagtataaactatacttaaactttctttgaaattctgatcttttttgcagaatttatcatgttgatgataccccttctggatcaatggatggtgtgcttgattatagtaaaaccattcaagtaggtgattttaatttttccagaaaatgttaatatctgtagatttgaaatattataattttattttgaaacctgaaaaataatttgtttccagtgaataggatattattttagcaattctttaaaaaaatcctcttctgtaaactttttcctagatcttaaaaattcttgaactaaaataaagatggtagatggtgataagtagctaatatactacttagaaaagcgtattatctaaaaatgttggaaaacataactcctcctctcctcctcacaccttcccatctgtctatctctttatctgtctatcagttgaccatggattggtctgtcatccctggcgtattagtgagtgcatatattttgcagataatatagttaactgtttctttatgaaaatatgcttctgtttacaaactaatataatttgctatttcagtaaatagtttttgaaatgattttctcagagtatttaaagtagtgcctattgtttataaaactgttgtctcatttcttatctgatgctcaaaactctgtaagtcaatcagcatgtcatctcctctcttacaggttagaaaactgttagagaagttcactgatttactcaggatcataaaatgattaagtaacaaagcaagggtgcaagcttaattttttgacagcagatctagtgatgtatctactagaatatgcaagagttcatctagtcatctacatatccatccatttttttcaattaagtattttttgaatgtctattaagtgtgagacattggagtaggtgctgggaatctactggtgaataagaaacagtcgctttaacagggaacttttgggaaattgattcatcatcaaccagcttttctcctgtaccctcacataaattaaaatagatgccatcttaaaaacaacagaagtttccattgtcaaccacgtattttcttccagctactaccttctctcttcctcctttcgtagctgagtttcttgaaagactattctgtattcatctttcccctactgttcattccatctgcacttcattgcattctggtttccagaagactttgaattgccaagtctgataggtctttttctgtctttatattatttgatctacacattacatttaatatttttaatgactacttttttcttcttggctttcatgaaactacactcctggttttatctctggcttttccttctgagtcttctttgtcaagggttcatctatgcatcacttattgtgatattccctaatgtctagccctaagccatctaccttttttaaaaaaacctgtgaatacactcttcttagtgttcttaatgaaatccacccctttaatcatatgccaacaactctaatatgtatatttccaatgttgagttttttcaatttctgaataacatatttcttgccatctggatagcaagaaattgagcagctcaaaagccccccaagtttaatctgcctgaggctgaactcagcttccctcccaattcatttctgcatatatattctatatatcagtggttggcttcactatctttgcaagaatcacgagaactcattttctcttgctttccacgtctaataaatcaccaagtcctctggattctgtttcctaaatttccctcaaattggctttcccctttccatctctgtatcagtttggtctaccattagtttctgcctaggtcactgcaataatttccatccagcactgcccaatggaacttcttgtgataatggaaatactgtgtgtctttgttctccagtgcagtagccattagccgtatgtggctattgagtacttaaaatatggctagttcaactgaagaactgaattttaaattttatttcactgtcattaattgaagtttacatttaaatggtgacatcaggttagtggctaccatatcgaacagtacagaatcgcctctctactttcagttttccccatccccactatgccaatccatttattatacttctggccactgcagcctttctaaaatataaatctaatcatgttacatatagttcctcccctccccaattacaagctttctgtaagttcccatatttttcagaataaagtataaatagccacaactactcctccttcctggcccacttcccagtatattcctcagataccagctgtggcaaacaacttgaaatttcctgaatgtatcatgctggtttttaaaattttttattaaaaaacattttaccttgaaataattgtaggctaatagaaaattgcaaaaagagtcccttcactcagctttcttcagtggtgtcatcttatgtaagtgtagtagagtattaaaaccaggaaactgacattggcgcagtactgtaactagactatgagcttattcagttttcagttgtttttacatgtgctaataggtgtgcgtgtgtaggtttatctagttttatcaccataagggatcttatctcttttttcttcattttcttttttttttttgagacagagtcttgcactgttgcccaggctggagtgcagtggtgtgatctcggctcactgcagcctccgcctcctgggttcaagtgattctcctgcctcagcctcccaagtagctgggcccagtcccattcttgtcccctggcaatcattaatatgttctccatctctatagttttgttactttgagaatgttttatggatagaatcatatagtatgtcaccttttgagattggctttttcactaagcgtaatgccctagagacttgtccaggttgttgcagtgcaatgccttcttacatttcctcaatttagtatgtgctggcccttctgctgtctgtttctcttttaggcctgagctcaggtttttgcctgtcttggtgtaagttaggtactcctttctctgcactgtcataacctctgtttctgtctcttcttttatcatactgtcctgcaatgaggtgtttagttcactgtcttcctcaatagacttttcagtaatctctgaagatgaagatttcatctgagtatctctagtgcttagttcagtggttggcacatagtaggtgctcagcagtaaataaatgttggttaagttaagaaatattgactgcttagtgaattgcatactacgttgtaccaggttagttattcaataaatgtttgttgaatagtaatgattgattaatttattttagggcacaagggatccaatttgtgccataactgcatcagataagatattgattgtggtaagttttgcaaaaacttattttatgaataataattctttaaattataaaaatatggtaggagccaggtgtgtgccccatgtctgtaatcacagttacttgggaagctgaagcaggaggattgcttgaggccaggaaatggagaccagactgagaacatagtgaaaccttgtctctaaaaaaaaaaattacagtaaagttttgaatatcatcttatgggcaccccaagttttactgtacccttaatataagttcagtatgagaaataagaaagttttaaagctttcctcgctaataattaaccatttgcaaaaacagctatttatgaaagcagtacagggatgaccagccgagaagacagagtacacttacagtttctaacactgagtggaatgagccctgtggaagattgcatggtggtggggcagattgcatggtggtagtaggtattagaaagtttcctcctttgtgtagcccgatattttatgttggagaaagtgctttggctcagccactttcataagacttagaatgacgaggtaacatgtttgccagccagtaaaatagagaagcctatggcaaatttcagctgaatttttcaaatagaatcaatggcatagtgtcttttcttgagctctgagatttgaaaatattaattttagaaattaatgtttacttttatacagtaatgctgctggggttattgcatttgaattatgtaaacaaaaccttaatagtatatattctagttctagttacattcctcagccatgagctaattttatggggggaaaaatcttaagagcatagtatttctatagtattccctcctttctgataagccctcatgagactcatgctgcaaaagcagtggttctctgattaaaaagaagagagttaaagatcagctgtgtggaggcttccactttgtcccactgccatggcctgaggcagccaagggtttgaaacccacagttctctcatggcattatacttactgttgatatgtcaaccttaagatttgccctgagttgtgtacaaaattatatgctgctttattttttaaacttatttaaactaatattcatagtatccctgcattcagatcatctttccttttgctaaaaactagtatcagatagatgactataggataaaaacctagaccttcagtaacagctatatcatatgatcttctgttgatttttcatatacatatgtaatttaactcaacaagtactttgctttagcttttaatgtgccctgaatattaacctagataacaaatataaataactttagattttttttccttgtgatgtaagaatttgtgtattgccaaagtgctaggtttctgacttctttcttgaatattacatgaaaaaaaattagaatacttcagctgtttgaaaaaggtttaattaaattagtaggtaaatatgtacttaaggacatgattcattatcagaattgagaatttgctgtgacatcactttggttttaagacaaatgtatagacatatagagaaatagttgtagtttaatattattatgatacacgtatgcttaagtaatcaaaagtaatttccatagggtcgtgaatctggcaccattcagagatacagtctacctaatgttggtttgattcaaaaatattcccttaattgtcgagcctaccagttatccttgaattgcaactctaggtaagcctgaaaactctgctcatgtagatgttagacttagaaatgatcagttgggattgttagatttatatgtaataaatgtaaagatgggcgtgtctattatgaacttgtcttaaaattcagtatggttttattttatactgcatttggtttaaatgtttttaattgggcgtgtggaagtactagtataattttttactcttaaagcttaaaagcattgagactattttatatttcataccaattggttttgtctctggatacattttcctcagaagagagcaaaatagagagtagtccatatcttgtatcaaatttatttaacaaaattttaagccatttctactgtattccaggtttctgataatctgattttgaagaagaatggctataggtaattattctcatgattgctgatagtaattttatagttttttccttttctctaattgcattgtttttagccgtcttgctatcatagacatctcaggagttctgactttctttgacttggatgctcgagtaacggacagtacgggacagcaagtagttggagagttgttaaaattggaacgaagagatgtctgggatatgaagtgggccaaagataatcctgatttgtttgcaatgatggagaagacaagaatgtatgttttcagaaacttggatcctgaggtaaaaacaagaaatgagtgttaacagtctataaataatgagccaaatataaaaacctggatgattcccctttgtttctttaagaagtttgttgaatagcagtgccaacacggctgcatgaatttctgaaaataaataggaacatttctgtggattttaaaaatatttggtcttaggtgagaaaatacttatctattaaatattctctactttctctgaaaggtacttcctaggcatttatttatctgttctttttaaaaggtggagccatggtcactagtctttttttcccttgtcaaacttaagaaaacaaaatcagtagccttatttaccagaatttctgaaaagaacctaataaaaaaatatagttttcaaatttccttgtccattcattcttttctgtctctttattcacttaattctgtgcttactttgtcttatttatatttacatttaccatatcgttcacaaatctgtgttctttcttaacccagatattcttacaaatatgcaagtgcaggcagggattaatctctcttttgggtatgaggaagtcccctttgttctcatttttaaggttacattttgattttctgcttgtggctcacatcagctaatttctatgctctggattttcctgtcacttagtctactctggccaactctcagctgttgtatttaggaaatacaaaataatccgagtggtaacctaatcaaaacatcattaagaagataaatcttttaaatgacttttgaattagtaatggattttcaatttggaattttctgtacttctgctgcactctactcttgtaaataataaggaataagttaataaatgctttatatcattaatttccatttgtttgcatgtatagcttccttcttgggaagaaattgcatgcttgggcccctgagctgccaaagggtttctggagtaatttatttagtggtaaactgtttgcccctttaatatcctaaattattttcacatttccccatttgagccctactcaggaattttggatgtaaaatgccctgatgtaaagtgctctcaaaggctctgttaaagcttttgcaagtcttaggatctttgtgattcttcgaccctgagaaatagttcaaaggaaaggcattcagattcagggctgctgcatttgacctgtaagcatgggcttctttcaagggtccaagggaaatttaatccttgccacaaaacaattgtgggaatttgacaggtaatccacaggataaccttgaggatgggagtagaatactttttgaatgttagagtctttttctatccttagccctgctcatactaaatcacaaatcatatgcctggagaagggagaatgtttgttgtatctggctgctcataatgtgtctatagcacacatggtcagtctggtaccaagtgaaatctttgcctggtggaaagagtttcttaagtagttttaacagaacagccttttaaaaacatactgttttttaatttgaatttgttttctggtttggcatgtagttcgatttctgttagatttagagctagatggatttcttaaaaccttcccactgtctggatttctttataaatgatgctgttctgttcttttcattcacaaggacactggcttactgtggtcgcagctggtttagctactctgctcatttgcttttctagctgcttattgatcatgtaagaacatgatctcttcaaagcagggaagatctgttttacatactattatatttaatagtatgttccttaaggacataatattctatattattatttaggagataagttattgtttgacagctaataacaatatatttttatccatttgagtgttgttaagaattctagattcatacctttatctaagctgtaaatcttcagattatttgagagaaaaagatgccatctgtgtgcaaagttttaattcactgataaagcacattttctttgagaattaggagatgaatatttaatgttgatattccctgcttgatcattttcatctccagtgaccctctgaaggtactgattgcgctgttttgttctttttcttggctcttccttctcattggcactacaatttcatttcatgtttctttcatatctcataatttcatcaatcagactcatttttgtacatttgcttctttttcaaacaaggggctcccaaacttttgaatatatgctcttatatatacatatttatttagaaattatttacatgacctaccatattaatatattatgaatcaaaatagaaattttaaaagaatgagaaaaataaattatactggatgacataactttatttttttaagtcatgctaattgctatggttcatgctaatgctacattagctagtatatcagcataatgcacattttagagcaaggttttgttaacttagttgataaaagtctcatgtgtcaaatactctacttgataactttgaccttttagctgacttttggtcagacctgataggattgtcattgttttcacgttgtaatgtgactgatacaagaatctgtgctaggagtaagagaggtgtcagttctgccttttcttgtttgcttgtgcttgcattattaatattacttttaatttacaagttctttgcaagaagctttttaagccatttgtccagtttgtgagagttagctaaaattaggtaatacaatccctcaattcacttagttaggtatatgcaaacctcagtatatcacaatatgctaaaattgtaggacaaataagggtacttcaggacactcattatatggcacatattatgaggccttctgacttgtgtgctggataagggtgccaatattttgtttggtgtagtatagattattaaagctttggttacttttttgataaaaagaattatcttgcatactggttaggtgagaccctgctggtctgaaacttcatatttaaatgaagtgtgtggatagatagctaatgaattttggaaactaaagaaaattggttaattccagcagaagttgaatttttaaaatctgaattacataaatgtatttacttatgcattaataatcagattatttttcaaacttttattttttgatctctctttccatctttctccctttctctttggtagtattttttttttcatggatcaacttttgtgagtttatcttttgattatcttgttttgttgcactgtcttgccaaaaataaaaaaaaaatttctaatgataaacatgtttatacaaggatctagggcagctactttataatatgcatatcctattctttatttcaacaagcctgtataacactaagataagagacagacatataacagtctcatgcacttacatattggcaaataaatcttcttaagagtaggagatatatcaggaatgcactctggctcatgttacagaggagagactgttcagtgtggtatgggatttctttgatagatgggcttacatacaaatatagtgtttatcagctttatgaagaattaactttaatattgaattgcatgaattagaattttcaagctatatgactttttattaattgagaaaattggactgaatattacatgctattagggaattaacattaattatgtaaggtgtgatcatagtaatgtggccatgtaagagaaaattcttatgttttaaagatatgtactgcagtatttagggataaaatatcatgatgtccgtaatttactgtatttactttacaatacttcagccaagaagttaaaggaagcatatttaccataatgctaattatttaggttatgggtatatgggtgttaatgacattctctcttttgtttacctgaaaactctgataataaaatttaaaagcatgatttgttaaaagagaggaaattttgcataatgaaacataactttttttttgtctttaaaggaacccattcagacctctggatatatttgtaattttgaggatttagaaattaaatctgttcttttggatgagatattaaaggtaattcctaaaaagttacatttctgttgctagcttgacaggggattatttctggaccaggcggtgtagtatcctggttagcagtgtggacccaggagtcagatatatctcaggcccagccctgctctcttattgttcagcctttaggtgggtgccaccatctttttgagcttcactttactttcctgtaaaatgatgataaaaatgtataagtctcagttattgcctttataaaatacagataataatactacttataggattgttattatataagataatgtgtgaaaagcaattagcacaatgctacggccaagtgagagctaaaaaaaatgagtgctaattgttgtcattgtttttgtagtaataacgaggtactgaagaaatatttaagagtttagcctttttctagttctaagatacttgagtaacttttttttagagatctatctttccattttttccaggttttgtaaaataatccttttttctctttttaaattcaagacatgattttgaacccctgcattggcctacattgaccatcctttgagaattttttttctctaagaatatactgagagaagttttaccttcattttagccaaagcattaactttttctatgtaatatatatatagtttttgaattgagatagtagatttaaaatttccagaagttagtggcaatttggaacaggtttgggtttgatatgtaaatcatatttgggtttgatatgtaaattatggctattttattgtgagccagtttcattttctatatcataaccaccatgcagcaatgggatgggcccccacccaaaatttggtttggatgtaaagattgatgataccatagggacaccaggaatgtatcttccataatgaaacttttaggggagagaagggcaggcttcccaagctgtcagaaatggctggacagcacagggaaaggagactggcttgagggtgtttttgttgttgttttgttgtttagtagttagggagtgggtctggggtgaggcttttaatggataggggcttatgtggtttgaaactcactggtgccccaaaggaagaagcatctgggctttctcattagcttgccccatgtgaggcagaggggaagagggagaggtgaggtttaaaagctgtcagcagccaaccatcaaaaatggagtctgggctgttattacctaggattatgtaacctccagatttgactatctgactcttctgtatatggatagcacttttcttgtccttgtttaaggtatatgacttgtaaaactaatgaacttatttcattgcccgtttaacttgaatacagtaccaccagataatattttttccttttccacacgtgtgaagaaagatatctattgtttttttttcagcagggtttctctctgttgcccaggctggagtgcagtggtgccatcatggctcactgtagcctctgccccccaggctcaagtgatcctcccatctcagcctcctgagtagctggggtcccaggcacaggccgccatgcttggctaattttttgtgtttttttttttttttttttgtagagatggggtttgccatgttggtcaagggtggtcttgaactcctgggctcaagtgaaactctcattttgccctcccaaaatgttaggattacaggcgtgagccaccatgcccagccagaagatgtctttctgcttgtcttctttcccaagagatttagggaaagaagacattatttatttatttcttttttttttattttgaatatgtggttcttccagggccctttaaaaagacagtcataagtgggtagaatcaaactaaggtgactgaaatattttgtctgcttttatatatttaatggtttgataaagagtgagcttcttgaatccctaacatttagcaaggatctgatacagagttggttttcagaaagtgttggttaaattgatttttaaaaatgaatactttactgcttttaaacaaatggcagaattatatagaattcaagcaaaacagaaaagtactgaaacaagttaaaaaaaaaccctaaatcccacaactcagatattttagtcactaccgttaaacatcagaacatcattccttacagctgcaagttatatatggaaatatggatttgatagatttagatagatgaacagaagtaattttataaaaatgacatcattgtacaatctacttttttagtgttaatttaattagataacctgaaagtgagtttaaaggaattctagacttttagataatatttttccactgcaaagaataccattttcctgggttgagaagaaaaattatgaaaaaccaggtagttattagtttaaattatattttaatttttaatagtattgtctttctactctaaaagataacattcttacattttcttctttttctccttttgtagatatattatttttacattgtcaagatttagaacatttaaatttcgtcttgtaatcctaatttccagttgtttagtcttaatagttaaatgaattcaaagctattagccaggctttttactacattttctccttacgtaagttcttaattttgattaatcttttagttggctggatttcattgttagtagtgtttttcaagaaaggctcataatttccgattgccttaagctctagaatgtgtgaaaatgcctgtggtgttgttgctgtaattgatgattatattgaattttgtgaagttttttagataaagaaggctctcagtggtaaaatgatttacctaaagttcatctattcattaattcagcaactattaattaacagccaataagacacagttcttggcctcaggtagaggagttgaggagatggataagtaattcagttgccagaaaagagttaggggtgctgtgattcagtcaggatggtacacctgttcaaaattctgaagcaggagagcattaggtcagttctgagatgatgaatattgaataccgtaaatagtacagctaggcattgtgctgaatctttgagacatgttatctcatttaaggtgaaaaaccaatctgagcagagggaaacagggatgcataactgtgagggaccccttctgccattttcctctgtccccttttcctcataggccgcctgattgcctcagaggtacttacttgtgggttttggtgttttccagataactcaaaagacccaaacgtgaagatttctcttccttattttgctcacaatatgatgccgttttttttcttgttttgttttgttctggtttttaatataaaaaagaaacacatactctaacggcaaatatttgaatttttttgttttcatctttacttttaggatccagaacatccaaacaaggattacctaattaactttgagattcggtctctgcgagatagccgagcactgattgagaaggttggaattaaagatgcatctcagttcatagaggacaatccacacccccgactttggtatttaaaaaaagcaaactctcccaggatgtcaggtgtaggtttttaaagtctcagttttgtccctctgctcccctactatcctcctgatgggagcagacttccattcttgctcttctttttaagatgtagaaattcatttagatttggaagtcctcaaaataagcagaaaattatttgaaaaatgtttgcatatgttactttccaccatgtttctctagaaataatagacataaatgataattgaattgtgattcaagactagtttgtcagtaatttgcaatatgcacttatgtttttgtttcttttaaaataaaggcgcctactggctgaagcagctcttcagaaactggatctatacactgcagagcaagcatttgtgcgctgcaaagattaccaaggcattaagtttgtgaagcgcttgggcaaactactgagtgagtcaatgaaacaggctgaagttgttggctacttcggcaggtttgaagaggctgaaagaacgtatctcgagatggacagaaggtaagttatgaaggcgcaggcatccctgagtactctatcagttcatcattgtattttttacaaatggaaactataaatacgttaggtgtatcatatggttccttttagaaattaattttaagtttggattacaacctagatattctacatgcctctcattcatttgtgtttcatagtctggaggcagttttcttctctgtttttacatctaataatggctgactctgggaggtttttttttcatttgatgggaaaaaatgatagaacaacagagactgggatcttttatatttaagacatagctcattattccagttctttctttctttgtttgtttaggccattatttaattttatgttgtcaagaggcataaaataaaaatcagaagtcgttagggatcgagattagttctttatatttcgtttttgatttatataaaataatgtatgaacttcataaatatttcttcttttgaattaaagaactgtaaggcctgtctccctggccaggaatcaaacccaggcagctgctatgaaagcagataatcttagccactgaaccacaaggcggagagtctgtctgaacttcatttcagctgttaagattgtatctcatcttcattaatgttatttatactcccaggagatctctaatatgacttaggctctgaaaagtcatataaaaccacatgtgttaacactgtgttttgttttgaaataaacctgatcacatctaagaaattgctgaaaagaaatgattgttatttaaatcattattttgctaaataaattgaaagtccaggaaagtaaaggcacaacattcttttccctgttctggtatcttagaccatttggggtgctataacaagatacctgagactggataatttataaagaacagaaatatttttctcacagttctggagactaggaagtctaagatcaaggcgtcaacaagtttggtgtctgttgagggccctgtctcactttcaagatggtgctttgaatgttatgcctttacatggtggaaagtgaaaagggcaaaaggggcaaattctctgaggagtcttttatgagggcattaatcaattcataagggcagacataatgacttcccagaaaccccacctcttaatactaccacaatggagattaagttttaacatgaattttggaggggacacacattcaaaccataacctccaggtgctcctcatttcctcttagctagtcaggctaagttcctaaagtgtttggagagtcacattgtccactgctgtgtagcctcagcacctagaactgtacctaacacacagcaggctcttggtaaatatttgtcaaatagatgaatgaatgaatgaatgcctttgtttgtagggatcttgctattggcctccggctgaaattgggggattggtttagagtactccagctcctgaaaactggatctggtgatgcagatgacagtctcctggaacaagccaacaatgccattggagactactttgctgatcgacaaaagtggtatgtaacttacctggagctcctcaagcattcatgcaacacttagtagctgcctgagtacttctctaggaagtaatcctggggaaatgaagatcattttcttgaattctaaaattcagatagctctagattcatcttggaaagccggagatcctgtgaagttgtgagatgagcactataaaaatttgtagtgtataaacacaaaacttgtacttagctccatgcagctttctgtttgagcctacagtatataattctaaggtgttttatgttttatttttgaacaaagtttaataagtgtttttatttgctttattatatattattttcagaaatcattggcttttcttttcattttgattagtatgcttatttatgtcagtgtttgggagtaaagcggtggtatttcttaagaatagtcaaggaatgttacaactagaatatgctttagatttttttccccatttatatgtttgaaagttattctgaagctgaagataatgaacagtaatactagtactgggaattatgatgaaggaacattttggttgaaagtgcttatttatcaggagattcacattagttttaaaagtttttccttttttctttctgtaggttgaatgctgtacaatattatgtacaaggacggaaccaggaacgcttagctgaatgttactatatgttagaggattatgaagggttagagaaccttgccatttcacttccagaaaaccacaagttacttccagtaggtattgcaaattttagtttttggaattgttaggttactttataaaattttatgttatagtcaggaaatatctcccacaataaagaaaattaaaaatctataatgttacagtttcttagacatggaatgatgcttttaattataaagtaatatataaataatatagaatgcttgaaaatggaggaggaaaagtcagtcacattcttactactttaatatgatcacagtcttgtgtagtctgttaagatttataaagttactttttaaatgttagctgaatacatgaaatatttacagatgaaatgatatgttgctagggatttgcttcaaaataatctgaaattgtttgaaatttgctataacttaagtttaaaaagatagctcaacttttattatgttatattaacaataaagtatttcaagttaaaagagaaatggaaagagttctcttgctttcaaatgttcttttagagcctcttaattaattgttgaatactgtaaataagtttgtattttatgtgtttaatatgtgaatcactgagccgtacaatacttgtagagctagcagctctcaactgcagagtctggatgtatatcagttgtgagttctgcttcaaaaattttgcctattagttaatagataagtatagaattacggtaattcagttttccctattgtgaatttgagcggctatctctgtaataatggtattttcactcacccttattttcaatcctatgggttttcttgagtaggaaagaaactgaaattacttagaaccctgggtatagggaagagtgtatactccgttagtgttgtctgttagatgtgtctgatgatgccgataaacattttgctgtagggtatccagaatagtaatattcctgagtaaatgctaagtaattataactataatgataattaaataactaaaatagtaaaaattttagtattataaattttaaatagagttatagaagaatttttaataatatgggaaggtgtacataatatttaagtaggaaatacaggatacaaaattgtgtattgtgtgattgtgtacaacacaaacatatacatatatattcaaaggacaaagatgttaagaaattgcaccagagtattgagagaggtttcttatgaatgataagattatgagtaatttcactttttcatgttttgtaattttttttacagatagaatgtattcccttataatcagaaaaatataagcattgttttaaaagttggatataaaatatactttcatacttccaaacatattgtagaagggttggggagggaaaacctctggctaattagatattctgaaagaaaaaaaatgatgttctgaaaatggccagtgcagctgaacaagtaggatttttttttgtggtggtggtggtggtggtggtggtggtggtggtggtggtggtggtggttcttcctcgctgccaccactttccaagagagctctgttttgggtctagcttcctgaactgattaatggcagacttctctaggacatttctttatagaggtatttgagggctctgtgaatgtgagatttcccttaagtctctagttaataattagcactgagtataccaccaattgatataaaggcttttttcacccactggtgggggatttgttttggagcttgacatttctgtatactagctactgaactgagataagaaaggagtgcagtgtcttcagggcctcgttaaaaatcagatccacagcctgagccagtgaagctctggatttatagaagtgatgccttagcatccattctcatttgtaccctctgcccttgcgtctgtttacaagaaaaatctgtaactaccagtaaatttaatactcatacgtccctacggagttttgttaaattaaaatataagtaatttccttggtctgtgaaatctgatagtattgttttgtttgtacaggaaatagcacaaatgtttgtcagagttggaatgtgtgaacaagcagtgactgcatttttgaaatgtagtcaaccaaaggcagcagtagatacctgcgtacatctcaaccaagtaggaactgaaactttctgtctgtgcagcttattgtcttaggaatttttaagtcaaagcaaggttaaagtcaccctagtgatcttttaaagattattgccaatgtaagggttggagagcctgacgtgaattttatgaaaggagccaggcgggtattctcttaattgaggatgaagaaagttgcacttacctcttaaagtgctaccatgaggggtgacccacttataaccagctcagtatgggagcatgaacaaaccttgtgcttttagtctggcaagtttttgtctggaagtaatttcataattttctgcattttgttagggttgggttttgtttccaaagaaggaccagttaatggttggtcattgcctgaggaaggaattgagttactttatcaatatttattttggcaaatctgttttccttttcctttatagaaaacatacatttgatatttttaagagctgaaattaaactaaaagtacagcgttattatagaaactattgactattaatgtattcttttaatatttttcttattttttaaaaccccttttgctttataaaaataatttttaaatgtcaaatctgataggacattttcacaactgaaattgcatattttgttttactataacatataaacaagtgtatctttcttgcttgaagttttgtaagattagaattacaagattaatatgattataaactttgaaactttcattttcacatgtatttcttaagcaaatcagagtcttagagatcatttaatcattttgacttatgcttataggacttatgttgctgtgattttaaatgctatttttaaaacataccagtttttaattttgaaatagtgtttttactataaacttaaagctttcttgtgtgaattttatgatatatgtgactgtaaatacttgggtgactgaatggtgtaatttttctcctagtggaacaaagctgttgaattggctaaaaatcatagtatgaaagaaattggatctctgttagctaggtatgcatctcatttactggaaaagaataaaactcttgatgccatagaactctatcggaaagccaattacttttttgatgcagctaaactgatgtttaaggtaatgtaaaaatcttggtgaatagttgatttgttccagtcacaatattttaatatatgaaagagcatattctccattttctttggaaacaaatatttattacagcaaaaccactctggttttattgcttctgtatacattattttagtattattgccttttaccaacaaagatgtttattaatattagcaacttactaatacctaaacaattgtatgttgaatactggagttaacaaagagttaaagaaattactgagactgcaaagtacaaatttctacaacagggaattaaattttgttgataatagagttttaagttcttttaaatgtacaagttttaaatattacgctttacataatatttatttggagggaagataattagtacagtattaatgtatacacattttaccttctattctattcccatttgatttaatgaaaaaggaaaaatatctcttaaaatcatttaaataagtttcagcttctgaaggatgcaccatgttaacactatcatatctttgtgtacttggaacattattttaaaaatctctgtattatggaaaataccagcatttcatcaatactttctttattctgatttagttatctaacagcacctctcattttctaagagtgatctgaattccaactcctagggctaaatactaaggaaagaactaaatacgaataggtactaatgaaaagaagataggttttaaaatatttatcttaaataggtgatatttatattccatcttactgattttcctccttttttagggaatcaaatttgttagttttggaaatgattgtatttgtacaaatatagataagaagtaacttccctcaattttgaaaaatgtttaagattgcagatgaagaggcaaagaaaggaagtaaacctttacgtgtcaagaagctctatgtactgtcagccttacttatagagcaataccatgaacagatgaagaatgcccagcgaggaaaagttaaaggaaaaagttcagaggtaaagtagcacgttaaataacattacatcattggaagtgtttaagaaaaaaaaaaaaagaaactattaaacttcttttatggataggtcatttaatttgcatgtctgcatttgtttccttaaatgatatgaagaaaaagaatgactgatttagaattagtattcaaattcagaaatttctacctagaaatattgaaattgtttttctgtaatactaatctgtagtctttgctttaatgtcttatttgttaaaaaattttacttacctgggaaatcttcctttctaatcagtgagttttaaaactttcctagatatattgttgtaatgcgtaaatgaatgtctgattaacttagaatctaattagttatattttaaaatttattcataccagtttaacaaactatgaattataataaaaaattaaatctgttttgggtgcagtgctcacaccagtgatctcagtgactagggaggcttaggtgggaggatcacttgagtccatgagtttgagactgcagtgagctatgaccacgcagctgcattccagcttgggtgacagagtaatacctcatctcttaaaaaaaaaagtctgtaatcatagttttgtaatgataaaatctgctcatttagatactttaatgactgttgttatttgggagttgacactgtgtgtgcgtttgtgctgacggaaagtgtgggctgactttactcgtaggccacttctgccttggctggtttgctggaagaagaagttctgtctacaacagatcgtttcacagataatgcatggagaggggcagaggcttaccacttctttatacttgcacagaggcagctctatgagggatgtgtggacactgcactgaagacaggtgggcatgtcacctaatgtgtatagtatgtctgaaaattataaattgagtatgaagggctagcttcttcaatttgcctttatgtggccttcaataacattttttcctatggatttattttcctatgtttgagtcttatgcattcccatttttgaagttttttttaatacagagaaaacacatagttcacagaaatgtcagaaatttgagataagcaaaaagaatgtgtatagcattcatatggctagccattcaaagttaaatacttcatagccaagttcatattttttggagaaaaatatatatacatatatctattaaatatatttaatacatatatttaaatacataaaatatatttaatacatacattcagataattttattctttacaatatcgggagtataacatggcatacttttatgtactttttgaaaaccacgttttaaaatatttccatattttacatatacattgctatagcatttatcttgtgttttataaaagtgatacttagctaataatttcaaccaaaacagaaaaatatgaagaaaaagtaaaataatctaacaatttgtaaaataatcctataactcagaggtaatcattgctaaggtttgggtaaacttccttttagatgtttctacatgcaggagcatggttttttccctcagcagtatattatagttctttcctcattcttaaacagagatctatataaacattctacctaatattccatggtatttgtatactgtaattgatagaagattatttacatgtaatatcaggatctcaatttcttcttaacaataacaatattctagaatttagatatatgaactccagagagtgtaattatgataattatgttgtaagccaaagcatatgaatgactaaatcattactggtctttcagcttggccatggcaagcactgtcatgacaccccacccttcctctgtcatattggtgacatcatccatttgctgctcacatcatcaccatatgcacatcacactctaatcctaatacatcatgaatctgtattccaatggcatgatattaaaagaaaacttttagagaaattcttttatttgaatatttctggaattctatagtgatgatttttatttttaattatttttataagagctctttagagaccatatgtaataatcgtctttcatatatattgattttccctagcatttataagttatagttacttgatcctttctaagcttcagtttctctttgtgtaaagaaaggataatagtatcttcctcagccgggtgcagtggctcatgcctgtaatcccagcactttgggaggctgaggcgggtggatcacaaggtcaggagatcgagagcatcccggctaacacggtgaaaccctgtctttactaaaaatacaaaaaaatcagctgggtgtagtggcaggtgcctgtagtcccagctacttgggaggctgaagcaggagaatggtgtgaacctgggaggcggagcttgcagtgagccgagatcgcaccgctgcactccagcctgggtgacagagcgagactccgtctcaaaaaaaaaaaaaaatagtatcttcctcatagagttattgtaatgattgagaagttcatgaaaagaaaactcacttattatgctgcctggacattgtgagccctcaataagtggtagctgtgatgatgatattaatagttttttttatatagtcatgtctgtcagtcttttacttcatatttttctcttaggaatcatgcttaggaattccttcaagtttgttaaatattatgtttattataacatctaagttatcctgaaaccatatattaggtttattctttcttaacagatttgaaattctaccttaatgtgccgagaccagctcagtcagggagaccctaaccaagcggcgctagaggaattaaagacacacacacagaaatatagaggtgtgaagtgggaaatcaggggtctcacaaccttcagagctgagagccccgaacagagatttacccacgtatttattaacagcaagccagtcattagccttgtttctatagatattcgattaactaaaagtatcccttatgggaaacgaagggatgggctgaaataaagggatgggtctggctagttatctgcagcaggagcatgtccttaaggcacagattgctcatgctactggttgtggtttaagaactcctttaagcagttttctgccctgggcgggccaggtgttccttgccctcattccagtaaaccacaaccttccagagtgggtgttatggccatcatgaacctgtcacagtgctgctgagattttgtttatggccagttttggggccagtttatggccagattttgaggggtgcctgttcccaacatgtcccccttctttgatttgcaaatctataaaggcaaggacagctttgtcatggtgagctacttcttgcaggagtcaggatccacatctgcagactatagaaagacaaacaacacagattaaaagcacaatcatcattgaaattacagggcttccaagtgtttttatccattttaatggattactagctgctaatctgtctgcagctcctttaagcactccagttcttggcattaaggtcaggtgtccctgggatgctttgaatatttgttcttttaattttgctatatccaaaaacaagtttgtagagtgtccttctagacgcttttttattctttcccaaattttgatcttgttaagagctattaatagtttccacaaatccttaatgtttagctcctagagcgggccatatcatttgaggttgaggtgccactataccaccatggttctagataataggaactcttgccgtacttcttattatatctaccacctgaacattttgttgagaccatctgaacataagtatggcatggcacacagactgagaagtgcaattcaagctaaacatccccttaggggaccaattaataatgattccataggaatcattgtgcagcacctctgcctgttctgcaatgcaatcttcctaaacaagtacgttcatttttttctggccaggttcaattttgtttacaaataggtttttgagggcggtatgcctcaattataggagcagatttattatggtaaatactgagatcagaaagctgtgtaacagcatcatagagtgattacatctaggcattattgccagccaagattgataaatatgcccaataagtgtaattgttccctgtgtcagcccttactgaaggaatactcatagcagtggtgataacagctatcatagctaccattaaattactcattgtgactggttgtcccactttcctcaggttttcttccgccatctgtgacagcttcttgatctgtccccagttagttgactgtgttcaacgggtgttgcttgcgacagctggggtcctcctcagtgtcagtctcgaaatggctgcaacagggtggggggggtcctcgagatcctcccggaatctcttccttggcatctggctcatgataaggtttcaggtgtcttgatggtatccaaatcggctgttgattttggcctggagaaacacaagcataacctctaccccaagttattattttacctatttcccaactttttgttatcagatctctccaccaaactagttgttctgcttctgtctttgcagctggtttctgtagatgctgttcagctgctgataacatctggcatttgggcaggctcaaaaaatttaaagttaataatgctagattcaattgtgtatgggctgtcccataatccctgtctctccctcttttttgtttttgtcatcagttgttcatctgtatgaaatcataactgagcattttcaattaactgtgtagaatgaaccacgtatgaagaatcagatatcacattaataggcaaatcaaaagcagtcattacctcaattacagctacaagcttcgctttttgaactgaagtatagggcgtctggaaaacttaaccttttgatccagaataagaagctttaccatgactagacccatctgtaaaacaatgaaaacacttagcaggctgcaggttgtttaccacaggaattgtaaatgcaaactgttcacagtcttgctcagctaaggggatagtaaagaaacagtattttaaatctatgactattaaaggccaattttttggaattatagcaggagaaggcaatcctggctgtaatgctcccataggttgtataactgaattgatggctcttaagtcagttaaaattctccatttacctgattttttcttaattacgcaaactggagaattccaaggggaaaatgttggagctatgtgcccattttctaattgtttagtaactaatttctctaaagcctccggtttctctttacttagtggccattgttctatccaaattggcttatctgttaaccattttaaaggtataggttctagaggcttaacaatgagcaccatcaaaaatgatatcctaatctttggcgggaactttgtctttccacttgaagcggttcttccaaagcttgcagatttttttctagtcccataccagggatataccccatttcatgcattatatgttgactttgagggctatataattgttctggaattagaacttgtgctccccattgttgcaataaatctctcccccataaatttataggtacagaagttataattggttgaatagtcccaggttgtccatcgggcccttcacaatgcaaaatataactactttgatatacttcaggggctttaccaactccaactatgttaagttgagtgggttgaattggccacgcagatggccagtgctgtagagaaatgattgaaatgtccgctcctgtatctaccaaacctttaaattcttttcccctaaatagttacttcacaggtaggacgtttatcagtaatttgatttacccagtaagctgctttgccttgtttatttgtgcttccaaatatattgtgttggtttaatttcacttttccccattcccacatatggcagaataaggagctatgctatgcactctcctggcttctttctagggaacagaagtagatataacaatttgaatttccccattgtattctgaatccatgactcctgtatgtatttataccccttttaaacttaaactagactttcctaaaagtaatcctattgtccccactggcaagggtccacagaccctttgcgggggttccccaggcagaaggctcacagttttgtgcagcataaatctactgcggcactactggctgtggcggggaacagacattgtacaggggtgagggaatggcctgagctggaaatgcaccagtttggaatggggcccaggacgggcccctcattgcatttcccaaaatcaggttcccatctttattaaacttagagtgacactgattagcccaatgtccttttttacattttggacatatttcaggctcagcagttttcttttttcagctatctggtggcctgacttgctgattttttctacattcttttttagtatgaccatgcttcccacagttaaaacaccctccaggaaatggagtatttcctttatccactcttagtcctgccattgctcgtgctagcagagtagccttatgcagattacctccgataacatcacaggccttgataaaatcaactaaatgtgctttccctctgataggtcgcagagcagcctggcagtcaggattagcattgtcaaaagctaataactgcagcactatatcctgagcagccaaatctgcaatcacctttttgttttgagatgaaatctcactctgtcgcccagcctggagtgcagtggcgcgatctcggctcactgcaagctccgcctcccgggttcacgccattctcctgcctcagcctcctgattagctggcactacaggcgcccgccaccacacccggcgaattttttgtatttttagtagagacggggtttcaccatgttagccaggatggtctcgatctcctgaccttgtgatccacccccctcagcctcccaaagtgctgggattacaggcatgagccactgcacccggcctgcaatcacctttttaagagactcctgtaaccaagctataaaatccacatatggttcttttggtccctgttttatagcactaaaggaagcgtattgttctccacataaagggattttttcccaagctctaatgcacactcctctaagctgttctatggcatcatcctgcatgaccacttgtgcatctaaaccagcccagccgccaacccccaaaagttggcctgtagttatattaatttgaggctgggcctgggcgttgcaagcagcctgaatggaagcttcatgggcccaccaagttttaaattgtaagaactgagcaggagttagacaagcctgagtaagagcatcccagtcagtaggaatcatctgactggaaacagcaacattctttaacagtcccattacaaaatgagaacctggtccatactgatttatagcttgtttaaattctttgagcaatttaaaaggaaaaggctcaaatgtagctgtaatatttccccgttgttctggggggtgtattctaacagggaactgtcaagcctgtaaatcaccctctcgtctagcttgctgaattcctgcctgaatagaactaagagcagtcactcaaggtgctgttcgaacagtcactggggcaactcttttcatccagtgtcctctggaaaagaaagatctggagggtcattttcttcaaaataataatgagggggtgcagaaggatagggatgaacctctctcctttaccgctttagctttagctggcaaataaacctgctctgtaatctcttctgttacttcgttatactctccttcctcctcatcatcagtgtgaaaaagttccaaggtggaacgaaccacagcccacacttttcccattgttaccctgatgcttccgagctccccttcttactcactacggggattgctttaagagtactcaggtgtcctccagctagttccacgttctccaactgttgctccagcgaccctttgatctggattcaagcccccacggtggatgccacttgccgagaccagctcggtcggggagaccctaactcagcggtgctaaaggaattaaaaacacacacacagaaatatagaggtgtgaagtgggaaatcaggggtctcacaaccttcagagctgagagccctgaacagagatttacccacgtatttattaacagcaagccagtcattagccttgtttctatagatatttgattaactaaaagtatcccttatgagaaatgaaggaatgggctgaaataaagggatgggtctggctagttatctgcagcaggagcatgtccttaaggcacagatcactcatgctactggttgtggtttaagaactcctttaagcagttttctgccctgggcgggccaggtgttccttgccctcattccagtaaacccacaaccttccagcgtgggtgttatggccatcatgaacctgtcacagtgctgctgagattttgtttatggccagttttggggctggtttatggccagattttgtggagcctgttcccaacattcatgaaactaaatttatacttttttttaattatactttaagttctggggtacatgtgcagaatgtgcaggtttgttacataggtatacacatgcgttggtggtttgctgcacccatcaacccgtcatctacattaggtatttctccttatgctatccctcccccagccccccaccccctacaggccccggtgtgtgatctcctgcccctgtgtccatgtgttctcattgttcgactcccacttatgaatgagaacctgaggtgtttggttttctgttcttgtgttagtttgctgagaatgatggtttccagcttcatccacgtccctccaaaggacatgaatgcatccttttttatggctgcatagtattccctggtgtatatgtgctgcattttctttatccagtctatcattgatgggcatttgggttggctccaaatctttgctattgtgaacagtgctgcagtaagcatacatgtgcatgtgtctttatagtagaatgatttataatcctttgggtatatacccagcaatgggattgctgggtcaaatggtatttctagttctagatccttgaggaatcgccacactgtcttccacaatggttgaactcatttacactcccaccaacagtgtaaaagcattcctatttctccacgtcctctctagcatctgttgtttcctgactttttaatgatcaccattctaactggtgtgagatggtatctcattgtggttttgatttgcatttctctaatgaccagtgatgatgagcttttcttcatgtttgttggatgcaaaaatgtcttcttttgaaaagtttctgttcatatccttcacccactttttgatggagttatttgtttttttcttgtaaatttgtttaagttctttgtagattctggatattagccctttgtcagatgggtaggttgcaaaaattttctgtaggttgccagttcactctgttgatagtttcttttgctgtgcagaagctctttagtttaatcagatcccatttttctattttggcttttgttgccactgcttttggtgttttagttatgaagtctttgcccatgcctatgtcctgaatggtattgcctaggttttcttctagggtttttatggttttaggtcttacgtttaagtctttaatccatcttgagttaatttttgtataaggtgtaaggaagggattcagtttcagctttctgcatatggctagccagttttgccaacatcatttattaaatagggaatcctttccccatttcttgtttttgtcaggtttctcaaagatcagatggttgtagatgtttggcgttgtttctgaggcctctattctgttccattggtctatatatctgttttggtaccagtaccgtgccgtttttgttgctgtagccttgtagtatagttcgtagtcagttagtgtgatgcttccagctttgttctttttgcttaggattgtgttggctatgcgggctcttttttggttccatatgaaatttaaagttctttctaattctgtaaagaaagtcaatggtagcttgattgggatagcattgaatctataaattactttgggcagtatggccattttgatgatattggttcttcctatccatgagcatggaatgtttttgcatttgtttgtgtcctctcttattttcttgagcagtggtttgtagttctccttgaagaggtccttcacatcccttgtaagttgtattcctaggtattttattctctttgtagccattgtgaatgggagttcactcacgatttggctctctgtttggctgtttttggtgtataggaatgcttgtgatttttgcacattgattttgtatcctgagactttgctgaagttacttatcagcttaaggagatttggggctgagatgatggagttttctaaatatacaatcatgtcatctgcaaacagggacaatttgacttcctcttttcctaattgaatatcctttatttctttctcctgcctgattgtcttggccagaacttccaatactgtgttgaataggagtggtgggagagggcatccttgtcttgtgctggttttcaaagggaatgcttccaggttatgcccattcagtatgatattggctgtgggtttgtcataaatggctgttattattttgagatatgttccatcagtgcctagtttgttgagagtttttcgtatgaaggctgttgaattttgttgaaggctttttctgcatctattgagataatcatgtggtttatgtcactggttttgtttatgtggtggattatatttattgatttgcgtatgttgaaccagccttgcatcccagggatgaagccaacttgatcatggtggataagctttctgatgcactgctggatttggtttgccagtattttattgaggattttctcatagatgttcatcagggatattggcctgaaattttttgttgttgttgtgtctctgccacattttggtatcaggatgatgctggcttcataaaatgagttagggaggattccctctttttctgttgtttggaatagtttcagaaggaatagtaccagctcctctttatacctctggtagaatttggctgtgaatccgtctggtcctggactttttttggttggtgggctatgaattgctgctcaatttcagaacttgttattgatctgttcagggattcgacttcttcctggtttagtcttgggagggtgtatgtgtccaggaatttatccatttcttctagattttctagtttatttgcatagaggtgttgatagtcttctctggtggtagtctgtatttctgtgggattggtggtgatatcccttttatcattttttattgcatctatttgattcttctctcttttcttctttcatagtctagtggtctatctattttgttgttgtattcaaaaaaccagttcctggattcattgattttttggagggttttttgtgtctctatctccttcagttctgctgtgatcttagttatttgttgtcttctgctagcttttgaatttgtttgctcttgcttctctagttcttttaattgtgatgttagggtgttgattttagatctttcctgctttcccatgtgggcatttagtgctataaattttcctctacacactgctttaaatgcattccagaaattctgctgcactgtgtctttgttctcattggtttcaaagaacatctttatttctgccttcatttcgttatttgcccagtagtcattcaggagaaggttgttcagtttccaggtagttatgtggtgttgatgagtttctttttttttctttttctttttcttttcttttttttttttttttttgagatggagccttgctctgtcacccaggttggagtgcagtggtgcaatctcagctcactgcaagctccgcctcctgggttcacaccattctcctgcctcagcctcccaagtagctgggactacaggcgcccgccaccatgcctggctaaattttttgtatttttagtagagatggggttttaccatgttagccaggatggtctcaatctcctgacctcgtgacccacccgccttggcctccgaaagtgctggaattacaggcgtgagccactgcgcctggccttgatgagtttcttaatcctgagttctaatttgattgcactgtggtctgagagaccatttgttatgatttctgtcttttgcatttgctgaggagtgttttacttccaattatgtggtcagttttagaataactgccatgtggtgctgagaagaatgtatattctgttgatttggggtggagagttccgtagatgtctattaagttcacttggtccacagctgagttcaagtcctgcatatccttgttaattttctgtcttgttgatctgtctaatattgacaacggggtgttgaagtctcccactattattgtgtgggagtctaagtctctttgtaggtttctaagaacttactttatgaatttgggtgttcctgtattgggtccatatatatttacgatggttagctcttcttgttgcattgatccctttaacattatgtaatgcccttctttgtctcttttgctttttgtcagttaaagtctgttttttcagagactaggattgcaacccctgcttttttttcttattattgctttccatttccttggtaaatattcctccatccctttgttttgagcctatgtatgtctttgcccgtgagatgggtctcctgcatacagcacaccaataggtcttgactatccaatttgccagtctgtgtcttttaattggggcatttagcccatttacatttaaggttaatattgttatgtgtgaatttgatcctgacattatgatgctagctggttattctgtctgttagctgatgcagtttcttcatagcttcgatgctctttacaatttggtatgtttttgcagtggctggtactggttgttcctttccatgtttagtgcttccttcaggagctcttgtaggagaggcctgatggtgacaaaatatctcagcatttccttgtctgtaaaggattttatttctcctttgcttatgaagcttagtttggttggatatgaaattctgggttgaaaattcttttctttaagaatgttgaatattggcccccactctcttctggcttttagggtttctgccgagagatctgctgttagtctgatgggcttccctttgtgggtagcccgacctttctctcaggttgcccttaacattttttccttcatttcaaccttggtgaatctgacaatatgtgtcttggtgttgctgttctcgaggagtatctttatggtgttctgtgtatttcctgaatttgaatgttggcctgccttgctaggttggggattttcctggataatatcctgaagagtgttttccaacttggttccattctccccatcactttcaggtacaccaatcaaacgtagatttggccttttcacatagtcccatacttcttggaggctttgttcatttcttttcactcttttttctctaatcttgttttcttgctttatttcattgagttgatcttcaatgtctgatatcctttcttctgcatgatcagtttggctattgatacttgtgtatgcttcacgaagttcttgtgctgtgtttttcagctccatcaggtcatttatgttcttctctaaactggttattctagttagcaattcgtctaaccttttttcaaggttcttagcttccttgcattaggttagaacatgctcctttaccttggaggagtttgttattacccaccttctgaagcctgcttctgtcagttaatcaaattcattctccatccagttttgttcccttgctggtgaggagttgtgatcctttgtaggagaagaggtgttttggtttttggaattttcagcctttttgctctggtttctccccatcttcatggatttgtctacctttggtctttgaagtcggtgaccttcggatggggtctctgagtggacatcctttttgttggtgttgatactattcctttctgtttgttagttttccttctaacagtcaggcccctctccagcaggtctgctggagtttgctggaggtccgctccagaccgtttgcctgggtatcaccagtggaggctgcagagcagcaaaggttgctgcctgttcctttggaagctttgtcccagaggggcacctgccagatgccagccaatgctgtcctgtatgtggtctctgtcggcccctacatgggtcccagggacccactgggaggtgtttcctagtcaggatacatgggggtcagggacacacttgaggaggcagtctgtcccttatcagagcttgaacactgtgctggttgatccgctgctgtcttcagagctgccaggtagggacgtttaagtctgttggagctgcgcccataaccacccctttccccaggtgctttgtcccagggaggtggaagttttatctataagtccctgactggggctgctggctttttttcagaggtgtcctgcccagagaggaggtagtctggctgcaggggccttgctgagctggggtgggctccgcccagtttcaacttcccagcggctttgtttacactgtgagggtgaaaccgcctactcaagcctcagcaatggtggatgcctcttcccccaccaagctcaagcatcccaggttagctcaaactgctgtgctaaccatgagactttcaagccagtggatcttgagcttactgggctccatgggggtgggacctgctgagccagaccccttggctccctggcttcagccccctttccaggggagtgaatggttctgtcttgctggtgttccaggcgccactggggtatgaagaaaaactcctgcagctagcttggtgtcagctcaaacggccgcccagttttgtgctggaaacccagggccccggtggcgtgggcagcgaaaggaatctcctggtctgcaggttgtgaagactgtgttaaaagtgcagtatctgggccggagtgcacggtacagtccctaatggcttcccttggctaggagagggatttccccatccccttgcatttcccaggtgaggtgacgccccaccctgctttggctcatcctccttgggctgcacccactgtccaactagtcccaatgagatgaacctggtacctcagttggaaatgcggaaatcacccaccttctgcgtcaatcttgctgggagctgcagaccagagctgttcctattcgtccatcttgccagccaaggccaactttttttttttttttttttttagatggagtctcgctctgttgccctggctagagtgcagtggcataatctcagctcactgcacgctccacgaggtcaacttttttagatctcacatatgagtgagaacatgtgatatttgtcagaaacatctattttagaaatcacttatgtatctgtgtttctaaagatttgtagttttcttcttgctggctctaaaggtttatttttcagtttattcctagttttgttgtcattaatgaggttgtaaattttttttctaattggttattagttatatataaaaattatttttatgtttattttataactgaccttcatttagttttaactaatatgtgacataaattatacactgtttaaaatagctgtatcatattcaatttatacaccatttctagacttaaaagaacaattagatctttaacttgagatgcttcacctactaggtgaagatctctatatgcctcattttttttttttttgtcagtggtaagacgatctgaaacaatgtatgacctgtaaattattacccttgaacctatcatgctttggatatgaaattagaggatattgtttattgtaactgagtgattacattttatatacattttttctactacactttgttgtaaatacttgaccccagaaccagtttataggattctgattgtaaaacacatgaatatattgtttaaactggttttatgaagtttttgatatactataacaaagtatttttaagattccaaggtctagagaagaaaaagatacaaattggttataaatctaatattaaatcttaatattaaatctaatattaagtctgctttttttattatactttaagttctggggtacatgtacagaacgtgcaggcttgttacataggtatacatgtgccatggtggtttgctgcacccatcaaccgtcatctacattaggtatttctcctaatgctatccctcccctagccccccacccccaacaggcccaagaatacagtgcattttaaagaaagattttttcatctccagtataattgcaaataagatgttgtaacttgaattgtctcaactgacaacttggagactcattttcactttttcacaaaatcctattgtttcagtgagacaaatgacaaaagcagtaatacaaaagcaataatctgtccaaagaaacacagagaaaagaacatattattttatactgttttgcttatattgttttgctttttttttaacaaaaggaaacatggtattatcttgcagtttatgactaacatagtataatactcatttgctttttatgaagtgccacagaaatttctaacataaattgaagaaaatgtggatgttttgacaccaaaattttgtgtgtgtgtgtgtatttctgagctggcacagccatgtgcagcatttggtggtggttcttttcataggatgagctgttagccagcttcacagaaccttggatatcatgtagcctagttcctcctcttggtgctcccatctctcaacactgtttgctttcagttcgttcatgcaagttcaatgaaatcaccttaacttagattcttcttttgcatgttcatgatatcataataattattttaaaaaaccccaattgccctgtttttcttagctcttcacctgaaagactatgaagacatcatccctcctgtggagatctactctctgctagcactctgcgcatgcgccagcagagcctttgggacttgttcaaaagctttcattaaacttaaatctttagagaccctcagttcagaacagaaacagcagtatgaagaccttgctttagaaatcttcaccaaacatacttcaaaagataacagaaaacctgaattggacagccttatggaagggtaggctaattttaattagtggatgccattctatcttattagaagcttggatttctagatgtacaatgtttaatgtaggaattaaagcactgaattttgaagcaattcacattaacattataccctattttatttatttttacaagtgtattcatgctttattttgtcattgtaagaaaggttttttcttgaaataacttttttaaatgaaagtatttgatgttcatctcagaagttttattctttaggttttttttaagtgtattaaataaagttagactaatgagaagttttaaagtataatgaattgtttctccccgttttacagtggagaagggaaactgccaacatgcgttgccacaggaagcccaatcactgagtatcaattctggatgtgcagtgtatgcaaacacggtgtccttgctcaggaaataagccactacagcttctgccccttatgccatagtccagtgggataaaggaatgataaactgtatataactgtaaaatatatgtagcatatatacatggctatatgctgtatatgtaataaggtttatttctgtgagttttgtatgaaaatcagcctcattatttatagttgaatttttgcacaaaatatatgttaataaaataatttttatggcaatacaactgtgaaataataaggccgattttcatataaatgtatgaaaatcaaccatgttttctctcaccctctctcttttgtttatcttgtttttggttacatgataccatggaaatattccaaataaatttttttctcacagtgtcaaggtaactgaattc

SEQ ID NO: 2 - protein, 74 aa, Homo sapiens
MPRPAPARRLPGLLLLLWPLLLLPSAAPDPVARPGFRRLETRGPGGSPGRRPSPAAPDGAPASGTSEPGRARGA

SEQ ID NO: 3 - protein, 189 aa, Homo sapiens
GVCKSRPLDLVFIIDSSRSVRPLEFTKVKTFVSRIIDTLDIGPADTRVAVVNYASTVKIEFQLQAYTDKQSLKQAVGRITPLSTGTMSGLAIQTAMDEAFTVEAGAREPSSNIPKVAIIVTDGRPQDQVNEVAARAQASGIELYAVGVDRADMASLKMMASEPLEEHVFYVETYGVIEKLSSRFQETFC

SEQ ID NO: 4 - protein, 42 aa, Homo sapiens
ALDPCVLGTHQCQHVCISDGEGKHHCECSQGYTLNADKKTCS

SEQ ID NO: 5 - protein, 42 aa, Homo sapiens
ALDRCALNTHGCEHICVNDRSGSYHCECYEGYTLNEDRKTCS

SEQ ID NO: 6 - protein, 42 aa, Homo sapiens
AQDKCALGTHGCQHICVNDRTGSHHCECYEGYTLNADKKTCS

SEQ ID NO: 7 - protein, 42 aa, Homo sapiens
VRDKCALGSHGCQHICVSDGAASYHCDCYPGYTLNEDKKTCS

SEQ ID NO: 8 - protein, 37 aa, Homo sapiens
ATEEARRLVSTEDACGCEATLAFQDKVSSYLQRLNTK

SEQ ID NO: 9 - protein, 18 aa, Homo sapiens
LDDILEKLKINEYGQIHR

SEQ ID NO: 10 - protein, 42 aa, Homo sapiens; identical to SEQ ID NO: 4

SEQ ID NO: 11 - protein, 42 aa, Homo sapiens; identical to SEQ ID NO: 5

SEQ ID NO: 12 - protein, 42 aa, Homo sapiens; identical to SEQ ID NO: 6

SEQ ID NO: 13 - DNA, 228 bp, Homo sapiens
atgccgcgcccggcccccgcgcgccgcctcccgggactcctcctgctgctctggccgctgctgctgctgccctccgccgcccccgaccccgtggcccgcccgggcttccggaggctggagacccgaggtcccgggggcagccctggacgccgcccctctcctgcggctcccgacggcgcgcccgcttccgggaccagcgagcctggccgcgcccgcggtgcaggtaca

SEQ ID NO: 14 - DNA, 707 bp, Homo sapiens
aggtgtttgcaagagcagacccttggacctggtgtttatcattgatagttctcgtagcgtacggcccctggaattcaccaaagtgaaaacttttgtctcccggataatcgacactctggacattgggccagccgacacgcgggtggcagtggtgaactatgctagcactgtgaagatcgagttccaactccaggcctacacagataagcagtccctgaagcaggccgtgggtcgaatcacacccttgtcaacaggcaccatgtcaggcctagccatccagacagcaatggacgaagccttcacagtggaggcaggggctcgagagccctcttctaacatccctaaggtggccatcattgttacagatgggaggccccaggaccaggtgaatgaggtggcggctcgggcccaagcatctggtattgagctctatgctgtgggcgtggaccgggcagacatggcgtccctcaagatgatggccagtgagcccctagaggagcatgttttctacgtggagacctatggggtcattgagaaactttcctctagattccaggaaaccttctgtggtaagttggtcagtctttgcttccaactagaaaggatgttatattcagacattgtgtacccagagaaaagagtattattctcttttttttggtggaaatttaatgaaaaacttaaatatatccaatgcgtgtgtgtgt

SEQ ID NO: 15 - DNA, 326 bp, Homo sapiens
catgggagacggtatctgagatgtttcctaagtgtcagtgctgactgttgtaaacttcctctcacagcgctggacccctgtgtgcttggaacacaccagtgccagcacgtctgcatcagtgatggggaaggcaagcaccactgtgagtgtagccaaggatacaccttgaatgccgacaagaaaacgtgttcaggtgaggcctgtgtagggggccgtgactgaatcaaatacttgggaacctatgctccaggttttggactggccttgtgctttgactctctgacccctttttacctaactgccttttggctggtttactggtgttc

SEQ ID NO: 16 - DNA, 261 bp, Homo sapiens
ccctcccttgtttcattagctctgtgtgtgtgtgtgtgtgtgtgtgtgtgtgtataactgtaagtggcgtggcccacaattatttctgccatgctctgcatgcatttacatttgtcccaatcatcttttgataagctcttgataggtgtgctcttaacacccacggatgtgagcacatctgtgtgaatgacagaagtggctcttatcattgtgagtgctatgaaggttataccttgaatgaagacaggaaaacttgttcag

SEQ ID NO: 17 - DNA, 130 bp, Homo sapiens
agctcaagataaatgtgctttgggtacccatgggtgtcagcacatttgtgtgaatgacagaacagggtcccatcattgtgaatgctatgagggctacactctgaatgcagataaaaaaacatgttcaggt

SEQ ID NO: 18 - DNA, 130 bp, Homo sapiens
agtccgtgacaagtgtgccctaggctctcatggttgccagcacatttgtgtgagtgatggggccgcatcctaccactgtgattgctatcctggctacaccttaaatgaggacaagaaaacatgttcaggt

SEQ ID NO: 19 - DNA, 199 bp, Homo sapiens
agccactgaggaagcacgaagacttgtttccactgaagatgcttgtggatgtgaagctacactggcattccaggacaaggtcagctcgtatcttcaaagactgaacactaaacatatcctttctggaaggttttctccttctgcctggagccaaccaaggtactacgatcgagccaaacactttaagacattccacagg

SEQ ID NO: 20 - DNA, 327 bp, Homo sapiens
gtttgaaagtgtgtttatattttctaatgtgtaaaaataaatggtgttggccttaacaacttgccacttgatgacattttggagaagttgaaaataaatgaatatggacaaatacatcgttaaattgctccaatttctcacctgaaaatgtggacagcttggtgtacttaatactcatgcattcttttgcacacctgttattgccaatgttcctgctaataatttgccattatctgtattaatgcttgaatattactggataaattgtatgaagatcttctgcagaatcagcatgattcttccaaggaaatacatatgcagatactt

SEQ ID NO: 21 - DNA, 22 bp, Artificial Sequence
  primer that hybridizes to the human MATN3 gene
ttaaacaagagcccacagcaaa

SEQ ID NO: 22 - DNA, 22 bp, Artificial Sequence
  primer that hybridizes to the human MATN3 gene
ttaaacaagagcccacagcaaa

SEQ ID NO: 23 - DNA, 21 bp, Artificial Sequence
  primer that hybridizes to the human MATN3 gene
gcgaggctcggatttaaaggt

SEQ ID NO: 24 - DNA, 18 bp, Artificial Sequence
  primer that hybridizes to the human MATN3 gene
gacgcgtgagcatcgaac

SEQ ID NO: 25 - DNA, 22 bp, Artificial Sequence
  primer that hybridizes to the human MATN3 gene
caggaatcaattgctgaccaaa

SEQ ID NO: 26 - DNA, 22 bp, Artificial Sequence
  primer that hybridizes to the human MATN3 gene
cgtaggataccccaggatcaag

SEQ ID NO: 27 - DNA, 22 bp, Artificial Sequence
  primer that hybridizes to the human MATN3 gene
ccatttcaaatctgccacttca

SEQ ID NO: 28 - DNA, 23 bp, Artificial Sequence
  primer that hybridizes to the human MATN3 gene
cccttctgcatctttctggataa

SEQ ID NO: 29 - DNA, 22 bp, Artificial Sequence
  primer that hybridizes to the human MATN3 gene
tagcagcaatttgattcccaag

SEQ ID NO: 30 - DNA, 20 bp, Artificial Sequence
  primer that hybridizes to the human MATN3 gene
ttgctaagcttcgctatgga

SEQ ID NO: 31 - DNA, 20 bp, Artificial Sequence
  primer that hybridizes to the human MATN3 gene
gagggaggcagatgaaagaa

SEQ ID NO: 32 - DNA, 20 bp, Artificial Sequence
  primer that hybridizes to the human MATN3 gene
aggccttctcccattgagtt

SEQ ID NO: 33 - DNA, 23 bp, Artificial Sequence
  primer that hybridizes to the human MATN3 gene
tccagaattttacttcattcctg

SEQ ID NO: 34 - DNA, 26 bp, Artificial Sequence
  primer that hybridizes to the human MATN3 gene
tgaagagatttattctgagacaaaca

SEQ ID NO: 35 - DNA, 18 bp, Artificial Sequence
  primer that hybridizes to the human MATN3 gene
atgggttgatgggtgcag

SEQ ID NO: 36 - DNA, 20 bp, Artificial Sequence
  primer that hybridizes to the human MATN3 gene
tctgggatacatgtgcagaa

SEQ ID NO: 37 - DNA, 21 bp, Artificial Sequence
  primer that hybridizes to the human MATN3 gene
aacaactccagcagccataaa

SEQ ID NO: 38 - DNA, 23 bp, Artificial Sequence
  primer that hybridizes to the human MATN3 gene
gcttttagagactgggtctcatt

SEQ ID NO: 39 - DNA, 20 bp, Artificial Sequence
  primer that hybridizes to the human MATN3 gene
gtttttagcctgctgcttgg

SEQ ID NO: 40 - DNA, 20 bp, Artificial Sequence
  primer that hybridizes to the human MATN3 gene
tgttggtaccttgcgattcc

SEQ ID NO: 41 - DNA, 18 bp, Artificial Sequence
  primer that hybridizes to the human MATN3 gene
ctgcaaccaccacctcct

SEQ ID NO: 42 - DNA, 18 bp, Artificial Sequence
  primer that hybridizes to the human MATN3 gene
ggcgcacacctgtagtcc

SEQ ID NO: 43 - protein, 42 aa, Homo sapiens; identical to SEQ ID NO: 7

SEQ ID NO: 44 - protein, 42 aa, Mus musculus
ALDQCMLGTHQCQHVCVSDGDGKHHCECSQGYTLNADGKTCS

SEQ ID NO: 45 - protein, 42 aa, Mus musculus
AIDKCALSTHGCEQICINDRNGSYHCECYGGYALNADRRTCA

SEQ ID NO: 46 - protein, 42 aa, Mus musculus
ALDKCASGTHGCQHICVNDGAGSHHCECFEGYTLNADKKTCS

SEQ ID NO: 47 - protein, 42 aa, Mus musculus
VRNKCALGTHGCQHICVSDGAVAYHCDCFPGYTLNDDKKTCS

SEQ ID NO: 48 - protein, 41 aa, Gallus gallus
AANTCALGTHDCEQVCVSNDGSYLCDCYEGYTLNPDKRTCS

SEQ ID NO: 49 - protein, 41 aa, Gallus gallus
AVDVCAPGRHECDQICVSNNGSYVCECFEGYTLNPDKKTCS

SEQ ID NO: 50 - protein, 41 aa, Gallus gallus
AMDVCAPGRHDCAQVCRRNGGSYSCDCFEGFTLNPDKKTCS

SEQ ID NO: 51 - protein, 41 aa, Gallus gallus
AVDVCAPGRHDCEQVCVRDDLFYTCDCYQGYVLNPDKKTCS

SEQ ID NO: 52 - DNA, 55 bp, Homo sapiens
aggctgcagccgcggatggctgccccccctgccccggtgttactgggaggacgga

SEQ ID NO: 53 - DNA, 54 bp, Homo sapiens
aggctgcagccgcggatggctgcccccccgccccggtgttactgggaggacgga

SEQ ID NO: 54 - DNA, 50 bp, Homo sapiens
cgggccggcctggcgctcgaggccccggggcggggcggggcccgaggccg

SEQ ID NO: 55 - DNA, 60 bp, Homo sapiens
cgggccggcctggcgctcgaggcccgggggcggggccggggcggggcggggcccgaggcc

SEQ ID NO: 56 - DNA, 50 bp, Homo sapiens
actttttttttgagatgatgtcttgctcttattgcccaggctggagtgca

SEQ ID NO: 57 - DNA, 51 bp, Homo sapiens
actttttttttgagatgatgtcttggctcttattgcccaggctggagtgca

SEQ ID NO: 58 - DNA, 50 bp, Homo sapiens
aaaccttctgtggtaagttggtcagtctttgcttccaactagaaaggatg

SEQ ID NO: 59 - DNA, 48 bp, Homo sapiens
aaaccttctgtggtaagttggtcagtttgcttccaactagaaaggatg

SEQ ID NO: 60 - DNA, 54 bp, Homo sapiens
gtgtacccagagaaaagagtattattcttttttggtggaaatttaatgaaaaac

SEQ ID NO: 61 - DNA, 50 bp, Homo sapiens
gtgtacccagagaaaagagtattatttttggtggaaatttaatgaaaaac

SEQ ID NO: 62 - DNA, 53 bp, Homo sapiens
catgggagacggtatctgagatgtttcctaagtgtcagtgctgactgttgtaa

SEQ ID NO: 63 - DNA, 51 bp, Homo sapiens
catgggagacggtatctgagatgttataagtgtcagtgctgactgttgtaa

SEQ ID NO: 64 - DNA, 64 bp, Homo sapiens
aaagaatactagtgggaaagttagttatatatatatatatttttttttcttctcttttttgaga

SEQ ID NO: 65 - DNA, 66 bp, Homo sapiens
aaagaatactagtgggaaagttagttatatatatatatatatttttttttcttctcttttttgaga

SEQ ID NO: 66 - DNA, 72 bp, Homo sapiens
cctgtatcatttccccatctgtgcttgtgtgtgtgtgtgtgtgtgtgcatgtgtctcctggtcagtcttttc

SEQ ID NO: 67 - DNA, 74 bp, Homo sapiens
cctgtatcatttccccatctgtgcttgtgtgtgtgtgtgtgtgtgtgtgcatgtgtctcctggtcagtcttttc

SEQ ID NO: 68 - DNA, 50 bp, Homo sapiens
tataattcttttggctttgaaacttgcaactttgagaacaaaacagtcct

SEQ ID NO: 69 - DNA, 52 bp, Homo sapiens
tataattcttttggctttgaaacttttgcaactttgagaacaaaacagtcct

SEQ ID NO: 70 - DNA, 20 bp, Artificial Sequence
  primer that hybridizes to the human MATN3 gene
agaatgagggatgccaactt

SEQ ID NO: 71 - DNA, 18 bp, Artificial Sequence
  primer that hybridizes to the human MATN3 gene
gccaaggtgggaggatca

SEQ ID NO: 72 - DNA, 22 bp, Artificial Sequence
  primer that hybridizes to the human MATN3 gene
tgagggatgccaacttaatctt

SEQ ID NO: 73 - DNA, 20 bp, Artificial Sequence
  primer that hybridizes to the human MATN3 gene
gacatgctcatgaagctgga

SEQ ID NO: 74 - DNA, 19 bp, Artificial Sequence
  primer that hybridizes to the human MATN3 gene
cctggtaatcccaacactg

SEQ ID NO: 75 - DNA, 20 bp, Artificial Sequence
  primer that hybridizes to the human MATN3 gene
cagctcactgcaagctctgt

SEQ ID NO: 76 - DNA, 20 bp, Artificial Sequence
  primer that hybridizes to the human MATN3 gene
ccaagcaatgatcagaccac

SEQ ID NO: 77 - DNA, 24 bp, Artificial Sequence
  primer that hybridizes to the human MATN3 gene
tcagacattgtgtacccagagaaa

SEQ ID NO: 78 - DNA, 22 bp, Artificial Sequence
  primer that hybridizes to the human MATN3 gene
ctggtccctgaatcctgttctt

SEQ ID NO: 79 - DNA, 22 bp, Artificial Sequence
  primer that hybridizes to the human MATN3 gene
ctattccaatgtcaccgtcagc

SEQ ID NO: 80 - DNA, 22 bp, Artificial Sequence
  primer that hybridizes to the human MATN3 gene
cccacccccagaatgttactta

SEQ ID NO: 81 - DNA, 24 bp, Artificial Sequence
  primer that hybridizes to the human MATN3 gene
tcagcatgcatttattgatctctg

SEQ ID NO: 82 - DNA, 22 bp, Artificial Sequence
  primer that hybridizes to the human MATN3 gene
ccagaaggaaaagattgccact

SEQ ID NO: 83 - DNA, 22 bp, Artificial Sequence
  primer that hybridizes to the human MATN3 gene
ttgggtttcaaagctagcacct

SEQ ID NO: 84 - DNA, 23 bp, Artificial Sequence
  primer that hybridizes to the human MATN3 gene
aagtcatccatacgctaaatcca

SEQ ID NO: 85 - DNA, 22 bp, Artificial Sequence
  primer that hybridizes to the human MATN3 gene
tccaggagcaataagatggaga

SEQ ID NO: 86 - DNA, 20 bp, Artificial Sequence
  primer that hybridizes to the human MATN3 gene
ttgctctctgggctcctttc

SEQ ID NO: 87 - DNA, 22 bp, Artificial Sequence
  primer that hybridizes to the human MATN3 gene
ggaacctatgctccaggttttg

SEQ ID NO: 88 - DNA, 22 bp, Artificial Sequence
  primer that hybridizes to the human MATN3 gene
ttgtagagatggggtctcacca

SEQ ID NO: 89 - DNA, 20 bp, Artificial Sequence
  primer that hybridizes to the human MATN3 gene
gttacttgggaggccgagat

SEQ ID NO: 90 - DNA, 20 bp, Artificial Sequence
  primer that hybridizes to the human MATN3 gene
cccattcctcattgactcca

SEQ ID NO: 91 - DNA, 22 bp, Artificial Sequence
  primer that hybridizes to the human MATN3 gene
agaggggtactcactggtctgg

SEQ ID NO: 92 - DNA, 19 bp, Artificial Sequence
  primer that hybridizes to the human MATN3 gene
cagctgggagttgggaacc

SEQ ID NO: 93 - DNA, 20 bp, Artificial Sequence
  primer that hybridizes to the human MATN3 gene
tacctacccggtgatggaag

SEQ ID NO: 94 - DNA, 23 bp, Artificial Sequence
  primer that hybridizes to the human MATN3 gene
tcacgtttctgagaagtactgga

SEQ ID NO: 95 - DNA, 24 bp, Artificial Sequence
  primer that hybridizes to the human MATN3 gene
tgaggaaagattagaaaaacctga

SEQ ID NO: 96 - DNA, 22 bp, Artificial Sequence
  primer that hybridizes to the human MATN3 gene
tcattcaatgcactttcctctc

SEQ ID NO: 97 - DNA, 22 bp, Artificial Sequence
  primer that hybridizes to the human MATN3 gene
cactgtttttgcaaccttttca

SEQ ID NO: 98 - DNA, 22 bp, Artificial Sequence
  primer that hybridizes to the human MATN3 gene
tctgaccttccacaattgcttt

SEQ ID NO: 99 - DNA, 20 bp, Artificial Sequence
  primer that hybridizes to the human MATN3 gene
aacttgcaactcaccagcaa

SEQ ID NO: 100 - DNA, 20 bp, Artificial Sequence
  primer that hybridizes to the human MATN3 gene
catgccatttccctttgtct

SEQ ID NO: 101 - DNA, 23 bp, Artificial Sequence
  primer that hybridizes to the human MATN3 gene
aaaaataccggacacctactttg

SEQ ID NO: 102 - DNA, 20 bp, Artificial Sequence
  primer that hybridizes to the human MATN3 gene
tgggagtgttgtctgcctta

SEQ ID NO: 103 - DNA, 20 bp, Artificial Sequence
  primer that hybridizes to the human MATN3 gene
gacctgggaaaacaaaccaa

SEQ ID NO: 104 - DNA, 21 bp, Artificial Sequence
  primer that hybridizes to the human MATN3 gene
caaaatgtcaaaaccccatct

SEQ ID NO: 105 - DNA, 24 bp, Artificial Sequence
  primer that hybridizes to the human MATN3 gene
tgtaccttaatactcacagcaacg

SEQ ID NO: 106 - DNA, 22 bp, Artificial Sequence
  primer that hybridizes to the human MATN3 gene
catggtatgttccccagcttta

SEQ ID NO: 107 - DNA, 20 bp, Artificial Sequence
  primer that hybridizes to the human MATN3 gene
aagcaatacacctgccttgg

SEQ ID NO: 108 - DNA, 20 bp, Artificial Sequence
  primer that hybridizes to the human MATN3 gene
taggttcctcctctgccatt

SEQ ID NO: 109 - DNA, 22 bp, Artificial Sequence
  primer that hybridizes to the human MATN3 gene
agcgtctcacatttcacaagaa

SEQ ID NO: 110 - DNA, 20 bp, Artificial Sequence
  primer that hybridizes to the human MATN3 gene
gatgctgcacaaaaagcact

SEQ ID NO: 111 - DNA, 21 bp, Artificial Sequence
  primer that hybridizes to the human MATN3 gene
cccatctgtgcttgtgtgtgt

SEQ ID NO: 112 - DNA, 21 bp, Artificial Sequence
  primer that hybridizes to the human MATN3 gene
gaggagaagcttccatttggt

SEQ ID NO: 113 - DNA, 22 bp, Artificial Sequence
  primer that hybridizes to the human MATN3 gene
tcagccctttaaggctatttcc

SEQ ID NO: 114 - DNA, 21 bp, Artificial Sequence
  primer that hybridizes to the human MATN3 gene
gagggtgggaatgagtgagtg

SEQ ID NO: 115 - DNA, 22 bp, Artificial Sequence
  primer that hybridizes to the human MATN3 gene
cccagattcactgcttcttcct

SEQ ID NO: 116 - DNA, 22 bp, Artificial Sequence
  primer that hybridizes to the human MATN3 gene
gcatgggttcagtgggatagtc

SEQ ID NO: 117 - DNA, 22 bp, Artificial Sequence
  primer that hybridizes to the human MATN3 gene
cccaggtttgagaaatggagtt

SEQ ID NO: 118 - DNA, 22 bp, Artificial Sequence
  primer that hybridizes to the human MATN3 gene
tggaattcagtcaggcagctat

SEQ ID NO: 119 - DNA, 22 bp, Artificial Sequence
  primer that hybridizes to the human MATN3 gene
ccagtcttcagacctgggaagt

SEQ ID NO: 120 - DNA, 22 bp, Artificial Sequence
  primer that hybridizes to the human MATN3 gene
tgaagtcaggagttcaagacca

SEQ ID NO: 121 - DNA, 22 bp, Artificial Sequence
  primer that hybridizes to the human MATN3 gene
acggggtttcactatgttgtcc

SEQ ID NO: 122 - DNA, 22 bp, Artificial Sequence
  primer that hybridizes to the human MATN3 gene
gaggcagaaggatcacttgagg

SEQ ID NO: 123 - DNA, 18 bp, Artificial Sequence
  primer that hybridizes to the human MATN3 gene
acaggcatgggccaccac

SEQ ID NO: 124 - DNA, 22 bp, Artificial Sequence
  primer that hybridizes to the human MATN3 gene
ctggtgcagtgccttattccta

SEQ ID NO: 125 - DNA, 22 bp, Artificial Sequence
  primer that hybridizes to the human MATN3 gene
tgccactatgcctggctaacta

SEQ ID NO: 126 - DNA, 20 bp, Artificial Sequence
  primer that hybridizes to the human MATN3 gene
tcctcagtggctgaagacaa

SEQ ID NO: 127 - DNA, 22 bp, Artificial Sequence
  primer that hybridizes to the human MATN3 gene
aagacattccacaggcaagtga

SEQ ID NO: 128 - DNA, 22 bp, Artificial Sequence
  primer that hybridizes to the human MATN3 gene
ccactcctctggggtacatagg

SEQ ID NO: 129 - DNA, 22 bp, Artificial Sequence
  primer that hybridizes to the human MATN3 gene
gccaagtggtcaaaaagcagat

SEQ ID NO: 130 - DNA, 22 bp, Artificial Sequence
  primer that hybridizes to the human MATN3 gene
tgttttctcaccaaaagttgaa

SEQ ID NO: 131 - DNA, 25 bp, Artificial Sequence
  primer that hybridizes to the human MATN3 gene
atcaaaagacagaagtttgattctc

SEQ ID NO: 132 - DNA, 22 bp, Artificial Sequence
  primer that hybridizes to the human MATN3 gene
cccaggagtttccaataaactg